SEQUENCE LISTING (124 sequences)

SEQ ID NO: 1 - DNA, 1167 bp, Escherichia coli
  source 1..1167: mol_type = genomic DNA
gccaacttacatgaatatcaggcaaaacaactttttgcccgctatggcttaccagcaccggtgggttatgcctgtactactccgcgcgaagcagaagaagccgcttcaaaaatcggtgccggtccgtgggtagtgaaatgtcaggttcacgctggtggccgcggtaaagcgggcggtgtgaaagttgtaaacagcaaagaagacatccgtgcttttgcagaaaactggctgggcaagcgtctggtaacgtatcaaacagatgccaatggccaaccggttaaccagattctggttgaagcagcgaccgatatcgctaaagagctgtatctcggtgccgttgttgaccgtagttcccgtcgtgtggtctttatggcctccaccgaaggcggcgtggaaatcgaaaaagtggcggaagaaactccgcacctgatccataaagttgcgcttgatccgctgactggcccgatgccgtatcagggacgcgagctggcgttcaaactgggtctggaaggtaaactggttcagcagttcaccaaaatcttcatgggcctggcgaccattttcctggagcgcgacctggcgttgatcgaaatcaacccgctggtcatcaccaaacagggcgatctgatttgcctcgacggcaaactgggcgctgacggcaacgcactgttccgccagcctgatctgcgcgaaatgcgtgaccagtcgcaggaagatccgcgtgaagcacaggctgcacagtgggaactgaactacgttgcgctggacggtaacatcggttgtatggttaacggcgcaggtctggcgatgggtacgatggacatcgttaaactgcacggcggcgaaccggctaacttccttgacgttggcggcggcgcaaccaaagaacgtgtaaccgaagcgttcaaaatcatcctctctgacgacaaagtgaaagccgttctggttaacatcttcggcggtatcgttcgttgcgacctgatcgctgacggtatcatcggcgcggtagcagaagtgggtgttaacgtaccggtcgtggtacgtctggaaggtaacaacgccgaactcggcgcgaagaaactggctgacagcggcctgaatattattgcagcaaaaggtctgacggatgcagctcagcaggttgttgccgcagtggaggggaaataa

SEQ ID NO: 2 - protein, 388 aa, Escherichia coli
ANLHEYQAKQLFARYGLPAPVGYACTTPREAEEAASKIGAGPWVVKCQVHAGGRGKAGGVKVVNSKEDIRAFAENWLGKRLVTYQTDANGQPVNQILVEAATDIAKELYLGAVVDRSSRRVVFMASTEGGVEIEKVAEETPHLIHKVALDPLTGPMPYQGRELAFKLGLEGKLVQQFTKIFMGLATIFLERDLALIEINPLVITKQGDLICLDGKLGADGNALFRQPDLREMRDQSQEDPREAQAAQWELNYVALDGNIGCMVNGAGLAMGTMDIVKLHGGEPANFLDVGGGATKERVTEAFKIILSDDKVKAVLVNIFGGIVRCDLIADGIIGAVAEVGVNVPVVVRLEGNNAELGAKKLADSGLNIIAAKGLTDAAQQVVAAVEGK

SEQ ID NO: 3 - DNA, 870 bp, Escherichia coli
  source 1..870: mol_type = genomic DNA
gcctccattttaatcgataaaaacaccaaggttatctgccagggctttaccggtagccaggggactttccactcagaacaggccattgcatacggcactaaaatggttggcggcgtaaccccaggtaaaggcggcaccacccacctcggcctgccggtgttcaacaccgtgcgtgaagccgttgctgccactggcgctaccgcttctgttatctacgtaccagcaccgttctgcaaagactccattctggaagccatcgacgcaggcatcaaactgattatcaccatcactgaaggcatcccgacgctggatatgctgaccgtgaaagtgaagctggatgaagcaggcgttcgtatgatcggcccgaactgcccaggcgttatcactccgggtgaatgcaaaatcggtatccagcctggtcacattcacaaaccgggtaaagtgggtatcgtttcccgttccggtacactgacctatgaagcggttaaacagaccacggattacggtttcggtcagtcgacctgtgtcggtatcggcggtgacccgatcccgggctctaactttatcgacattctcgaaatgttcgaaaaagatccgcagaccgaagcgatcgtgatgatcggtgagatcggcggtagcgctgaagaagaagcagctgcgtacatcaaagagcacgttaccaagccagttgtgggttacatcgctggtgtgactgcgccgaaaggcaaacgtatgggccacgcgggtgccatcattgccggtgggaaagggactgcggatgagaaattcgctgctctggaagccgcaggcgtgaaaaccgttcgcagcctggcggatatcggtgaagcactgaaaactgttctgaaataa

SEQ ID NO: 4 - protein, 289 aa, Escherichia coli
ASILIDKNTKVICQGFTGSQGTFHSEQAIAYGTKMVGGVTPGKGGTTHLGLPVFNTVREAVAATGATASVIYVPAPFCKDSILEAIDAGIKLIITITEGIPTLDMLTVKVKLDEAGVRMIGPNCPGVITPGECKIGIQPGHIHKPGKVGIVSRSGTLTYEAVKQTTDYGFGQSTCVGIGGDPIPGSNFIDILEMFEKDPQTEAIVMIGEIGGSAEEEAAAYIKEHVTKPVVGYIAGVTAPKGKRMGHAGAIIAGGKGTADEKFAALEAAGVKTVRSLADIGEALKTVLK

SEQ ID NO: 5 - DNA, 1161 bp, Advenella mimigardefordensis
  source 1..1161: mol_type = genomic DNA
atgaaaattcacgaataccagggtaaggaacttctcaagaagtttggagtcacagtaccacgtgggattccagccttcagtgtcgatgaggctgtggcggctgcggaaaagttaggcgggccagtgtgggtcgtgaaagcacaaatccacgccggcgggcgcgggaaaggtggtggtgttaaattagcgaaatcgatcgatgaggtacgccaactgtccagtgagatccttggtatgcaattagttacccatcaaactggaccggaaggccaaaaggttaatcgcctcttaatcgaggaaggtgcagatattaagaaagagtattatgttggcattgttactgaccgttcaagtcaacgtgtttgtgtcatggcaagctctgagggtggtatggatgtggaagaggtcgcagaaaagacgcctgagaaaatcttaaaggtattcgtagacccggcgaagggcttcactgacgacgaggcgcgtgagcttgcccgcggaattggggtgcctgatgcgagtgtggaaaaggcggcggtcgagttccagaagctctaccaagcatattgggacaccgattcgagcttagcagagattaatccgttaatcctcactggaagcggcgacattatcgcattggatgcgaagtttaatttcgatagtaatgcgcttttccgccatcctgagattgtggaatatcgtgacttagatgaagaagatccggcggagattgaggcatctaagttcgacttagcatatatccaattagatggaaacattgggtgtcttgtgaatggcgcaggcctggcaatggctacgatggacactattaaacttttcggtggggagccggcgaattttcttgacgttggcggcggggccacggccgagaaagtaactgaggcgtttaaaattatgctgaagaataaagaggttaaggcaatcttggttaacatcttcggtgggattatgcgttgtgacgtaattgcagagggtgttattgccgcgtgtaaggcggtgaatctgtcagtgccactcgtcgtccgtatgaaaggcactaacgaggaaatggggaagaagatgttggcagaatcgggtctgccgattatttcggcagacactatggccgaggctgccaccaaggttgtcgcagcagtgaaatga

SEQ ID NO: 6 - protein, 386 aa, Advenella mimigardefordensis
MKIHEYQGKELLKKFGVTVPRGIPAFSVDEAVAAAEKLGGPVWVVKAQIHAGGRGKGGGVKLAKSIDEVRQLSSEILGMQLVTHQTGPEGQKVNRLLIEEGADIKKEYYVGIVTDRSSQRVCVMASSEGGMDVEEVAEKTPEKILKVFVDPAKGFTDDEARELARGIGVPDASVEKAAVEFQKLYQAYWDTDSSLAEINPLILTGSGDIIALDAKFNFDSNALFRHPEIVEYRDLDEEDPAEIEASKFDLAYIQLDGNIGCLVNGAGLAMATMDTIKLFGGEPANFLDVGGGATAEKVTEAFKIMLKNKEVKAILVNIFGGIMRCDVIAEGVIAACKAVNLSVPLVVRMKGTNEEMGKKMLAESGLPIISADTMAEAATKVVAAVK

SEQ ID NO: 7 - DNA, 882 bp, Advenella mimigardefordensis
  source 1..882: mol_type = genomic DNA
atgtcaattctgatcaataaagactccaaagtcatcactcagggtattaccggtaaaaccggccagttccacacacgtatgtgccgtgattacgccaatggtaaagaagcttttgttgctggtgtgaatcctaaacgcgcaggcgaggatttcgaaggcgtgcctatttacgcttctgtcaaggacgcgaaagaacaaacgggcgccaatgtatctgttatctatgttccaccagcaggtgccgctgatgccatctgggaagcggtagacgctgacctggatctggtgatctgcattaccgaaggtattcctgttcgcgatatgctggctgtccgcaaccgcatgcgccaggaaaacaaaaaaacactgctgctgggccctaactgccctggtctgattacgcccgatgagctgaaaatcggcattatgccagcgcacatctgcaaaaaaggccgcattggtgtggtcagccgttcaggcacgcttacatacgaagccgttgcccagttgacggagctcggcctgggacaatccagtgcagttggtattggtggtgacccgatcaatggtctcaaacacatcgatatcctgaaaatgttcaacgacgatccagagaccgatgcggttgtgatgattggtgagatcggcgggccggacgaagtcaatgccgccgaatgggccaaagacaatatgagcaagccagtagtgggctttatcgccggtgttactgctcctccaggaaaacgcatgggccacgccggtgcgctgatctccggtggcgccgatacggccgatgccaagctggaaatcatggaagcttgcggtatccgtaccacacgcaatccatctgaaatgggcaaactgctcaaatcagtactgtaa

SEQ ID NO: 8 - protein, 293 aa, Advenella mimigardefordensis
MSILINKDSKVITQGITGKTGQFHTRMCRDYANGKEAFVAGVNPKRAGEDFEGVPIYASVKDAKEQTGANVSVIYVPPAGAADAIWEAVDADLDLVICITEGIPVRDMLAVRNRMRQENKKTLLLGPNCPGLITPDELKIGIMPAHICKKGRIGVVSRSGTLTYEAVAQLTELGLGQSSAVGIGGDPINGLKHIDILKMFNDDPETDAVVMIGEIGGPDEVNAAEWAKDNMSKPVVGFIAGVTAPPGKRMGHAGALISGGADTADAKLEIMEACGIRTTRNPSEMGKLLKSVL

SEQ ID NO: 9 - DNA, 1170 bp, Alcanivorax borkumensis
  source 1..1170: mol_type = genomic DNA
atgaacctgcatgaatatcagagcaaacagctgttcgcagattatggtctgccggttagtaccggcttcgcctgcgataccccggaagaagttgccgcaaaagccaaagaaattggcggcgataaatgggttgtgaaagcacaggtgcacgctggcggccgtggtaaagccggcggtgttaaactggtggataccccggaggatgcagcagccttcgcaaaaaaatggctgggtaaacagctggttaccttccagaccgatgaacatggtcagccggttagcaaaattctggttgaaacctgcaccgatattgccaaagaactgtatctgagcgccgtgctggatcgcgccagccgtcgtgttgtgttcatggccagtaccgaaggtggtgtggatattgaaaccgtggcagaagaaacacctgaaaaaattctgaaagccgaaattgatccgctggttggcgcacagccgtatcaggcacgcgaaattgcattcaaactgggtctggaaggcaaacaggttaatcagttcgccaaaatcttcgtgggtctggccaaactgttcgttgataaagatctggccctgattgaagtgaatccgctggttattaccgataccggtgatctgcattgcctggatgccaaaattaatgtggatggtagtgccctgtatcgccatccggatattaaagcactggatgatccgagccaggaagatgaacgcgaacgtcgtgccgcagaatgggatctgaattatgttgccctggaaggtaatattggctgcatggttaatggcgcaggtctggcaatgggcacaatggatctggttaaactgaaaggcggcgccccggcaaacttcctggatgttggcggcggtgcaaccaaagaacgcgttaccgaagcattcaaaattattctgagcgatgatcaggtgaaaggtgtgctggttaatatcttcggcggtattgttcgttgtgatctgattgccgaaggcattattggtgcagttgaagaagttggtgttaccgttccggtggtggttcgcctggaaggtaacaatgccgaactgggcagtcagaaactggccgaaagtggcatgaatattattgcagcccagagcttcgatgatgccgccgaacaggttgtgaaagcggtgggcggcgcagcctaa

SEQ ID NO: 10 - protein, 389 aa, Alcanivorax borkumensis
MNLHEYQSKQLFADYGLPVSTGFACDTPEEVAAKAKEIGGDKWVVKAQVHAGGRGKAGGVKLVDTPEDAAAFAKKWLGKQLVTFQTDEHGQPVSKILVETCTDIAKELYLSAVLDRASRRVVFMASTEGGVDIETVAEETPEKILKAEIDPLVGAQPYQAREIAFKLGLEGKQVNQFAKIFVGLAKLFVDKDLALIEVNPLVITDTGDLHCLDAKINVDGSALYRHPDIKALDDPSQEDERERRAAEWDLNYVALEGNIGCMVNGAGLAMGTMDLVKLKGGAPANFLDVGGGATKERVTEAFKIILSDDQVKGVLVNIFGGIVRCDLIAEGIIGAVEEVGVTVPVVVRLEGNNAELGSQKLAESGMNIIAAQSFDDAAEQVVKAVGGAA

SEQ ID NO: 11 - DNA, 879 bp, Alcanivorax borkumensis
  source 1..879: mol_type = genomic DNA
atgggcagtgtgctgattgataaaaacaccaaagttatctgtcagggcttcaccggcagccagggcaccctgcatagtgaacaggccattgaatatggtacccagatggtgggcggtgtgacccctggtaaaggcggccagacccatctgggtctgccggtgttcaataccgtggcagaagccgttgccgaaaccggcgccaccgccagtgttatctatgtgccggcaccgttctgtaaagatagtattctggaagcagcagatgcaggcattgaactgattgtgtgtattaccgaaggcgttccggcactggatatgctgttcgttaaagaatatattagccgtaaagacggcattcgcctgattggtccgaattgtccgggtgttattacccctggtgaatgtaaaattggcattatgccgggccatattcatacccctggtaaggtgggtattgtgagccgcagcggcaccctgacctatgaagcagtgaatcagaccaccaaactgggcttcggtcagagtagttgcattggcattggcggtgatccgattccgggcatgaccttcattgatgccctggaactgttccagaatgatccgcagaccgaagcaattgtgatggtgggcgaaattggtggtaccgccgaagatgaagcagcagcctatattaaagaacatgttaccaaaccggtggttagttatattgcaggcgtgaccgccccggccggcaaaagaatgggccatgccggcgccattattgcaggcggtaaaggcaccgcagatgaaaaattcgccgttctggaagcagcgggcgtgaaaaccgtgcgcaatccggccgaaattggcaaagccgtgaaagaagcaaccggttggtaa

SEQ ID NO: 12 - protein, 292 aa, Alcanivorax borkumensis
MGSVLIDKNTKVICQGFTGSQGTLHSEQAIEYGTQMVGGVTPGKGGQTHLGLPVFNTVAEAVAETGATASVIYVPAPFCKDSILEAADAGIELIVCITEGVPALDMLFVKEYISRKDGIRLIGPNCPGVITPGECKIGIMPGHIHTPGKVGIVSRSGTLTYEAVNQTTKLGFGQSSCIGIGGDPIPGMTFIDALELFQNDPQTEAIVMVGEIGGTAEDEAAAYIKEHVTKPVVSYIAGVTAPAGKRMGHAGAIIAGGKGTADEKFAVLEAAGVKTVRNPAEIGKAVKEATGW

SEQ ID NO: 13 - DNA, 1173 bp, Methylorubrum extorquens
  source 1..1173: mol_type = genomic DNA
atggacgttcacgagtaccaagccaaggagctgctcgcgagcttcggggtcgccgtcccgaagggcgccgtggctttcagcccggatcaagcggtctatgcggcgaccgagctcggcggctcgttctgggcggtgaaggctcagatccatgccggcgcgcgcggcaaggcgggcgggatcaagctttgccgcacctacaatgaagtgcgcgacgccgcccgcgacctgctgggaaaacgcctcgtgacgctccagaccggccccgagggcaagccggtgcagcgcgtctacgtcgagaccgccgacccgttcgagcgtgaactctatctcggctacgtgctcgatcggaaggccgagcgcgtccgtgtcatcgcctcccagcgcggcggcatggatatcgaggagatcgccgccaaggagcccgaggcgctgatccaggtcgtggtcgagccggcggtgggcctgcagcagttccaggcccgcgagatcgcgttccagctcggcctcaacatcaagcaggtctcggccgcggtgaagaccatcatgaacgcctaccgggcgttccgcgactgcgacggcaccatgctggagatcaacccgctcgtcgtcaccaaggacgaccgggttctggcactcgacgccaagatgtccttcgacgacaacgccctgttccgccgccgcaacatcgcggacatgcacgatccatcgcagggcgatccccgcgaggcccaggctgccgagcacaatctcagctatatcggcctcgagggcgaaattggctgcatcgtcaacggcgcgggtctggccatggcgaccatggacatgatcaagcacgcgggcggcgagccggcaaacttcctggatgtgggcggcggtgccagcccggaccgcgtcgccacggccttccgcctcgttctgtcggaccgcaacgtgaaggcgatcctcgtcaacatcttcgccggcatcaaccgctgcgactgggtcgcggagggcgtggtcaaggccgcgcgcgaggtgaagatcgacgtgccgctcatcgtgcggctcgccggcacgaacgtcgatgaaggcaagaagatcctcgccgagagcgggctcgacctcatcaccgccgacacccttacggaagccgcgcgcaaggctgtcgaagcctgccacggcgccaagcactga

SEQ ID NO: 14 - protein, 390 aa, Methylorubrum extorquens
MDVHEYQAKELLASFGVAVPKGAVAFSPDQAVYAATELGGSFWAVKAQIHAGARGKAGGIKLCRTYNEVRDAARDLLGKRLVTLQTGPEGKPVQRVYVETADPFERELYLGYVLDRKAERVRVIASQRGGMDIEEIAAKEPEALIQVVVEPAVGLQQFQAREIAFQLGLNIKQVSAAVKTIMNAYRAFRDCDGTMLEINPLVVTKDDRVLALDAKMSFDDNALFRRRNIADMHDPSQGDPREAQAAEHNLSYIGLEGEIGCIVNGAGLAMATMDMIKHAGGEPANFLDVGGGASPDRVATAFRLVLSDRNVKAILVNIFAGINRCDWVAEGVVKAAREVKIDVPLIVRLAGTNVDEGKKILAESGLDLITADTLTEAARKAVEACHGAKH

SEQ ID NO: 15 - DNA, 891 bp, Methylorubrum extorquens
  source 1..891: mol_type = genomic DNA
atgagcattctcatcgacgagaagaccccgatcctggtccagggcatcacgggcgacaagggcaccttccacgccaaggagatgatcgcctacggctcgaacgtcgtcggcggcgtcaccccgggcaagggcggcaagacccattgcggcgtgccggtgttcaacaccgtcaaggaggccgtggaggcgaccggcgccaccacctcgatcaccttcgtggcgccccccttcgcggcggacgcgatcatggaggcggccgacgccggcctcaagctcgtctgctcgatcaccgacggcatccccgctcaggacatgatgcgggtgaaacgctacctccggcgctatccgaaggagaagcgcacgatggtggtgggcccgaactgcgcgggcatcatctcgcccggcaagtcgatgctcggcatcatgcccggccacatctacctcccgggcaaggtcggcgtcatctcccgttccggcacgctgggctacgaggccgccgcgcagatgaaggagctcggcatcggcatctcgacctccgtcggcatcggcggcgatccgatcaacggctcctccttcctcgaccacctcgctctgttcgagcaggatcccgagacggaagccgtgctgatgatcggcgagatcggcgggccgcaggaggccgaggcctcggcctggatcaaggagaacttttccaagcccgtgatcggcttcgtggcgggcctcaccgcccccaagggccgccgcatggggcatgccggcgcgatcatctcggcgaccggcgacagcgccgcggagaaggccgagatcatgcgctcctatggcctgaccgtggcgcccgatccgggctccttcggcagcaccgtggccgacgtgctcgcccgcgcggcgtga

SEQ ID NO: 16 - protein, 296 aa, Methylorubrum extorquens
MSILIDEKTPILVQGITGDKGTFHAKEMIAYGSNVVGGVTPGKGGKTHCGVPVFNTVKEAVEATGATTSITFVAPPFAADAIMEAADAGLKLVCSITDGIPAQDMMRVKRYLRRYPKEKRTMVVGPNCAGIISPGKSMLGIMPGHIYLPGKVGVISRSGTLGYEAAAQMKELGIGISTSVGIGGDPINGSSFLDHLALFEQDPETEAVLMIGEIGGPQEAEASAWIKENFSKPVIGFVAGLTAPKGRRMGHAGAIISATGDSAAEKAEIMRSYGLTVAPDPGSFGSTVADVLARAA

SEQ ID NO: 17 - DNA, 1182 bp, Thermobifida fusca
  source 1..1182: mol_type = genomic DNA
atggatttatttgagtatcaagctaaagcgctgtttgctgagtacggcgtgccagtgcctcagggcaaagttgcaagtactccggaagaagtccgcgcaattgcggaagaatttgcagccgccggtaaaccgcgtgttgttgtgaaggcgcaagttaaaactggtggacgcggtaaagcaggtggagtgaaagttgcagatgggccggatgatgcggttgcgaaagcgaaacaaatcttaggtatggatattaaaggtcatactgtacatcgtgttttggtagaggaagcgtccgatattgcggaagaatattatttctcgtttcttttggatcgcgccaatcgcagctttctgagcatttgttcagcggaaggcggcatggaaattgaagaagtggccgccacgaatcctgatgctgtcgcaaaagtaccgatttccccattaaaaggcgcacctgcagatgtggcagcagatattgtggcgcagggtaagttgccggaagccgccgcgcaaggagcggtggatgtgattactaaattgtggaaggtgtttgtcgaaaaggatgcgaccttagttaatgtgaatcctctgattttgacgaaagatgggcgtgtagtggcattagatggaaaagttactctcgatgataatgcagaatttcgtcaagatttagaaagcctggcgagcgcagcagagggagatcctttagaggtgaaagcgaaagagaaaggtctgaattatgtaaaactggatggcgaagtgggtattattggcaatggggcgggcctggttatgagtaccttggatgtagttgcgtatgcgggcgaacaacatggtggtgtaaaaccggcgaattttctggatattggtggtggtgcgagtgcggaagtgatggcgaatggtttagaaattattttgtcagatccggccgttaaatcggtatttgttaatgtctttggcggcattacggcctgtgatgcagtggcgaatggaattgtccaagctttagagctcttggagtcgcgtggagaagacgttacaaaaccacttgttgtccgtcttgatggcaataatgcagagcttgggcgttctattctgaatgagcgcaatcatccggcagttcgtcaagtagatacgatggatggtgccgcggcgctggctgcagagttagcggctaaatga

SEQ ID NO: 18 - protein, 393 aa, Thermobifida fusca
MDLFEYQAKALFAEYGVPVPQGKVASTPEEVRAIAEEFAAAGKPRVVVKAQVKTGGRGKAGGVKVADGPDDAVAKAKQILGMDIKGHTVHRVLVEEASDIAEEYYFSFLLDRANRSFLSICSAEGGMEIEEVAATNPDAVAKVPISPLKGAPADVAADIVAQGKLPEAAAQGAVDVITKLWKVFVEKDATLVNVNPLILTKDGRVVALDGKVTLDDNAEFRQDLESLASAAEGDPLEVKAKEKGLNYVKLDGEVGIIGNGAGLVMSTLDVVAYAGEQHGGVKPANFLDIGGGASAEVMANGLEIILSDPAVKSVFVNVFGGITACDAVANGIVQALELLESRGEDVTKPLVVRLDGNNAELGRSILNERNHPAVRQVDTMDGAAALAAELAAK

SEQ ID NO: 19 - DNA, 879 bp, Thermobifida fusca
  source 1..879: mol_type = genomic DNA
atggcgatctttcttactaaggacagtaaggtattagtacagggcatgaccggtagcgagggcactaagcacacccgtcgcatgttagctgccggaactaacatcgttggtggcgtcaaccctcgcaaggcagggcaggtggttgacttcgacggcacccaggtcccagtattcggatcggtcgcagagggtatgaaggctaccggggcggacgttacagttattttcgttccgccgaagttcgcgaaggacgcagtgatcgaagcgatcgacgcagagatcggcttagccgtcgtgatcaccgaaggtatcccggtgcacgacaccgccacgttctgggcccacgcgtgctcaaagggtaacaagacccgcatcatcgggcccaactgccctggacttatcactcctggtcagtccaacgcgggcatcatccctgccgacatcaccaagccgggccgtatcggtctggtgagcaagtcgggtactctgacctaccagatgatgtacgagctgcgtgacattggtttctccacctgcgttggcatcggcggagaccccatcatcggcaccactcacatcgacgcgctcgccgcgttcgaagctgaccctgacaccgacgtcatcgtgatgatcggtgagatcggtggtgacgctgaagaacgcgccgccgaatacatcaagaagcacgtaaccaagccggtagtcggctacatcgcgggcttcaccgcgcctgagggtaagaccatgggacatgccggcgcaattgttagcggatccagtggtaccgccgcggcaaagaaggaagcattagaggctgtcggggtcaaggtaggtaagaccccgagtgaggctgcgaagttagttcgtagtctgttctaa

SEQ ID NO: 20 - protein, 292 aa, Thermobifida fusca
MAIFLTKDSKVLVQGMTGSEGTKHTRRMLAAGTNIVGGVNPRKAGQVVDFDGTQVPVFGSVAEGMKATGADVTVIFVPPKFAKDAVIEAIDAEIGLAVVITEGIPVHDTATFWAHACSKGNKTRIIGPNCPGLITPGQSNAGIIPADITKPGRIGLVSKSGTLTYQMMYELRDIGFSTCVGIGGDPIIGTTHIDALAAFEADPDTDVIVMIGEIGGDAEERAAEYIKKHVTKPVVGYIAGFTAPEGKTMGHAGAIVSGSSGTAAAKKEALEAVGVKVGKTPSEAAKLVRSLF

SEQ ID NO: 21 - DNA, 1203 bp, Pseudomonas aeruginosa
  source 1..1203: mol_type = genomic DNA
atgacacaatcccaccgaccgcgcccgctcgacggcatcaccgtggtcagcctggagcacgccatcgccgcgccgttctgcacccgccaactggccgatctcggcgcgcgggtgatcaagatcgaacgccccggcgtcggcgatttcgcccgcggttacgacgagcgggtcaacggcctgtcgtcgcacttcgtctggaccaatcgttccaagcagagcctcagcctcgacctcaagcaggccgaggcgctgccgttgctggagaagctgctggccggcgccgacgtgctggtgcagaacctcgcgccgggcgccgccgcgcgcctgggcctgtcctacgaggcgttgcgcgagcgcttcccgcgactgatcgtctgcgatatctccggctacggcgaaggcggtccctacgagaagaagaaggcctacgacctgctgatccagagcgagggcggcttcctttcggtgaccggcggcgccgggccgaacgagatggccaaggccggctgttcgatcgccgatatcgccgccggcatgtacgcctacagcagcatcctttcggccctgctgctgcgcgagcgcaccggcgagggcagccgcatcgacgtgtcgatgctggagagcctggtggagtggatgggctacccgatgtactacgcctaccaggacgcgccgccgccgccgcgcgccggcgcttcgcactcgaccatctacccctatgggccattccccgccggcgacggcggcacggtgatgctcggcctgcagaacgagcgcgagtggcgggccttctgcgacaaggtcctgcgccgcccggaactggccgacgacgaacgcttctcggcgaacttcaagcgctcggccaaccgcgaggagctgcgtgcgctgatcgtcgaagccttcgccgccttgccggccgccgaggtggtggcgcgcctggaacaggcgcagatcgccagcgcccacgtcaacgacatggccggggtctgggcgcacccgcagttacaggcgcggcaacgctggacctccatcgacagcccggccgggcgcctgccggcgttgttgccgccggccagcagcagcgccttcgttccgcgcatggatgcggtgccggcgctgggcggcgacagcgacgcgctgcttgccgaactgggctgtaccgcggccgatatccagcgcctgcgcgcgaccggaacggtatga

SEQ ID NO: 22 - protein, 400 aa, Pseudomonas aeruginosa
MTQSHRPRPLDGITVVSLEHAIAAPFCTRQLADLGARVIKIERPGVGDFARGYDERVNGLSSHFVWTNRSKQSLSLDLKQAEALPLLEKLLAGADVLVQNLAPGAAARLGLSYEALRERFPRLIVCDISGYGEGGPYEKKKAYDLLIQSEGGFLSVTGGAGPNEMAKAGCSIADIAAGMYAYSSILSALLLRERTGEGSRIDVSMLESLVEWMGYPMYYAYQDAPPPPRAGASHSTIYPYGPFPAGDGGTVMLGLQNEREWRAFCDKVLRRPELADDERFSANFKRSANREELRALIVEAFAALPAAEVVARLEQAQIASAHVNDMAGVWAHPQLQARQRWTSIDSPAGRLPALLPPASSSAFVPRMDAVPALGGDSDALLAELGCTAADIQRLRATGTV

SEQ ID NO: 23 - DNA, 1323 bp, Yersinia pestis
  source 1..1323: mol_type = genomic DNA
atggatattcgcgcgctgtatgacgagaaactcacaacgccagaagaggccgtatcctcaattgcgtcaggttcacacctgagcatgggtatgtttgcagcagaaccgccagccctgctgaaagcattagcggatcgcgcaacgcgtggggacattggcgaccttcgcgtttactacttcgaaacggcgaagattgcgggtgacactattctgcggtacgaattgaacaaccgcatcaaaccctactcgatgtttgttacggccgttgaacgcgcgctgattcgtcgtggcatcgaagatggaggtcggaaagtggtgaactatgtgccctctaacttccatcaagctcctcgcctgttggcggaggaaattggcatcgacaccttcatgcataccgtatctccgatggattgccacgggtatttctcgctcggtgtgggtaacgattacagctcacgcattgcccgtagtgcacgtcgcttcattgtggaagtgaatcgttatatgccacgcgtccaaggcgaagcagcagctattcacatctcggaagttgatgcgattgtcgaaaatcacgtacccttaatcgagatgccagtgcgctctgcgatccctgagtacacttccatttcccatattatcgcagacctggttccggatggtgcttgtctgcagatgggcgtgggagccttgccgaatctggtgtgtggcgtactcaaggatcgtaacgatctgggtatccataccgaagttctgaatccgggcctggtggatctgatccgtcgtggtgttgttaccaatcagcggaaaaccttggatcgtgggcgcagcgtgtttacgtttgctatggggcaacaggaaatgtatgagtatcttaacgatcatccggccattttctcgcgtccggtagactatgtcaatgatccgcatatcattgcgcagaatgacaacgtcgtgtccatcaacgccactcttcagattgacttaactggcgcgtgcaactctgaacacatgcttggccatcagtatagcgcgagtggtggccagctcgattttgtgcgcggggcctatgcgagtaaaggaggccgtagcattattgcgacccctagtaccgctgctaaaggcacagttagccgcatcattccgcgcattgatggaccggtcaccacaccgcgcatcgatacccactacattgtcaccgagtttggcgccgtcaatctgaaaggtctgagcagcacggaacgtgccctgcgcatcatcgaactggcacatccggactttcgcgatgaattaacccaagccgcgaagaaaatgcatctgatctaa

SEQ ID NO: 24 - protein, 440 aa, Yersinia pestis
MDIRALYDEKLTTPEEAVSSIASGSHLSMGMFAAEPPALLKALADRATRGDIGDLRVYYFETAKIAGDTILRYELNNRIKPYSMFVTAVERALIRRGIEDGGRKVVNYVPSNFHQAPRLLAEEIGIDTFMHTVSPMDCHGYFSLGVGNDYSSRIARSARRFIVEVNRYMPRVQGEAAAIHISEVDAIVENHVPLIEMPVRSAIPEYTSISHIIADLVPDGACLQMGVGALPNLVCGVLKDRNDLGIHTEVLNPGLVDLIRRGVVTNQRKTLDRGRSVFTFAMGQQEMYEYLNDHPAIFSRPVDYVNDPHIIAQNDNVVSINATLQIDLTGACNSEHMLGHQYSASGGQLDFVRGAYASKGGRSIIATPSTAAKGTVSRIIPRIDGPVTTPRIDTHYIVTEFGAVNLKGLSSTERALRIIELAHPDFRDELTQAAKKMHLI

SEQ ID NO: 25 - DNA, 1362 bp, Clostridium kluyveri
  source 1..1362: mol_type = genomic DNA
atgagtaatgaagtatctataaaagaattaattgaaaaggcaaaggtggcacaaaaaaaattggaagcctatagtcaagaacaagttgatgtactagtaaaagcactaggaaaagtggtttatgataatgcagaaatgtttgcaaaagaagcagttgaagaaacagaaatgggtgtttatgaagataaagtagctaaatgtcatttgaaatcaggagctatttggaatcatataaaagacaagaaaactgtaggcataataaaagaagaacctgaaagggcacttgtttatgttgctaagccaaagggagttgtggcagctactacgcctataactaatccagtggtaactcctatgtgtaatgcaatggctgctataaagggcagaaatacaataatagtagcaccacatcctaaagcaaagaaagtttcagctcatactgtagaacttatgaatgctgagcttaaaaaattgggagcaccagaaaatatcatacagatagtagaagcaccatcaagagaagctgctaaggaacttatggaaagtgctgatgtagttattgctacaggcggtgctggaagagttaaagctgcttactccagtggaagaccagcttatggcgttggacctggaaattcacaggtaatagttgataagggatacgattataacaaagctgcacaggatataataacaggaagaaaatatgacaatggaattatatgttcttcagagcaatcagttatagctcctgctgaagattatgataaggtaatagcagcttttgtagaaaatggggcattctatgtagaagatgaggaaacagtagaaaagtttagatcaactttatttaaagatggaaaaataaacagcaagattataggtaaatccgtccaaattattgcggatcttgcaggagtaaaagtaccagaaggtactaaggttatagtacttaagggtaaaggtgcaggagaaaaagatgtactttgtaaagaaaaaatgtgtccagttttagtagcattgaaatatgatacttttgaagaagcagttgaaatagctatggctaattatatgtatgaaggagctggtcatacagcaggcatacattctgacaatgacgagaacataagatatgcaggaactgtattacctataagcagattagttgtaaatcagcctgcaactactgctggaggaagtttcaataatggatttaaccctactactacactaggctgcggatcatggggcagaaacagtatttcagaaaatcttacttacgagcatcttataaatgtttcaagaatagggtatttcaataaagaagcaaaagttcctagctatgaggaaatatggggataa

SEQ ID NO: 26 - protein, 453 aa, Clostridium kluyveri
MSNEVSIKELIEKAKVAQKKLEAYSQEQVDVLVKALGKVVYDNAEMFAKEAVEETEMGVYEDKVAKCHLKSGAIWNHIKDKKTVGIIKEEPERALVYVAKPKGVVAATTPITNPVVTPMCNAMAAIKGRNTIIVAPHPKAKKVSAHTVELMNAELKKLGAPENIIQIVEAPSREAAKELMESADVVIATGGAGRVKAAYSSGRPAYGVGPGNSQVIVDKGYDYNKAAQDIITGRKYDNGIICSSEQSVIAPAEDYDKVIAAFVENGAFYVEDEETVEKFRSTLFKDGKINSKIIGKSVQIIADLAGVKVPEGTKVIVLKGKGAGEKDVLCKEKMCPVLVALKYDTFEEAVEIAMANYMYEGAGHTAGIHSDNDENIRYAGTVLPISRLVVNQPATTAGGSFNNGFNPTTTLGCGSWGRNSISENLTYEHLINVSRIGYFNKEAKVPSYEEIWG

SEQ ID NO: 27 - DNA, 3657 bp, Chloroflexus aurantiacus
  source 1..3657: mol_type = genomic DNA
agcggaacaggacgactggcaggaaagattgcgttaattaccggtggcgccggcaatatcggcagtgaattgacacgtcgctttctcgcagagggagcgacggtcattattagtgggcggaatcgggcgaagttgaccgcactggccgaacggatgcaggcagaggcaggagtgtcggcaaagcgcatcgatctcgaagtcatggatgggagtgatccggtcgcggtacgtgccggtatcgaagcgattgtggcccgtcacggccagatcgacattctggtcaacaatgcaggaagtgccggtgcccagcgtcgtctggccgagattccactcactgaagctgaattaggccctggcgccgaagagacgcttcatgccagcatcgccaatttacttggtatgggatggcatctgatgcgtattgcggcacctcatatgccggtaggaagtgcggtcatcaatgtctcgaccatcttttcacgggctgagtactacgggcggattccgtatgtcacccctaaagctgctcttaatgctctatctcaacttgctgcgcgtgagttaggtgcacgtggcatccgcgttaatacgatctttcccggcccgattgaaagtgatcgcatccgtacagtgttccagcgtatggatcagctcaaggggcggcccgaaggcgacacagcgcaccattttttgaacaccatgcgattgtgtcgtgccaacgaccagggcgcgcttgaacgtcggttcccctccgtcggtgatgtggcagacgccgctgtctttctggccagtgccgaatccgccgctctcaccggtgagacgattgaggttacgcacggaatggagttgccggcctgcagtgagaccagcctgctggcccgtactgatctgcgcacgattgatgccagtggccgcacgacgctcatctgcgccggcgaccagattgaagaggtgatggcgctcaccggtatgttgcgtacctgtgggagtgaagtgatcatcggcttccgttcggctgcggcgctggcccagttcgagcaggcagtcaatgagagtcggcggctggccggcgcagactttacgcctcccattgccttgccactcgatccacgcgatccggcaacaattgacgctgtcttcgattgggccggcgagaataccggcgggattcatgcagcggtgattctgcctgctaccagtcacgaaccggcaccgtgcgtgattgaggttgatgatgagcgggtgctgaattttctggccgatgaaaccaccgggacaattgtgattgccagtcgcctggcccgttactggcagtcgcaacggcttacccccggcgcacgtgcgcgtgggccgcgtgtcatttttctctcgaacggtgccgatcaaaatgggaatgtttacggacgcattcaaagtgccgctatcggtcagctcattcgtgtgtggcgtcacgaggctgaacttgactatcagcgtgccagcgccgccggtgatcatgtgctgccgccggtatgggccaatcagattgtgcgcttcgctaaccgcagccttgaagggttagaatttgcctgtgcctggacagctcaattgctccatagtcaacgccatatcaatgagattaccctcaacatccctgccaacattagcgccaccaccggcgcacgcagtgcatcggtcggatgggcggaaagcctgatcgggttgcatttggggaaagttgccttgattaccggtggcagcgccggtattggtgggcagatcgggcgcctcctggctttgagtggcgcgcgcgtgatgctggcagcccgtgatcggcataagctcgaacagatgcaggcgatgatccaatctgagctggctgaggtggggtataccgatgtcgaagatcgcgtccacattgcaccgggctgcgatgtgagtagcgaagcgcagcttgcggatcttgttgaacgtaccctgtcagcttttggcaccgtcgattatctgatcaacaacgccgggatcgccggtgtcgaagagatggttatcgatatgccagttgagggatggcgccataccctcttcgccaatctgatcagcaactactcgttgatgcgcaaactggcgccgttgatgaaaaaacagggtagcggttacatccttaacgtctcatcatactttggcggtgaaaaagatgcggccattccctaccccaaccgtgccgattacgccgtctcgaaggctggtcagcgggcaatggccgaagtctttgcgcgcttccttggcccggagatacagatcaatgccattgcgccgggtccggtcgaaggtgatcgcttgcgcggtaccggtgaacgtcccggcctctttgcccgtcgggcgcggctgattttggagaacaagcggctgaatgagcttcacgctgctcttatcgcggctgcgcgcaccgatgagcgatctatgcacgaactggttgaactgctcttacccaatgatgtggccgcactagagcagaatcccgcagcacctaccgcgttgcgtgaactggcacgacgttttcgcagcgaaggcgatccggcggcatcatcaagcagtgcgctgctgaaccgttcaattgccgctaaattgctggctcgtttgcataatggtggctatgtgttgcctgccgacatctttgcaaacctgccaaacccgcccgatcccttcttcacccgagcccagattgatcgcgaggctcgcaaggttcgtgacggcatcatggggatgctctacctgcaacggatgccgactgagtttgatgtcgcaatggccaccgtctattaccttgccgaccgcaatgtcagtggtgagacattccacccatcaggtggtttgcgttacgaacgcacccctaccggtggcgaactcttcggcttgccctcaccggaacggctggcggagctggtcggaagcacggtctatctgataggtgaacatctgactgaacaccttaacctgcttgcccgtgcgtacctcgaacgttacggggcacgtcaggtagtgatgattgttgagacagaaaccggggcagagacaatgcgtcgcttgctccacgatcacgtcgaggctggtcggctgatgactattgtggccggtgatcagatcgaagccgctatcgaccaggctatcactcgctacggtcgcccagggccggtcgtctgtacccccttccggccactgccgacggtaccactggtcgggcgtaaagacagtgactggagcacagtgttgagtgaggctgaatttgccgagttgtgcgaacaccagctcacccaccatttccgggtagcgcgcaagattgccctgagtgatggtgccagtctcgcgctggtcactcccgaaactacggctacctcaactaccgagcaatttgctctggctaacttcatcaaaacgacccttcacgcttttacggctacgattggtgtcgagagcgaaagaactgctcagcgcattctgatcaatcaagtcgatctgacccggcgtgcgcgtgccgaagagccgcgtgatccgcacgagcgtcaacaagaactggaacgttttatcgaggcagtcttgctggtcactgcaccactcccgcctgaagccgatacccgttacgccgggcggattcatcgcggacgggcgattaccgtgtaa

SEQ ID NO: 28 - protein, 1218 aa, Chloroflexus aurantiacus
SGTGRLAGKIALITGGAGNIGSELTRRFLAEGATVIISGRNRAKLTALAERMQAEAGVSAKRIDLEVMDGSDPVAVRAGIEAIVARHGQIDILVNNAGSAGAQRRLAEIPLTEAELGPGAEETLHASIANLLGMGWHLMRIAAPHMPVGSAVINVSTIFSRAEYYGRIPYVTPKAALNALSQLAARELGARGIRVNTIFPGPIESDRIRTVFQRMDQLKGRPEGDTAHHFLNTMRLCRANDQGALERRFPSVGDVADAAVFLASAESAALTGETIEVTHGMELPACSETSLLARTDLRTIDASGRTTLICAGDQIEEVMALTGMLRTCGSEVIIGFRSAAALAQFEQAVNESRRLAGADFTPPIALPLDPRDPATIDAVFDWAGENTGGIHAAVILPATSHEPAPCVIEVDDERVLNFLADETTGTIVIASRLARYWQSQRLTPGARARGPRVIFLSNGADQNGNVYGRIQSAAIGQLIRVWRHEAELDYQRASAAGDHVLPPVWANQIVRFANRSLEGLEFACAWTAQLLHSQRHINEITLNIPANISATTGARSASVGWAESLIGLHLGKVALITGGSAGIGGQIGRLLALSGARVMLAARDRHKLEQMQAMIQSELAEVGYTDVEDRVHIAPGCDVSSEAQLADLVERTLSAFGTVDYLINNAGIAGVEEMVIDMPVEGWRHTLFANLISNYSLMRKLAPLMKKQGSGYILNVSSYFGGEKDAAIPYPNRADYAVSKAGQRAMAEVFARFLGPEIQINAIAPGPVEGDRLRGTGERPGLFARRARLILENKRLNELHAALIAAARTDERSMHELVELLLPNDVAALEQNPAAPTALRELARRFRSEGDPAASSSSALLNRSIAAKLLARLHNGGYVLPADIFANLPNPPDPFFTRAQIDREARKVRDGIMGMLYLQRMPTEFDVAMATVYYLADRNVSGETFHPSGGLRYERTPTGGELFGLPSPERLAELVGSTVYLIGEHLTEHLNLLARAYLERYGARQVVMIVETETGAETMRRLLHDHVEAGRLMTIVAGDQIEAAIDQAITRYGRPGPVVCTPFRPLPTVPLVGRKDSDWSTVLSEAEFAELCEHQLTHHFRVARKIALSDGASLALVTPETTATSTTEQFALANFIKTTLHAFTATIGVESERTAQRILINQVDLTRRARAEEPRDPHERQQELERFIEAVLLVTAPLPPEADTRYAGRIHRGRAITV

SEQ ID NO: 29 - DNA, 1080 bp, synthetic construct
  source 1..1080: mol_type = other DNA
atgatcctgatgcgccgcaccctgaaagcagcaattctgggtgccaccggtctggttggtattgaatatgtgcgcatgttaagtaatcatccgtatattaaaccggcatatctggccggtaaaggcagtgttggtaaaccgtatggcgaagttgtgcgttggcagaccgtgggccaggtgccgaaagaaattgccgatatggaaattaaaccgaccgatccgaaactgatggatgatgtggatattatcttcagtccgctgccgcagggcgcagcaggtcctgttgaagaacagttcgcaaaagaaggcttcccggttattagcaatagcccggatcatcgcttcgatccggatgtgccgctgctggttccggaactgaatccgcataccattagtctgattgatgaacagcgtaaacgccgcgaatggaaaggcttcattgtgaccaccccggtgtgcaccgcccagggtgcagcaattccgctgggcgcaatcttcaaagattataaaatggatggcgccttcattaccaccattcagagcctgagtggtgccggttatccgggcattccgagcctggatgtggttgataatattctgccgctgggtgatggttatgatgcaaaaaccattaaagaaatcttccgcattctgagcgaagtgaaacgtaatgtggatgaaccgaaactggaagatgttagcctggcagccaccacccatcgcattgccaccattcatggtcattatgaagtgctgtatgtgagcttcaaagaagaaaccgcagccgaaaaagttaaagaaaccttagaaaacttccgtggtgaaccgcaggatctgaaactgccgaccgcaccgagtaaaccgattattgttatgaatgaagacacccgcccgcaggtgtacttcgatcgttgggccggtgatattccgggtatgagtgttgttgttggtcgtctgaaacaggtgaataaacgtatgattcgcctggtgagtctgattcataataccgttcgtggcgccgcaggcggtggtattctggcagcagaactgctggtggaaaaaggttatattgaaaaataa

SEQ ID NO: 30 - protein, 359 aa, synthetic construct
MILMRRTLKAAILGATGLVGIEYVRMLSNHPYIKPAYLAGKGSVGKPYGEVVRWQTVGQVPKEIADMEIKPTDPKLMDDVDIIFSPLPQGAAGPVEEQFAKEGFPVISNSPDHRFDPDVPLLVPELNPHTISLIDEQRKRREWKGFIVTTPVCTAQGAAIPLGAIFKDYKMDGAFITTIQSLSGAGYPGIPSLDVVDNILPLGDGYDAKTIKEIFRILSEVKRNVDEPKLEDVSLAATTHRIATIHGHYEVLYVSFKEETAAEKVKETLENFRGEPQDLKLPTAPSKPIIVMNEDTRPQVYFDRWAGDIPGMSVVVGRLKQVNKRMIRLVSLIHNTVRGAAGGGILAAELLVEKGYIEK

SEQ ID NO: 31 - DNA, 1080 bp, synthetic construct
  source 1..1080: mol_type = other DNA
atgatcctgatgcgccgcaccctgaaagcagcaattctgggtgccaccggtctggttggtattgaatatgtgcgcatgttaagtaatcatccgtatattaaaccggcatatctggccggtaaaggcagtgttggtaaaccgtatggcgaagttgtgcgttggcagaccgtgggccaggtgccgaaagaaattgccgatatggaaattaaaccgaccgatccgaaactgatggatgatgtggatattatcttcagtccgctgccgcagggcgcagcaggtcctgttgaagaacagttcgcaaaagaaggcttcccggttattagcaatagcccggatcatcgcttcgatccggatgtgccgctgctggttccggaactgaatccgcataccattagtctgattgatgaacagcgtaaacgccgcgaatggaaaggcttcattgtgaccaccccggcctgcaccgcccagggtgcagcaattccgctgggcgcaatcttcaaagattataaaatggatggcgccttcattaccaccattcagagcctgagtggtgccggttatccgggcattccgagcctggatgtggttgataatattctgccgctgggtgatggttatgatgcaaaaaccattaaagaaatcttccgcattctgagcgaagtgaaacgtaatgtggatgaaccgaaactggaagatgttagcctggcagccaccacccatcgcattgccaccattcatggtcattatgaagtgctgtatgtgagcttcaaagaagaaaccgcagccgaaaaagttaaagaaaccttagaaaacttccgtggtgaaccgcaggatctgaaactgccgaccgcaccgagtaaaccgattattgttatgaatgaagacacccgcccgcaggtgtacttcgatcgttgggccggtgatattccgggtatgagtgttgttgttggtcgtctgaaacaggtgaataaacgtatgattcgcctggtgagtctgattcataataccgttcgtggcgccgcaggcggtggtattctggcagcagaactgctggtggaaaaaggttatattgaaaaataa

SEQ ID NO: 32 - protein, 359 aa, synthetic construct
MILMRRTLKAAILGATGLVGIEYVRMLSNHPYIKPAYLAGKGSVGKPYGEVVRWQTVGQVPKEIADMEIKPTDPKLMDDVDIIFSPLPQGAAGPVEEQFAKEGFPVISNSPDHRFDPDVPLLVPELNPHTISLIDEQRKRREWKGFIVTTPACTAQGAAIPLGAIFKDYKMDGAFITTIQSLSGAGYPGIPSLDVVDNILPLGDGYDAKTIKEIFRILSEVKRNVDEPKLEDVSLAATTHRIATIHGHYEVLYVSFKEETAAEKVKETLENFRGEPQDLKLPTAPSKPIIVMNEDTRPQVYFDRWAGDIPGMSVVVGRLKQVNKRMIRLVSLIHNTVRGAAGGGILAAELLVEKGYIEK

SEQ ID NO: 33 - DNA, 1080 bp, synthetic construct
  source 1..1080: mol_type = other DNA
atgatcctgatgcgccgcaccctgaaagcagcaattctgggtgccaccggtctggttggtattgaatatgtgcgcatgttaagtaatcatccgtatattaaaccggcatatctggccggtaaaggcagtgttggtaaaccgtatggcgaagttgtgcgttggcagaccgtgggccaggtgccgaaagaaattgccgatatggaaattaaaccgaccgatccgaaactgatggatgatgtggatattatcttcagtccgctgccgcagggcgcagcaggtcctgttgaagaacagttcgcaaaagaaggcttcccggttattagcaatagcccggatcatcgcttcgatccggatgtgccgctgctggttccggaactgaatccgcataccattagtctgattgatgaacagcgtaaacgccgcgaatggaaaggcttcattgtgaccaccccgacctgcaccgcccagggtgcagcaattccgctgggcgcaatcttcaaagattataaaatggatggcgccttcattaccaccattcagagcctgagtggtgccggttatccgggcattccgagcctggatgtggttgataatattctgccgctgggtgatggttatgatgcaaaaaccattaaagaaatcttccgcattctgagcgaagtgaaacgtaatgtggatgaaccgaaactggaagatgttagcctggcagccaccacccatcgcattgccaccattcatggtcattatgaagtgctgtatgtgagcttcaaagaagaaaccgcagccgaaaaagttaaagaaaccttagaaaacttccgtggtgaaccgcaggatctgaaactgccgaccgcaccgagtaaaccgattattgttatgaatgaagacacccgcccgcaggtgtacttcgatcgttgggccggtgatattccgggtatgagtgttgttgttggtcgtctgaaacaggtgaataaacgtatgattcgcctggtgagtctgattcataataccgttcgtggcgccgcaggcggtggtattctggcagcagaactgctggtggaaaaaggttatattgaaaaataa

SEQ ID NO: 34 - protein, 359 aa, synthetic construct
MILMRRTLKAAILGATGLVGIEYVRMLSNHPYIKPAYLAGKGSVGKPYGEVVRWQTVGQVPKEIADMEIKPTDPKLMDDVDIIFSPLPQGAAGPVEEQFAKEGFPVISNSPDHRFDPDVPLLVPELNPHTISLIDEQRKRREWKGFIVTTPTCTAQGAAIPLGAIFKDYKMDGAFITTIQSLSGAGYPGIPSLDVVDNILPLGDGYDAKTIKEIFRILSEVKRNVDEPKLEDVSLAATTHRIATIHGHYEVLYVSFKEETAAEKVKETLENFRGEPQDLKLPTAPSKPIIVMNEDTRPQVYFDRWAGDIPGMSVVVGRLKQVNKRMIRLVSLIHNTVRGAAGGGILAAELLVEKGYIEK

SEQ ID NO: 35 - DNA, 1080 bp, Sulfolobus tokodaii
  source 1..1080: mol_type = genomic DNA
atgatcctgatgcgccgcaccctgaaagcagcaattctgggtgccaccggtctggttggtattgaatatgtgcgcatgttaagtaatcatccgtatattaaaccggcatatctggccggtaaaggcagtgttggtaaaccgtatggcgaagttgtgcgttggcagaccgtgggccaggtgccgaaagaaattgccgatatggaaattaaaccgaccgatccgaaactgatggatgatgtggatattatcttcagtccgctgccgcagggcgcagcaggtcctgttgaagaacagttcgcaaaagaaggcttcccggttattagcaatagcccggatcatcgcttcgatccggatgtgccgctgctggttccggaactgaatccgcataccattagtctgattgatgaacagcgtaaacgccgcgaatggaaaggcttcattgtgaccaccccgctgtgcaccgcccagggtgcagcaattccgctgggcgcaatcttcaaagattataaaatggatggcgccttcattaccaccattcagagcctgagtggtgccggttatccgggcattccgagcctggatgtggttgataatattctgccgctgggtgatggttatgatgcaaaaaccattaaagaaatcttccgcattctgagcgaagtgaaacgtaatgtggatgaaccgaaactggaagatgttagcctggcagccaccacccatcgcattgccaccattcatggtcattatgaagtgctgtatgtgagcttcaaagaagaaaccgcagccgaaaaagttaaagaaaccttagaaaacttccgtggtgaaccgcaggatctgaaactgccgaccgcaccgagtaaaccgattattgttatgaatgaagacacccgcccgcaggtgtacttcgatcgttgggccggtgatattccgggtatgagtgttgttgttggtcgtctgaaacaggtgaataaacgtatgattcgcctggtgagtctgattcataataccgttcgtggcgccgcaggcggtggtattctggcagcagaactgctggtggaaaaaggttatattgaaaaataa

SEQ ID NO: 36 - protein, 359 aa, Sulfolobus tokodaii
MILMRRTLKAAILGATGLVGIEYVRMLSNHPYIKPAYLAGKGSVGKPYGEVVRWQTVGQVPKEIADMEIKPTDPKLMDDVDIIFSPLPQGAAGPVEEQFAKEGFPVISNSPDHRFDPDVPLLVPELNPHTISLIDEQRKRREWKGFIVTTPLCTAQGAAIPLGAIFKDYKMDGAFITTIQSLSGAGYPGIPSLDVVDNILPLGDGYDAKTIKEIFRILSEVKRNVDEPKLEDVSLAATTHRIATIHGHYEVLYVSFKEETAAEKVKETLENFRGEPQDLKLPTAPSKPIIVMNEDTRPQVYFDRWAGDIPGMSVVVGRLKQVNKRMIRLVSLIHNTVRGAAGGGILAAELLVEKGYIEK

SEQ ID NO: 37 - DNA, 3693 bp, Porphyrobacter dokdonensis
  source 1..3693: mol_type = genomic DNA
atggccaaatcgaaagctgctgccgccgcgcccgccttgcagggcgatgttccgggccgtctggctgggaaaatcgcggtcgtcaccggcgctgcaggcaatctcggcgggcatatcgtcacgcactatcttgcagagggtgcaaccgtagtgatgacgggccgcacgcccgatcgcaccaaggcggctgctgatgcgctgctcaagtcgactggcgcagatccgtcgcggcttgccacggtcgccttggacggcggagacatagcatcggtgcgcgccgcgattgccgaggtggtgcagaaattcggccggatcgatatcctcgtcaacaacgccggaagcgccgggccgaagcagccgatcgagaacctgccgctctcgccggaagaactggcggcgctgcagaagaccggcagcaccgacagcgagactgtcgccgatgctttgcgcaacattttcggagtcgcatggaatgtcgcgcgggtggctgcgccgcatattccggaaggcggttcgatcatcaacgtgtcgaccatcttcagccgcacgccctattacgcgcgcgctgcctacgtcgtccccaaagcggcaatgaatgcgtggagccgcgagctgagccttgagctcgggcctaaaggcatccgcgtgaacctcgtctatcccggcccgatcgagagcgagcgcatccgctctgtctttgccgcgatggatgcagcgcgcggggacgaggcaggcaccactgccacacagttcttcgacatgatgagccttgaacgggccaccggcgggaacgagaaggccaagaccttccccacgcccgaggacatcgccaccacctgcgtattcctcggctcggatgaaagcgcggcctacaacggccacgatttcgaagtcacccacggcatgagtgtgaggaaggagcagcgctcgacctatcttgcgcggccgaccatgcgctcgatggacggcacgggtttggccgtgctgatcgcggcgggcgatgactgggaggaagcgctggaaatcgcgcaggtgcagctggcctgcggggcgcaggtggtgctcggcctgccccgcgctgccgatgttgccattgccgagaagcgctgcaaggcgctgggccttaccgaaggcctctcgatcatccgcttcagccgcaaggatccggcggcaatggaagccgcgctcgaggaatataccaggggcggcacgccgatcagcggcgcgctgttcatgcctgcgctgggtgcgggcgagctttcgggcgccgtcaccgaggcggaggacaatgcagttgaggctctgatggatgccgaacttgcgggcaacatggccctggcgcggacaatgagccgctactggaagcgccacgacaacctgctgcagccgccgcgcttcgtgttcgtcagccatgcgagcgacgggaagggcgatatctatggtcacatcctgcgcgccgccacggaacagttgatccgcatctggcgcgacgaatccgaaatcgacaccgcccatggtcgccgccgacaggccgagtggggcaaccagatcgtgcgcttcaccaacaccgaggcggagaacatccgcttcaccgcgggtcacgcggcgcgcatcctgctgaaggaaagcaagctcggagagatcacgctctacgtgcccgccaatatcggcgaggcgaccggggcgaggaaggccatgccgggcttctccgagaacatcaccgggctgcacttgggcaaggtggcgctgattaccggcgggtcggcgggcatcggcgggcaggtcgcgcgcctgctggcgctcgcgggcggcaaggtgatgatggtcgcccgccgtgaaagcgagctggcggtggcgcgggcgcggatcgtctcggagctggaggacatcggtttcgccggggtcgagcgccgcgtgcagaccctagccaacgtcgatgtcagcaactttgaatccctcaaaggcgcggtcgacgcgacgctcaaggccttcgggcggatcgactacctcatcaataacgcaggcgtggccggcgcggaggacatggttgtcgacatgggcgtggatgcgtgggactacacgctcgatgcgaacctcgtgtcgaactacttcctgatgcaccacgtcgcgccgctgatgaaggcgcagggatcgggatatatcctcaatgtctcgtcctatttcggcggagagaagtatcttgccgtcgcctatcccaaccgcgcggattacgccgtgtccaaggccgggcagcgcgcgatggtggagagcatggcgcgctatctggggccggaggtgcagttcaacgcgatcgcgccggggccggtggatggcgaccgcctctcgggcacgggcggcaagcccggcctgttcgagcggcgcgggaagctgattctcgaaaacaagcgtttgaacgccgtccacgccgccgcgatcaaggcgatccggcgcggggtgcgggtcgaggcagtgctggcgcggctcgcgcgcaacgataccgtcaagatgagccacgacaccaacaacccgcgcgagttgcgtgaactcgcgctggcctgcgcgcgcgagggggacggcacctgtacctgggaccagtatctcctgaccccgcagatcgctgccgcgctggtcagccgcttgcgtcaggcgggtctgttcctcgatgcgcccgaatggtccgaacgtccggtaaccgaagacggcgactggctgctgcgcgtgccgccggaggacgcgcctttccttcccgccgacaagatcgcggccgaagccaagaaggtcggcggcggggtgctgtccaagctctatctcggcaagatgccgaccgagcatgacgtggcgcaggcgacggtgttcttcctcgcggaccgcgctgtgtcgggtgaaaccttcatgccttcgggcggcctcagcgtggagcgctcgaccaccgagcgcgagctgttcggcagtcccaagcaggagcggctcgaccagatgcggggcaagacagtgtggattatcggcgaacacctcgtcgattacctcgccgaaaccgcgcgcgccttcatcgaggattgccatgccgccaacgtggtgctgatcacccgcaccgccgaaggtttcgatgccgtcgaggcgcaactcgacgaggacgtcgcgcagtcgctgacctcgctggttgtttcgagcgatatcgaggcggcgatggacgaggcgctcagccagtggggcaggccgaccacgatcctctccacgccgttcaccgcactgccgggcaagctgttcgaagcgcaagacccgctgacgccggacgaattccgcgaagttgtggccgacaatctcacccaccacttccgcgtctcgcgccgcgcctcgctttatgatgattgccagctggtgctgaccagtcccgacgtggccatgggcgacaagtccccggccttcgcgctggcgaacttcatcaagacgacgctccacgccttcaccgcgacgctggcggtcgaaaacgaacgtctggttcacgatgtgccggtcaaccagatcaacctcacccgccgggtgcagtcggaggagccacgcgatctcgacgagcatcttgaagaggtgcggcgctttgcccgcgctgtgctgctggtcggcgcgccgctgccggacgccgaagattcgcgttatcgcgcgcggatctatcgcggcatgtcgatgacggtgtga

SEQ ID NO: 38 - protein, 1230 aa, Porphyrobacter dokdonensis
MAKSKAAAAAPALQGDVPGRLAGKIAVVTGAAGNLGGHIVTHYLAEGATVVMTGRTPDRTKAAADALLKSTGADPSRLATVALDGGDIASVRAAIAEVVQKFGRIDILVNNAGSAGPKQPIENLPLSPEELAALQKTGSTDSETVADALRNIFGVAWNVARVAAPHIPEGGSIINVSTIFSRTPYYARAAYVVPKAAMNAWSRELSLELGPKGIRVNLVYPGPIESERIRSVFAAMDAARGDEAGTTATQFFDMMSLERATGGNEKAKTFPTPEDIATTCVFLGSDESAAYNGHDFEVTHGMSVRKEQRSTYLARPTMRSMDGTGLAVLIAAGDDWEEALEIAQVQLACGAQVVLGLPRAADVAIAEKRCKALGLTEGLSIIRFSRKDPAAMEAALEEYTRGGTPISGALFMPALGAGELSGAVTEAEDNAVEALMDAELAGNMALARTMSRYWKRHDNLLQPPRFVFVSHASDGKGDIYGHILRAATEQLIRIWRDESEIDTAHGRRRQAEWGNQIVRFTNTEAENIRFTAGHAARILLKESKLGEITLYVPANIGEATGARKAMPGFSENITGLHLGKVALITGGSAGIGGQVARLLALAGGKVMMVARRESELAVARARIVSELEDIGFAGVERRVQTLANVDVSNFESLKGAVDATLKAFGRIDYLINNAGVAGAEDMVVDMGVDAWDYTLDANLVSNYFLMHHVAPLMKAQGSGYILNVSSYFGGEKYLAVAYPNRADYAVSKAGQRAMVESMARYLGPEVQFNAIAPGPVDGDRLSGTGGKPGLFERRGKLILENKRLNAVHAAAIKAIRRGVRVEAVLARLARNDTVKMSHDTNNPRELRELALACAREGDGTCTWDQYLLTPQIAAALVSRLRQAGLFLDAPEWSERPVTEDGDWLLRVPPEDAPFLPADKIAAEAKKVGGGVLSKLYLGKMPTEHDVAQATVFFLADRAVSGETFMPSGGLSVERSTTERELFGSPKQERLDQMRGKTVWIIGEHLVDYLAETARAFIEDCHAANVVLITRTAEGFDAVEAQLDEDVAQSLTSLVVSSDIEAAMDEALSQWGRPTTILSTPFTALPGKLFEAQDPLTPDEFREVVADNLTHHFRVSRRASLYDDCQLVLTSPDVAMGDKSPAFALANFIKTTLHAFTATLAVENERLVHDVPVNQINLTRRVQSEEPRDLDEHLEEVRRFARAVLLVGAPLPDAEDSRYRARIYRGMSMTV

SEQ ID NO: 39 - DNA, 1164 bp, Escherichia coli
  source 1..1164: mol_type = genomic DNA
atggctaacagaatgattctgaacgaaacggcatggtttggtcggggtgctgttggggctttaaccgatgaggtgaaacgccgtggttatcagaaggcgctgatcgtcaccgataaaacgctggtgcaatgcggcgtggtggcgaaagtgaccgataagatggatgctgcagggctggcatgggcgatttacgacggcgtagtgcccaacccaacaattactgtcgtcaaagaagggctcggtgtattccagaatagcggcgcggattacctgatcgctattggtggtggttctccacaggatacttgtaaagcgattggcattatcagcaacaacccggagtttgccgatgtgcgtagcctggaagggctttccccgaccaataaacccagtgtaccgattctggcaattcctaccacagcaggtactgcggcagaagtgaccattaactacgtgatcactgacgaagagaaacggcgcaagtttgtttgcgttgatccgcatgatatcccgcaggtggcgtttattgacgctgacatgatggatggtatgcctccagcgctgaaagctgcgacgggtgtcgatgcgctcactcatgctattgaggggtatattacccgtggcgcgtgggcgctaaccgatgcactgcacattaaagcgattgaaatcattgctggggcgctgcgaggatcggttgctggtgataaggatgccggagaagaaatggcgctcgggcagtatgttgcgggtatgggcttctcgaatgttgggttagggttggtgcatggtatggcgcatccactgggcgcgttttataacactccacacggtgttgcgaacgccatcctgttaccgcatgtcatgcgttataacgctgactttaccggtgagaagtaccgcgatatcgcgcgcgttatgggcgtgaaagtggaaggtatgagcctggaagaggcgcgtaatgccgctgttgaagcggtgtttgctctcaaccgtgatgtcggtattccgccacatttgcgtgatgttggtgtacgcaaggaagacattccggcactggcgcaggcggcactggatgatgtttgtaccggtggcaacccgcgtgaagcaacgcttgaggatattgtagagctttaccataccgcctggggcctatgcggccgctaa

SEQ ID NO: 40 - protein, 387 aa, Escherichia coli
MANRMILNETAWFGRGAVGALTDEVKRRGYQKALIVTDKTLVQCGVVAKVTDKMDAAGLAWAIYDGVVPNPTITVVKEGLGVFQNSGADYLIAIGGGSPQDTCKAIGIISNNPEFADVRSLEGLSPTNKPSVPILAIPTTAGTAAEVTINYVITDEEKRRKFVCVDPHDIPQVAFIDADMMDGMPPALKAATGVDALTHAIEGYITRGAWALTDALHIKAIEIIAGALRGSVAGDKDAGEEMALGQYVAGMGFSNVGLGLVHGMAHPLGAFYNTPHGVANAILLPHVMRYNADFTGEKYRDIARVMGVKVEGMSLEEARNAAVEAVFALNRDVGIPPHLRDVGVRKEDIPALAQAALDDVCTGGNPREATLEDIVELYHTAWGLCGR

SEQ ID NO: 41 - DNA, 1164 bp, Escherichia coli
  source 1..1164: mol_type = genomic DNA
atgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtgctggaatttggcggtattgagccaaacccggcttatgaaacgctgatgaacgccgtgaaactggttcgcgaacagaaagtgactttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagccggtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctaatcgaagatggtccgaaagccctgaaagagccagaaaactacgatgtgcgcgccaacgtcatgtgggcggcgactcaggcgctgaacggtttgattggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttccgatgatgagcgtattgacgccgcgattgccgcaacccgcaatttctttgagcaattaggcgtgccgacccacctctccgactacggtctggacggcagctccatcccggctttgctgaaaaaactggaagagcacggcatgacccaactgggcgaaaatcatgacattacgttggatgtcagccgccgtatatacgaagccgcccgctaa

SEQ ID NO: 42 - protein, 387 aa, Escherichia coli
MNNFNLHTPTRILFGKGAIAGLREQIPHDARVLITYGGGSVKKTGVLDQVLDALKGMDVLEFGGIEPNPAYETLMNAVKLVREQKVTFLLAVGGGSVLDGTKFIAAAANYPENIDPWHILQTGGKEIKSAIPMGCVLTLPATGSESNAGAVISRKTTGDKQAFHSAHVQPVFAVLDPVYTYTLPPRQVANGVVDAFVHTVEQYVTKPVDAKIQDRFAEGILLTLIEDGPKALKEPENYDVRANVMWAATQALNGLIGAGVPQDWATHMLGHELTAMHGLDHAQTLAIVLPALWNEKRDTKRAKLLQYAERVWNITEGSDDERIDAAIAATRNFFEQLGVPTHLSDYGLDGSSIPALLKKLEEHGMTQLGENHDITLDVSRRIYEAAR

SEQ ID NO: 43 - DNA, 969 bp, Staphylococcus aureus
  source 1..969: mol_type = genomic DNA
atgtcacaacaagatttacctacattattttatagcgggaagtccaatagtgctgttccaattatatctgaaagtgaattacaaacaattacagctgaaccatggcttgaaatttccaaaaaaggattgcaactagaaggattgaactttgatcggcagggacaactctttttattggatgtattcgaaggcaatattttcaaaatcaatcctgaaacgaaggaaatcaaacgaccttttgtaagtcacaaagcgaatcctgcagcaatcaaaatacataaagatggccgattattcgtttgttatttaggagattttaaatctacaggaggcatttttgcagctacagaaaatggtgacaacttacaagatattattgaagatctttcaacagcatattgtattgatgacatggtatttgattctaaaggtggattttattttacagattttagaggatactctaccaatccactaggaggcgtttattatgtttcgccggactttagaacagtgacgcctatcattcaaaatattagcgtagcaaatggtattgctttaagtacagatgaaaaagtactatgggtaacagaaacaacagccaatcgattacatcgcattgcacttgaagatgatggtgtgacgatacaaccatttggagctaccataccgtactattttacaggtcatgaaggaccagactcatgttgtattgatagtgacgataatttatacgtagcaatgtatggtcaaggtcgagtgttagtttttaataaaaggggttatccaataggacaaatattgataccaggccgagatgaagggcatatgttacgttctactcatccgcaatttatacctggaacaaatcaactcatcatttgttccaatgatatagaaatgggcggaggatctatgctttatacagttaatggctttgcaaaaggtcatcaaagttttcagtttcaa

SEQ ID NO: 44 - protein, 323 aa, Staphylococcus aureus
MSQQDLPTLFYSGKSNSAVPIISESELQTITAEPWLEISKKGLQLEGLNFDRQGQLFLLDVFEGNIFKINPETKEIKRPFVSHKANPAAIKIHKDGRLFVCYLGDFKSTGGIFAATENGDNLQDIIEDLSTAYCIDDMVFDSKGGFYFTDFRGYSTNPLGGVYYVSPDFRTVTPIIQNISVANGIALSTDEKVLWVTETTANRLHRIALEDDGVTIQPFGATIPYYFTGHEGPDSCCIDSDDNLYVAMYGQGRVLVFNKRGYPIGQILIPGRDEGHMLRSTHPQFIPGTNQLIICSNDIEMGGGSMLYTVNGFAKGHQSFQFQ

SEQ ID NO: 45 - DNA, 822 bp, Saccharopolyspora erythraea
  source 1..822: mol_type = genomic DNA
agcgggactcccgcccgggaagcgagcagcgctcttcgcgacggctaccggcaggcgggcgtgtcgggcagggtccggtcctacctcgacctgctggcggggctgtcggacttccgcgagcacttcgacggctccgacgggttctccctcgatctcgtggacatggccgacggtcccggagaggtcacggtgatctgctgcgcgggaacggcggcgatctccggtccgcacgagttcacccggctcgccggggcgctgcgcggaatcgctccggttcgggccgtgccccagcccggctacgaggagggcgaacctctgccgtcgtcgatggcggcggtggcggcggtgcaggccgatgcggtcatcaggacacagggggacaagccgttcgtggtggccggtcactccgcgggggcactgatggcctacgcgctggcgaccgaactgctcgatcgcgggcacccgccacgcggtgtcgtcctgatcgacgtctacccgcccggtcaccaggacgcgatgaacgcctggctggaggagctgaccgccacgctgttcgaccgcgagacggtgcggatggacgacaccaggctcaccgccctgggcgcctacgaccgcctcaccggtcagtggcgaccccgggaaaccgggctgccgacgctgctggtcagcgccggcgagccgatgggtccgtggcccgacgacagctggaagccgacgtggcccttcgagcacgacaccgtcgccgtccccggcgaccacttcacgatggtgcaggaacacgccgacgcgatcgcgcggcacatcgacgcctggctgggcgga

SEQ ID NO: 46 - protein, 274 aa, Saccharopolyspora erythraea
SGTPAREASSALRDGYRQAGVSGRVRSYLDLLAGLSDFREHFDGSDGFSLDLVDMADGPGEVTVICCAGTAAISGPHEFTRLAGALRGIAPVRAVPQPGYEEGEPLPSSMAAVAAVQADAVIRTQGDKPFVVAGHSAGALMAYALATELLDRGHPPRGVVLIDVYPPGHQDAMNAWLEELTATLFDRETVRMDDTRLTALGAYDRLTGQWRPRETGLPTLLVSAGEPMGPWPDDSWKPTWPFEHDTVAVPGDHFTMVQEHADAIARHIDAWLGG

SEQ ID NO: 47 - DNA, 831 bp, Streptomyces amphibiosporus
  source 1..831: mol_type = genomic DNA
cccgtattttgggttcatcacggaaatgggggcgttgagagctacgcagcggttgcggaatgctgtggtcgtccgttctatggtattcagccgcgtggctggaccggctccgaagatatcctgaccggtcaagaggcaatggctgcgtactacgtggacatcattcgtgcggtgcagcctgagggtccgtatgatgtgggtggcttcagcctgggtggcctgttcgcctacgaggtggtgcgccagctgcaattgcaagacgcgaccgttgatacgctggttatgttggacactctggacgccgcaagcaccaatttggcgaacagcttaatgaccggtggccgtcaggacgacgcggatgtcgtggccaaagtttccgcgtttcgtgcggtgaacctgatgctaggaaacgattctctcgacgcgcgcgagggcaccagctctgttctgcaccgtgatgaagtcgacaccgcactcgacccggacgctttcctggactcgctggtggaagcggcggttgctcgcggcatccacaagaccccggcgcaattgagaacgcgtgtgcgtcaacttgcccgctatttcgatgctgtgcacggcgaacgtcatgttgtccatccgttgccacgtcgcgaggaagtccgctgctattacctgcgtaatgcaggtggccagtttttcggtccgtttgaagagtatatggttctgtttccggagccggatctgccggcggtcgatggtaccccgtactggcgtgagtgggcagatgccgttgacgattttttcgtgattgacgtagacactgagacgcatgcgcaggttatgaccgaa

SEQ ID NO: 48 - protein, 277 aa, Streptomyces amphibiosporus
PVFWVHHGNGGVESYAAVAECCGRPFYGIQPRGWTGSEDILTGQEAMAAYYVDIIRAVQPEGPYDVGGFSLGGLFAYEVVRQLQLQDATVDTLVMLDTLDAASTNLANSLMTGGRQDDADVVAKVSAFRAVNLMLGNDSLDAREGTSSVLHRDEVDTALDPDAFLDSLVEAAVARGIHKTPAQLRTRVRQLARYFDAVHGERHVVHPLPRREEVRCYYLRNAGGQFFGPFEEYMVLFPEPDLPAVDGTPYWREWADAVDDFFVIDVDTETHAQVMTE

SEQ ID NO: 49 - DNA, 651 bp, Streptomyces sp.
  source 1..651: mol_type = genomic DNA
gtttgtgtatgtccccctttagcccttaatggccctcaaacttataatcgctttgccgcggagctcgcggcggattggaaagttagcgctcttgttgcgccaggttttgccgccgatgctccattacccgaaaattcacgtgtcttaattgccaccttagcggatgctgttcaagaatatgtaggggattcggcttttgcactcgcgggactctcatcaggtggagtcctggcacatgaaattgctcgtgagctcgaacgtcgcggtagtgatcctttagccgtcgtcctgttagatagttacccaatggattcgcctgttttgaaacgctggaagaatgaaatggcgcgtgtgggattagaacgtgcgcgtgcgcttgatcgtcgtcaagcggatgaaggttttcgcttggaagaaattactgcgagcgcatggatttgtggtgagcttcttctggattggcgccctgaagggttgacagctccaagctgtttagtgcgtgcaagtgaaccagttgttgctgaagatggtgcgggatggcaaactcccgtagcagctagtgaagttattgatgtacctggagatcatttctcaattatggatcgccaatttgccgcatcgacggccgcggcagtccgtgcctggctcgcc

SEQ ID NO: 50 - protein, 217 aa, Streptomyces sp.
VCVCPPLALNGPQTYNRFAAELAADWKVSALVAPGFAADAPLPENSRVLIATLADAVQEYVGDSAFALAGLSSGGVLAHEIARELERRGSDPLAVVLLDSYPMDSPVLKRWKNEMARVGLERARALDRRQADEGFRLEEITASAWICGELLLDWRPEGLTAPSCLVRASEPVVAEDGAGWQTPVAASEVIDVPGDHFSIMDRQFAASTAAAVRAWLA

SEQ ID NO: 51 - DNA, 4690 bp, Nocardia iowensis
  source 1..4690: mol_type = genomic DNA
acggcgtaggcgatcacgtaaccggccacgatcagctgcaaggcagcgccggaggtgtgcagggaatcgcgaatcgacggaatcgcgacattgacgatggaggagtcgagcaccgccatgaactggccggtgaggatcaccgtgaggatggccagggtgcgccgcgcaccgtacgcggccggtggagctgccgggggcgcgacaaccggcgatgcggtggccgggtccggcgacaagatctgggtcatatgctgatcttgcgagctcgatgacaccggtaacaggagcccattaaaactggtaccggcaatacctggataagcggtcggatcctgggccgctgcggtggagtggccgccgttccggcccgatgtggccaagaccactcgagtcaccgccgcgtatcaccttcccggaagtatttacttaggctaacgtgttttacgggttgcagggcttttcctacttatgacaagggaggcttgccatggcagtggattcaccggatgagcggctacagcgccgcattgcacagttgtttgcagaagatgagcaggtcaaggccgcacgtccgctcgaagcggtgagcgcggcggtgagcgcgcccggtatgcggctggcgcagatcgccgccactgttatggcgggttacgccgaccgcccggccgccgggcagcgtgcgttcgaactgaacaccgacgacgcgacgggccgcacctcgctgcggttacttccccgattcgagaccatcacctatcgcgaactgtggcagcgagtcggcgaggttgccgcggcctggcatcatgatcccgagaaccccttgcgcgcaggtgatttcgtcgccctgctcggcttcaccagcatcgactacgccaccctcgacctggccgatatccacctcggcgcggttaccgtgccgttgcaggccagcgcggcggtgtcccagctgatcgctatcctcaccgagacttcgccgcggctgctcgcctcgaccccggagcacctcgatgcggcggtcgagtgcctactcgcgggcaccacaccggaacgactggtggtcttcgactaccaccccgaggacgacgaccagcgtgcggccttcgaatccgcccgccgccgccttgccgacgcgggcagcttggtgatcgtcgaaacgctcgatgccgtgcgtgcccggggccgcgacttaccggccgcgccactgttcgttcccgacaccgacgacgacccgctggccctgctgatctacacctccggcagcaccggaacgccgaagggcgcgatgtacaccaatcggttggccgccacgatgtggcaggggaactcgatgctgcaggggaactcgcaacgggtcgggatcaatctcaactacatgccgatgagccacatcgccggtcgcatatcgctgttcggcgtgctcgctcgcggtggcaccgcatacttcgcggccaagagcgacatgtcgacactgttcgaagacatcggcttggtacgtcccaccgagatcttcttcgtcccgcgcgtgtgcgacatggtcttccagcgctatcagagcgagctggaccggcgctcggtggcgggcgccgacctggacacgctcgatcgggaagtgaaagccgacctccggcagaactacctcggtgggcgcttcctggtggcggtcgtcggcagcgcgccgctggccgcggagatgaagacgttcatggagtccgtcctcgatctgccactgcacgacgggtacgggtcgaccgaggcgggcgcaagcgtgctgctcgacaaccagatccagcggccgccggtgctcgattacaagctcgtcgacgtgcccgaactgggttacttccgcaccgaccggccgcatccgcgcggtgagctgttgttgaaggcggagaccacgattccgggctactacaagcggcccgaggtcaccgcggagatcttcgacgaggacggcttctacaagaccggcgatatcgtggccgagctcgagcacgatcggctggtctatgtcgaccgtcgcaacaatgtgctcaaactgtcgcagggcgagttcgtgaccgtcgcccatctcgaggccgtgttcgccagcagcccgctgatccggcagatcttcatctacggcagcagcgaacgttcctatctgctcgcggtgatcgtccccaccgacgacgcgctgcgcggccgcgacaccgccaccttgaaatcggcactggccgaatcgattcagcgcatcgccaaggacgcgaacctgcagccctacgagattccgcgcgatttcctgatcgagaccgagccgttcaccatcgccaacggactgctctccggcatcgcgaagctgctgcgccccaatctgaaggaacgctacggcgctcagctggagcagatgtacaccgatctcgcgacaggccaggccgatgagctgctcgccctgcgccgcgaagccgccgacctgccggtgctcgaaaccgtcagccgggcagcgaaagcgatgctcggcgtcgcctccgccgatatgcgtcccgacgcgcacttcaccgacctgggcggcgattccctttccgcgctgtcgttctcgaacctgctgcacgagatcttcggggtcgaggtgccggtgggtgtcgtcgtcagcccggcgaacgagctgcgcgatctggcgaattacattgaggcggaacgcaactcgggcgcgaagcgtcccaccttcacctcggtgcacggcggcggttccgagatccgcgccgccgatctgaccctcgacaagttcatcgatgcccgcaccctggccgccgccgacagcattccgcacgcgccggtgccagcgcagacggtgctgctgaccggcgcgaacggctacctcggccggttcctgtgcctggaatggctggagcggctggacaagacgggtggcacgctgatctgcgtcgtgcgcggtagtgacgcggccgcggcccgtaaacggctggactcggcgttcgacagcggcgatcccggcctgctcgagcactaccagcaactggccgcacggaccctggaagtcctcgccggtgatatcggcgacccgaatctcggtctggacgacgcgacttggcagcggttggccgaaaccgtcgacctgatcgtccatcccgccgcgttggtcaaccacgtccttccctacacccagctgttcggccccaatgtcgtcggcaccgccgaaatcgtccggttggcgatcacggcgcggcgcaagccggtcacctacctgtcgaccgtcggagtggccgaccaggtcgacccggcggagtatcaggaggacagcgacgtccgcgagatgagcgcggtgcgcgtcgtgcgcgagagttacgccaacggctacggcaacagcaagtgggcgggggaggtcctgctgcgcgaagcacacgatctgtgtggcttgccggtcgcggtgttccgttcggacatgatcctggcgcacagccggtacgcgggtcagctcaacgtccaggacgtgttcacccggctgatcctcagcctggtcgccaccggcatcgcgccgtactcgttctaccgaaccgacgcggacggcaaccggcagcgggcccactatgacggcttgccggcggacttcacggcggcggcgatcaccgcgctcggcatccaagccaccgaaggcttccggacctacgacgtgctcaatccgtacgacgatggcatctccctcgatgaattcgtcgactggctcgtcgaatccggccacccgatccagcgcatcaccgactacagcgactggttccaccgtttcgagacggcgatccgcgcgctgccggaaaagcaacgccaggcctcggtgctgccgttgctggacgcctaccgcaacccctgcccggcggtccgcggcgcgatactcccggccaaggagttccaagcggcggtgcaaacagccaaaatcggtccggaacaggacatcccgcatttgtccgcgccactgatcgataagtacgtcagcgatctggaactgcttcagctgctctgacggatatcaggccgccgcgcgcacctcgtcggtgcgttcggcgccttcgcgccggaggcgaaacaggaataccgccgagccacccaggacagcggcgtagacgatgacgaagctgttgatcaggacctgggcgaccggccaccacggcgggaacaggaacagcccgacgacaacgtagtccgggctgtattcccacgtccacgcgccgatcgagacgaagagcgcggccgaggcaagccaccaccacggctgcgactgcgccctgtgcagtagatagacgaacaggggaacgaaccacacccagtggtggtcccaggagaacggcgagaccgcgcaggcggtgaggccggcgagggtgaccgcgaggagctgttcgccacgccgatacaggccgatggtgacggccagactcgccagcgcgacggagcccgcgatgagcagccacagccacaccggcgccgggtgatgggtcaggtgcgcgatggcgccgcggatggattgattggacgggtgcatatcgtccgcgatccgattggactggaagaacgtcgaggtccagtactgccgggaatcggcgggcagcacgatccaggcgaggacgatggacgcgatgaacaccgccacggcggtgcacgcggaccgccactgccgcaacgcgaggaattgcacgacgaagta

SEQ ID NO: 52 - protein, 1173 aa, Nocardia iowensis
MAVDSPDERLQRRIAQLFAEDEQVKAARPLEAVSAAVSAPGMRLAQIAATVMAGYADRPAAGQRAFELNTDDATGRTSLRLLPRFETITYRELWQRVGEVAAAWHHDPENPLRAGDFVALLGFTSIDYATLDLADIHLGAVTVPLQASAAVSQLIAILTETSPRLLASTPEHLDAAVECLLAGTTPERLVVFDYHPEDDDQRAAFESARRRLADAGSLVIVETLDAVRARGRDLPAAPLFVPDTDDDPLALLIYTSGSTGTPKGAMYTNRLAATMWQGNSMLQGNSQRVGINLNYMPMSHIAGRISLFGVLARGGTAYFAAKSDMSTLFEDIGLVRPTEIFFVPRVCDMVFQRYQSELDRRSVAGADLDTLDREVKADLRQNYLGGRFLVAVVGSAPLAAEMKTFMESVLDLPLHDGYGSTEAGASVLLDNQIQRPPVLDYKLVDVPELGYFRTDRPHPRGELLLKAETTIPGYYKRPEVTAEIFDEDGFYKTGDIVAELEHDRLVYVDRRNNVLKLSQGEFVTVAHLEAVFASSPLIRQIFIYGSSERSYLLAVIVPTDDALRGRDTATLKSALAESIQRIAKDANLQPYEIPRDFLIETEPFTIANGLLSGIAKLLRPNLKERYGAQLEQMYTDLATGQADELLALRREAADLPVLETVSRAAKAMLGVASADMRPDAHFTDLGGDSLSALSFSNLLHEIFGVEVPVGVVVSPANELRDLANYIEAERNSGAKRPTFTSVHGGGSEIRAADLTLDKFIDARTLAAADSIPHAPVPAQTVLLTGANGYLGRFLCLEWLERLDKTGGTLICVVRGSDAAAARKRLDSAFDSGDPGLLEHYQQLAARTLEVLAGDIGDPNLGLDDATWQRLAETVDLIVHPAALVNHVLPYTQLFGPNVVGTAEIVRLAITARRKPVTYLSTVGVADQVDPAEYQEDSDVREMSAVRVVRESYANGYGNSKWAEVLLREAHDLCGLPVAVFRSDMILAHSRYAGQLNVQDVFTRLILSLVATGIAPYSFYRTDADGNRQRAHYDGLPADFTAAAITALGIQATEGFRTYDVLNPYDDGISLDEFVDWLVESGHPIQRITDYSDWFHRFETAIRALPEKQRQASVLPLLDAYRNPCPAVRGAILPAKEFQAAVQTAKIGPEQDIPHLSAPLIDKYVSDLELLQLL

SEQ ID NO: 53 - DNA, 1002 bp, Chlamydia pneumoniae
  source 1..1002: mol_type = genomic DNA
atgcgtattgcagttctgggtgtgaccggcctggttggccagaaattcgtggcactgctgcataaatggtatcgtgattgggttattgccgaagttgtggccagtaatagtaaatatggtcagagttatggtgatgcatgtatctggcaggaaccgattggtccgatgccggaaatggttcgcgatctgccgattcgcaaaattgaagaagtgcagagtgatattgttgtgagcttcctgccgagtagcgcagaaagtatggaagcctattgtctgagccagggtaaaattgtgttcagtaatgccagcacctatcgtatgcatagcagcgtgccgattattattccggaagttaatagtgatcacttccagctgctggaagaacagccgtatccgggcaaaattattaccagcccgaattgttgcgtgagtggtattaccctggccctggcaccgctgcgtaaattcagtctggatcatgttcatattgttaccctgcaaagcgcaagcggcgcaggctatccgggcgtgcctagcctggatctgctggcaaataccgttccgcatattgtgggcgaagaagaaaaaattctgcgtgaaaccgtgaaaattctgggtagcagcaaacagccgctgccgtgtaaactgagtgtgaccgttcatcgcgttccggttgcatacggtcataccctgagcctgcatgttaccttcagcaaagatgtggatctggatgaaattctgtatagttatcaggaaaagaacaaggagttcccgaatacctatcagctgtatgataatccgtggagcccgcaggcacgcaaacatctgagccatgatgatatgcgcgtgcatctgggcccgattacctatggcggtgacttccgcaccattaaaatgaatgtgctgattcataacctggttcgcggcgccgccggcaccttactggcttcaatggaaaattacttcttcgattatctgaagcgcgaaatgtgtctgcgctaa

SEQ ID NO: 54 - protein, 333 aa, Chlamydia pneumoniae
MRIAVLGVTGLVGQKFVALLHKWYRDWVIAEVVASNSKYGQSYGDACIWQEPIGPMPEMVRDLPIRKIEEVQSDIVVSFLPSSAESMEAYCLSQGKIVFSNASTYRMHSSVPIIIPEVNSDHFQLLEEQPYPGKIITSPNCCVSGITLALAPLRKFSLDHVHIVTLQSASGAGYPGVPSLDLLANTVPHIVGEEEKILRETVKILGSSKQPLPCKLSVTVHRVPVAYGHTLSLHVTFSKDVDLDEILYSYQEKNKEFPNTYQLYDNPWSPQARKHLSHDDMRVHLGPITYGGDFRTIKMNVLIHNLVRGAAGTLLASMENYFFDYLKREMCLR

SEQ ID NO: 55 - DNA, 1116 bp, Enhygromyxa salina
  source 1..1116: mol_type = genomic DNA
atgcatctgaccaccaaactgagcgccagcggccgtgaagcactgccgcgtgctgaacatcgtagtaccaccctgccgcgcatgagaatctgtattctgggcgcaaccggtctggttggtggtgaactgctgaccctgattgatcgcgcatggccggatgcagaactggcactgtatgccagtcgtgatcagaccctggaacatgcaggtcgcacccatcaggtgtatgccgccagtagtctggaacaggccgatacccgtggtggtgaattagccttcgttgcactggatgatcagtatagcgcccgctattgcccgcgtctggttgaactgggttatcgcgttattgataaaagcaatacctatcgcatgaatccggaagttccgctgattgttgcaggcgtgaatcatcgtcgtgccaccgcagataccaaactggtggccaatccgaattgcaccaccattccgctgagtctggccctggaaccgctgcgtgcacattatggcctgggcgaagtgaccgttagtagctatcaggcaattagcggcgccggcctggcccctctgaccagcttcgttgatgcaaccattgccggcttcagtcagatgcagcgtgcaggcgaactgagtggcgccctgctggatgccagcgcctatccgggtaatgtggtgccgcataatggcagtaccgatgatagcggcttcagtagtgaagaacgtaaactggttaatgaaagccgtaaaattctggaactgccgaaactgggcctgagtgcccagtgctgtcgcgttccggttccggtgggtcattatgaaaatgtgtgggttgaactggaacgtccggccgatgcagcaggcattaccgaactgctgggtgcagcaccgttcgtgcgctacttccctggtccggaaggtacccatctgaccgcactggcaaccgtgcagcatcgcgatgatgccctggtgggccgtgtgcgccgtgatggtcgtgataccaatggtcgtcgctggtgcctgaccgtggcagcagataatctgcgcctgggtgccgccaccaatgccgtgcgtattgcaagcgcctggttcgatgccgccgatccgagtctgcgtgtttaa

SEQ ID NO: 56 - protein, 371 aa, Enhygromyxa salina
MHLTTKLSASGREALPRAEHRSTTLPRMRICILGATGLVGGELLTLIDRAWPDAELALYASRDQTLEHAGRTHQVYAASSLEQADTRGGELAFVALDDQYSARYCPRLVELGYRVIDKSNTYRMNPEVPLIVAGVNHRRATADTKLVANPNCTTIPLSLALEPLRAHYGLGEVTVSSYQAISGAGLAPLTSFVDATIAGFSQMQRAGELSGALLDASAYPGNVVPHNGSTDDSGFSSEERKLVNESRKILELPKLGLSAQCCRVPVPVGHYENVWVELERPADAAGITELLGAAPFVRYFPGPEGTHLTALATVQHRDDALVGRVRRDGRDTNGRRWCLTVAADNLRLGAATNAVRIASAWFDAADPSLRV

SEQ ID NO: 57 - DNA, 939 bp, Planctomycetes bacterium
  source 1..939: mol_type = genomic DNA
atgaccccgccggctcagcctcgccgtggttggaaactgccggcacagccgcgtattgcagtggtgggtgcccgtggtctggttggtaaagcactgctggcagtgctgcatcagcgtgagttcccgcatcagggcgttgccgcaatgcgtgttggcggcttctgtccgcagaaagttgccgatgccgatctgatcttcctggccggtccggcaagtctggcacataaattcgcccgcgaactgaccagtgaaaccagagtggttattgatctgagcgaagcatggcgtatggaccctagcgttccgctggtgctggatggcctgaatctggatgatgtgcatcagcatcagggtctgattagtagtccgaattgtaccaatggtggcctgctgcgtgttctggatattctgcgtcgtgattgtaccctgggtcaggtggtggtgaccaccttccaggcagcaagcggcggtggtcgtaaactgctgcaaagcattgccgataccgatgatccgctgcacgctaatgttattccgcagtgcgatgcattcgcagaagatggcgcaaccctggaagaacatcgtgtggttgcagaaagtctgcgcctgctggaagatgcaagcctgaatattagcgcaacctgtgttcgcgtgccggttgaagtgggtcatggtctgagtgtgtggattgaagcaagtgaagacttcgatctgaccgccgttgccgaaaaactgcgtgccgaagatggcatggtgctgagccatagccaggaagattatccgaccccgaaacagattcgtggtaaagaaggcgtgttcctgggtcgcctgcgtcatggtgcaaaaccgaatcagctgcaactgtggattgtgaccgataatctgctgaccggtgccgccagcaatgccgttgcaattgccgaagcactgctgccgagttaa

SEQ ID NO: 58 - protein, 312 aa, Planctomycetes bacterium
MTPPAQPRRGWKLPAQPRIAVVGARGLVGKALLAVLHQREFPHQGVAAMRVGGFCPQKVADADLIFLAGPASLAHKFARELTSETRVVIDLSEAWRMDPSVPLVLDGLNLDDVHQHQGLISSPNCTNGGLLRVLDILRRDCTLGQVVVTTFQAASGGGRKLLQSIADTDDPLHANVIPQCDAFAEDGATLEEHRVVAESLRLLEDASLNISATCVRVPVEVGHGLSVWIEASEDFDLTAVAEKLRAEDGMVLSHSQEDYPTPKQIRGKEGVFLGRLRHGAKPNQLQLWIVTDNLLTGAASNAVAIAEALLPS

SEQ ID NO: 59 - DNA, 1023 bp, Cuniculiplasma divulgatum
  source 1..1023: mol_type = genomic DNA
atggataagctgagtgttggtgtgctgggttgtaccggtctggtgggtgaaaccttcctgaaactgctggaaaatcatccgtggttccgtgtggatggcatctatgcaagtaaaaatagcgcaggcctgctgatgaaagccggtagtattgaaagtaatgaactgaccgtgaaagatagtaccgtggaaaatattgttaacgcaaaacatgatattgtgttcagcgccattagcgatctgaatgccggtccgattgaactggaactgagcaaacatggccagtgggtgttcaccaatgccagtgccaatcgtctgaatgaagcaattccgctggttgttccggaaattaatagtgatcatctgaatgttatcaacgaagatgatggcttcattgtggccaatggtaattgtagcaccattggtctgagcctgggcattgatccgatcttcgatctgatgccgcagcatctgaccgttaccaccctgcaaagtattagcggcgccggttatccgggtattccgagcatggatattctggcaaatgttattccgttcattaatggcgaagaaggcaaactggaaaaagaaaccaaaaaaatcttcggcaaaatcagtaatggtaaaatgcatgaaagcagcctgaatattagtgcaaccgcaacccgtgtgccggtgaaaattggtcatattattagtgccaccgtggaagttcgtgaagatgttgatgaaaaactggtgctgaataaaattaagaactacggtaatggcagtctgaaaggccacttcccgaccctgccgaaagaaagtattatgttcgtgccgggtaatgatcgtccgcagccgctgctggatagtaatagcgatgatgttctgaaagaaatgcaggttaaagtgggccgcattcgcgtgagcggtaaatatattagtctgattattctggtgaacaacctggttcgcggcgccgcaggcgcaagcattctgaatgccgaaattgtgagtaaaatgcgcggtgccattagttaa

SEQ ID NO: 60 - protein, 340 aa, Cuniculiplasma divulgatum
MDKLSVGVLGCTGLVGETFLKLLENHPWFRVDGIYASKNSAGLLMKAGSIESNELTVKDSTVENIVNAKHDIVFSAISDLNAGPIELELSKHGQWVFTNASANRLNEAIPLVVPEINSDHLNVINEDDGFIVANGNCSTIGLSLGIDPIFDLMPQHLTVTTLQSISGAGYPGIPSMDILANVIPFINGEEGKLEKETKKIFGKISNGKMHESSLNISATATRVPVKIGHIISATVEVREDVDEKLVLNKIKNYGNGSLKGHFPTLPKESIMFVPGNDRPQPLLDSNSDDVLKEMQVKVGRIRVSGKYISLIILVNNLVRGAAGASILNAEIVSKMRGAIS

SEQ ID NO: 61 - DNA, 1062 bp, Geobacillus sp.
  source 1..1062: mol_type = genomic DNA
atggaaggcgaatggctgatggcccagaaacagtatcatgttgccgtggtgggcgcaaccggtgccgttggtcagcagatggtgcgtaccctggaagatcgtaacttcccggtgggcaaactgaccctgctgagtagcgaacgtagcgcaggcaaaaaaatgcgcttccgtagcgaagaaattgaagtgcaggccgccagcccggaacgcttcaatggtgtggatattgcactgttcagtgcaggtggcgccgtgagcaaagccctggcaccggaagccgttcgtcgcggtgcaattgttattgataataccagcgccttccgtatggaagaaaatgtgccgctggttgttccggaagttaatgaaagcgatctgacctggcataatggtattattgcaaatccgaattgcagtaccattcagatggttgttgccctggaaccgattcgcaaagccttcggcctggaacgtgtgattgtgagtacctatcaggccgtgagcggtgccggcgcacaggcaattgaagaactgaatgaacagaccaaagccgtgctggaaggtaaaccggttgaagcaaatattctgccggttaaaagcgatcgtaaacattatccgattgcattcaatgccattccgcagattgataaattccaggataatggcttcaccttcgaagaaatgaaaatgattaacgaaaccaagaagatcatgcacatgccggaactgaaagttgccgccacctgcgtgcgtattccggttgcaagtggtcatagtgaaagcgtgtatattgaaattgaacaggatggtgtgaccgccagcgatctgcaagcagtgctgcgcgaagcccctggtgttgtgctgcaagatgatccgagtgaacagctgtatccgatgccggcccattgtgtgggcaaatatgatgtgttcgttggccgtattcgccgtgatctggataatcgccgtggcttccatctgtggattgtggccgataatctgctgaaaggtgcagcaagcaatagcgttcagattgcagaaagtctgctgaaactgggtctgatttaa

SEQ ID NO: 62 - protein, 353 aa, Geobacillus sp.
MEGEWLMAQKQYHVAVVGATGAVGQQMVRTLEDRNFPVGKLTLLSSERSAGKKMRFRSEEIEVQAASPERFNGVDIALFSAGGAVSKALAPEAVRRGAIVIDNTSAFRMEENVPLVVPEVNESDLTWHNGIIANPNCSTIQMVVALEPIRKAFGLERVIVSTYQAVSGAGAQAIEELNEQTKAVLEGKPVEANILPVKSDRKHYPIAFNAIPQIDKFQDNGFTFEEMKMINETKKIMHMPELKVAATCVRIPVASGHSESVYIEIEQDGVTASDLQAVLREAPGVVLQDDPSEQLYPMPAHCVGKYDVFVGRIRRDLDNRRGFHLWIVADNLLKGAASNSVQIAESLLKLGLI

SEQ ID NO: 63 - DNA, 1002 bp, Roseovarius azorensis
  source 1..1002: mol_type = genomic DNA
atgatcccggtggtggttctgggcgcaagtcgcctgattggtcaggccctgctgcaacgcctggcaggccatccggcattcgcagttcgtgcactgtgcgatctgggtccggaacgtccgccgatgccgtatggtgaagtgtgcgattggcgcatgaccagtagtatgccgaccgaactggtgaccctggatcatcatagtggcccggcaagtagtctgagccgcctgagcaaaccgggtctgctggtgctgagtgttctgccggatggccagagcgcagaagcagatcgtgcattcgccgaaggcggcgcccgcgttattacccatgccgaagaatggcgtctgcatccggatattccgctgattattccggatattcatccgattccgagtgaaccgggtattattgccaccccgaattgtaccaccgtgatgctgagtctgctgctgaccgcactgcgcaatgaacatcgtattgatgccgttagcgtggttgcactgcaagcactgagcggcgccgatctgaccggtccgagcgctatggatatgctgggtaatgttgatccgcatctggatggcgaagcacgtgcactggaacgtgaaaccggccgcatcttcagcgaagagttcccggttagcgcacgctgcgtgcgcgtgcctgtgaatgttggtcataccctgctggttagcttcaaaaccagacgtccgagcgaactgggtgatattctgacctgcctggatgacttccgcctgaccagcgcacgtgcccgtaccgaaaccgcaatggaacgtccgttcgtgctgttccgcaatccgggccgcccgcgtcctgtgcctgatgcagatgcaggtcgtggcatggccctgagcattggtggtgttgcccgttgcccggttctggattggcgctgcgttctggttggcaataatgtggaacgcggtagcgcagccacaatggttctgaccgcagaaatgctggcccagagcctggtggacttctaa

SEQ ID NO: 64 - protein, 333 aa, Roseovarius azorensis
MIPVVVLGASRLIGQALLQRLAGHPAFAVRALCDLGPERPPMPYGEVCDWRMTSSMPTELVTLDHHSGPASSLSRLSKPGLLVLSVLPDGQSAEADRAFAEGGARVITHAEEWRLHPDIPLIIPDIHPIPSEPGIIATPNCTTVMLSLLLTALRNEHRIDAVSVVALQALSGADLTGPSAMDMLGNVDPHLDGEARALERETGRIFSEEFPVSARCVRVPVNVGHTLLVSFKTRRPSELGDILTCLDDFRLTSARARTETAMERPFVLFRNPGRPRPVPDADAGRGMALSIGGVARCPVLDWRCVLVGNNVERGSAATMVLTAEMLAQSLVDF

SEQ ID NO: 65 - DNA, 987 bp, Chlamydia sp.
  source 1..987: mol_type = genomic DNA
atggaaaagaagaagaagaccattagcgtgattggcgcaagtggtgccgttggcaaagaaattgttcgcctgctgattgaacgtcgcttcccgttcaaaaaactgctgctgttcggcagcagtcagagtgccggtaaacgtatcttcagtcgcccggtgaaagaactggccaccctggaaggcgtggatctggcattcttcgccgcaggcaatgaaattagcaaagaatggattccgaaagccgaatgcacctgcattgatagtagtagcgtgttccgtaaaaccttcccgctgattattccggaaattaatgcacatgccatgcagggtaatcgcttcgcaagtccgaattgtgttgccaccctgctgctgatgccgctgttcccgctgcatcagaaactgcgtgccaaacgtattattctgagtacctatcaggcagccagcggtgccggttatccgatgatgaaacatctggaaaatgaaaccaaagcctgtctgaaaaaacgcgcattcccgccgctgctgccgttcccttatgcattcaatctgttcccgcataatagcgaactggatcatagtggttatgtggatgaagaaattaaaatccgcgaagaaacacaaattatcctggaagataataagctgcaaattagtgcaacctgcgttcgtgtgccggtgctgcgcgcccatagtctgagcgttaatgttgagttcgccaaaccgttcagtctgccggatatatatcatattctgagcagccagccgggcctgcaaatcttcgaaaatcgcggcaaaaatcgcttcccgaccccgtgggatgccaccggtaaagaagatgtgtattgtggtcgcttccgtctggaccctacccgtaaaaataccctggagttctgggttgttggcgatcagctgctgaaaggcgccgccctgaatgcagtgcagattgcagaacgttgcatgatgaatcagccggccgccaaaaaataa

SEQ ID NO: 66 - protein, 328 aa, Chlamydia sp.
MEKKKKTISVIGASGAVGKEIVRLLIERRFPFKKLLLFGSSQSAGKRIFSRPVKELATLEGVDLAFFAAGNEISKEWIPKAECTCIDSSSVFRKTFPLIIPEINAHAMQGNRFASPNCVATLLLMPLFPLHQKLRAKRIILSTYQAASGAGYPMMKHLENETKACLKKRAFPPLLPFPYAFNLFPHNSELDHSGYVDEEIKIREETQIILEDNKLQISATCVRVPVLRAHSLSVNVEFAKPFSLPDIYHILSSQPGLQIFENRGKNRFPTPWDATGKEDVYCGRFRLDPTRKNTLEFWVVGDQLLKGAALNAVQIAERCMMNQPAAKK

SEQ ID NO: 67 - DNA, 2325 bp, Polaribacter filamentous
  source 1..2325: mol_type = genomic DNA
atgaagatcaagtaccagcaggcagcaggccgtggtctgatgaatcagaccgccttcgatctgcgcatgaattatctggaaaccttatcaaaagacatcagtaaagttgttaagagcgaaagtattgcactgagccagattcagaataatattgaatcattcatcggcaccgttgaaattccgctgggtctgattggcccgctgctgttcattgataaaaataataaaaccgagctggtgcatagtgcaattgccaccaccgaaggcgcactggttgccagtctgaatcgtggtgccaaagccattagcgaaagcggtggcttcgaagcccatattgtgcatcagaaaatggttcgtaccccgatgtataccttcaaacgcctgagcgaaagtgttgcattcgatgaatggattaaaagcaacttcaaaaagatcaaggaccaggcacagatgcatagtaatcatgcagaactgctggaaattgcaagcgtgattctgggcaaaattgttcatctgaaattcgtgtatagcaccagcgatgccagcggccagaatatgaccaccagctgcacctggaatgcctgtctgtggattgaagaaaacttcgaactggcaaccagtattgaaattctgaacttcgttattgagggtaatggcgccagtgataaaaaagttagcttctatgccatgcagaatggccgtggttgtcatgtgattagtgaatgcttcctgaccaatgaagtgattgaaaaaaccttacgcaccaatgcaaaagaaatgttcagtagttatacccatagtctgagtattagccgtctggatggtatggttggtcataatgtgaatgttgccaatgccattgccggtatctatgccagcaccggtcaggatctggcatgtattcatgaaagcagtattggtattctgcaaattgaactgaccgatgaaggtctgtatctgagcctggttctgccgaatctggtggttggtaccgtgggtggtggcacccatctgccggtgccgagcaaaattctggaactgatgggttgcaaaggtgcaggtaaaattgaacgcttcgcaaaactgattgccggcttcgccctgagcattgaaattagtaccctggccgccattgttagcggccagttcgcacgtgcacatcagaaactgggtcgcaataaaccggtgaaatggctgctgcgtagtgaagtggatgccgacttcattaaaacacatgtgccgtacttccatgccgaaattagtagtgtgagcttcaataatgaaatcgaagttgaaaacggtatcctgaccgatctgaccaaaaaaattaccaaaaaagccattggcttcattcaggcagatctgcatgatattgatggtaaaaaacatccgctgctgctgaaaagcaaagccctgggtaaagaagttctggatggtctgcacttcatggcaagtaatgttagcgtgggtctggcagatattctggccaaacattatgaagtgctggaatataaagacaatcataccaaagaaatcgcagtgtatgaagccctgcaacatattggttatccgttcatgccgattgtgtatggtaccaaaaaagatagcgaacgtgaaatctatctgattctgatggaacgcctggccagtgaaaatatgctgctgattaatagtgaaagtaccccggaaaaatggaccctgccgattattaaaaaaaccattgatagcatccacctggtgcataccaacttcacctatgaaaccaataaaattctgagtatcgcaccgttcgatattgaaaaagtgctggaactgtataccgcattcgttgccctgaatcgcaaagattatgattatctgattgccgatgatcgcttcgatgaactgaccagcttcattaatgattggagcaccaatggttatcagccgaaaagcaaactgaccctgattcataatgacttcaatccgcgcaatattgcaattcgtgccaatggtgatccgtgcatctatgattgggaactggccacctatggcattccgcagcgcgatatattcgaactgctggcattcaccctgaccccgaacttcgaaagtagtgatgcaattgatgttctgaaacatcacttcaaacaggttcagagtctgaataatcaggattatagttggagcgattatctgtatgatctgaaactgggcggtcaggccttcctggttagccgtgtgaacttctatctgaccggtagcattctgatgaattatccgttcattgaacgcgtgttcgcaaccagcttcaaaattctggatctgctgaaaaaaaccacctaa

SEQ ID NO: 68 - protein, 774 aa, Polaribacter filamentous
MKIKYQQAAGRGLMNQTAFDLRMNYLETLSKDISKVVKSESIALSQIQNNIESFIGTVEIPLGLIGPLLFIDKNNKTELVHSAIATTEGALVASLNRGAKAISESGGFEAHIVHQKMVRTPMYTFKRLSESVAFDEWIKSNFKKIKDQAQMHSNHAELLEIASVILGKIVHLKFVYSTSDASGQNMTTSCTWNACLWIEENFELATSIEILNFVIEGNGASDKKVSFYAMQNGRGCHVISECFLTNEVIEKTLRTNAKEMFSSYTHSLSISRLDGMVGHNVNVANAIAGIYASTGQDLACIHESSIGILQIELTDEGLYLSLVLPNLVVGTVGGGTHLPVPSKILELMGCKGAGKIERFAKLIAGFALSIEISTLAAIVSGQFARAHQKLGRNKPVKWLLRSEVDADFIKTHVPYFHAEISSVSFNNEIEVENGILTDLTKKITKKAIGFIQADLHDIDGKKHPLLLKSKALGKEVLDGLHFMASNVSVGLADILAKHYEVLEYKDNHTKEIAVYEALQHIGYPFMPIVYGTKKDSEREIYLILMERLASENMLLINSESTPEKWTLPIIKKTIDSIHLVHTNFTYETNKILSIAPFDIEKVLELYTAFVALNRKDYDYLIADDRFDELTSFINDWSTNGYQPKSKLTLIHNDFNPRNIAIRANGDPCIYDWELATYGIPQRDIFELLAFTLTPNFESSDAIDVLKHHFKQVQSLNNQDYSWSDYLYDLKLGGQAFLVSRVNFYLTGSILMNYPFIERVFATSFKILDLLKKTT

SEQ ID NO: 69 - DNA, 1209 bp, Lactobacillus kefiranofaciens
  source 1..1209: mol_type = genomic DNA
atgaagttctaccagctgaccccgaaacagcgtcgcgaaattctgcaaagcgaaggcattaaactggaaaaaattgatgatcaggtgctggcacgtctggatcagctgagtgaaaatgtgattggccagctgcgcctgccgctgggcgttgttcagaatctgcaagtgaatggtcataaatattgggtgccgatgaccgttgaagaaccgagcgttgttgccgccgccaatcatggtgccaatatcttcgcccagaatggtggtgtgcaggccgttagcaaacgcgatggcatctatggccagattgtgctgcgcgttaatgatgacttcgttctggcagaactggaaaatgccttcccgcatctgattgccctggcaaatcagaaattcgcaagtctggtgaaacatggtggtggcacccgcgaaattaccgcaaaacagattaataaactggtgtatctgcgtgttctggttgatccggccgaagcaatgggtgccaataaaaccaatagcattctggagttcctgagcaccaaactggttgaactgccgggtgtggcagaaaaactgttcgcaattctgagtaattatccgagccagctgaccaccgcaaaagtgaaactggaccctgcaaccctgggcggtaaacgcgtggccgaacgtattgccctgctgagtaaaattggttatgaagatccgtatcgcgcagttaccaataataaaggtattatgaacggtgccgatgcagttctgctggccaccggtaatgattatcgtgccattgaaagtgcaaccgccgttctggcaagccagaccggtcagtatcgcagcctgagtaaatggacaatggaagatggtctgctggtgggcgaactgaccctgccgatggcaattggcgttgttggcggcagtattaaagcccgtgaagatgtgcagcagagcttcgccattctgggccaggttaccagcaaacagctggcagaaattattaccggcgtggcactggccaataatctggcagcactgctggccattagtaccgttggcattcaggcaggccacatgaaactgcaagcacgtaacttcgttaccgaactggatgcaaatgcagatgaaaaaaaacaggtgctggcggaaatgattgcccgtaaagattatagcgaaagttatgcacgcaaaattctgcaagaaattcgcaaagataagtaa

SEQ ID NO: 70 - protein, 402 aa, Lactobacillus kefiranofaciens
MKFYQLTPKQRREILQSEGIKLEKIDDQVLARLDQLSENVIGQLRLPLGVVQNLQVNGHKYWVPMTVEEPSVVAAANHGANIFAQNGGVQAVSKRDGIYGQIVLRVNDDFVLAELENAFPHLIALANQKFASLVKHGGGTREITAKQINKLVYLRVLVDPAEAMGANKTNSILEFLSTKLVELPGVAEKLFAILSNYPSQLTTAKVKLDPATLGGKRVAERIALLSKIGYEDPYRAVTNNKGIMNGADAVLLATGNDYRAIESATAVLASQTGQYRSLSKWTMEDGLLVGELTLPMAIGVVGGSIKAREDVQQSFAILGQVTSKQLAEIITGVALANNLAALLAISTVGIQAGHMKLQARNFVTELDANADEKKQVLAEMIARKDYSESYARKILQEIRKDK

SEQ ID NO: 71 - DNA, 1206 bp, Pseudobacteriovorax antillogorgiicola
  source 1..1206: mol_type = genomic DNA
atgagtaccaaaaccgaaagtgttaaagccgccccgcagccgcgtacccagcctaaaaatgaacgcagcgaaagtagtaaaagtagtctggatctgtggctggaaaaaggtctggtgctgaatagtagcctggatcatattcgtgatacccatctgtgcgccgaaaaaaccagaggtaatattgaaaacttcctgggtgcagtgcagattccggtgggtctggccggtccgctgctgttccagggtgaatgggcccagggcagtatcttcgccccgttcgccaccaccgaaggcgcactggtggcaagcgcaagtcgtggcgccaaagcactgagcgaaagtggcggcgttcatacccgtgttctgagccagaatatgtatcgcgcaccgagcttcgtgtgtaaatcattcctggaagccaaaaaactgggcgacttcctggtggatcatattgaagccctgcaagatcaggtgagcaaagtgacccgttatggtaaaattctgaatattgaaccgcattataacggtcgtaccctgcacgctctgattcgctatagcaccggtaatgcagcaggtcagaatatggttaccattgccagtgcacagctgggcaaatgggtgatggaagaagccccgcgtcagctgggtattgaaattgcagagttccatattgaaggcggtctgaatggcgataaaaaagtgaacttcctgaacttcattagcggccgtggttgtcgcgtggttgccgaagttgaactgccgaatattgtggttgaacaggtgctgaaaaccaatgcagatgatctggcccatcattatcagcgcggtgccgcaacctgtctgatgaatggtggtgttggcgttaatgtgaatattgcaaatgcaattgccgcaatcttcattgcaaccggtcaggatgccgccagcgtgcatgaaagcagtctgggtcatctgaacttcgaacgtaccaaagatggcctgtatgccagcctgatgctgccgagtctggcaattggcaccgtgggtggcggcacccgcctgcctggtcaaagtgaagcactgagtattatgggttgtcagggtcaggatgtgcgtcgcctggccgaaattattgcaggttattgcctgggtctggatctgagcacctggagcgccattgcaagtcatagcttcgttcaggcacatcagaccctgggtcgccgctaa

SEQ ID NO: 72 - protein, 401 aa, Pseudobacteriovorax antillogorgiicola
MSTKTESVKAAPQPRTQPKNERSESSKSSLDLWLEKGLVLNSSLDHIRDTHLCAEKTRGNIENFLGAVQIPVGLAGPLLFQGEWAQGSIFAPFATTEGALVASASRGAKALSESGGVHTRVLSQNMYRAPSFVCKSFLEAKKLGDFLVDHIEALQDQVSKVTRYGKILNIEPHYNGRTLHALIRYSTGNAAGQNMVTIASAQLGKWVMEEAPRQLGIEIAEFHIEGGLNGDKKVNFLNFISGRGCRVVAEVELPNIVVEQVLKTNADDLAHHYQRGAATCLMNGGVGVNVNIANAIAAIFIATGQDAASVHESSLGHLNFERTKDGLYASLMLPSLAIGTVGGGTRLPGQSEALSIMGCQGQDVRRLAEIIAGYCLGLDLSTWSAIASHSFVQAHQTLGRR

SEQ ID NO: 73 - DNA, 1281 bp, Lactobacillus paracasei NRIC 0644
  source 1..1281: mol_type = genomic DNA
atgaagttctacgaactgagcccggaaaaacgtcgtgatcagctggttcaggaaggttggctgaccacccaggatgccgcactgctggccggtacccatagtctgccggaagttaccggcgcacgcctgattgaaaatgccattggcgagttcccgctgccgctgggtgttgcacgtaatctgctggtgaatggtcagctgcatcaggtgccgattgcagatgaagaaccgagtgtgattgccgccgccagtaatggtgcccgtctggccaccgcaaatggtggcgtgcgtacccatgttgccgcacatcgcgttgttgccgaagtggttctgaccaatctgaccgatctggtgcaggcacgccagaccattctggcacatcagaccgatattcagaaagtgattgccgtggcccatccgagtatgattcagcgtggcggtggcctggatcagctgaccgtggaaagtctgggtgcccagttcctgaaaattcgtctgaccctggaccctcagcaggcaatgggcgcaaattatgcaaataccgtggccgaagcagttgccgccgcagttaccagttgggttgatggtgatgtgctggttagcattctgaccaatgcaccgaccgaactggtgaccgcagaagttagcctggaaccggttagcctggcaaccaaagcagtgagtggcgatgtgattgccaaaaaaattgtgcagctgagtgatctggcattcgtggatgcagaacgtgcagtgacccataataaaggtattctgaatggtattatcggcgccgttctggcaaccggcaatgatacccgtgccgtggccgcaagcattggcgcattcgcctgcgccagtggtcgttatcagccgctgagccgttggtatatggatcagggtcatctggtgggtcatctgcaactgccgctgccgatgggcgcagttggtggtgccattggtgccctgccgatggcccaggtggtgcgtcgtctgggcggttatcagaatctggcaattatgcagcaggttattgccgcactgggtctggttcagaatctggccgcaatgcgtgccctggcaggtccgggtattcaggccggtcacatgaaactgcaagccaatgcactggccattgcagcaggtgccaccgaaaccgaactgccgatgctggttaatgccctgcgtcagggcagtatggatctgaaacatgcacagcagtatctgaccaccattcgcctgaataaaaaagttggtcagagcaaagatgaaaatcgcgattaa

SEQ ID NO: 74 - protein, 426 aa, Lactobacillus paracasei NRIC 0644
MKFYELSPEKRRDQLVQEGWLTTQDAALLAGTHSLPEVTGARLIENAIGEFPLPLGVARNLLVNGQLHQVPIADEEPSVIAAASNGARLATANGGVRTHVAAHRVVAEVVLTNLTDLVQARQTILAHQTDIQKVIAVAHPSMIQRGGGLDQLTVESLGAQFLKIRLTLDPQQAMGANYANTVAEAVAAAVTSWVDGDVLVSILTNAPTELVTAEVSLEPVSLATKAVSGDVIAKKIVQLSDLAFVDAERAVTHNKGILNGIIGAVLATGNDTRAVAASIGAFACASGRYQPLSRWYMDQGHLVGHLQLPLPMGAVGGAIGALPMAQVVRRLGGYQNLAIMQQVIAALGLVQNLAAMRALAGPGIQAGHMKLQANALAIAAGATETELPMLVNALRQGSMDLKHAQQYLTTIRLNKKVGQSKDENRD

SEQ ID NO: 75 - DNA, 1278 bp, Methanocella sp.
  source 1..1278: mol_type = genomic DNA
atggaaaagagcagtcagattagcggcttctataaactgccgccgcatgatcgcctgaaaaccgtgcagcagctggccggtctgaccgatgaagaagttgcaaccctgggtaataccggtgcactgaaaccggaacaggtggatcgtatggttgaaaatgtgattggtgttatgccggtgccgctgggcctggcagtgaacttccagattaatggtaaagattatctggttccgatggcaattgaagaaccgagtgttattgcagcagccagtaatgccgcaaaaatggcccgtgttgccggcggcttcaccaccagtagcaccggcccggtgatgcgtggtctggttcaggttgtgggcattaaagatccgcatagtgcacgcttcgcagtgctggcagaaaaagcaaaaattatggaaatctgtaacgcagtggaccctattctggttaaattcggcggtggctgcaaagatgttgaagcctatgttcgtcaggtgccgaataaaccgaaacatagcatgttagttgtgcatctgattgttgattgccgcgatgcaatgggtgcaaatgcagtgaatacaatggctgaaaccgttggcccgtatattgaaaaagtgaccggtggcaaagtgtgcctgcgtattattagtaatctggcagatcgtcgtctgatgcgcgccaaagcagtgtggagcaaagatgccattggcggcgaagatgtggtggatgcagtgattcgcgcctatgagttcgccctggccgatccgtatcgcaccgcaacccataataaaggtattatgaatggtatcagcgcagttaccctggcaaccggcaatgatacccgtgccattgaagccggcgcacatgcatacgctgccattaccggtacctatctgccgctgacctgctatgaaaaaaatgaaaatggtgatctggtgggcaccattgaactgccgctggccgttggcctggtgggcggtgcaaccgcttcacatccgaccgcaaaagcctgtgttcgtattctgggcgttaaaaccgcaagcgaactgggtgaaattatggccgcagttggcctggcccagaacttcgccgccctgcgtgccctggccaccgaaggtattcagcgcggtcacatgcagctgcatagccgtaatgtggcaattcaggcaggcgcaaccggcgatctgattgataaagtggccgaagcaatggttcgcgaaaaaaaagtgcgtgcagatcgtgcaaaagaactgctggaagaactgaaacagaaataa

SEQ ID NO: 76 - protein, 425 aa, Methanocella sp.
MEKSSQISGFYKLPPHDRLKTVQQLAGLTDEEVATLGNTGALKPEQVDRMVENVIGVMPVPLGLAVNFQINGKDYLVPMAIEEPSVIAAASNAAKMARVAGGFTTSSTGPVMRGLVQVVGIKDPHSARFAVLAEKAKIMEICNAVDPILVKFGGGCKDVEAYVRQVPNKPKHSMLVVHLIVDCRDAMGANAVNTMAETVGPYIEKVTGGKVCLRIISNLADRRLMRAKAVWSKDAIGGEDVVDAVIRAYEFALADPYRTATHNKGIMNGISAVTLATGNDTRAIEAGAHAYAAITGTYLPLTCYEKNENGDLVGTIELPLAVGLVGGATASHPTAKACVRILGVKTASELGEIMAAVGLAQNFAALRALATEGIQRGHMQLHSRNVAIQAGATGDLIDKVAEAMVREKKVRADRAKELLEELKQK

SEQ ID NO: 77 - DNA, 1308 bp, Capnocytophaga sp.
  source 1..1308: mol_type = genomic DNA
atgaagcagattaacggcttcagtaaactgaccaaagcagaaaaaattcagtggctgtgcgatacctacttcccggaagtggaaaatgcagcagccttcttcgaaaaatatcataatccggatgccgaactgcaaaaacgccatgatgagttcattgaaaataccgtgaccaattattacctgccgttcgcagtggccccgaacttcctgattaatggccgcacctataccgtgccgatggccattgaagaaagcagtgtggtggccgccgcaagtcgcgccgcaaaattctggcagggccgtggtggcttccataccgaagttctgagtaccgaaaaaaccggtcatgttcacttcttcttctatggtgaaaaagcacgtctggaagcattcttcgcacgtgttcagccgctgatgctggcaaatgccaaaccgattaccgccaatatggaaaaacgtggcggcggcattaccagtctgaccctggttgataaaaccgaagccctggcagattattatcagctgagcgcaaccttcgaaaccttagatgcaatgggtgcaaacttcattaatagttgcctggaatcattcgccgaaaccttccgtgaagaagcagaaaaagatgccaccattcgtggtcagtatgaaattgttatgagcattctgagtaactatgttccgaattgcctggttcgcgcagaagttcattgcccggtggatgaactggaaattgcccatctgaaaggtatggccgcagcaaaaaaattcgttcaggccctggccattgcaaatgccgatgtgtatcgtgccaccacccataataaaggtattatgaatggcattgacagtgttattctggccaccggtaatgacttccgtgccattgaagcaggcgtgcacgcttatgcagcacgtagtggcaattataaaagcctgagcaatgcctggattgaaggtgataccttccacttcgccctggaactgccgctggcactgggtaccgtgggcggtctgaccaccctgcatccgctggttaaaaccgccctggaaattctggaaaaaccgaatgcccgcgaactgatgcagattgtggcctgcgtgggtctggcacagaacttcagtgccgttagcagtctgattagcctgggtattcagaaaggccacatgaaaatgcatctgaataatatcttcaaccagctgggcgcaaccgatgaagaaaaacagcagctgaccgcatacttcaaagataaaaccgtgagccatagcgaagtggcagaagcactgaatcatctgaaaagccagaaataa

SEQ ID NO: 78 - protein, 435 aa, Capnocytophaga sp.
MKQINGFSKLTKAEKIQWLCDTYFPEVENAAAFFEKYHNPDAELQKRHDEFIENTVTNYYLPFAVAPNFLINGRTYTVPMAIEESSVVAAASRAAKFWQGRGGFHTEVLSTEKTGHVHFFFYGEKARLEAFFARVQPLMLANAKPITANMEKRGGGITSLTLVDKTEALADYYQLSATFETLDAMGANFINSCLESFAETFREEAEKDATIRGQYEIVMSILSNYVPNCLVRAEVHCPVDELEIAHLKGMAAAKKFVQALAIANADVYRATTHNKGIMNGIDSVILATGNDFRAIEAGVHAYAARSGNYKSLSNAWIEGDTFHFALELPLALGTVGGLTTLHPLVKTALEILEKPNARELMQIVACVGLAQNFSAVSSLISLGIQKGHMKMHLNNIFNQLGATDEEKQQLTAYFKDKTVSHSEVAEALNHLKSQK

SEQ ID NO: 79 - DNA, 1545 bp, Longimonas halophila
  source 1..1545: mol_type = genomic DNA
atgttcatcccgagcgccctgctgaaaaaactgtataccttcggtagcctggaaaataccgatcagggcgttcagttcggtattaaaaatcgcctgcaagatgcaaccctgaatgaactgaaacatgttgttattgatggcaccaaagtgccgctgagtgatgttcatctgcatctgggcgatggtaccaccattgatgccaccgccctggaagatagcaccgccattgacttcccgctgcgccgcaccctgagtgttgtgtgtaatcgcgaagccctggatgttggtaaacatgaaattagcattgcattcaaaagcaaaccgttcggcaccctggacttcaccgttgaagatgccattagcgaagcagatgaaaccttactgcgtattccgcgcaataaagatgatgattatagtgatgaagcgattgccgaacgccagaaattcattcgtagcttcaccgataccgatccggaacatagcttccagcatagcttcgatccgcatgtgaccaaaggtaatgccgaacacttcgtgggtgtgagtcaggtgccgctgggtattgccggcccgctgcaagtgaatggtgaacatgcccagggtgagttcctgattccgatggcaaccagtgaaggcaccctggtggcaagctataatcgtggtattaaagttgttaacctgagtggcggtgtgaaaagcaccgttgttaatgattgtatgcaacgtgccccggtgttcgtgttcagtgatgcccgcgcagcccgcgagttccgtgattgggttgataatcacatggatgaagttcgtcgcgaagcagaaagcaccagcagcgttgccaaactgctgtatattgatacctatctgagcaatgacttcgcctatctgcgcttcaattatcgcaccggcgatgccgccggtcagaatatggtgggtcgcgccaccttcgccgcctgcggttggattattgatcagtatccggaaccgattgaacacttctatctggaaagtaacttcgcaaccgataaaaaagcaagtcaggtgaatgttatgcgcacccgtggtaaacgtgtgaccgcagaagcaattattgatcgtagcgttctggaacagcacatgcgcgttgatccggaaagcctgcatgatcattggaatgttagcaccattggcgcattcctgagcggcgcaaataataatggtgcccatagtccgaatgcaattaccgccatgttcattgcaaccggccaggatgtggccaatgttagtgaaagtagcgcaggtgtgctgtataccaatgttaccgatgatggtgatctgaaaattagtctgaccattccgagcctgattgtggcaacctatggtggcggcaccggcctgccgacccagcacgagtgtctggaaatgatgggttgctatggtaaaggtaaagttcataaattcgcagaaatcgttgcaggcgttgccctggccggcgaactgagtctgggtgcagcaattagtagtagcgattgggttagtagccatgaaagttatggtcgcaatcgttaa

SEQ ID NO: 80 - protein, 514 aa, Longimonas halophila
MFIPSALLKKLYTFGSLENTDQGVQFGIKNRLQDATLNELKHVVIDGTKVPLSDVHLHLGDGTTIDATALEDSTAIDFPLRRTLSVVCNREALDVGKHEISIAFKSKPFGTLDFTVEDAISEADETLLRIPRNKDDDYSDEAIAERQKFIRSFTDTDPEHSFQHSFDPHVTKGNAEHFVGVSQVPLGIAGPLQVNGEHAQGEFLIPMATSEGTLVASYNRGIKVVNLSGGVKSTVVNDCMQRAPVFVFSDARAAREFRDWVDNHMDEVRREAESTSSVAKLLYIDTYLSNDFAYLRFNYRTGDAAGQNMVGRATFAACGWIIDQYPEPIEHFYLESNFATDKKASQVNVMRTRGKRVTAEAIIDRSVLEQHMRVDPESLHDHWNVSTIGAFLSGANNNGAHSPNAITAMFIATGQDVANVSESSAGVLYTNVTDDGDLKISLTIPSLIVATYGGGTGLPTQHECLEMMGCYGKGKVHKFAEIVAGVALAGELSLGAAISSSDWVSSHESYGRNR

SEQ ID NO: 81 - DNA, 4692 bp, Enterovibrio norvegicus
  source 1..4692: mol_type = genomic DNA
atgaagatgttcaccaaaggtatggaagtgagcgcattcgttgaagcactgcgcaatcgcggtgttcagctgtgggttgaaggtgaagcactgaaattcaaagccccgaaaggtgtgatgaccgccgaactgaaaagcattctgctggcaaaaaaaagcgaagtgattggcttcctgaatcatgtgccggatgaagttaccctgaccgcatatccggaagatcgctatgaaccgttcccgctgaccgatctgcaactggcctatattgtgggccgccgcaataattatgaactgggtggcgttggctgtcatagctatattgaactggatatgccgcgcctggatcatgcacgtctggaaaatgcatggcataaactgattctgcgtcatgaaatgctgcgcgccgtgattggcaccgatggcaaacagcagattctgaaacatgttaccctgccggaagtgatctgtcatgataccaaaggcctgagcgccagtgcattcgaaaccgccattattcaggcacgtaatgaaatggcatatcgccgttatgatagtacctgttggccgctgtatgaactgcgtctgacccagcatgatgatcgcagcattctgcatcatagcaccgatctgctgattgcagacttcgccagcattcagattctgctggcggaactgggtgaactgtatcagcatccggaaaaagaactggatgccattaatgtgagcttccgcgatattgtgctgtatcagcaggcatgtcatagtcagcaggaaagtgatagtaaatatcaggttgatcgccagtattggcagcagcgtattccgagcctgccgggtgccccggaactgcctattcatcagaatattcatccgccggcacatccgattccggatggtgaaccgagcttcgaacgcctgagcctggaagttagcccgccggaatggcaggcactgtgtcagattgcaaccagccatcatattaccccgagtggcgccgtgatgaatgccttcgccgaagtgctgcaacgttggagccgcaatgatgacttctgcattaatctgaccctgctgaatcgtaaagcaccgcatccggatgtgaatcgtattgtgggtgacttcattgcagttaatgttctggaaattgcacatgatcgtggtggtgacttcaaagaacgtgccgttgcactgcaacatcagctgtggaaagatctggaacatgatagctataccggcattgatgttctgcgtgatatgaccaaagatcgtaaaagcaataccctgattccggtggtgtataccagtaccgttggcgttagcggtgaagccctgccgaataatgcattcatgcatgatgcaaaactgaacttcggtattacccagaccccgcaggtgtggctggattgtcaggcaaccgaacgcaaaggcgccctgtgcgtggattgggatattcgtaaaggcgtgttcctgccgggtgtgattgaacaggccttcgatgcattcaaaaccttactgctgagcctgagtaaaagtaaaggtcattggaatagtcgtatgccggtgagcctgaatagtgattgtattgccctgcgtaaaaaactgaataccgaacgtaatgcaccgctgccggaaggcttcctgcacgcttcattctgtcatcatgccattaccagcccggataaagtggccctgattcatgatgcccagaccccgattaccttcacctatcgtgatatggccgaatggagcctgagcattgcagataccctgctggataaaggcctgcaaccgggcgaaccggttgcaatcttcctggataaaggtccgcagcagattgccgcaattatgggtattctgctggccggtggcgcctatgtgccgattgatgttattcagccggaaaaacgccgcaataccattctgcatgatgcctgcattcgtctgattctgaccgcacagaccgatgcaaatctgaattggccggcagatattcgtacaatggttattgcagaaccgccgaccctggaacgcaatgatctggccctgaaacagcgtctgagtcgtattattgatcagggcctgcaaagcgatacccgtaatcagctggcatatattctgtataccagcggtaccaccggccgcccgaaaggcgtgatgctgacccatgaaggcgttggtaataccattgcaggcttcaatgccgagttcggtattgaaagcgatgataaaaccttagccctggtgaattatagcttcgatctgagtgtgctggatatattctgcaccctgagtgccggcgcaaccctggttctgccgggtgatggttggcgtaataatccggaagtgtgggcaaaaaccattcagcatcagcgtgtgaccatctggaatagtgtgccggcccacatgcagatgctgatgagtctgtgtgaaaatagtcgtagtatggatctgagcaccctgcgtctggccttcattagcggcgattggattccggtgagtctgccgccgaaaattcatgaaatgttaccgaccctgagcctgaatagcctgggtggtccgaccgaaattagcgtgacctgtattgccaatcagattaaagatgttccggaaggtgcaaccagtattccgtatggtctgccgctgcgtaatcatcgcctgtatattcagaataatcagtatgaagactgcccggattgggttccgggtgaaatgctggttggtggtccgggcgtggcaaaaggcttcattaatgatccgaaacgtacccgcgaacgcttcattcttcatccggttagtggtgaacgcatgtataaaaccggcgatatatgtcgttatcgtccggatggtgttattgaaattctgggccgccaggataatcaggttaaaattcgtggtcatcgtattgaactgggtgaggtggaagcagccctgaatgaactgagctatgtgcagcaggccgtggcactggtgcgtagcggccctgccgaactggtgtgtgcactggtgttcaataccgaagttctgcatggtgaagataaagtggaagaagccaccgatgatatgattcatcgtgttcagaatgatctggcggcactgctgccgggctatatgattccgagcaatgttattaccatgagcagtattccgctgagcaataatggtaaagtggatcgcattagcctgaaagcctgcttcgataccctgattgttaataaacataccgaagaactgccgctggaaacacctctggaactgaaactggccgaaatctggaaagcactgctgaaaaccgatcatatctatcgcagtgatgacttcttcctgaaaggtggcagcagcctgaccgcagtggaactgctgaatcgcctgagcgaagcaggcttcattgtgaatattgatatgatcttcaacaacgcagtgttcgaaaaaatggttgatgtgctggaagccagcgaagatgcagaagaagcattccgccagagtattgatctggatagtctgtgtgcaaatgcactgcgtaacttcccgaatgcaaccccgctggaaaaacagcgcgcacagagtggtaatgtgttcatgaccggtgcaaccggttatctgggtgtgtatattctgaaaatgctgctggataagaccgatgataccctgtattgcctggtgcgctgtcgtgatgaaagcgaaggctataatcgtattcgcaaagttattgccgatcagggcttagattgggaaattgataccaaacgcattaatattatccctggtgatctgaccaaagaacgctatggcctggatgatgcacagtatcagagcctgagtcagaatattgataaagtgctgcatattgcagcactgattagtctgattgcaccgctgagtgaactgtatccgattaatgtgaaaggtagtgccaatgcaattgaactggcaaccaccggcaaaattaaaccgattcattatatgagtaccatcggcgtgcattatcgcctgccgtatgaagaaaatgaaccgccggtgctggaaagcgtggcaccggatggcccgtggcataaaccggaactgacctatgaacataccaaatatatggccgaacagatcttctatcgcgcccgcgaaaaaggtgttccggttaatatctatcgcagcggcgcaattacctgggataataccctggaagccccgttcattaatgacgatgccttcgtgaaattcttccgcacctgcctgagcattgccgcctatccggaaagtcgcattctgattagtaccaccccggttaatattgttgcagatgcaattgttgaaatcagccagatgtatattcagggcaaaggtgaaaattatcatgttgtgagtcgccatagtcataccggtgaacagatctattgttggctgaatgaattaggttgtcacttcaaaagtgtggacttcgccacctgggataaacagctggccgatagcttcggtcgcggcttcattaaccgttacttcaaacatggtatggaacagggcggccatcatcagtatcgtattgataataccgaaagtgcactggcaaccctggataaaacaccgttcgtgattaccaaagaatacttcgaaccgctggtggcacattataaagaagccattaatcgtgtgttcgtggaaaaagtggatagtgttaaagccagtaaacagaaagaaggtgcctaa

SEQ ID NO: 82 - protein, 1563 aa, Enterovibrio norvegicus
MKMFTKGMEVSAFVEALRNRGVQLWVEGEALKFKAPKGVMTAELKSILLAKKSEVIGFLNHVPDEVTLTAYPEDRYEPFPLTDLQLAYIVGRRNNYELGGVGCHSYIELDMPRLDHARLENAWHKLILRHEMLRAVIGTDGKQQILKHVTLPEVICHDTKGLSASAFETAIIQARNEMAYRRYDSTCWPLYELRLTQHDDRSILHHSTDLLIADFASIQILLAELGELYQHPEKELDAINVSFRDIVLYQQACHSQQESDSKYQVDRQYWQQRIPSLPGAPELPIHQNIHPPAHPIPDGEPSFERLSLEVSPPEWQALCQIATSHHITPSGAVMNAFAEVLQRWSRNDDFCINLTLLNRKAPHPDVNRIVGDFIAVNVLEIAHDRGGDFKERAVALQHQLWKDLEHDSYTGIDVLRDMTKDRKSNTLIPVVYTSTVGVSGEALPNNAFMHDAKLNFGITQTPQVWLDCQATERKGALCVDWDIRKGVFLPGVIEQAFDAFKTLLLSLSKSKGHWNSRMPVSLNSDCIALRKKLNTERNAPLPEGFLHASFCHHAITSPDKVALIHDAQTPITFTYRDMAEWSLSIADTLLDKGLQPGEPVAIFLDKGPQQIAAIMGILLAGGAYVPIDVIQPEKRRNTILHDACIRLILTAQTDANLNWPADIRTMVIAEPPTLERNDLALKQRLSRIIDQGLQSDTRNQLAYILYTSGTTGRPKGVMLTHEGVGNTIAGFNAEFGIESDDKTLALVNYSFDLSVLDIFCTLSAGATLVLPGDGWRNNPEVWAKTIQHQRVTIWNSVPAHMQMLMSLCENSRSMDLSTLRLAFISGDWIPVSLPPKIHEMLPTLSLNSLGGPTEISVTCIANQIKDVPEGATSIPYGLPLRNHRLYIQNNQYEDCPDWVPGEMLVGGPGVAKGFINDPKRTRERFILHPVSGERMYKTGDICRYRPDGVIEILGRQDNQVKIRGHRIELGEVEAALNELSYVQQAVALVRSGPAELVCALVFNTEVLHGEDKVEEATDDMIHRVQNDLAALLPGYMIPSNVITMSSIPLSNNGKVDRISLKACFDTLIVNKHTEELPLETPLELKLAEIWKALLKTDHIYRSDDFFLKGGSSLTAVELLNRLSEAGFIVNIDMIFNNAVFEKMVDVLEASEDAEEAFRQSIDLDSLCANALRNFPNATPLEKQRAQSGNVFMTGATGYLGVYILKMLLDKTDDTLYCLVRCRDESEGYNRIRKVIADQGLDWEIDTKRINIIPGDLTKERYGLDDAQYQSLSQNIDKVLHIAALISLIAPLSELYPINVKGSANAIELATTGKIKPIHYMSTIGVHYRLPYEENEPPVLESVAPDGPWHKPELTYEHTKYMAEQIFYRAREKGVPVNIYRSGAITWDNTLEAPFINDDAFVKFFRTCLSIAAYPESRILISTTPVNIVADAIVEISQMYIQGKGENYHVVSRHSHTGEQIYCWLNELGCHFKSVDFATWDKQLADSFGRGFINRYFKHGMEQGGHHQYRIDNTESALATLDKTPFVITKEYFEPLVAHYKEAINRVFVEKVDSVKASKQKEGA

SEQ ID NO: 83 - DNA, 3006 bp, Nostoc carneum NIES-2107
  source 1..3006: mol_type = genomic DNA
atgaagatgaacttcctgaatctgccgcagctgctgcgcaaacaggcagaagaaacacctaatagcattgcaattgtggatggtgataccagcattacctataaagaactgaatattctgaccgatagtctggccggctatctgcaacagcagggtgtgagcttcaataaactgaccggtatcttcatggaaaaaagtgccgaatatattatcgcctgcttcgccgcactgaaagccggtggcgcatatatgccgctggatctggcctatccggatgcaattctgagtaaaattgttagccaggataaaccgattgttattattaccaaatcattctacagcagtcgcctggaagccaataccagtaccattattctggatattgataccaataccgcctggcgcaattatgtgtgtaatcgtgatgccgtgagcctgatgagtccggataatctggcaatggttatctataccagtggtagcaccggtgaaccgaaaggcgtgctgctgagccatcgcgccattattcatagctattatacccatctgtacaatattagcttctatattgcaggcgatcgtagcgccttcaatgcattcttcccgctggaaatgctgaaaccgttcttccgtggcgccgcatgtcatattattccgaatgaagttgcatacgatccgaaactgctgctggacttcattgccacctatcagattaccgaaaccgttctgaccccgagcttcctgcaactgctgattaatcgcgtggaaccggcagttattcagaccctgctgagcagtctgaaagttctgtggatctgtggcgaagtgctgaccaccgaactgaaaaataaaattctgaaaatcttcccgcacaatctgtgtctgttcaattggtatggcatgagcgagttcccgagtgttagtctgtatcgcctggaagaaagtgaagacttcattagcgagttctgtccggtgggcttcccgctggaggaagtgaaagtgtgtctgctggatgaaaatcagaaagcagttagccctggcttccctggtgaactgtatgtgagtggtagctgtctggccagcggctatctgaatcgtccggaactgattgccgaacgcttcgtggtgttcaaaaaaaataaattctacaagaccggcgatattgccaccattctgccgaatggtatgctggaaattcgcggccgttgtgatagcaccgttaaaattcgcggttatagtattaatctgaaggcaattgaaaacgcactgcaaaaaaccggtctggtgaaattctgtctggttattaccgtgggcaaagaaggcgaaaataaacagattattgcctatattctgaagcaggaaaatctggacttcaaaattgatccgagcaatggtaattgcgttgaaattcaggaaaaaattaagagttacctgccgaaatatatgattccgagtaaatatatcgagctggccgaaattccgctgaatagcaatggtaaagttgatcagaaagcactgccgctgccgattccgaattatgaatatgatctgagcaatattcaggccaaagaaaccgttagtccggaagataaagaagccattctgaaaaaactgctggaacgcgttctgttcagtggtcgtggtcagattgaaaaagatgccaacttcttcgacttcggtctgaatagtctgctgagcgttgaactggccgccctgattggtaaaatgttccagattgaactgagtgttcgcgatatatatgattatccgaccattaataagctgctggaatacttcaataagggtgaaaaaaacttcagcaccgttattctgatgcgtaaagatagtcatctggatagcggcattaccccgattacccgcaccaaaccgctgagcattcgcgatgcaaaaagcattctggttaccggtgcaaccggcttcctgggtgccttcctgctggatgagctgctgcgcctgaccaatgatgatgtgaaactgtattgtctggttcgcacccagagtggcattgaagatgtgggcaaacagaaaattattaataacctgaagcgctattacctgtggaatcaggaaattgaaaaacgtattatcccgattgttggtgatctgagccaggaatatctgggcctgagtattgaacgcttcgaagaatataccgaaaccattgattatatcttccattgcgcagcaccggtgaacttcctgtatagctatgatattctgcgtgccccgattgtgaatggtacccgtgaaattattcgtctggccagcacctatgtgaccaaaccgttacagtatattagcaccgtgggtatcttcccgttcaaaagtcatcaggaactgttcctggaaaataatgaaattgatagcttcgcaaacaagctgaccggtggttatgcacgcgcaaaatgggtggcagaaaaactggtgtgggaagcaattgcccgtggcctgccggcctgcatctatcgtccgggctatattggtcatcatagtattaccggcatgtataacttcagcgacttccagttcctggtgattgatgcatgtaaaaaaattcagtgcgccccggaaaccagtcaggtgttcttcgaaatgattccggttaacttcctggttaaaagcattgcagagttcgccaataatgattgtaattacttcaaggtgtataacgttacccagcgtcagccgattccggcacgcttcgtgttcgatctgctgaaagaacagaattatattagtaagtacgtgagcgtggaagaatggaaagcaaaactgattgcaaaagcagataaagatggtgatacaattctgaatatcatcgcccagagcttcgaagaaaatatgggtctgtatccgcgtggcgaaagcgtgtatgattgcagcctgttcgaaaaagcactgagcaatcataaaattcagccgccggttattgataccgattatattaaaaaggccatgttcctgaatgaaggcattaattgctaa

SEQ ID NO: 84 - protein, 1001 aa, Nostoc carneum NIES-2107
MKMNFLNLPQLLRKQAEETPNSIAIVDGDTSITYKELNILTDSLAGYLQQQGVSFNKLTGIFMEKSAEYIIACFAALKAGGAYMPLDLAYPDAILSKIVSQDKPIVIITKSFYSSRLEANTSTIILDIDTNTAWRNYVCNRDAVSLMSPDNLAMVIYTSGSTGEPKGVLLSHRAIIHSYYTHLYNISFYIAGDRSAFNAFFPLEMLKPFFRGAACHIIPNEVAYDPKLLLDFIATYQITETVLTPSFLQLLINRVEPAVIQTLLSSLKVLWICGEVLTTELKNKILKIFPHNLCLFNWYGMSEFPSVSLYRLEESEDFISEFCPVGFPLEEVKVCLLDENQKAVSPGFPGELYVSGSCLASGYLNRPELIAERFVVFKKNKFYKTGDIATILPNGMLEIRGRCDSTVKIRGYSINLKAIENALQKTGLVKFCLVITVGKEGENKQIIAYILKQENLDFKIDPSNGNCVEIQEKIKSYLPKYMIPSKYIELAEIPLNSNGKVDQKALPLPIPNYEYDLSNIQAKETVSPEDKEAILKKLLERVLFSGRGQIEKDANFFDFGLNSLLSVELAALIGKMFQIELSVRDIYDYPTINKLLEYFNKGEKNFSTVILMRKDSHLDSGITPITRTKPLSIRDAKSILVTGATGFLGAFLLDELLRLTNDDVKLYCLVRTQSGIEDVGKQKIINNLKRYYLWNQEIEKRIIPIVGDLSQEYLGLSIERFEEYTETIDYIFHCAAPVNFLYSYDILRAPIVNGTREIIRLASTYVTKPLQYISTVGIFPFKSHQELFLENNEIDSFANKLTGGYARAKWVAEKLVWEAIARGLPACIYRPGYIGHHSITGMYNFSDFQFLVIDACKKIQCAPETSQVFFEMIPVNFLVKSIAEFANNDCNYFKVYNVTQRQPIPARFVFDLLKEQNYISKYVSVEEWKAKLIAKADKDGDTILNIIAQSFEENMGLYPRGESVYDCSLFEKALSNHKIQPPVIDTDYIKKAMFLNEGINC

SEQ ID NO: 85 - DNA, 2346 bp, Xenorhabdus japonica
  source 1..2346: mol_type = genomic DNA
atggtggttgatccgtatctgttccaggcaatgctggaaaaattcagtgtgagcttcatgattattaccaccctgttcaatctggttgccaatacctgtccgcaggccttccgtggtctgcgctatctgattccgggtggtgaagcactgaatccgtataccctgcgtcaggttctgcaaagtgcaccgccgcagcatctgattaatggctatggtccgaccgaaagtaccgttatgagcctgattcatgaaattaccctggatcagctgaccgaagaaaatattccgattggtaaaccgattgcaaatagcgaaatctatattctggatgatcagcagcagctggttgcaccgggccagattggtgaaatctgtctgggcggcgaaggtctggcacgtggctattggaatcgtccggaaattaatagtcagtgcttcattatggttgatattgaaaataacaacgagctgaaacgcatctatcgcaccggcgatctgggctggcagcgtagcgatggcatcttcatgtatgcaggtcgcatggataatcagattaaaattcgtggctatcgcattgaaattggcgaaattgaaacacaactgctgaaaagccagctgctgaaaagtaccgcagtgtgcgtggttaaaaaagaaaatattgaaccgtacctgatcgccttcattgttccgaaagatctgaataccttcagcacccagatgctgaaacagtggattaatctgtatctgccggattatatgctgccgcgtctggtggttgttgaacatattccgctgaccttcaatggtaaaaccgatcgcccgcgcctgctggccgaatatgccgaaaatcagaaacagcagcagttaagccagcagaaaaccaaaatgagcaaagaagaaagtatcgttctgaatagctggcgccaggcactggatagctatgatattaccctggacagcaacttcttccagctgggcggtaatagtctgcaagccacccgtctgattattgatattggccgtcagttccagcgtagcctgccggtgcagctgctgtatgatagtccgaccccgcgtaaactgattgaagccctgcaacagaataaactggaaccggcagatctgtgtaatattatgctgaaagatagcctgctgccgagtcatattcagccgctgagtcagccgctgcaaccgtggctgaccccggataccggtcgcgtgctgctgaccggtaccaccggcttcctgggcgccttcctgctgcgtgatctgctgctgcaagcaggtattaatcagattacctgcctggttcgtgcccataataatgatagtgcactgctgcgtgtgaaagataatctgaaacagtatggtctgtggaaagatgagttcctgttcaaactgcaagtgctggccagcgatctgaccaaaccgaaactggccctgagccagcagatctatgaacagctgagtgttgaatgtgatgttatcttccatctggcagcacatgtgaattatattcagccgtatagtgcacattatagtggcaatgtgctgggcaccctgaatgttctgcacttcgccgttagcaaaaaaattaaaccgctgcattatgttagcaccctggccgtgttcggtccggcaaaactgtgtagcagcattacccgtatctatgaagatgatgatattattgcctacctgaatggcctgaaatatgatagcggttatagccagagccagtgggttgttgaacgcatgatctggcaggcccgcgatcgcggcattccgctggcagtgtatcgccctggcttcattatgggcgatagcctgaccggtgccagcaatccgaaagacttcgtgagtcgcctggtgcagggttgcattaaaattggcgcatatccgctgctggccaatcagcgcaaagagttcgtgccggttaattatgttagccaggccctgctgaatattagtatggataatcatcgcctgggcaaagcctatcatctgggtaccccgaataatcagcagagcgtggatatgaatccgttcttcgaactgctgaatcagtgtggttatcagctgaaaggcctgcattatagcgaatggctggataaactggaaagcgatccgaaactggttgaaaatccgctgatgccgctgctgccggttctgagtgaaaaaatctatggtcagctgacccgttgggaagtgtatgaaaatatgccggtgtatgatgccaaaaataccgaaagtgcactgaaaggtaaatatgatctgtatctgcctatggataattatctgctggaacgctatctgagctattggattcagaccggcttcattagctaa

SEQ ID NO: 86 - protein, 781 aa, Xenorhabdus japonica
MVVDPYLFQAMLEKFSVSFMIITTLFNLVANTCPQAFRGLRYLIPGGEALNPYTLRQVLQSAPPQHLINGYGPTESTVMSLIHEITLDQLTEENIPIGKPIANSEIYILDDQQQLVAPGQIGEICLGGEGLARGYWNRPEINSQCFIMVDIENNNELKRIYRTGDLGWQRSDGIFMYAGRMDNQIKIRGYRIEIGEIETQLLKSQLLKSTAVCVVKKENIEPYLIAFIVPKDLNTFSTQMLKQWINLYLPDYMLPRLVVVEHIPLTFNGKTDRPRLLAEYAENQKQQQLSQQKTKMSKEESIVLNSWRQALDSYDITLDSNFFQLGGNSLQATRLIIDIGRQFQRSLPVQLLYDSPTPRKLIEALQQNKLEPADLCNIMLKDSLLPSHIQPLSQPLQPWLTPDTGRVLLTGTTGFLGAFLLRDLLLQAGINQITCLVRAHNNDSALLRVKDNLKQYGLWKDEFLFKLQVLASDLTKPKLALSQQIYEQLSVECDVIFHLAAHVNYIQPYSAHYSGNVLGTLNVLHFAVSKKIKPLHYVSTLAVFGPAKLCSSITRIYEDDDIIAYLNGLKYDSGYSQSQWVVERMIWQARDRGIPLAVYRPGFIMGDSLTGASNPKDFVSRLVQGCIKIGAYPLLANQRKEFVPVNYVSQALLNISMDNHRLGKAYHLGTPNNQQSVDMNPFFELLNQCGYQLKGLHYSEWLDKLESDPKLVENPLMPLLPVLSEKIYGQLTRWEVYENMPVYDAKNTESALKGKYDLYLPMDNYLLERYLSYWIQTGFIS

SEQ ID NO: 87 - DNA, 3423 bp, Pseudomonas sp. NFACC24-1
  source 1..3423: mol_type = genomic DNA
atgcgccgtctggatattgcagttccgggcaccagtcaggccatgaccgaactggcacatgaactggaactgcatggccatgccgtggttcgccatagcccggcaggccatgatgccgttgatctgctgattgatgatggcaccaccccgcaggccattgaagcacgcagtcgcctgcaaattcgcgtggccctgtgtaccctgccggccgaagcactgccgcagcctgtgctggtgttccgcgatggcggccagcgcctgctgtgccaagaagttattgccccgcagcgtgatggcaatggtcagagcctgcgcctgcgtaccctggcaaccctggttgaaaccggtgcacgtctggttagccagctgagccgtgatgaaacctgcttcgatcattggccgaccgttgatgcagatgccgtggatcagccgctgctgggtctggatgccctggaaccgctggcattcgaacatggcctgaatcgtccggccgtgccgtggctgctggcagcagcagatgtgccggttatgcatagtattgaacagcgcctgttagatgatgccacccagccggccctgtgggtggatggtcagttcattgattataaaagcctgcgccgcctggcactgggcctgcaacagcctatgctgcaacgtctgagtggcgcagccaaacgtccgctggtgattggcgtgtgtctgccgaaaagtgcccatctgtatgcagcaattctggcagttctgggctgcggtgccgtgtatctgccgctggaccctcagcatccggcacagcgtcgccgcttcattctggaaaatgcccaggcagatctgctgctgcatgatggtaatagtgatctgggcgatctgagcgtgccgaccctgaatgttcatcagctgccggcaccgaaaccgggtattccggccagtctgattcgtcgtaccgttaatattgatgaaccggcagttgcaatctataccagcggcaccaccggtcagccgaaaggtgtgctgctgagccatcgtaatctgagccacttctgtgcatggtatggtgattatgttggtctgcaacgcgatagtcgcgcactgcaattcagcaccattaacttcgatgcaagcctgctggatattctgccgaccttcattcatggcgccctgctggtggttccgagtgaagatcagcgccgtgatccgcagcagctggttgcactgattcatcagcagcatattagccatgcattcctgccgccggccctgctgagtgttatgccgctggataccccgctgggtctgacccatctgattaccggtggtgatgtgtgcgaaccgttcgtgattgaacagctggccccgcagtgtcagttccataatatctatggcccgaccgaaaccaccgtgctggcaagcacccgtcagttcggtgttggcgataataatcgtaatctgggtgttccgattgccaatggtcgtgttctgattctggatgaacagctgcaaccggtgccgcagggtgttgcaggtgaactgtatattgcaggccctggtgttggcctgggctatctgaatgatccggcactgaccgcaacccgttatcatgatctgaccctgccgaccggccaggccctgagagtgtatcgcaccggtgatattggtcagtggaccgataatggcattgaactgtgtggtcgtcgtgataatcaggttaaaattcgtggcttccgtgtggaaccggaagaaattgaacattgtctgcgtgatagccagctgttccgccaggttgccgtgggtgtggatgaaaaacgtcgtattctggcattcctggttcatccggaacaggcacatccgggtgccgcactgcaagcactgcgtgaacatgttcagaatagtctgccgagctatatgcatccggccgcatacgttgaactgccgagcctgccgtataccagcaatggtaaagttgatcgccgtgcactgctggcaatggcagtgcaggtgcatgttgatggccagcgccgtcagccgcagaatgcactggaaacacaactgcaacagctgtgggcagaactgctggaactgccggtggatgatattgcaaccgatgaatcattcttcaatctgggtggccatagcattctgctgagccgtctgctgctgggtattcgccaggtgttcggccgcagtctgagtattaatcgcttcattgaagccccgaccctgatgaccctggccaaactgctggatgatccggaacatagcaatgcaagtgtgctgagcccgcaggcattcattgatgccgaagcagaactgaatctggaccctctgccgattagtcagtgtggtgatgtgcataaagttgtggttaccggtgcaaatagcttcctgggcgttcatattgttgaagcactgctggcgtggggtgcaaccgaagttgcctgtctggtgcgcgccagtgccgaacatagtgcagccgaacgcttcagtcaggcactgcgtgataatcatctgacccatcttgatctggatcgcgtgagcgtgtatgccgcagatattacccagccgcagctgggcctgccggatgatgtgtatgcacgtattgatagcaccttcggtgcactggtgcataatgccgcacatgttaatcatgtgctggattatggcagtctggcacgtgataatgtggaaccgatcttcgaatgtctgcgtctgtgtgaaggtcgcagtaaaaaaatcttcaacttcgtgagcaccctgagcgcaagtagcgccctggatgcagatggccaggtgctggaacagccggccgccgaaacacctccgatctatattaaaaatggttataacctgagcaagtgggttggtgaacgcattctgcaacgcgcccgcgatcgcggcgtgtgggttaatctgttccgccctggtaatattagcttcaatagtctgaccggtgtgtgccagccgcataaaaatcgtctgatgctgatgctgaaaggcagtattcagctgggtcaggttccggatctgagcctgaacttcgatctgatgccggtggacttcctggcccgcttcattgcattccatgcaagtcgctatcagccgacccaggcagtgttcaatctgcataatccgcagccgctgagctgggatgcatacgttgccagcttccgtgatagcggtcgtgagttcagcctggtgagtgtggccgaatggcagcgtcagctggcacgtgtggatgccgataatgccctgttcggtgtgctgggcttctatctgaatggcttcgaagaagatattggcgatattagcctgattagccatgataatgcacgcgcaggcgtgcgtcagatgggtgccagctatccggaaaaaagcccggcactgctgcgtcgtggcgcagattatctgaaagccattgacttcatttaa

SEQ ID NO: 88 - protein, 1140 aa, Pseudomonas sp. NFACC24-1
MRRLDIAVPGTSQAMTELAHELELHGHAVVRHSPAGHDAVDLLIDDGTTPQAIEARSRLQIRVALCTLPAEALPQPVLVFRDGGQRLLCQEVIAPQRDGNGQSLRLRTLATLVETGARLVSQLSRDETCFDHWPTVDADAVDQPLLGLDALEPLAFEHGLNRPAVPWLLAAADVPVMHSIEQRLLDDATQPALWVDGQFIDYKSLRRLALGLQQPMLQRLSGAAKRPLVIGVCLPKSAHLYAAILAVLGCGAVYLPLDPQHPAQRRRFILENAQADLLLHDGNSDLGDLSVPTLNVHQLPAPKPGIPASLIRRTVNIDEPAVAIYTSGTTGQPKGVLLSHRNLSHFCAWYGDYVGLQRDSRALQFSTINFDASLLDILPTFIHGALLVVPSEDQRRDPQQLVALIHQQHISHAFLPPALLSVMPLDTPLGLTHLITGGDVCEPFVIEQLAPQCQFHNIYGPTETTVLASTRQFGVGDNNRNLGVPIANGRVLILDEQLQPVPQGVAGELYIAGPGVGLGYLNDPALTATRYHDLTLPTGQALRVYRTGDIGQWTDNGIELCGRRDNQVKIRGFRVEPEEIEHCLRDSQLFRQVAVGVDEKRRILAFLVHPEQAHPGAALQALREHVQNSLPSYMHPAAYVELPSLPYTSNGKVDRRALLAMAVQVHVDGQRRQPQNALETQLQQLWAELLELPVDDIATDESFFNLGGHSILLSRLLLGIRQVFGRSLSINRFIEAPTLMTLAKLLDDPEHSNASVLSPQAFIDAEAELNLDPLPISQCGDVHKVVVTGANSFLGVHIVEALLAWGATEVACLVRASAEHSAAERFSQALRDNHLTHLDLDRVSVYAADITQPQLGLPDDVYARIDSTFGALVHNAAHVNHVLDYGSLARDNVEPIFECLRLCEGRSKKIFNFVSTLSASSALDADGQVLEQPAAETPPIYIKNGYNLSKWVGERILQRARDRGVWVNLFRPGNISFNSLTGVCQPHKNRLMLMLKGSIQLGQVPDLSLNFDLMPVDFLARFIAFHASRYQPTQAVFNLHNPQPLSWDAYVASFRDSGREFSLVSVAEWQRQLARVDADNALFGVLGFYLNGFEEDIGDISLISHDNARAGVRQMGASYPEKSPALLRRGADYLKAIDFI

SEQ ID NO: 89 - DNA, 4248 bp, Clostridium sp. CAG:508
  source 1..4248: mol_type = genomic DNA
atggaaaagaacctgtatgatctgaccaatccgcagaaaagtatctggtataccgaacagttcttcaaaaataccagcgtgaataatatcggcggtaccattattattaaggataaaattagcttcgacctgctgaaaaaaagtatctatgaatatgtgaagaccaacgatagcttcctgattaaactgtgcttcgatgaaaaaggcgatattaaacagtatatcggcgagttcagtccgttcaatgtggattatattagcttcagcagcctggatgaactgtataaattcgaaaaaaacgactgtgaacgcaaattcgatatgattaatagtaacctgttccacttcaccatgttcagcctgccggatgcaaccggcggtgttgttctgaccgttcatcatattattagcgatgcacagaccgtgaatcagattgccccgaaactgatgaacttctatagtagcttcctgaataataccaccccggagttcctgcaaaccagctatgttgattatattaatagcgaacagaagtacctgagcagcgaaaaatataataatgataagatgttctgggagaacctgttcgaaaatccgttcaccccggccagtctgagtaccaaaagtattgatagcttcgcaatgtgcagtagtagccgtgaatgcttcctgattgataaagaaaaaatgaacaagatcaacgactactgcaaactgaataatattagctgctatcacttcttcatgagtgtgttcagtctgtatattggtaaagttaccaatgaaaagaacttcgtgattggcaccccgattctgaatcgtaccaacttcaaagaaaaaaataccgcaggcatgttcattagcaccgtgccgttcctggttaatctggaagaaaataccctgttcagtgagttcgccagcaaaatgagcgtggaaaccttatcaatcttccgtcatcagaaatatccgtatcaggccattctggaatatgtgcgtgcccaggatagtagcattccgaatctgtataatattatgatcagctatcagatcaacaagagtaatggtagtattaatgacatcccgtataccacccgctggacccatagtaataccaatatggatgaactggatattcatctgttcgatattaatgataccggcgaactgagtattgcctatgattatcagctggccaaatataccgaaaaagatattcagcagctgcatcagcgtattattagtattattaaccaggttatcaacaacccgaatattagtaccaatcagctggaactggttctgccggaagaaaaacagttcattctgaatacctataacaagccgattaatctgccgattaccaaaaccgttattgatctgttcgaagaaaaagtggccgaaaatccggataaactggccctgtgcttcggtaataccaaattcacctataaagaactgaacaattgcgtgaattacctggcagattatctgtatgaaaataatatcgagaagggcgataaagtgtgcctgttcttcagtaatagcattgaactggttgtgagcattctggcagtgctgaaactgggcgcatgttatattccgattgatgtgagctatccgattgaacgtgttaaatatattgcacagaacagtagttgcaaaaaaattctgaccaataccaaactggaagatctggataatctgtgtctgggcattagcctgccgaatgccaccaccatctatgaaaataaatataaaccggacctgcaagatctggcatatattatctataccagtggtagcaccggcaatccgaaaggcgttaaaattgcccatgaaagtctgagcaattatattatgtgggccaataaagaatacgttaatggtgaaaccaccaacttcccgctgtatagtagtattagcttcgatctgaccgtgaccagcgtgttcaccccgctggtgagcggtaataccatctatatctatgaaaacagtaacccgcagctgctgctgaaagaaattattgatgataaaaaggtgcagatcatcaaactgaccccggcacatctgaatctgctgcaagatcttgcaagcccggaaagtgttgttaccaaactgattgttggcggcgatattctgaccaacgaaagctgtcagaaaattagcaccgcattcgttcatccgattcatatcttcaatgaatatggtccgaccgaagccaccgtgggctgtatgatctatgaatacaccgaaaatgaatacgccaccgtgccgattggtattccggcagataataccaatctgttcgttctgaatagtgatctgaatctggttccgttcggctataatggtcagctgtatattgccggcaaaggcctgagcaaaggttatgttgaactgccgaaaatgaccgcaagccgcttcattgcctgcccgttcattgaaaatggtctgatgtatgatagcggtgatattgtgaaactgtatgataccggtattatggaatgcattggccgtagcgacttccaggtgaaaattaatggcttccgcattgaaattggcgaaattcagagtcgcattctgaattatccgaatattaaagattgctacgtgagcgcactggatattaaaggtaccaaagttctgtgtgcatattatgttagtaacaaaaacatcaacgagaagaatctgaaaaactatctgcaaaaactgctgccgatgtatatggttccgaaatattatgtgagcctgcaagaactgccgctgaccggcaatggcaaaattaataaaaaactgctgcctctgccggccctgaatgaaagtaaagaactggttaaaccgagcaataaagtggaacataccctgcataatatcttctgtgaactgctgcaagtgccggaactgagcgtgaccgcaaatatcttcgattattatgtggatagcctgaccattattaaagcacagaccaaactgtatagtattggctataatatcaacacccaggacttctatgattatcaaaccattcgtgaactggcaagccatattctgcgtaatagcaatagtaatagcggtgcaagtcagaatgaaattccgattattgaaaacctgatcaaaccgattaacagcaccaataccttcaataatatcctgctgttcggcgtgaccggcttcctgggcagccatattctgtatgaactgctgaccaaaaccaaaagtcgcatctattgtgttattcgcgaaaaagataacatcaatgccattgaacgcttctatgaaaaattccacttctacttcccgaatgaaaatctgcgtaattatagtcatcgcattaatctgattaccggtaatattctgaaggataacttcaatctgccggatgaacagtatgacttcctgggcaataatattaattgcgtgattagtagtgcggcactggttaaacattatggcaaatatgaagagttctacaataccaatgtggcaggtaccaaaaaaattaccaacttctgcattaagtacaacattccgctgcattatattagcaccattagtgtgagcggctatggcctggtgaaaagcccggaaattaccttcaccgaaaaagacttctatattggtcagagctatgaagataatgtgtatgtgaaaagcaagttcgaagcagaaaaactgattctgaatgcctgcaaaaatgataatctgtgcgcaaccatctatcgcatgggtaatattaccaatcgcttcagtgatggtgtgttccagcagaatgccaatgaaaatgccttcctgaatcgcattagcgcagttattaatctgcattgcattccggaagaactgctgcacttcccggttgagttcaccccggttgattattgcgccaacttcattgtgagcctgctgattaatgatacacgtaaaaataacctgaccatctatcatctgttcaatcagaattatctggacttcaattacctgttcgagttcctgaaaaaacataatattaacatgaagacctgctctattgaagagttcgaaaaactgatcctgaatagtaaaaacaactacttcggtattaccggctatctggcaaatattaaaaccaataatctgaacggtgtgattctggataatacctgcaccaatagcattctgaaaaaactgaatctgagctggccgattgtgaccgaagattatctgcaagcagttattaactatctgatgaaaaacaagttcatcggtggttaa

SEQ ID NO: 90 - protein, 1415 aa, Clostridium sp. CAG:508
MEKNLYDLTNPQKSIWYTEQFFKNTSVNNIGGTIIIKDKISFDLLKKSIYEYVKTNDSFLIKLCFDEKGDIKQYIGEFSPFNVDYISFSSLDELYKFEKNDCERKFDMINSNLFHFTMFSLPDATGGVVLTVHHIISDAQTVNQIAPKLMNFYSSFLNNTTPEFLQTSYVDYINSEQKYLSSEKYNNDKMFWENLFENPFTPASLSTKSIDSFAMCSSSRECFLIDKEKMNKINDYCKLNNISCYHFFMSVFSLYIGKVTNEKNFVIGTPILNRTNFKEKNTAGMFISTVPFLVNLEENTLFSEFASKMSVETLSIFRHQKYPYQAILEYVRAQDSSIPNLYNIMISYQINKSNGSINDIPYTTRWTHSNTNMDELDIHLFDINDTGELSIAYDYQLAKYTEKDIQQLHQRIISIINQVINNPNISTNQLELVLPEEKQFILNTYNKPINLPITKTVIDLFEEKVAENPDKLALCFGNTKFTYKELNNCVNYLADYLYENNIEKGDKVCLFFSNSIELVVSILAVLKLGACYIPIDVSYPIERVKYIAQNSSCKKILTNTKLEDLDNLCLGISLPNATTIYENKYKPDLQDLAYIIYTSGSTGNPKGVKIAHESLSNYIMWANKEYVNGETTNFPLYSSISFDLTVTSVFTPLVSGNTIYIYENSNPQLLLKEIIDDKKVQIIKLTPAHLNLLQDLASPESVVTKLIVGGDILTNESCQKISTAFVHPIHIFNEYGPTEATVGCMIYEYTENEYATVPIGIPADNTNLFVLNSDLNLVPFGYNGQLYIAGKGLSKGYVELPKMTASRFIACPFIENGLMYDSGDIVKLYDTGIMECIGRSDFQVKINGFRIEIGEIQSRILNYPNIKDCYVSALDIKGTKVLCAYYVSNKNINEKNLKNYLQKLLPMYMVPKYYVSLQELPLTGNGKINKKLLPLPALNESKELVKPSNKVEHTLHNIFCELLQVPELSVTANIFDYYVDSLTIIKAQTKLYSIGYNINTQDFYDYQTIRELASHILRNSNSNSGASQNEIPIIENLIKPINSTNTFNNILLFGVTGFLGSHILYELLTKTKSRIYCVIREKDNINAIERFYEKFHFYFPNENLRNYSHRINLITGNILKDNFNLPDEQYDFLGNNINCVISSAALVKHYGKYEEFYNTNVAGTKKITNFCIKYNIPLHYISTISVSGYGLVKSPEITFTEKDFYIGQSYEDNVYVKSKFEAEKLILNACKNDNLCATIYRMGNITNRFSDGVFQQNANENAFLNRISAVINLHCIPEELLHFPVEFTPVDYCANFIVSLLINDTRKNNLTIYHLFNQNYLDFNYLFEFLKKHNINMKTCSIEEFEKLILNSKNNYFGITGYLANIKTNNLNGVILDNTCTNSILKKLNLSWPIVTEDYLQAVINYLMKNKFIGG

SEQ ID NO: 91 - DNA, 3561 bp, Minicystis rosea
  source 1..3561: mol_type = genomic DNA
atgctgccgctggaaggttgcgatccgattcagctgattgatgcaagctatggtcgctatacccgccagagtagtagtagcgcccagccgttcagtggctggatgttcgcaaccgaaccggaaccggacccttattgggaaatctatctgggcgaaagcatgttcgttgaacgtattgcactgtggctggcaccggtggcaccggataccgaagttcgtgttgttgcacatgcaattccgaccccgcagaatgaaccggccgcaggtagcgccacctgggttagccgtattgatgaactgccggttgcaagcgatggtagtgcagttctgtggattggtgccgataccgttgcacgcttcgtgcgcgttagtctgcatcgcgccgatggcgccccggttaccctgttagttcgcggcgttgaaattctggccgccccgctgttcctggaaagcctggcagcatcattcgcacgcgcattcaccctgttcgccgatcgtccgctgttcgccgcacgcattggtgaaggtccgttcaccgtgacccatcgttatcgtgatgtgtggaccgcagcacgccgtctggcaggcgcactggctcatctgaccccgagcgatggtgaaaccagagtgttcattggcctgtgtacccgtaatcgtccggaatggctgatgggtgatctggccgccgtgattctgggccatgtgaccgttccgctgagcccggatgatagtgatgatcgcctggcaaccattctgggtcgctgtccgctggcagcagtgctggtggaaccgggtgatggtgatcgcttcgcccgtctggcagcccgctgccctagtctgcgcctgattattgaatgtgatccggcaccggccgatgccccgagcagagcaagccctgcaaatgaaccggaacgcattggcttcaccgatctgctggcccgtgccgcagaacgcccggcacctagtgcccctgcacgcgatggtcgtgatctgtgtaccgttctgttcaccagcggtagtagtggtgtgccgaaaggcgcaatgcgtagttatgccaccttcctggcaatggtgagtggctatggcgttgcagaaccggcaattcatctgagcttccagccgctgagtcatgttagcgaacgtatgtatctgccggcagtggtgctgcgtggcggtctggtgggcttcagtcgtggtggtgcccgcgtgctgagtgatctgcgcgccctggaaccgaccattgtgggcagtgttccgcgtctgttcgaactggttcatgcagcccatcgccgtcgtctggaagcagcactgcgtgccgatgatcataccccgccggatgaaattgaagcccgcgttctggccgaaagtcgcgcagtgttcggtagtcgtctgcaaggtgtgggcattggcagcgccccgtgtagtccggaagttctggccttcctgcgtcgctgcttcgccgatctgtgggttagcgaaggctatggcagtaccgaacatggcaccattgccagtagtggtctggttcagccgaatgttgaagtgaaactggtgccgctggcaggcgtgagtgaaggtgccccggcacgcggtgaaatctgggttcgcaccccgcatgttattgatggttattatggcgatccggcagccaccgcagccagcagagatgcagatggcttcttccgtaccggtgatctgggcgaactggatgccgatggtcgtgtgcgcgttgttggtcgtgtgcagaatgttattaaactgggtcagggtgagttcgttgccgtggatcgtattgaagccgccctggccggtgccccgattgtggatcagatcttcgttcatccggatgccgccagcgccagcctgctggcagttgttattccggccggtgatgtgctgcgtgccgaactgggcgttaccagcgcaagccgtgcagaactggccagtcatccggatgcagcccgtaccgtgctgcgtgcactgcacgctcatggtcgccgtgttggtctggcccgctatgaactgccgcgtgcagtgctggttgatccggaccctatgaccgtggataccggtctgctgaccgcaagcggcaaactggcacgtccggcagtgattgcacgttatgcaagcgcactggcagcactggatcgcaccgcaccgccgcctgctgccgatggtagtctggcagcccagctggcagcagttattggcgttgttgttggccgtaccgttgatattgatgaaccgctgcatgaaggtctgggtattgatagcctggcaattgccgaagccattcatgcagcaagcaccagtctgggccatgatattccgctgggcatgtggttcgaagcacgcaccctggaagatctggcagcccgtctggatcgcggtaccctgggccgtctggcccctaatgatgatcagccggcccgtgatctggcactggaccctgcacgtggcgcccatctggccgctgcacgtgttccgttcgaaaccattctgctgaccggcgcaaccggtctgttaggtgcccatctgctggaaagcattgcagcacgcgcagatgcacatgttctgtgcctggttcgcgcaccgagcgatgataccgcagcagaacgcgttcgtgcaaccctggcccgttatgccattccgagtccggcaccgagtcgctggagtgcaattgccgcagatctggccgcaccgaatctgggtctggaccctgcccgcttccgtgcactggcagcgcgtgctgatgcaattgttcatgcaggcgcaaccgttagctggctgcacgcttatgatgttctgcgtgccccgaatgtgctgggcacccagaccctgctggaactggccatgcacgctcgcgaaattccgttccacttcgtgagtaccctgagcaccgccccggcagatggcgatgaaagtaccattctgagcttcgcagaagcccgtgcaggcggcggctatggtctgagtaaatgggtggccgaacagctggttcgtcgtgccggcgatcgcggtcatccggttgcagtgtatcgcccgagcatgattaccggccatagctgccgtggtctgggcaatccggatgattatgtgcatcgttatctgcgtgcctgtgtgcagttcggccgcttcctggatggtgatggtgaacgcctggatatgaccccggtggacttcgttgccgatggcattgcagcactgatgctgagccgcccgcgtggtggtgcaacccatcatctgaccaatattgatcagagcatgagttataccgatctgggtcatgcattcagcaatgccggttatgcatgtaccaaaaccagctatgcaggcttccgtgcaaccgccgtgctggcaagcggtagcccgctgcgtgccctggctgcatacttcccggaagccggcttcggtctgcgcatgggcccgtggcctagtgcacgtacccgcgatgaactggccaccctgggcgttgtgtgtccgcgtgtggatgccaccctgattgaaacctatctggcaggtctggcacatcgtggtctgctgcgcgatccggcaaaataa

SEQ ID NO: 92 - protein, 1186 aa, Minicystis rosea
MLPLEGCDPIQLIDASYGRYTRQSSSSAQPFSGWMFATEPEPDPYWEIYLGESMFVERIALWLAPVAPDTEVRVVAHAIPTPQNEPAAGSATWVSRIDELPVASDGSAVLWIGADTVARFVRVSLHRADGAPVTLLVRGVEILAAPLFLESLAASFARAFTLFADRPLFAARIGEGPFTVTHRYRDVWTAARRLAGALAHLTPSDGETRVFIGLCTRNRPEWLMGDLAAVILGHVTVPLSPDDSDDRLATILGRCPLAAVLVEPGDGDRFARLAARCPSLRLIIECDPAPADAPSRASPANEPERIGFTDLLARAAERPAPSAPARDGRDLCTVLFTSGSSGVPKGAMRSYATFLAMVSGYGVAEPAIHLSFQPLSHVSERMYLPAVVLRGGLVGFSRGGARVLSDLRALEPTIVGSVPRLFELVHAAHRRRLEAALRADDHTPPDEIEARVLAESRAVFGSRLQGVGIGSAPCSPEVLAFLRRCFADLWVSEGYGSTEHGTIASSGLVQPNVEVKLVPLAGVSEGAPARGEIWVRTPHVIDGYYGDPAATAASRDADGFFRTGDLGELDADGRVRVVGRVQNVIKLGQGEFVAVDRIEAALAGAPIVDQIFVHPDAASASLLAVVIPAGDVLRAELGVTSASRAELASHPDAARTVLRALHAHGRRVGLARYELPRAVLVDPDPMTVDTGLLTASGKLARPAVIARYASALAALDRTAPPPAADGSLAAQLAAVIGVVVGRTVDIDEPLHEGLGIDSLAIAEAIHAASTSLGHDIPLGMWFEARTLEDLAARLDRGTLGRLAPNDDQPARDLALDPARGAHLAAARVPFETILLTGATGLLGAHLLESIAARADAHVLCLVRAPSDDTAAERVRATLARYAIPSPAPSRWSAIAADLAAPNLGLDPARFRALAARADAIVHAGATVSWLHAYDVLRAPNVLGTQTLLELAMHAREIPFHFVSTLSTAPADGDESTILSFAEARAGGGYGLSKWVAEQLVRRAGDRGHPVAVYRPSMITGHSCRGLGNPDDYVHRYLRACVQFGRFLDGDGERLDMTPVDFVADGIAALMLSRPRGGATHHLTNIDQSMSYTDLGHAFSNAGYACTKTSYAGFRATAVLASGSPLRALAAYFPEAGFGLRMGPWPSARTRDELATLGVVCPRVDATLIETYLAGLAHRGLLRDPAK

SEQ ID NO: 93 - DNA, 3561 bp, Mycobacteroides chelonae
  source 1..3561: mol_type = genomic DNA
atgaccgttaataacgataccgatccgcagctggaacagctgacccgccgcattgaaaatctgcgtgaaagcgatctgcaattccgtgataccctgccggaccctgccgttgcacagcaggttctgcgtccgggtctgcatctgagtgaagcaattgccaccctgatgaccggctatgcagcacgtccggccctgggtgaacgtgcccgtgaactggttaccgatcatgatggccgcaccattcagcgcctgctgccgcgcttcgaaaccaccacctatggcgaactgtggagtcgcaccaccagcgtggccgccagctggcatcatgatccggcacatccggttaaaggcggcgatctggttgcaaccctgggcttcaccagcattgattataccgttctggatctggcaattatgattctgggcggcgttgccgtgccgctgcaaaccagtgcaccggcaagtcagtggaccaccattctggcagaagcagaaccgaataccctggcagtgagtgtggaactgattggcgttgcactggaaagtgtgctggcaaccccgagtattaaacaggtggtggtgttcgattataccccggaaattgatgcacagcgtgaagcctatgaagcagccggtgcccgcctggccggtacaggtattgcaattgaaaccttagatgccgttattgcacgtggtagtgatctgccggcagccccgctgtatgccccgagtcctggtgatgatccgctggcactgctgatctataccagtggtagcaccggtgcaccgaaaggtgccatgcatagcgaaaatattgttcgccgctggtggattcgcgaagatgttatggccggtaccgaaaatctgccgatgattggcctgaacttcatgccgatgagtcatattatgggccgtggcaccctgaccagcaccctgagtaccggtggcctgggctacttcgccgccagtagtgatatgagcaccctgttcgaagatatggaactgattcgcccgaccgcactggccctggtgccgcgtgtgtgtgatatggtgttccagcgcttccagaccgaagttgatcgccgcctggccggcgatcgcaccgcagacgcagatgccgtggccgctgaagtgaaagcagaaattcgcaatggtctgttcggtggtcgcgtgctggcagttatggttggtagtgccccgctgagcgatgaactgggtgagttcattgaaagctgcttcgaactgcatctgaccgatggctatggtagtaccgaagcaggcatggtgttccgtgatggtattgtgcagcgcccgccggttattgaatataaactggttgatgtgccggaactgggctacttcagtaccgatcagccgcatccgcgtggtgaactgctgctgaaaaccgatggtatgttcctgggttattataaacgtccggaagttaccgccggcgtgttcgatgaaaatggcttctatatgaccggtgatattgttaccgaactggcacatgataatattcgcattgtggatcgccgcaataatgtgctgaaactgagccagggtgagttcgtggcagttgcaaccttagaagccgaatatgcaaatagcccggttgtgcatcagatctatgtgtatggcagtagtgaacgtagctatctgctggcagtggtggtgccgaccccgcaggctgtggcagcagctaaaggtgatgaaaccgcactgaaagccaccattgccgaaagcctgcaagatattgcccgcgaactgcaactgcaaagttatgaaattccgcgcgacttcattattgaaccgcagccgttcacccagggtaatggtctgctgaccggcattgcaaaactggcacgtccgaatctgaaagcacattatggcgatcgtctggaacagatgtatgcagatattgccgaacagcaggccgccgaactgcgtgcactgcatagcgttgatccggataaaccggcactggaaaccgttctgaaagcagcacaggccctgctgggtgttagtagtgcagaactggcagccgatgcacacttcaccgatctgggcggtgatagcctgagtgcactgagcttcagcgatctgctgcgcgatatattcggcgtggaagttccggttggcgttattgttagcgcagccaatgatctgggcagtgtggcccagttcattgatgaacagcgccatagtggtggtacccgcccgaccgcagataccgttcatggtgcaggccataccgaaattcgtgcagccgatctgaccctggataaattcattgatgaggcaaccctgcgcgcagccccggcattaccgcgtgcaaccggcaccccgcagaccgttctgctgaccggtagtaatggctatctgggtcattatctggccctggaatggctggaacgtctggataaaaccgatggcaaactgattgtgattgttcgcggcaaagatagtggtgccgcatatcgtcgcctggaagaagcattcgataccggtgataccggtctgctgacacacttccgcgcactggccgataaacatctggaagttctggccggcgacattggcgatccgaatctgggcctggatgccgatacctggcagcgtctggcagataccgtggatgcaattgtgcatccggcagcactggtgaatcatgttctgccgtatcgtcagctgttcggcccgaatgtggtgggcaccgcagaaattattaaactggccctgaccaccaaaattaaaccggtgacctatctgagcaccgttgcagtggccgcctatgttgatccgagtaccttcgatgaagaaagtgatattcgtctgattagtgccgttcgtccggtggatgaactgtatgcaaatggctatggcaatagcaaatgggccggtgaagtgctgctgcgcgaagcccatgatctgtgtggtctgccggttgccgtgttccgtagcgatatgattctggcccatagtcattataccggtcagctgaatgtgccggatcagttcacccgtctgattctgagtctgattgccaccggcattgcgccgggtagcttctatcagccgcaggccaccggtgatcgcccgttagcacattatgatggcctgccggccgacttcaccgccagtgctattaccttcctgggcattgcagtggcagccagtgaatcattccatacctatgatagtgttaatccgcatagtgatggcattagcctggataacttcgtggattggctgattgatgcaggctatccgattcagcgcattgataattatagtgattggttcgatcgcttcgataccgccattcgtggtctgccggaaaaacagaaacagcatagcctgctgccgctgctgcacgcattcgaacagccgagcggtgcagaagatcatggtgtggtgccggccaaacgcttccagcacgctgtgcaggcagcccgtattggcccggtgggtcaggatggcaccaccgatattccgcatctgagccagcagctgattgttaaatatgcaaccgatctggaacagttaggtctgctgtaa

SEQ ID NO: 94 - protein, 1186 aa, Mycobacteroides chelonae
MTVNNDTDPQLEQLTRRIENLRESDLQFRDTLPDPAVAQQVLRPGLHLSEAIATLMTGYAARPALGERARELVTDHDGRTIQRLLPRFETTTYGELWSRTTSVAASWHHDPAHPVKGGDLVATLGFTSIDYTVLDLAIMILGGVAVPLQTSAPASQWTTILAEAEPNTLAVSVELIGVALESVLATPSIKQVVVFDYTPEIDAQREAYEAAGARLAGTGIAIETLDAVIARGSDLPAAPLYAPSPGDDPLALLIYTSGSTGAPKGAMHSENIVRRWWIREDVMAGTENLPMIGLNFMPMSHIMGRGTLTSTLSTGGLGYFAASSDMSTLFEDMELIRPTALALVPRVCDMVFQRFQTEVDRRLAGDRTADADAVAAEVKAEIRNGLFGGRVLAVMVGSAPLSDELGEFIESCFELHLTDGYGSTEAGMVFRDGIVQRPPVIEYKLVDVPELGYFSTDQPHPRGELLLKTDGMFLGYYKRPEVTAGVFDENGFYMTGDIVTELAHDNIRIVDRRNNVLKLSQGEFVAVATLEAEYANSPVVHQIYVYGSSERSYLLAVVVPTPQAVAAAKGDETALKATIAESLQDIARELQLQSYEIPRDFIIEPQPFTQGNGLLTGIAKLARPNLKAHYGDRLEQMYADIAEQQAAELRALHSVDPDKPALETVLKAAQALLGVSSAELAADAHFTDLGGDSLSALSFSDLLRDIFGVEVPVGVIVSAANDLGSVAQFIDEQRHSGGTRPTADTVHGAGHTEIRAADLTLDKFIDEATLRAAPALPRATGTPQTVLLTGSNGYLGHYLALEWLERLDKTDGKLIVIVRGKDSGAAYRRLEEAFDTGDTGLLTHFRALADKHLEVLAGDIGDPNLGLDADTWQRLADTVDAIVHPAALVNHVLPYRQLFGPNVVGTAEIIKLALTTKIKPVTYLSTVAVAAYVDPSTFDEESDIRLISAVRPVDELYANGYGNSKWAGEVLLREAHDLCGLPVAVFRSDMILAHSHYTGQLNVPDQFTRLILSLIATGIAPGSFYQPQATGDRPLAHYDGLPADFTASAITFLGIAVAASESFHTYDSVNPHSDGISLDNFVDWLIDAGYPIQRIDNYSDWFDRFDTAIRGLPEKQKQHSLLPLLHAFEQPSGAEDHGVVPAKRFQHAVQAARIGPVGQDGTTDIPHLSQQLIVKYATDLEQLGLL

SEQ ID NO: 95 - DNA, 3546 bp, Mycobacteroides abscessus
  source 1..3546: mol_type = genomic DNA
atgactgaaacgatctccacagcggctgtccccactacggatctcgaagagcaggtgaagcgacgcatcgagcaggtcgtgtccaacgatccgcagctggcggcgcttctcccggaagattcggtcaccgaggcggtcaacgagcccgatctaccgctggtcgaggtgatcaggcgactgctggagggctacggtgaccgcccggcactcggccagcgcgccttcgagttcgtcaccggggacgacggtgcgaccgtgatcgcgctgaagcccgaatacaccaccgtctcctaccgcgagttgtgggaacgtgccgaggctatcgctgccgcgtggcacgagcagggcatccgtgacggcgacttcgtcgctcagttgggtttcaccagcacggacttcgcgtcgctcgacgtcgcgggattgcgtctgggcaccgtctcggtgcccctgcagacgggcgcgtcgctgcagcagcgcaacgcgattctcgaagagacccggcccgcagtctttgccgcgagtatcgaataccttgatgccgccgtcgattcggtgcttgcgaccccctcggtgcgactcctctcggttttcgactatcacgcggaggtcgacagccagcgcgaagcgctggaggctgtgcgggcccggcttgagagtgccggccggacgatcgtcgtcgaggccctggcggaggctctcgcgcgggggcgggacctgcccgccgcgccgctgcccagtgcagatcccgatgccttgcgtctgctcatctacacctccggcagcaccggtacccccaagggcgccatgtatccgcaatggctggtcgccaacttgtggcagaagaagtggctcaccgacgatgtgattccgtccataggcgtgaacttcatgcccatgagccacctggcgggtcgcctcactctcatgggcaccctttccggtggcggaaccgcctactacatcgcttcgagcgatctttcgactttcttcgaggacatcgcgctcatccgcccctccgaagtgctcttcgtgccgcgtgtggtggagatggtgttccagcgttttcaggcagaattggaccggtcccttgccccgggtgagagcaactccgagatcgcggagcgaatcaaggtccgcatccgggaacaggacttcggcgggcgtgtgctcagtgctggctccgggtcggccccgttgtctcctgagatgacggagttcatggagtcgctgctgcaggtgccgttgcgcgacgggtatgggtccaccgaggccggtggtgtgtggcgtgacggagtcctgcagcgtccgcccgtcaccgactacaagctggttgacgttccggaactcggatacttcaccacagattcgccgcatccccgtggcgagctgcggttgaagtcggagacgatgttccccggctactacaagcgcccggagaccactgccgatgtcttcgatgacgaggggtactacaagaccggtgacgtggtcgccgagctcgggccggatcacctcaagtacctcgaccgcgtcaagaacgtcctcaagctcgcgcagggagagtttgtcgcggtgtcaaagctggaggccgcttacaccggcagcccgctggtccggcagatctttgtgtacgggaacagtgaacgctcgttcctgctggctgtcgtggtcccgacacccgaagtccttgagcggtacgcagattcgccagatgcgctcaagcccttgatccaggattcgctgcagcaggtcgccaaggacgcggagctgcaatcctanctgcaatcctatgagataccgcgcgacttcatcgttgagacggtgccgttcaccgtcgagtccggattgctatcggacgcgcgaaagctgctgcgccccaagctgaaggatcactacggagagaggctggaggcgctgtacgccgaactggcggaaagccagaatgagcggctgcgccagttggccagggaggcagccacgcgcccggtcctggagacggtgaccgatgcggccgccgcgctgctgggcgcatcgtcctcggatctggctcctgatgtgcgattcatcgacctcggtggcgactcactgtcggcgctgtcgtactccgagctgctgcgcgacatctttgaggtggacgttccggtgggcgtcatcaacagcgtcgccaacgaccttgccgcgatcgcccggcacatcgaggcgcagcggaccggcgccgctacgcagccgacctttgcgtcggtccacggcaaggacgcgacggtcatcaccgccggtgaactcaccctcgacaagttcttggacgagtcactgttgaaagcggccaaggacgttcagccggcaacggccgatgtcaagaccgttctagtgaccggcggcaacggctggttgggtcgttggctggtgctcgattggctggagcggttggcacccaatggtggcaaggtctacgccctcattcgtggcgccgatgccgaagcagcccgggcacggttggacgccgtgtacgaatcgggtgatcccaagctgtccgcgcattatcgtcagctggcgcaacagagtctggaagttatcgccggcgatttcggcgaccaggatctcggtctatcccaggaagtttggcagaagctggccaaggacgtggacctgatcgtgcactccggtgccttggtgaaccacgtgctgccgtacagccagttgttcggtccgaatgtggcgggtaccgccgagatcatcaagctggcaatttcggagcggctcaagccggtcacctacctgtcgacggtgggcatcgccgaccagattccggtgacggagttcgaggaagactccgatgttcgtgtgatgtcggccgagcgccagatcaatgacggctacgcgaacggatacggcaactcaaaatgggccggcgaggtgctgttgcgggaggctcatgacctagcggggctgccggtgcgtgtgttccgctccgacatgatcctggcgcacagtgactaccacggacagctcaacgtcaccgacgtgttcacccggagcatccagagtctgctgctcaccggtgttgcaccggccagcttctatgaattggatgccgacggcaatcggcagcgcgctcactatgacggtgtgcccggcgatttcaccgccgcatcgatcaccgccatcggcggtgtgaacgtggtagacggttaccgcagcttcgacgtgttcaacccgcaccatgacggtgtctcgatggataccttcgtcgactggctgatcgacgcaggctacaagatcgcgcggatcgacgattacgaccagtggctcgcccggttcgagctggccctcaagggattgcccgagcagcagcggcaacagtcggtgttgccacttctcaagatgtacgagaagccgcaaccggcgatcgacggaagtgcacttccgaccgcagaattcagtcgcgccgtgcacgaggcgaaggtcggagacagcggtgagataccgcacgtcaccaaggagctgatcctcaagtacgccagcgatattcagctgttgggcctggtg

SEQ ID NO: 96 - protein, 1182 aa, Mycobacteroides abscessus
MTETISTAAVPTTDLEEQVKRRIEQVVSNDPQLAALLPEDSVTEAVNEPDLPLVEVIRRLLEGYGDRPALGQRAFEFVTGDDGATVIALKPEYTTVSYRELWERAEAIAAAWHEQGIRDGDFVAQLGFTSTDFASLDVAGLRLGTVSVPLQTGASLQQRNAILEETRPAVFAASIEYLDAAVDSVLATPSVRLLSVFDYHAEVDSQREALEAVRARLESAGRTIVVEALAEALARGRDLPAAPLPSADPDALRLLIYTSGSTGTPKGAMYPQWLVANLWQKKWLTDDVIPSIGVNFMPMSHLAGRLTLMGTLSGGGTAYYIASSDLSTFFEDIALIRPSEVLFVPRVVEMVFQRFQAELDRSLAPGESNSEIAERIKVRIREQDFGGRVLSAGSGSAPLSPEMTEFMESLLQVPLRDGYGSTEAGGVWRDGVLQRPPVTDYKLVDVPELGYFTTDSPHPRGELRLKSETMFPGYYKRPETTADVFDDEGYYKTGDVVAELGPDHLKYLDRVKNVLKLAQGEFVAVSKLEAAYTGSPLVRQIFVYGNSERSFLLAVVVPTPEVLERYADSPDALKPLIQDSLQQVAKDAELQSXLQSYEIPRDFIVETVPFTVESGLLSDARKLLRPKLKDHYGERLEALYAELAESQNERLRQLAREAATRPVLETVTDAAAALLGASSSDLAPDVRFIDLGGDSLSALSYSELLRDIFEVDVPVGVINSVANDLAAIARHIEAQRTGAATQPTFASVHGKDATVITAGELTLDKFLDESLLKAAKDVQPATADVKTVLVTGGNGWLGRWLVLDWLERLAPNGGKVYALIRGADAEAARARLDAVYESGDPKLSAHYRQLAQQSLEVIAGDFGDQDLGLSQEVWQKLAKDVDLIVHSGALVNHVLPYSQLFGPNVAGTAEIIKLAISERLKPVTYLSTVGIADQIPVTEFEEDSDVRVMSAERQINDGYANGYGNSKWAGEVLLREAHDLAGLPVRVFRSDMILAHSDYHGQLNVTDVFTRSIQSLLLTGVAPASFYELDADGNRQRAHYDGVPGDFTAASITAIGGVNVVDGYRSFDVFNPHHDGVSMDTFVDWLIDAGYKIARIDDYDQWLARFELALKGLPEQQRQQSVLPLLKMYEKPQPAIDGSALPTAEFSRAVHEAKVGDSGEIPHVTKELILKYASDIQLLGLV

SEQ ID NO: 97 - DNA, 1218 bp, Methanococcus thermolithotrophicus
  source 1..1218: mol_type = genomic DNA
atggaaaaaggaaataacgaaatacttgaaaaacttttaaaaaaagaaataaaaccatatcaactggatgaccttgtaggggaaaaagaagccattgagttaagaagaaaatacattgaaaaaatctcccaagtggaaaccaaacacattgggcactatactatcgatgaaaaagaagccatgaagaagaacattgaaaacatgattggggcagttcagattccgcttggctttgcaggtcccttaaagataaatggaaaatatgcaaacggagagttttatgtgcccctagctacaacagagggagctcttgtagcttctgtaaacagggggtgcagtattgtaacaaaatgtgggggagtaacggttagagtgatagatgacaagatgaccagagctccagttataaaaacagaatctgtaattgatgcggttaaactaaaggagtggataaaagaaaacttccaaagaataaaagaagttgcagaaagtaccaccagacatggtaaattaatagacataaatccaattttaatagttggaagatatgtatatccaaggtttgtttataaaaccggggatgccatgggcatgaacatggtgacaattgccacagaaaaagcctgtaatttcattgaagaagaacttaaaaaggaaaatataaacatccatactgtggcattaagtgggaatgcctgtgtggacaaaaaacctgctggaataaacttaatagaaggtaggggaaaaagtataatcgcagaggtatttttaaaagaagaagagataaaaaaatacttaaaaacaacatcaaaggcgatcgaacaggtaaatatgtacaaaaacctaataggttctgcaataagtaattcaatgggtttcaatgcacactatgcaaatataataggagctctgtttttggcaactggccaggacgaagcccatatagttgaaggtagtttaggaataactgttgcagaatgtaccgaagatggagtttatttctcagttacacttcctgacgtaccggttggaaccgttggagggggtacaagggtagaaactcagaaggaatgtttagaactgttgggatgccatggtggagataaggcattgaagttcgccgaaatagttggtgcaacagttttagctggagaactatctttaataggagctctatcagtaggtcatctagccagagctcatgcccaattaggcaga

SEQ ID NO: 98 - protein, 407 aa, Methanococcus thermolithotrophicus
MMEKGNNEILEKLLKKEIKPYQLDDLVGEKEAIELRRKYIEKISQVETKHIGHYTIDEKEAMKKNIENMIGAVQIPLGFAGPLKINGKYANGEFYVPLATTEGALVASVNRGCSIVTKCGGVTVRVIDDKMTRAPVIKTESVIDAVKLKEWIKENFQRIKEVAESTTRHGKLIDINPILIVGRYVYPRFVYKTGDAMGMNMVTIATEKACNFIEEELKKENINIHTVALSGNACVDKKPAGINLIEGRGKSIIAEVFLKEEEIKKYLKTTSKAIEQVNMYKNLIGSAISNSMGFNAHYANIIGALFLATGQDEAHIVEGSLGITVAECTEDGVYFSVTLPDVPVGTVGGGTRVETQKECLELLGCHGGDKALKFAEIVGATVLAGELSLIGALSVGHLARAHAQLGR

SEQ ID NO: 99 - DNA, 1419 bp, Microbacterium radiodurans
  source 1..1419: mol_type = genomic DNA
atggcaagcccgacgctggacgccgcgatcggcggcgaggtggcggagaccaccacgacctcgccggagcaggtgcagcgcctcgtctcgcgggcgcgggccgcacaggccgtgttcgagaccttctcccaggagaaggtcgacgcgatcgtgcgcgacttcgccaagtacgtctacgacaacgccgaggcgctggccggcatggcccacagggagaccggcctcggcgtctacgaggacaaggtcgccaaggccaagggcaagtcccgtgtcatctggaacaacctcaagggcaagcgctcccgcggcatcatcggcgaggacgcggaggaggggctcatcctggtggccaaacccatgggcgtggtcggggcggtctgcccggtgacgaacccgatcgtcacgccgatgtgcaacgcgatgttcgccctgaaggccggcaacgccattatcttcgcgccacacccgaaggccgacgattccacctggcacctgaccagggagtaccagcggatcgtgcgcgagcacggcggacccgagcatctcgtccaggcggtgcggcgcggctcggtccagaccacccagaccctgatgcaggcggtggacgtggtggtggccaccggcggcggcgccatggtcaagtcggcctactcctcgggcaagccctcctatggggtcggggccggcaacgtgccggtggtgatcgaccgcggcgtcgacctgcgggaggcggcggagaagatcgtggcgggcgcctcgttcgacaacggcatcatctgctcgcacgagcagttcgtgctgacccccgaggagcgctacgccgacacggtcgcggccttccgcgacaccgggcgggtcttcttcaccgacgacccggccgaggtcgaccgggtgcgcgccgtggtgttcgaccacggccatctcaacaaggacgtggtcgggcgctcggcccaggagatcggcgcgcgggccggccttgccgtgcccgagggcgcgcggatcatcctcgtcccggcggacggggcgggcaacgacgacgtgctggccaaggagaagctctgccccgtggtcgcgatcctgccctacggaagcttcgaggaggcggtggaactcgccaaggccaacctcctggtcgagggcgccgggcactcggcggccctgcactcggcggacgaggcgcatatccgcgcgatgggcgtggcgttgccggtgagccggctggtggtcaaccagtcctcggcgctgaccgcgggcggctcgctgacgaacggcttcgcgccgaccacgacgctgggctgcggctcctggggcggcaactcgatctcggaaaacctcgactacaagcacctgatgaacgtctcccgcatcggcaacgtcatcagccgaccggtgccgagcgacgaggagatctgggcggcc

SEQ ID NO: 100 - protein, 473 aa, Microbacterium radiodurans
MASPTLDAAIGGEVAETTTTSPEQVQRLVSRARAAQAVFETFSQEKVDAIVRDFAKYVYDNAEALAGMAHRETGLGVYEDKVAKAKGKSRVIWNNLKGKRSRGIIGEDAEEGLILVAKPMGVVGAVCPVTNPIVTPMCNAMFALKAGNAIIFAPHPKADDSTWHLTREYQRIVREHGGPEHLVQAVRRGSVQTTQTLMQAVDVVVATGGGAMVKSAYSSGKPSYGVGAGNVPVVIDRGVDLREAAEKIVAGASFDNGIICSHEQFVLTPEERYADTVAAFRDTGRVFFTDDPAEVDRVRAVVFDHGHLNKDVVGRSAQEIGARAGLAVPEGARIILVPADGAGNDDVLAKEKLCPVVAILPYGSFEEAVELAKANLLVEGAGHSAALHSADEAHIRAMGVALPVSRLVVNQSSALTAGGSLTNGFAPTTTLGCGSWGGNSISENLDYKHLMNVSRIGNVISRPVPSDEEIWAA

SEQ ID NO: 101 - DNA, 912 bp, Escherichia coli K12
  source 1..912: mol_type = genomic DNA
atggcagatctcaattgggcagcaatcgcgtttatcggtttaggacaaatgggttcgccaatggcgagcaatttattgcagcaagggcatcaacttcgcgtctttgatgtgaacgccgaagctgtgcggcatctggtagacaaaggtgcgactcccgccgccaacccggcgcaggccgctaaagatgccgaatttatcattaccatgttgccgaatggcgatctggtgcgcaacgtgttgttcggtgaaaacggcgtttgcgaaggcttatctaccgatgcgctggtcattgatatgtccaccatccatccgctgcaaaccgataaattgattgccgatatgcaagccaaaggcttcagcatgatggatgttccggtaggccgtacttctgcaaatgccattaccggtactctgttactgctggctggcggcaccgctgaacaagttgaacgtgccacaccgatcctgatggcgatgggcagtgagttgatcaacgctggcggtccgggcatggggatccgcgttaagctcatcaacaactacatgagcatcgcgctcaatgcgctttcggcagaagccgccgttttgtgcgaagccctgaatcttcccttcgatgttgccgtcaaagtgatgagcggtaccgccgccggtaaaggtcacttcaccacttcctggccaaacaaagtcctcagcggagatctttctcccgccttcatgatcgatcttgcccataaggatcttggcatcgcccttgatgtcgccaaccagctgcatgtgccaatgccgctgggggccgcctcacgggaggtttatagccaggcgcgcgcagcgggtcgcggtcgccaggactggtccgccattctggaacaggtccgtgtcagtgccgggatgactgccaaagtaaaaatgtaa

SEQ ID NO: 102 - protein, 303 aa, Escherichia coli K12
MADLNWAAIAFIGLGQMGSPMASNLLQQGHQLRVFDVNAEAVRHLVDKGATPAANPAQAAKDAEFIITMLPNGDLVRNVLFGENGVCEGLSTDALVIDMSTIHPLQTDKLIADMQAKGFSMMDVPVGRTSANAITGTLLLLAGGTAEQVERATPILMAMGSELINAGGPGMGIRVKLINNYMSIALNALSAEAAVLCEALNLPFDVAVKVMSGTAAGKGHFTTSWPNKVLSGDLSPAFMIDLAHKDLGIALDVANQLHVPMPLGAASREVYSQARAAGRGRQDWSAILEQVRVSAGMTAKVKM

SEQ ID NO: 103 - DNA, 1077 bp, Homo sapiens
  source 1..1077: mol_type = genomic DNA
atgctgagtgccgcgtctcgcgtagtctcccgcgccgccgtccactgcgcgcttcgctctccgccgcccgaggcccgcgcgctcgccatgtcccggccaccgccaccgcgggtcgcctcggtgctgggcaccatggagatggggcgccgcatggacgcgcccgccagcgccgcggccgtgcgcgcctttctggagcgcggccacaccgaactggacacggccttcatgtacagcgacggccagtccgagaccatcctgggcggcctggggctcgggctgggcggtggcgactgcagagtgaaaattgccaccaaggccaacccttgggatggaaaatcactaaagcctgacagtgtccggtcccagctggagacgtcattgaagaggctgcagtgtccccaagtggacctcttctacctacacgcacctgaccacggcaccccggtggaagagacgctgcatgcctgccagcggctgcaccaggagggcaagttcgtggagcttggcctctccaactatgctagctgggaagtggccgagatctgtaccctctgcaagagcaatggctggatcctgcccactgtgtaccagggcatgtacaacgccaccacccggcaggtggaaacggagctcttcccctgcctcaggcactttggactgaggttctatgcctacaaccctctggctgggggcctgctgactggcaagtacaagtatgaggacaaggacgggaaacagcctgtgggccgcttctttgggaatagctgggctgagacctacaggaatcgcttctggaaggagcaccacttcgaggccattgcgttggtggagaaggccctgcaggccgcatatggcgccagcgcccccagtgtgacctcggctgccctccggtggatgtaccaccactcacagctgcagggtgcccacggggacgcggtcatcctgggcatgtccagcctggagcagctggagcagaacttggcagcaacagaggaagggcccctggagccggctgtcgtggatgcctttaatcaagcctggcatttggttgctcacgaatgtcccaactacttccgc

SEQ ID NO: 104 - protein, 359 aa, Homo sapiens
MLSAASRVVSRAAVHCALRSPPPEARALAMSRPPPPRVASVLGTMEMGRRMDAPASAAAVRAFLERGHTELDTAFMYSDGQSETILGGLGLGLGGGDCRVKIATKANPWDGKSLKPDSVRSQLETSLKRLQCPQVDLFYLHAPDHGTPVEETLHACQRLHQEGKFVELGLSNYASWEVAEICTLCKSNGWILPTVYQGMYNATTRQVETELFPCLRHFGLRFYAYNPLAGGLLTGKYKYEDKDGKQPVGRFFGNSWAETYRNRFWKEHHFEAIALVEKALQAAYGASAPSVTSAALRWMYHHSQLQGAHGDAVILGMSSLEQLEQNLAATEEGPLEPAVVDAFNQAWHLVAHECPNYFR

SEQ ID NO: 105 - DNA, 1923 bp, Saccharomyces cerevisiae
  source 1..1923: mol_type = genomic DNA
agcgtgtgaggactactcattggcttgcgatttacggtttttatattttttgccgcacatcattttttggcctggtattgtcatcgcgttgagcggactctgaatataatcctattgttttttatggatctctggaagcgtctttttgaagccaacccaacaaaaattcgagacaagaaaataaaaaacggcacttcatcagtatcacaaataccatcaatttatcagctctcatgaatgaaatcgatgagaaaaatcaggcccccgtgcaacaagaatgcctgaaagagatgattcagaatgggcatgctcggcgtatgggatctgttgaagatctgtatgttgctctcaacagacaaaacttatatcggaacttctgcacatatggagaattgagtgattactgtactagggatcagctcacattagctttgagggaaatctgcctgaaaaatccaactcttttacatattgttctaccaataagatggccaaatcatgaaaattattatcgcagttccgaatactattcacggccacatccagtgcatgattatatttcagtattacaggaattgaaactgagtggtgtggttctcaatgaacaacctgagtacagtgcagtaatgaagcaaatattagaagaattcaaaaatagtaagggttcctatactgcaaaaatttttaaacttactaccactttgactattccttactttggaccaacaggaccgagttggcggctaatttgtcttccagaagagcacacagaaaagtggaaaaaatttatctttgtatctaatcattgcatgtctgatggtcggtcttcgatccacttttttcatgatttaagagacgaattaaataatattaaaactccaccaaaaaaattagattacattttcaagtacgaggaggattaccaattgttgaggaaacttccagaaccgatcgaaaaggtgatagactttagaccaccgtacttgtttattccgaagtcacttctttcgggtttcatctacaatcatttgagattttcttcaaaaggtgtctgtatgagaatggatgatgtggaaaaaaccgatgatgttgtcaccgagatcatcaatatttcaccaacagaatttcaagcgattaaagcaaatattaaatcaaatatccaaggtaagtgtactatcactccgtttttacatgtttgttggtttgtatctcttcataaatggggtaaatttttcaaaccattgaacttcgaatggcttacggatatttttatccccgcagattgccgctcacaactaccagatgatgatgaaatgagacagatgtacagatatggcgctaacgttggatttattgacttcaccccatggataagcgaatttgacatgaatgataacaaagaaaaattttggccacttattgagcactaccatgaagtaatttcggaagctttaagaaataaaaagcacctccatggcttagggttcaatatacaaggcttcgttcaaaaatatgtgaatattgacaaggtaatgtgcgatcgtgccatcgggaaaagacgcggaggtacattgttaagcaatgtaggtctgtttaatcagttagaggagcccgatgccaaatattctatatgcgatttggcatttggccaatttcaaggatcctggcaccaagcattttccttgggtgtttgttcgactaatgtaaaggggatgaatattgttgttgcttcaacaaaaaatgttgttggtagccaagaatctctcgaagagctttgctccatttataaagctctccttttaggcccttagatctcacatgatgcttgactgatattattcgacaatatgattatgtcgtgtaaataacccactttcatgttgtcactccctcggctttggttggttaaagggacttattggt

SEQ ID NO: 106 - protein, 525 aa, Saccharomyces cerevisiae
MNEIDEKNQAPVQQECLKEMIQNGHARRMGSVEDLYVALNRQNLYRNFCTYGELSDYCTRDQLTLALREICLKNPTLLHIVLPTRWPNHENYYRSSEYYSRPHPVHDYISVLQELKLSGVVLNEQPEYSAVMKQILEEFKNSKGSYTAKIFKLTTTLTIPYFGPTGPSWRLICLPEEHTEKWKKFIFVSNHCMSDGRSSIHFFHDLRDELNNIKTPPKKLDYIFKYEEDYQLLRKLPEPIEKVIDFRPPYLFIPKSLLSGFIYNHLRFSSKGVCMRMDDVEKTDDVVTEIINISPTEFQAIKANIKSNIQGKCTITPFLHVCWFVSLHKWGKFFKPLNFEWLTDIFIPADCRSQLPDDDEMRQMYRYGANVGFIDFTPWISEFDMNDNKENFWPLIEHYHEVISEALRNKKHLHGLGFNIQGFVQKYVNIDKVMCDRAIGKRRGGTLLSNVGLFNQLEEPDAKYSICDLAFGQFQGSWHQAFSLGVCSTNVKGMNIVVASTKNVVGSQESLEELCSIYKALLLGP

SEQ ID NO: 107 - DNA, 2623 bp, Saccharomyces cerevisiae
  source 1..2623: mol_type = genomic DNA
gacccttttctcgatatgtttttctgtgatttttttttttcaattgttcgaaataacctgtattgttcaatactggacaattgttctgatatatatatatatatataattttcaatgaaaaaaaaaaaaaaaagaaagaaaattagaagctagaaagaaataaggaaaaggaaaagcaaacaacaataggaaaaaaaagcatagaaacatataatagattaaatacaaaagatttattatacatgcgaggtaagactattgtctaagacttatacccgaccgagagcagccacggcatgcatcgactacgagcgtattgtgttatatcagctctgtccgatacactgcttgccttttgtacgagctcggccgagctatacgaaggcccgctacggcagtatcgcattaaagcgacgcaaattcgccgatggtttggtagaagagcttgcagaacgattcccattcgccacgatcccgtagagtgccctgaaccacgctcatgtccatgttcataccattcacgtttgtggagcaaacgtttaggccgaacgcaaatctcaaggcgcctgcagactgcgagaagattaaatcccgaacgtagtacggcttggtcgtatcttgcgagacaagtcctgtattactgatgattgcaccgcctctttgctggtgcaaataagagttggcaagaaggctgtctatattcttgttttcaacgataaaatcaagttgtaggacccccagaccaatcaagtggtcaccgttgtcgtacgattctaaaaagcggtcatagtagtattcgacaagactccaaaacttgtcattgtcaccttcgggaatgtcgaatgaggagattagataggcgtaatgcgaacctccaacgttggagccgtatttataagaatctcttaactcttcatcgtttggtaaaaaccttcttgcgttgcttggaatagccacatcgaacccatactcgagaagagattttgtgaacagcttatccagtctatacagggctacaagaaagcacgcttgaatgaagggtgttagggtgcacccatcgtgaacatttttcttgatctgagctctgatttgttctacttgcttagtactgaaatgcaacaagtaattgtaactagcattagcattactagaggggctatatctagctgtaagtgtagattcgctggaggttttaaaattacaatgttcataaatgaaggttgttaaaaagaatttgggtaatgaagtcaatgctggcttgtagtcaatacgatccgtaatcggttttggcaatttagaaatttcagtgtagtctcgatcatagtcaatgatgacttgatcttcgatgaactcttcatcataatcaaaccctttttcttcgattttacaaaagagtagagctaaatctttgaaaaaattcgatccactgacaccgtcggagccgcagtggttagtgacataaacgaagtttttccacgtttcaaacccatcagtatccttacctggtaaacaaatcaatctccaattaggcctcttcggattacccagaggtatgattacagggctaatcaattcgattaacctattggtgactttgaaatcatcctttttgaactgttctgagattttctccatgacttctctgtattctggttgattattcataatcaagtcatcgaattcaagatgagaaatcacctttatgaaatcatgctgtggaaaaggtttactaaggtactcttcgctagagtagtacgcttcatgatgaggataatgcttaggaatgattgtatgcgcaagagttgagtatttttgaagtaatactttcaagacaagcattagttgtctcttattaacacctttattcaattccgcgtaaacagtaaaattcgagtacattttctgcctactcaaaacagcaaagtagttttccaagtggcccatacgtcttgcatggccacggtctatcaactcttgagtgatatgtggttcgtatccttctatatcttccataattaacctggacaatttttattgctgaagttattattcgtttgaaagtctcagtactgtttgtacgcattaggaatgacattgctgttactgcagaattgccctacacatttgttgttgtcttttaagtaacaatttgctctattttcacagacctcgaagaaacgtaaatcttgacaacaaatgtttacgatcgtttaggtgtatgaaaaaatatagaataccaaaataggaaactcgttgaattcgtttactcattatcccgcaatatttatcggtggcctacagagttcaatgtagcctgagattgtggcgattgaaatactgattacttaaaatttgctaaagcctgttctatctgaagaaaaaaaaaaaaaatggatgagccttccagttcattgaaattcttcagttcttatttttttttcaaaaagaaaagtttcagtaccctgttccaaaaaaaatgaaaaaaatgaaaatccagtatccttcccataaaaattaaaagttccattatttatgggtaagtaagtaccactgattatataccatatttataaagtgcatgaatttattactatatattgctttttctgacgtgcttccttca

SEQ ID NO: 108 - protein, 535 aa, Saccharomyces cerevisiae
MEDIEGYEPHITQELIDRGHARRMGHLENYFAVLSRQKMYSNFTVYAELNKGVNKRQLMLVLKVLLQKYSTLAHTIIPKHYPHHEAYYSSEEYLSKPFPQHDFIKVISHLEFDDLIMNNQPEYREVMEKISEQFKKDDFKVTNRLIELISPVIIPLGNPKRPNWRLICLPGKDTDGFETWKNFVYVTNHCGSDGVSGSNFFKDLALLFCKIEEKGFDYDEEFIEDQVIIDYDRDYTEISKLPKPITDRIDYKPALTSLPKFFLTTFIYEHCNFKTSSESTLTARYSPSSNANASYNYLLHFSTKQVEQIRAQIKKNVHDGCTLTPFIQACFLVALYRLDKLFTKSLLEYGFDVAIPSNARRFLPNDEELRDSYKYGSNVGGSHYAYLISSFDIPEGDNDKFWSLVEYYYDRFLESYDNGDHLIGLGVLQLDFIVENKNIDSLLANSYLHQQRGGAIISNTGLVSQDTTKPYYVRDLIFSQSAGALRFAFGLNVCSTNVNGMNMDMSVVQGTLRDRGEWESFCKLFYQTIGEFASL

SEQ ID NO: 109 - DNA, 1397 bp, Saccharomyces cerevisiae
  source 1..1397: mol_type = genomic DNA
tagcaatgtctgcagagactttctccgcttgcataaccttataaacttataacgatacctacttatttcatatcttatgtatagttctccttcaaggagacgttgttttaatacttttatctattccatttatcattatctactttgtaatcaaagatcgaagcactgctgtgtgcgttgatttgggcctgacacgagaagaaagtgcgttacgtacatcaagattttattcaaaatccagcgtctcctccgcaaaaaagttcagaatacatttgaatgagtcctctggattctccaacagtaaattatgcccagtattaaattctcggacatcagcacagggaaagttatacctcaggagggaataatcattgttaataaagttagactgcaggcctttcatgaataaaactttacgactggctgtgcctttttggaagaagtccctttggttcgatagatccggccattttttcacttcatccaacaggttcggcatgctggagagaggcattgagtaatttatataaggatcatgctgttgttgttcatagtgtaactgtgcttggtgtacattattggattttacagaaagaaaaccgttggcaaagtagagtgccacagctccaccacatttcctcttattacattccaaacttttgaaaagctcaagaacttttttctgccagctgggggaacctcttaaaatcttgacatctctgcggataatccgcattattaagtcatagttttgtaatatcatgggatctaattctggggtttcataaggcggtaaatcaattgatatacactttcgtatattaatattcttgtacaatgtggttaataatgcaattttaccacccatagagaatcccagtaaataaataggtctctccaagccaatatgagtttcaataaaataaatcaaatcattggtcagtgtagtgtagtcataaggtattgcttttggtgaaatgccatgatttcttacatcaacagaaaaaatatccgcgtcaagcttccttgaaagaagcttattgagagaatgaaacatcacgtgtgagcccaaaaggccatggatgttaataattgcaggtctttgctcgaattttattagcgatgaaacatctgagggtaaacgcgttctatggaatgataaatcgacaattattttataaggtagcgctttattatgcgctagtctcgacatttgtaagccctcttcccacggttccttttattttgcagtttgtatacgtctcgaccttggcatgagacaaaaacagtttccccaaaagacgcgctgtggtattcagtaaaggctggtttctatgataggttgagtttagaataatttttaaacaggatactaagataaagcaaatatttcaatc

SEQ ID NO: 110 - protein, 328 aa, Saccharomyces cerevisiae
MSRLAHNKALPYKIIVDLSFHRTRLPSDVSSLIKFEQRPAIINIHGLLGSHVMFHSLNKLLSRKLDADIFSVDVRNHGISPKAIPYDYTTLTNDLIYFIETHIGLERPIYLLGFSMGGKIALLTTLYKNINIRKCISIDLPPYETPELDPMILQNYDLIMRIIRRDVKILRGSPSWQKKVLELFKSLECNKRKCGGAVALYFANGFLSVKSNNVHQAQLHYEQQQHDPYINYSMPLSSMPNLLDEVKKWPDLSNQRDFFQKGTASRKVLFMKGLQSNFINNDYSLLRYNFPCADVREFNTGHNLLLENPEDSFKCILNFFAEETLDFE

SEQ ID NO: 111 - DNA, 648 bp, Mycobacterium smegmatis
  source 1..648: mol_type = genomic DNA
atggccaagcgaattctgtgtttcggtgattccctgacctggggctgggtccccgtcgaagacggggcacccaccgagcggttcgcccccgacgtgcgctggaccggtgtgctggcccagcagctcggagcggacttcgaggtgatcgaggagggactgagcgcgcgcaccaccaacatcgacgaccccaccgatccgcggctcaacggcgcgagctacctgccgtcgtgcctcgcgacgcacctgccgctcgacctggtgatcatcatgctgggcaccaacgacaccaaggcctacttccggcgcaccccgctcgacatcgcgctgggcatgtcggtgctcgtcacgcaggtgctcaccagcgcgggcggcgtcggcaccacgtacccggcacccaaggtgctggtggtctcgccgccaccgctggcgcccatgccgcacccctggttccagttgatcttcgagggcggcgagcagaagaccactgagctcgcccgcgtgtacagcgcgctcgcgtcgttcatgaaggtgccgttcttcgacgcgggttcggtgatcagcaccgacggcgtcgacggaatccacttcaccgaggccaacaatcgcgatctcggggtggccctcgcggaacaggtgcggagcctgctg

SEQ ID NO: 112 - protein, 216 aa, Mycobacterium smegmatis
MAKRILCFGDSLTWGWVPVEDGAPTERFAPDVRWTGVLAQQLGADFEVIEEGLSARTTNIDDPTDPRLNGASYLPSCLATHLPLDLVIIMLGTNDTKAYFRRTPLDIALGMSVLVTQVLTSAGGVGTTYPAPKVLVVSPPPLAPMPHPWFQLIFEGGEQKTTELARVYSALASFMKVPFFDAGSVISTDGVDGIHFTEANNRDLGVALAEQVRSLL

SEQ ID NO: 113 - DNA, 1110 bp, Arthrobacter nitroguajacolicus
  source 1..1110: mol_type = genomic DNA
atgcattcacaggtgatcgccccaggtttcgaacccgtcgccgaactcttcggcgtctttcttgagcaggatccggactactctgcgcaagtggccgcgtatcaccgtggcgtcaaagtgctggacctcagcggcggcccccacatccgcccggattcagtcaccggcgtcttctcgtgttccaaaggcatggccggtttggtcatggcgttgctcgttcaggacggcgaactggaccttgaagccgaggtggtcaagtactggcccgagttcggcgttgaaggcaagtcctccatcacggttgcgcagctgctttcgcaccgagcggggctcttgggagtggaaggcggcctgacgctccacgaggtcaacaactctgagctcgcagcagccaaactcgccgagctgccgcccctatggaaacccggtaccgcgttcggttaccacgcgctgaccattggcatcttcatggaagagctgtgccggcgcatcaccggctccacgctgcaggaggtcttcgagcagcgcatccgtgcggtgactggtgcgaacttctaccttggtttgcccgagtccgaagagtcccgcttcgcgcagttccgctgggcggccgatccgtcatggccttgggtagacccggccagccactttggcctggctgccaacgcggcagtgggcgacatcctggatctgcccaacatccgggaagtccgggccgcgggcctgagctcggcggccggcgtggccagcgcggaaggcatggcgcggatctacgcggcggcattgactggcttggagggtaaatccgccatgccgcccctcctgaccgaggagactatccggacggtgtcggctgagcaggtgttcggaatcgatcgcgtttttggtgagaccggatgcttcggcaccgttttcatgaagtcccacaccaggatgccgttcggcagttaccgcgcgttcgggcacgacggcgccagcgcatcgttggggttcgccgaccctgtgtatgaactcggctttggatatgtcccgcagacggctgagcctggaggggtgggctgccgtaacttccagctgagctcggcagtaagggaagttatcgccggtttc

SEQ ID NO: 114 - protein, 372 aa, Arthrobacter nitroguajacolicus
MHSQVIAPGFEPVAELFGVFLEQDPDYSAQVAAYHRGVKVLDLSGGPHIRPDSVTGVFSCSKGMAGLVMALLVQDGELDLEAEVVKYWPEFGVEGKSSITVAQLLSHRAGLLGVEGGLTLHEVNNSELAAAKLAELPPLWKPGTAFGYHALTIGIFMEELCRRITGSTLQEVFEQRIRAVTGANFYLGLPESEESRFAQFRWAADPSWPWVDPASHFGLAANAAVGDILDLPNIREVRAAGLSSAAGVASAEGMARIYAAALTGLEGKSAMPPLLTEETIRTVSAEQVFGIDRVFGETGCFGTVFMKSHTRMPFGSYRAFGHDGASASLGFADPVYELGFGYVPQTAEPGGVGCRNFQLSSAVREVIAGFAA

SEQ ID NO: 115 - DNA, 1164 bp, Uncultured bacterium
  source 1..1164: mol_type = genomic DNA
atgtcgatagcggatcagtcattagcaaaaagagtgcagggcgttagccaacaggcgattgatgaagggcgtatcgttggcagcgtggtgctgatcgctcggcacggtcgcgtgatttacgccaatgccagcggctatgccgatcgtgaacagaagaaacctatggtgcgtgagacccaatttcggctgtcgtcggtgtccaagccttatattacgctggcggccatgcgtatgatcgaacagcagaagctggggctggatgataccgtcagccgttggttgccgtggtttaccccggcgctggccgatggggttcgcccgccaattaaaatccgtcacttgttgagccacactgccggcctggattatcgtttgagccaacctgcggaaggaccgtatcatcgactcggtattaaagacggtatggaactgtcgtcgttaacgctggaacagaatctgcgcctgttggcgcaggcggatctgttggccgagccgggcagcgagtttcgatattcactggcaatcgatgtgctgggggcggtgctggaacaggtggcgggcgagcccttgccgcaggtgttcaaccattgggttgcccaacctttggggttgcgtaataccggtttttacaccaccgatgtcgataatctggcaacggcgtatcacgacaccgccgcggagccggaacctatacgagatggcatgttgctgaccctgccggaagggttcggcttcgagattgaactggcaccctcgcgcgcactggacgctcaggcctatccttctggcggcgctggcatggtcggcgatgcagacgatgtgttgcagttggtggaaaccttgcgcactggcaaggaaggcattttacagccggccaccgcagcgctgatgcgtcaagcgcatgtcgggtcgcacgccgagactcaggggcccggctgggggtttggtttcggcggtgcggtactggaagatgcgcagttggcggcgacgcctcagcacaatggcactctgcagtggggcggtgtctatggccacagttggttttacgatccgcaagcggcgatcagcgtggtagccttgaccaatacggcctttgaaggcatgagtggacgttatccactgcaaatccgcgatgctgtttacgggacaaacgaacctactcgc

SEQ ID NO: 116 - protein, 388 aa, Uncultured bacterium
MSIADQSLAKRVQGVSQQAIDEGRIVGSVVLIARHGRVIYANASGYADREQKKPMVRETQFRLSSVSKPYITLAAMRMIEQQKLGLDDTVSRWLPWFTPALADGVRPPIKIRHLLSHTAGLDYRLSQPAEGPYHRLGIKDGMELSSLTLEQNLRLLAQADLLAEPGSEFRYSLAIDVLGAVLEQVAGEPLPQVFNHWVAQPLGLRNTGFYTTDVDNLATAYHDTAAEPEPIRDGMLLTLPEGFGFEIELAPSRALDAQAYPSGGAGMVGDADDVLQLVETLRTGKEGILQPATAALMRQAHVGSHAETQGPGWGFGFGGAVLEDAQLAATPQHNGTLQWGGVYGHSWFYDPQAAISVVALTNTAFEGMSGRYPLQIRDAVYGTNEPTR

SEQ ID NO: 117 - DNA, 1080 bp, Homo sapiens
  source 1..1080: mol_type = genomic DNA
atgctgagtgccgcgtctcgcgtagtctcccgcgccgccgtccactgcgcgcttcgctctccgccgcccgaggcccgcgcgctcgccatgtcccggccaccgccaccgcgggtcgcctcggtgctgggcaccatggagatggggcgccgcatggacgcgcccgccagcgccgcggccgtgcgcgcctttctggagcgcggccacaccgaactggacacggccttcatgtacagcgacggccagtccgagaccatcctgggcggcctggggctcgggctgggcggtggcgactgcagagtgaaaattgccaccaaggccaacccttgggatggaaaatcactaaagcctgacagtgtccggtcccagctggagacgtcattgaagaggctgcagtgtccccaagtggacctcttctacctacacgcacctgaccacggcaccccggtggaagagacgctgcatgcctgccagcggctgcaccaggagggcaagttcgtggagcttggcctctccaactatgctagctgggaagtggccgagatctgtaccctctgcaagagcaatggctggatcctgcccactgtgtaccagggcatgtacaacgccaccacccggcaggtggaaacggagctcttcccctgcctcaggcactttggactgaggttctatgcctacaaccctctggctgggggcctgctgactggcaagtacaagtatgaggacaaggacgggaaacagcctgtgggccgcttctttgggaatagctgggctgagacctacaggaatcgcttctggaaggagcaccacttcgaggccattgcgttggtggagaaggccctgcaggccgcatatggcgccagcgcccccagtgtgacctcggctgccctccggtggatgtaccaccactcacagctgcagggtgcccacggggacgcggtcatcctgggcatgtccagcctggagcagctggagcagaacttggcagcaacagaggaagggcccctggagccggctgtcgtggatgcctttaatcaagcctggcatttggttgctcacgaatgtcccaactacttccgctag

SEQ ID NO: 118 - protein, 359 aa, Homo sapiens; identical to SEQ ID NO: 104

SEQ ID NO: 119 - DNA, 2772 bp, Saccharomyces cerevisiae
  source 1..2772: mol_type = genomic DNA
atgtcaagcggcagtactattgtttcgtctgacaagtcggggcgaacatttaagcatgaggaagaactacccaagttaccattacctaaactctgtgacactttgcagcgtctgaaggagagtttagagccattgtattatgctgatggttactatcaacatcctttagatccagaacaaattgagaagctgtcatccataataagagattttgaagagaatcccgtaagtgagaagctgcaatctaaactacagagctaccatgacactagggattgctaccttgatgaattacacttagacatcaataaccagacctccactagggagattcaggacgatgtcctacccaggaatcccttcttggttctcgcagatgacgctttgccaaacatcactcaagctgataggtcagccgtcctagtgcactctgcggccaggtttatttctgctttgaaacaagatttgttgccaccagatatcaatgccaccaatggcaaaccactgtcaatggctccatttctgaacttgttcggtactactcgtagtcctgtttttcagcgcggtgaggttgaaaattttgatttgaacaaaccttacacagcatctgatttagaagaccctgattattcaagcgatgaggacgataacgacgaacccacccaaaaagactttgatgaccgtaaaagaaaacacgaagaagacatattcactggcaatggtataactataaaaagacatccggatagcaaacacatcttgatcatctccaggggccaatactatacactggaagtacttgattcgacgaataaaattatatacactgctgcagaattaaccacaattttcaaccatataataaaagattcttctggcattgaaaaatcaactgcgttaggtagtttgacctctcactcttttagaaactggaaatatgcaaggaagagactacagaaaaggtatcctaatgaattacatcgcatagactctgctctgttcgtgttagtgctggacgaatcacaagaagaaactaccaatgatggcgatgataccgctgatatcagtcaaatgtttaacaggactatcactgagcgagacaagaaatgtacttccgccaattgtaagagagtgttttacggtacttctataataaatagcaaaggccatcaagtgggttcatgtgtttctcgttggtatgataaattacagttagttgtcactgcagatgcaaaagccacagtgatttgggattccttcacatgtgacgggtcagtcgtgcttcggtttacttctgaaatttatacagaatcagtcttgagattagctagagacgttaacgccggtgacccacagttttcactctggcctaacgtgacacaaatggatcctgaaactaaaaaactgatgacggcgaccataagtgccgacggcggaggcccttctgaaattgaccctaagttggttgtaaataaaatcgattggtctttcagtaacatcctgaacacacatgtccatttgtcagaaaccaaactagctgatctgatttccaagtatgatattgttcgcgcttctattccattgggcagaaggtcagctcaaagattaggagtcaagcccgattcgatggttcaagtagctttacaaatagctcactatgctttgtatggaagaatggtgtttggattagaaccagtatccacacgtggtttcaaaaactcgagatcatcttttattaacattcaaagtcaagcgctgttggaactatgtcaattgtttatttctagctccattgatgggacagataaattagacaaatttatccaaacctgcgaaacacataataacatggttaaacatgcaaagtctggggtaggttatgaaaaacacttcaatgcattgaaatatctcttcaaatttcatgaccattttggtatccacttaagtggtgatgaatcatctgctgcgaaagatcttttcgaaaatccacttgttttacctttctcacagcctgagttgattgttgccaactgtggtaatgctgctaccacgacatttggtataactcctgctgtacctcatggctttggaatagggtacatcattaaagatgatcaagtagatttgaccgtgacatcgcaatttcgacagggtgacaggttaatgtttatgttaagttgggtgttaggcgaaattcgttcctactggaggatgtcacgcggcacttctcataataaaactggcgtgaagatcagtccagtggtcgataaattgtatgaaatggacaatgcagtcaataatcctccgaaacgtaacggccatacagtcaatggctcccgcaaaacatcttcttcatcacaggtaaatttgaacaggtatggcgggtttttcgatttggaaggccacattgacagtaggaatatatctaaaacaccatcgatgaagaatttgcagaaaaccttcaatggcttgaccatgagcgcagacaatgatcattcaagttccgcagtttccgttcccacagaaaaggaaaaattaaatacaggccatgaaattttacagatccaaccgcgggaagtggcaagtaatgggttggaagcagatgatgagacagatattgaaatagttgctggcaatgctgatggcacatcctcttcggcatcttcagctacatctctaaattccaagaaacgtaacgtcatcaactcaagattcgatattgattttgaccgcagtcgtgtgggtagaaaggtggcgaccttagatcaataa

SEQ ID NO: 120 - protein, 923 aa, Saccharomyces cerevisiae
MSSGSTIVSSDKSGRTFKHEEELPKLPLPKLCDTLQRLKESLEPLYYADGYYQHPLDPEQIEKLSSIIRDFEENPVSEKLQSKLQSYHDTRDCYLDELHLDINNQTSTREIQDDVLPRNPFLVLADDALPNITQADRSAVLVHSAARFISALKQDLLPPDINATNGKPLSMAPFLNLFGTTRSPVFQRGEVENFDLNKPYTASDLEDPDYSSDEDDNDEPTQKDFDDRKRKHEEDIFTGNGITIKRHPDSKHILIISRGQYYTLEVLDSTNKIIYTAAELTTIFNHIIKDSSGIEKSTALGSLTSHSFRNWKYARKRLQKRYPNELHRIDSALFVLVLDESQEETTNDGDDTADISQMFNRTITERDKKCTSANCKRVFYGTSIINSKGHQVGSCVSRWYDKLQLVVTADAKATVIWDSFTCDGSVVLRFTSEIYTESVLRLARDVNAGDPQFSLWPNVTQMDPETKKLMTATISADGGGPSEIDPKLVVNKIDWSFSNILNTHVHLSETKLADLISKYDIVRASIPLGRRSAQRLGVKPDSMVQVALQIAHYALYGRMVFGLEPVSTRGFKNSRSSFINIQSQALLELCQLFISSSIDGTDKLDKFIQTCETHNNMVKHAKSGVGYEKHFNALKYLFKFHDHFGIHLSGDESSAAKDLFENPLVLPFSQPELIVANCGNAATTTFGITPAVPHGFGIGYIIKDDQVDLTVTSQFRQGDRLMFMLSWVLGEIRSYWRMSRGTSHNKTGVKISPVVDKLYEMDNAVNNPPKRNGHTVNGSRKTSSSSQVNLNRYGGFFDLEGHIDSRNISKTPSMKNLQKTFNGLTMSADNDHSSSAVSVPTEKEKLNTGHEILQIQPREVASNGLEADDETDIEIVAGNADGTSSSASSATSLNSKKRNVINSRFDIDFDRSRVGRKVATLDQ

SEQ ID NO: 121 - DNA, 612 bp, Escherichia coli K12
  source 1..612: mol_type = genomic DNA
ttgaacatgccaatgaccgaaagaataagagcaggcaagctatttaccgatatgtgcgaaggcttaccggaaaaaagacttcgtgggaaaacgttaatgtatgagtttaatcactcgcatccatcagaagttgaaaaaagagaaagcctgattaaagaaatgtttgccacggtaggggaaaacgcctgggtagaaccgcctgtctatttctcttacggttccaacatccatataggccgcaatttttatgcaaatttcaatttaaccattgtcgatgactacacggtaacaatcggtgataacgtactgattgcacccaacgttactctttccgttacgggacaccctgtacaccatgaattgagaaaaaacggcgagatgtactcttttccgataacgattggcaataacgtctggatcggaagtcatgtggttattaatccaggcgtcaccatcggggataattctgttattggcgcgggtagtatcgtcacaaaagacattccaccaaacgtcgtggcggctggcgttccttgtcgggttattcgcgaaataaacgaccgggataagcactattatttcaaagattataaagttgaatcgtcagtttaa

SEQ ID NO: 122 - protein, 203 aa, Escherichia coli K12
MNMPMTERIRAGKLFTDMCEGLPEKRLRGKTLMYEFNHSHPSEVEKRESLIKEMFATVGENAWVEPPVYFSYGSNIHIGRNFYANFNLTIVDDYTVTIGDNVLIAPNVTLSVTGHPVHHELRKNGEMYSFPITIGNNVWIGSHVVINPGVTIGDNSVIGAGSIVTKDIPPNVVAAGVPCRVIREINDRDKHYYFKDYKVESSV

SEQ ID NO: 123 - DNA, 1247 bp, Homo sapiens
  source 1..1247: mol_type = genomic DNA
ggaattcgccgcccgctccgagcctggtgcttccgaccgctgcgcgcggctcctgggctgtcacagtctcccgttgccgccgtcatgtcccggcagctgtcgcgggcccggccagccacggtgctgggcgccatggagatggggcgccgcatggacgcgcccaccagcgccgcagtcacgcgcgccttcctggagcgcggccacaccgagatagacacggccttcgtgtacagcgacggccagtccgagaccatccttggcggcctggggctccggctgggcggcagcgactgcagagtgaaaattgataccaaggccattccactgtttgggaactccctgaagcctgacagtctccggttccagctggagacgtcactgaagcggctgcagtgtccccgagtggacctcttctacctgcatatgccagaccacagcaccccggtggaagagacactgcgtgcctgccaccagctgcaccaggagggcaagttcatggagcttggcctctccaactatgcagcctgggaagtggccgagatctgtaccctctgcaagagcaacggctggatcctgcccactgtgtaccagggcatgtacaatgccatcacccggcaggtggaaacggagctcttcccctgcctcaggcactttggactgaggttctatgccttcaaccctctggctgggggcctgctgaccggcaagtacaagtatgaggacaaggatgggaaacagcctgtgggccgcttctttgggaatacctgggcagagatgtacaggaatcgctactggaaggagcaccactttgagggcattgccctggtggagaaggccctgcaggccgcgtatggcgccagcgcccccagcatgacctcggccaccctccggtggatgtaccaccactcacagctgcagggtgcccacggggacgcggtcatcctgggcatgtccagcctggagcagctggagcagaacttggcagcggcagaggaagggcccctggagccggctgtcgtggacgcctttaatcaagcctggcatttggttgctcacgaatgtcccaactacttccgctaggcccatcgtttctcaggctgcccaaggctcttctgtaacatcttttgttactcacattttctttaatttagaactgcctcagtaaattcttagggatggaagtntttggacaaaaacctaatagaagagtcaccaccaaatgaagaataaaactcccaaaaaaaaaa

SEQ ID NO: 124 - protein, 331 aa, Homo sapiens
MSRQLSRARPATVLGAMEMGRRMDAPTSAAVTRAFLERGHTEIDTAFVYSEGQSETILGGLGLRLGGSDCRVKIDTKAIPLFGNSLKPDSLRFQLETSLKRLQCPRVDLFYLHMPDHSTPVEETLRACHQLHQEGKFVELGLSNYAAWEVAEICTLCKSNGWILPTVYQGMYNAITRQVETELFPCLRHFGLRFYAFNPLAGGLLTGKYKYEDKNGKQPVGRFFGNTWAEMYRNRYWKEHHFEGIALVEKALQAAYGASAPSMTSATLRWMYHHSQLQGAHGDAVILGMSSLEQLEQNLAAAEEGPLEPAVVDAFNQAWHLVTHECPNYFR

(19) United States
(12) Patent Application Publication
(10) Pub. No.: US 2025/0313871 A1
(43) Pub. Date: Oct. 9, 2025

(54) BIOCHEMICAL PATHWAY FOR THE PRODUCTION OF TULIPALIN A VIA ITACONIC ACID

(71) Applicants: BASF SE, Ludwigshafen am Rhein (DE); MAX-PLANCK-GESELLSCHAFT ZUR FOERDERUNG DER WISSENSCHAFTEN E.V., Muenchen (DE)

(72) Inventors: Barbara NAVE, Ludwigshafen (DE); Oskar ZELDER, Ludwigshafen (DE); Michael BREUER, Ludwigshafen (DE); Srividhya SUNDARAM, Marburg (DE); Tobias ERB, Marburg (DE)

(21) Appl. No.: 18/691,525

(22) PCT Filed: Sep. 29, 2022
(86) PCT No.: PCT/EP2022/077180

§ 371 Date: Mar. 13, 2024

(30) Foreign Application Priority Data
Oct. 1, 2021 (EP) 21200581.3

(51) Int. Cl. C12P 17/04; C12N 9/00; C12N 9/02; C12N 9/04; C12N 9/10; C12N 9/18; C12N 15/70; C12R 1/19

(52) U.S. Cl. CPC C12P 17/04; C12N 9/0006; C12N 9/0008; C12N 9/1029; C12N 9/13; C12N 9/18; C12N 9/93; C12N 15/70; C12R 2001/19; C12Y 101/01001; C12Y 101/01021; C12Y 101/01077; C12Y 102/01024; C12Y 102/0103; C12Y 203/01007; C12Y 203/01018; C12Y 203/01084; C12Y 208/03; C12Y 301/01001; C12Y 301/01025; C12Y 301/02; C12Y 602/01004; C12Y 602/01009

(57) ABSTRACT

Disclosed herein are methods for producing tulipalin A (α-methylene-γ-butyrolactone), recombinant cells or organisms for producing tulipalin A, enzymes needed for producing tulipalin A, and nucleic acids for expression of those enzymes.

### CROSS-REFERENCE TO RELATED APPLICATIONS

This application is a U.S. National Phase Application of International Patent Application No. PCT/EP22/77180, filed Sep. 29, 2022, which claims priority to European Patent Application No 21200581.3 filed Oct. 1, 2021, and each of which is hereby incorporated by reference herein.

### REFERENCE TO AN ELECTRONIC SEQUENCE LISTING

The contents of the electronic sequence listing (sequence_listing_27843-2550.xml; Size: 236.2 KB; and Date of Creation: Apr. 17, 2025) is herein incorporated by reference in its entirety.

### FIELD OF THE INVENTION

The present invention relates to the field of biochemical synthesis. Provided are methods for the production of tulipalin A, recombinant cells or organisms for the production of tulipalin A, enzymes for the production of tulipalin A, and nucleic acids for expression of those enzymes.

### BACKGROUND OF THE INVENTION

Tulipalin A (α-methylene-γ-butyrolactone) is a naturally occurring vinyl monomer found in the tulip Tulipa gesneriana, as well as in the genera Tulipa, Erythronium, Gagea, Alstroemeria, Bomarea and Spiraea. Tulipalins function as defensive chemicals in plants and can elicit allergic reactions in humans.

Tulipalin's exo-methylene double bond allows for chain growth polymerization of the monomers to form the polymeric compound poly(tulipalin A). Tulipalin A polymerizes in a manner similar to methyl methacrylate (MMA), a polymer used in the production of polymethyl methacrylate acrylic plastics (PMMA), also known as acrylic glass, Perspex or Plexiglas, and methacrylate-butadiene-styrene (MBS). Hence, tulipalin A is considered a cyclic analog of methyl methacrylate and has the potential to replace oil-based MMA monomers as a sustainable alternative. As a naturally occurring vinyl, tulipalin A lends biocompatibility, biodegradability, eco-friendly, and renewable characteristics to the resulting polymers. Tulipalin A readily copolymerizes with copolymerizing agents such as styrene, methacrylate monomers, or acrylonitrile. In polymer producing industries, tulipalin is used in the production of materials such as thermoplastics, coatings and aliphatic polyesters, a technologically important class of biodegradable polymers. Compositions comprising tulipalin copolymers or copolyesters are used for example in cast glass and molding materials, automotive coats and finishes, thermoplastic resins and implantable medical devices.

In plants, tulipalins are derived from tuliposides, which are sugar esters composed of D-Glucose and 4′-hydroxy-2′-methylenebutanoyl and/or 3′,4′-dihydroxy-2′-methylenebutanoyl side chains. 6-tuliposide A and B can spontaneously form their lactonized aglycons, tulipalin A and B. Tulipalin A and B show antimicrobial and insecticidal activity and serve as a chemical defense mechanism in plants. Tuliposides are stored in all parts of the plants, and only seem to be converted to tulipalins upon infection or wounding of the plant, when a tuliposide-converting enzyme (TCE) catalyzes the conversion of tuliposides to tulipalins. Therefore, tulipalin levels in plants are typically low or barely detectable, and extraction of tulipalin A is not an economically viable option of tulipalin A production.

WO2016/196962 suggests a recombinant microorganism to produce functionalized alpha-substituted acrylates and C4-dicarboxylates.

Due to its potential as a sustainable alternative to methyl methacrylate, tulipalin A is an important industrial polymer. Hence, there is a need for an improved method for producing tulipalin A on an industrial scale, including materials needed for such a production process such as enzymes, recombinant cells or organisms, and nucleic acids for expression of enzymes used in production methods. The invention described herein provides methods of tulipalin A production, recombinant cells or organisms for tulipalin A production, enzymes used in these methods or by these cells or organisms and nucleic acids encoding these enzymes. The inventors have surprisingly found that the recombinant cells or organisms of the invention produce tulipalin A from fermented raw materials in a one-pot biosynthesis.

### Objectives and Summary of the Invention

The invention relates to a method for producing tulipalin A from itaconic acid. The pathway to derive tulipalin A from itaconic acid involves three enzymatically catalyzed reaction steps, hence, the invention comprises a first, second and third enzyme. Optionally, a fourth enzyme can be used.

The first reaction involves the formation of the intermediate itaconyl-CoA from itaconic acid and a source of CoA. Alternatively, the first reaction can also involve formation of the intermediate itaconate semialdehyde from itaconic acid.

Hence, in a first aspect, the invention relates to a method for producing tulipalin A from itaconic acid, the method comprising contacting a reaction mixture comprising itaconic acid with a first enzyme selected from at least one Acyl-CoA synthetase, at least one CoA-transferase and at least one Carboxylic acid reductase.

In a preferred embodiment, the Acyl-CoA synthetase is selected from the group consisting of Succinyl-CoA synthetase (SucCD) and Malate-CoA ligase (MtkAB). In another embodiment, the CoA-transferase is Itaconate-CoA transferase (Ict).

After synthesis of itaconyl-CoA from itaconic acid, the itaconyl-CoA is reacted further to form the intermediate itaconate semialdehyde.

Hence, in a further aspect of the invention, the method for producing tulipalin A further comprises contacting the reaction mixture with a second enzyme, wherein the second enzyme is at least one Oxidoreductase, preferably an Acyl-CoA reductase. In one embodiment, the Oxidoreductase is an Acyl-CoA reductase, preferably selected from Succinyl-CoA reductase (Scr) and Malonyl-CoA reductase (Mcr).

Thus, itaconate semialdehyde is formed either via the two-step reaction using Acyl-CoA synthetase or CoA-transferase as the first enzyme and using the second enzyme, or via direct formation using Carboxylic acid reductase as the first enzyme and no second enzyme. Once itaconate semialdehyde is present, it is reacted using a third enzyme catalyzing the formation of 2-methylene-4-ol-butyric acid.

Hence, in a further aspect of the invention, the method for producing tulipalin A further comprises contacting the reaction mixture with a third enzyme, wherein the third enzyme is at least one Oxidoreductase selected from the group consisting of Alcohol dehydrogenase, Lactaldehyde reductase, 3-sulfolactaldehyde reductase, succinate semialdehyde reductase and Aldose/Aldehyde reductase. In a preferred embodiment, the third enzyme is Alcohol dehydrogenase. In another preferred embodiment, the third enzyme is 3-sulfolactaldehyde reductase. 2-methylene-4-ol-butyric acid is able to form tulipalin A spontaneously via internal lactonization.

However, optionally, a fourth enzyme may be provided to catalyze lactone formation. Hence, in another aspect of the invention, the method for producing tulipalin A optionally further comprises contacting the reaction mixture with a fourth enzyme selected from at least one thioesterase and at least one lactonase.

In one embodiment, the fourth enzyme is a thioesterase and the formation of tulipalin A occurs via the intermediate 2-methylene-4-ol-butyryl-CoA, preferably wherein the formation of 2-methylene-4-ol-butyryl-CoA from 2-methylene-4-ol-butyric acid is catalyzed by the first enzyme of the invention (FIG. 4B).

2-methylene-4-ol-butyric acid can also be contacted with enzymes to make its ester. Therefore, in an alternative embodiment a fourth enzyme may be provided to catalyze ester formation. Hence, in an alternative aspect of the invention, the method for producing tulipalin A optionally further comprises contacting the reaction mixture with a fourth enzyme selected from at least one acyltransferase, at least one carboxyesterase, at least one carnitine acetyltransferase, at least one galactoside O-acetyltransferase and at least one alcohol acetyl transferase, thereby producing 4-acetyloxy-2-methylene butanoic acid (FIG. 16). The 4-acetyloxy-2-methylene butanoic acid can be converted to tulipalin A spontaneously, chemically or by the use of lactonases and thioesterases.

One or more, or all, steps of the production of tulipalin A may take place in a recombinant cell or organism comprising at least one of the enzymes of the invention.

Hence, one aspect of the invention relates to a recombinant cell or organism capable of synthesizing tulipalin A, comprising one or more nucleic acid molecules encoding a first enzyme, wherein the first enzyme is selected from at least one Acyl-CoA synthetase, at least one CoA-transferase and at least one Carboxylic acid reductase.

Another aspect of the invention relates to the recombinant cell or organism capable of synthesizing tulipalin A, wherein the recombinant cell or organism further comprises one or more nucleic acid molecules encoding a second enzyme, wherein the second enzyme is at least one Oxidoreductase. In one embodiment, the Oxidoreductase is an Acyl-CoA reductase, preferably selected from Succinyl-CoA reductase (Scr) and Malonyl-CoA reductase (Mcr).

A further aspect of the invention relates to the recombinant cell or organism capable of synthesizing tulipalin A, wherein the recombinant cell or organism further comprises one or more nucleic acid molecules encoding a third enzyme, wherein the third enzyme is at least one Oxidoreductase selected from the group consisting of Alcohol dehydrogenase, Lactaldehyde reductase, 3-sulfolactaldehyde reductase, succinate semialdehyde reductase and Aldose/Aldehyde reductase. In a preferred embodiment, the third enzyme is Alcohol dehydrogenase. In another preferred embodiment, the third enzyme is 3-sulfolactaldehyde reductase.

Another aspect of the invention relates to the recombinant cell or organism capable of synthesizing tulipalin A, wherein the recombinant cell or organism optionally further comprises one or more nucleic acid molecules encoding a fourth enzyme selected from at least one thioesterase and at least one lactonase.

An alternative aspect of the invention relates to the recombinant cell or organism capable of synthesizing tulipalin A, wherein the recombinant cell or organism optionally further comprises one or more nucleic acid molecules encoding a fourth enzyme selected from at least one acyltransferase, at least one carboxyesterase, at least one carnitine acetyltransferase, at least one galactoside O-acetyltransferase and at least one alcohol acetyl transferase.

In one embodiment, the recombinant cell or organism capable of synthesizing tulipalin A uses itaconic acid as a substrate for tulipalin A synthesis. In another embodiment, itaconic acid is derived from fermentation of a carbohydrate source selected from the group consisting of cellulose, hemicellulose, starch, sucrose, glucose, fructose, lactose, corn syrup, molasses, sugar beets, sugar cane or sugar palm by the recombinant cell or organism. In another embodiment, the itaconic acid is derived from fermentation of a carbohydrate source derived from a raw material comprising cellulose, hemicellulose and/or starch by the recombinant cell or organism. In a preferred embodiment, itaconic acid is derived from fermentation of glucose by the recombinant cell or organism. In another embodiment, glucose, sucrose, fructose, lactose or any other carbohydrate source may be provided in a feeding solution or derived from the raw material comprising cellulose, hemicellulose and/or starch.

In one embodiment, the recombinant cell organism is selected from the group consisting of Escherichia coli, Gluconobacter oxydans, Streptomyces coelicolor, Streptococcus thermophiles, Pseudomonas putida, Bacillus licheniformis, Bacillus subtilis, Corynebacterium glutamicum, Pseudozyma tsukubaensis, Ustilago maydis, Aspergillus niger, Aspergillus terreus, Trichoderma reesei, Pichia pastoris, Saccharomyces cerevisiae, Saccharomyces pombe and Yarrowia (candida) lipolytica. In a preferred embodiment, the recombinant cell or organism is Escherichia coli wildtype, Escherichia coli strain Ita23, Escherichia coli strain Ita36A or Pseudozyma tsukubaensis. In a more preferred embodiment, the recombinant cell or organism is Escherichia coli strain Ita36A or Pseudozyma tsukubaensis.

In one embodiment, the recombinant cell or organism produces itaconic acid.

The invention relates to a method of producing tulipalin A and a recombinant cell or organism capable of synthesizing tulipalin A, wherein the first enzyme is (i) Acyl-CoA synthetase, wherein the Acyl-CoA synthetase is selected from the group consisting of Succinyl-CoA synthetase (SucCD), and Malate-CoA ligase (MtkAB); (ii) CoA-transferase, wherein the CoA-transferase is Itaconate-CoA transferase (Ict); or (iii) Carboxylic acid reductase.

In a preferred embodiment, the first enzyme is Succinyl-CoA synthetase SucCD, wherein SucCD consists of two subunits SucC and SucD, wherein the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4. In one embodiment, SucCD is from Escherichia coli strain K12.

In one embodiment, wherein the SucC subunit comprises an amino acid sequence with at least 75%, at least 80%, at least 85%, at least 90%, at least 95%, at least 98%, at least 99% or 100% identity to an amino acid sequence according to SEQ ID NO: 2 and wherein the SucD subunit comprises an amino acid sequence with at least 75%, at least 80%, at least 85%, at least 90%, at least 95%, at least 98%, at least 99% or 100% identity to an amino acid sequence according to SEQ ID NO: 4.

In another preferred embodiment, the second enzyme is Succinyl-CoA reductase Scr, wherein Scr comprises an amino acid sequence with at least 70% identity to an amino acid sequence according to SEQ ID NO: 26. In one embodiment, Scr comprises an amino acid sequence with at least 85%, at least 90%, at least 95%, at least 98%, at least 99% or 100% identity to an amino acid sequence according to SEQ ID NO: 26. In one embodiment, Scr is from Clostridium kluyveri.

In another preferred embodiment, the second enzyme is HMG-CoA reductase HMGR, wherein HMGR comprises an amino acid sequence with at least 70% identity to an amino acid sequence according to SEQ ID NO: 98. In one embodiment, Scr comprises an amino acid sequence with at least 85%, at least 90%, at least 95%, at least 98%, at least 99% or 100% identity to an amino acid sequence according to SEQ ID NO: 98. In one embodiment, HMGR is from Methanothermococcus thermolithotrophicus.

In another preferred embodiment, the third enzyme is an Alcohol dehydrogenase, wherein the Alcohol dehydrogenase comprises an amino acid sequence with at least 70% identity to an amino acid sequence according to SEQ ID NO: 42. In one embodiment, the Alcohol dehydrogenase comprises an amino acid sequence with at least 75%, at least 80%, at least 85%, at least 90%, at least 95%, at least 98%, at least 99% or 100% identity to an amino acid sequence according to SEQ ID NO: 42. In one embodiment, the Alcohol dehydrogenase is YqhD from Escherichia coli strain K12.

In another preferred embodiment, the third enzyme is a 3-sulfolactaldehyde reductase, wherein the 3-sulfolactaldehyde reductase comprises an amino acid sequence with at least 70% identity to an amino acid sequence according to SEQ ID NO: 102. In one embodiment, the 3-sulfolactaldehyde reductase comprises an amino acid sequence with at least 75%, at least 80%, at least 85%, at least 90%, at least 95%, at least 98%, at least 99% or 100% identity to an amino acid sequence according to SEQ ID NO: 102. In one embodiment, the 3-sulfolactaldehyde reductase is YihU from Escherichia coli strain K12.

In another preferred embodiment, the third enzyme is a succinate semialdehyde reductase, wherein the succinate semialdehyde reductase comprises an amino acid sequence with at least 70% identity to an amino acid sequence according to SEQ ID NO: 118. In one embodiment, the succinate semialdehyde reductase comprises an amino acid sequence with at least 75%, at least 80%, at least 85%, at least 90%, at least 95%, at least 98%, at least 99% or 100% identity to an amino acid sequence according to SEQ ID NO: 118. In one embodiment, the succinate semialdehyde reductase is AKR7A2 from Homo sapiens.

In another preferred embodiment, the fourth enzyme is a lactonase, wherein the lactonase comprises an amino acid sequence with at least 70% identity to an amino acid sequence according to SEQ ID NO: 44. In one embodiment, the lactonase comprises an amino acid sequence with at least 75%, at least 80%, at least 85%, at least 90%, at least 95%, at least 98%, at least 99% or 100% identity to an amino acid sequence according to SEQ ID NO: 44. In one embodiment, the lactonase is Drp35 from S. aureus.

In another preferred embodiment, the fourth enzyme is a thioesterase, wherein the thioesterase comprises an amino acid sequence with at least 70% identity to an amino acid sequence according to SEQ ID NO: 46. In one embodiment, the thioesterase comprises an amino acid sequence with at least 75%, at least 80%, at least 85%, at least 90%, at least 95%, at least 98%, at least 99% or 100% identity to an amino acid sequence according to SEQ ID NO: 46. In one embodiment, the thioesterase is DEBS-TE from S. erythrea.

In another preferred embodiment, the fourth enzyme is a thioesterase, wherein the thioesterase comprises an amino acid sequence with at least 70% identity to an amino acid sequence according to SEQ ID NO: 48. In one embodiment, the thioesterase comprises an amino acid sequence with at least 75%, at least 80%, at least 85%, at least 90%, at least 95%, at least 98%, at least 99% or 100% identity to an amino acid sequence according to SEQ ID NO: 48. In one embodiment, the thioesterase is Ltmg-TE from S. amphibiosporus.

In another preferred embodiment, the fourth enzyme is a thioesterase, wherein the thioesterase comprises an amino acid sequence with at least 70% identity to an amino acid sequence according to SEQ ID NO: 50. In one embodiment, the thioesterase comprises an amino acid sequence with at least 75%, at least 80%, at least 85%, at least 90%, at least 95%, at least 98%, at least 99% or 100% identity to an amino acid sequence according to SEQ ID NO: 50. In one embodiment, the thioesterase is RevD-TE from S. spec SN-593.

In another preferred embodiment, an alternate fourth enzyme is an acyltransferase or an alcohol acetyl transferase.

In one embodiment, the acyltransferase comprises an amino acid sequence with at least 70% identity to an amino acid sequence according to SEQ ID NO: 112. In one embodiment, the acyltransferase comprises an amino acid sequence with at least 75%, at least 80%, at least 85%, at least 90%, at least 95%, at least 98%, at least 99% or 100% identity to an amino acid sequence according to SEQ ID NO: 112. In one embodiment, the acyltransferase is MsAcT from Mycolicibacterium smegmatis.

In one embodiment, the alcohol acetyl transferase comprises an amino acid sequence with at least 70% identity to an amino acid sequence according to SEQ ID NO: 106. In one embodiment, the alcohol acetyl transferase comprises an amino acid sequence with at least 75%, at least 80%, at least 85%, at least 90%, at least 95%, at least 98%, at least 99% or 100% identity to an amino acid sequence according to SEQ ID NO: 106. In one embodiment, the alcohol acetyl transferase is ATF1 from Saccharomyces cerevisiae.

In one embodiment, the alcohol acetyl transferase comprises an amino acid sequence with at least 70% identity to an amino acid sequence according to SEQ ID NO: 108. In one embodiment, the alcohol acetyl transferase comprises an amino acid sequence with at least 75%, at least 80%, at least 85%, at least 90%, at least 95%, at least 98%, at least 99% or 100% identity to an amino acid sequence according to SEQ ID NO: 108. In one embodiment, the alcohol acetyl transferase is ATF2 from Saccharomyces cerevisiae.

In one embodiment, the alcohol acetyl transferase comprises an amino acid sequence with at least 70% identity to an amino acid sequence according to SEQ ID NO: 110. In one embodiment, the alcohol acetyl transferase comprises an amino acid sequence with at least 75%, at least 80%, at least 85%, at least 90%, at least 95%, at least 98%, at least 99% or 100% identity to an amino acid sequence according to SEQ ID NO: 110. In one embodiment, the alcohol acetyl transferase is Eat1 from Saccharomyces cerevisiae.

In another preferred embodiment, an alternative fourth enzyme is a carnitine acetyltransferase or a galactoside O-acetyltransferase.

In one embodiment, the carnitine acetyltransferase comprises an amino acid sequence with at least 70% identity to an amino acid sequence according to SEQ ID NO: 120. In one embodiment, the carnitine acetyltransferase comprises an amino acid sequence with at least 75%, at least 80%, at least 85%, at least 90%, at least 95%, at least 98%, at least 99% or 100% identity to an amino acid sequence according to SEQ ID NO: 120. In one embodiment, the carnitine acetyltransferase is YAT2 from Saccharomyces cerevisiae.

In one embodiment, the galactoside O-acetyltransferase comprises an amino acid sequence with at least 70% identity to an amino acid sequence according to SEQ ID NO: 122. In one embodiment, the galactoside O-acetyltransferase comprises an amino acid sequence with at least 75%, at least 80%, at least 85%, at least 90%, at least 95%, at least 98%, at least 99% or 100% identity to an amino acid sequence according to SEQ ID NO: 122. In one embodiment, the galactoside O-acetyltransferase is LacA from Escherichia coli.

### BRIEF DESCRIPTION OF THE FIGURES

FIG. 1: Pathway of tulipalin A production indicating the reactions catalyzed by the first, second, third and fourth enzyme of the invention.

FIG. 2: Pathway of tulipalin A production indicating the reactions catalyzed by Carboxylic acid reductase, third and fourth enzyme of the invention.

FIG. 3: Pathway of tulipalin A production indicating enzymes that can be used to catalyze the intermediate reactions of the pathway.

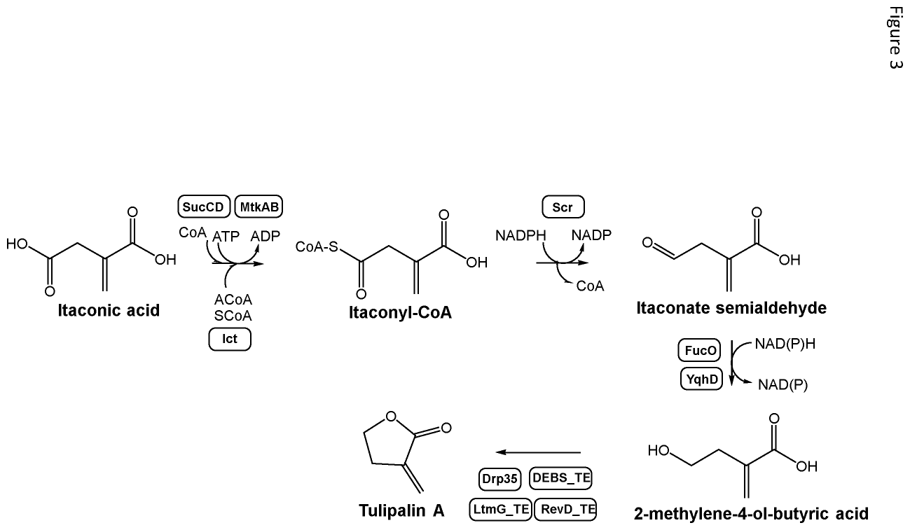

FIG. 4: A: Production of tulipalin A from 2-methylene-4-ol-butyric acid by direct lactonisation by Drp35. B: Production of tulipalin A via the 2-methylene-4-ol-butyryl-CoA intermediate by thioesterases.

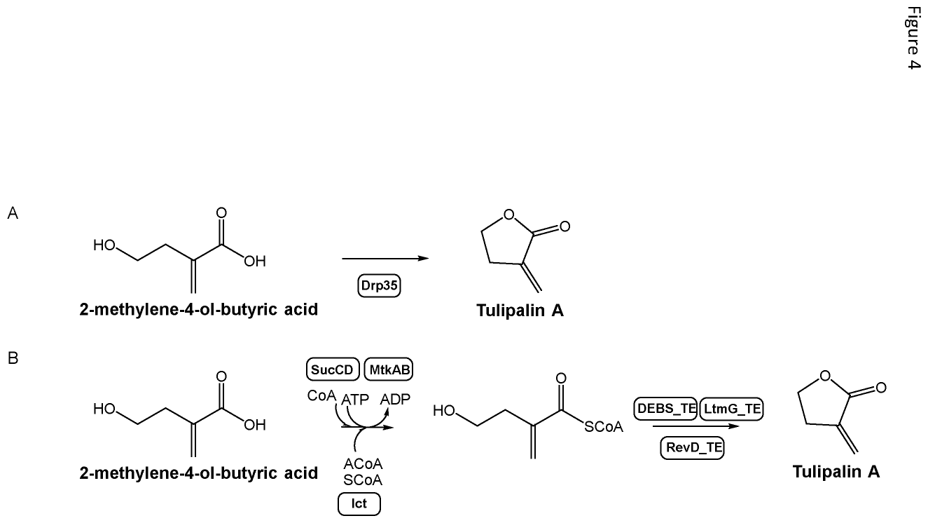

FIG. 5: Production of itaconyl-CoA by SucCD, MtkAB and Ict.

FIG. 6: One-pot in vitro tulipalin A production up to 20 h. The product is validated against the tulipalin A standard. A: Tulipalin A production using either SucCD, MtkAB or Ict as the first enzyme. B: Scheme indicating the enzymes used.

FIG. 7: One-pot in vitro tulipalin A production up to 48 h in the presence of thioesterases and lactonase and with SucCD as the first enzyme. The product is validated against the tulipalin A standard. A: Tulipalin A production using either DEBST_TE, Lmt_TE, Drp35 or Rev_TE as the fourth enzyme. B: Scheme indicating the enzymes used.

FIG. 8: One-pot in vitro tulipalin A production up to 48 h in the presence of thioesterases and lactonase and with Ict as the first enzyme. The product is validated against the tulipalin A standard. A: Tulipalin A production using either DEBST_TE, Lmt_TE, Drp35 or Rev_TE as the fourth enzyme. B: Scheme indicating the enzymes used.

FIG. 9: SDS-PAGE of expressed and purified his-tagged SucCD from Escherichia coli. Lane 1 size marker, lane 2 whole cell lysate, lane 3 soluble protein, lane 4 flow through, lanes 5 and 6 wash, lanes 7-11 elution fractions, lane 12 purified protein.

FIG. 10: SDS-PAGE of soluble protein fraction from bacterial lysates of cells expressing Yplct or Mcr. M: Marker, Lane 6: Mcr L152V, Lane 7: Mcr L152A, Lane 8: Mcr L152T, Lane 9: Mcr wildtype, Lane 10: Yplct, Lane 11: no expression, Lane 12: untransfected.

FIG. 11: Graph showing HPLC measurement of Itaconate and Succinate over time in samples incubated with Yplct or without enzyme (blank).

FIG. 12: SDS-PAGE of NiCar after Ni-NTA purification.

FIG. 13: Graph showing HPLC measurement of Benzoic acid and Benzaldehyde over time in samples incubated with NiCar or without enzyme (blank).

FIG. 14: Pathway of tulipalin A production indicating the reactions catalyzed by the first, second, and an YihU as the third enzyme of the invention.

FIG. 15: Graph showing the HPLC measurement of the formation of Tulipalin A in the presence of YihU as the third enzyme and in the absence of any fourth enzyme.

FIG. 16: Pathway of tulipalin A production indicating the reactions catalyzed by the first, second, third and an alternate fourth step (catalyzed by acyltransferases or alcohol acetyl transferases) to form 4-acetyloxy-2-methylene butanoic acid which can either be chemically lactonized to tulipalin A or via the action of lactonases/thioesterases to tulipalin A.

### DETAILED DESCRIPTION OF THE INVENTION

### General Definitions

Before the invention is described in detail with respect to some of its preferred embodiments, the following general definitions are provided.

The present invention as illustratively described in the following may suitably be practiced in the absence of any element or elements, limitation or limitations, not specifically disclosed herein.

The present invention will be described with respect to particular embodiments and with reference to certain figures but the invention is not limited thereto but only by the claims.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group which preferably consists only of these embodiments.

For the purposes of the present invention, the term “obtained” is considered to be a preferred embodiment of the term “obtainable”. If hereinafter e.g. a compound is defined to be obtainable from a specific source, this is also to be understood to disclose a compound which is obtained from this source.

Where an indefinite or definite article is used when referring to a singular noun, e.g. “a”, “an” or “the”, this includes a plural of that noun unless something else is specifically stated. The terms “about” or “approximately” in the context of the present invention denote an interval of accuracy that the person skilled in the art will understand to still ensure the technical effect of the feature in question. The term typically indicates deviation from the indicated numerical value of ±10%, and preferably of ±5%.

Technical terms are used by their common sense. If a specific meaning is conveyed to certain terms, definitions of terms will be given in the following in the context of which the terms are used.

The term “expression” or “gene expression” as used herein refers to the process of synthesis of a gene product, preferably a functional RNA or protein. Gene expression generally comprises DNA transcription, optionally RNA processing and in the case of protein-expressing genes, RNA translation.

For the purposes of the invention, “recombinant” (or transgenic) with regard to a cell or an organism means that the cell or organism contains a heterologous polynucleotide which is introduced by man by gene technology and with regard to a polynucleotide includes all those constructions brought about by man by gene technology/recombinant DNA techniques in which either
(a) the sequence of the polynucleotide or a part thereof, or
(b) one or more genetic control sequences which are operably linked with the polynucleotide, including but not limited thereto a promoter, or
(c) both a) and b)
are not located in their wildtype genetic environment or have been modified.

The term “heterologous” (or exogenous or foreign or recombinant or non-native) polypeptide is defined herein as a polypeptide that is not native to the host cell, a polypeptide native to the host cell in which structural modifications, e.g., deletions, substitutions, and/or insertions, have been made by recombinant DNA techniques to alter the native polypeptide, or a polypeptide native to the host cell whose expression is quantitatively altered or whose expression is directed from a genomic location different from the native host cell as a result of manipulation of the DNA of the host cell by recombinant DNA techniques, or whose expression is quantitatively altered as a result of manipulation of the regulatory elements of the polynucleotide by recombinant DNA techniques e.g., a stronger promoter; or a polynucleotide native to the host cell, but integrated not within its natural genetic environment as a result of genetic manipulation by recombinant DNA techniques.

The terms “nucleic acid” or “nucleic acid molecule” or “nucleic acid sequence” or “nucleotide sequence” are used interchangeably herein to refer to a biomolecule composed of nucleotides. The nucleic acid molecule can be comprised within an eukaryotic or prokaryotic organism, a eukaryotic or prokaryotic cell, a cell nucleus or a cell organelle, as part of a genome or as an individual molecule; or it can be comprised within a plasmid, a vector, an artificial chromosome; a nucleic acid can also exist outside of a cell, in vesicles, viruses or freely circulating, it can be isolated in a suitable composition, in a fixed or frozen tissue or cell culture, or dried. The nucleic acid can be synthesized or naturally occurring, i.e. isolated from nature.

The terms “sequence Identity”, “% sequence identity”, “% identity”, “% identical” or “sequence alignment” are used interchangeably herein and refer to the comparison of a first nucleic acid sequence to a second nucleic acid sequence, or a comparison of a first amino acid sequence to a second amino acid sequence and is calculated as a percentage based on the comparison. The result of this calculation can be described as “percent identical” or “percent ID.” A sequence identity may be determined by a program, which produces an alignment, and calculates identity counting both mismatches at a single position and gaps at a single position as non-identical positions in final sequence identity calculation. The sequence identity is determined over the entire length of the first and second nucleic acid sequence.

According to this invention, a pairwise global alignment is produced, meaning that two sequences are aligned over their complete length, which is usually produced by using a mathematical approach, called alignment algorithm.

According to the invention, the alignment is generated by using the algorithm of Needleman and Wunsch (J. Mol. Biol. (1979) 48, p. 443-453). Preferably, the program “NEEDLE” (The European Molecular Biology Open Software Suite (EMBOSS)) is used for the purposes of the current invention, with using the programs default parameter (polynucleotides: gap open=10.0, gap extend=0.5 and matrix=EDNAFULL; polypeptides: gap open=10.0, gap extend=0.5 and matrix=EBLOSUM62). After aligning two sequences, in a second step, an identity value is determined from the alignment produced. For this purpose, the %-identity is calculated by dividing the number of identical residues by the length of the alignment region which is showing the respective sequence of the present invention over its complete length multiplied with 100: %-identity=(identical residues/length of the alignment region which is showing the respective sequence of the present invention over its complete length)*100.

For calculating the percent identity of two nucleic acid sequences the same applies as for the calculation of percent identity of two amino acid sequences with some specifications. For nucleic acid sequences encoding for a protein the pairwise alignment shall be made over the complete length of the coding region of the sequence of this invention from start to stop codon excluding introns. Introns present in the other sequence, to which the sequence of this invention is compared, shall also be removed for the pairwise alignment. After aligning two sequences, in a second step, an identity value is determined from the alignment produced. Percent identity is calculated by %-identity=(identical residues/length of the alignment region which is showing the sequence of the invention from start to stop codon excluding introns over its complete length)*100.

Moreover, the preferred alignment program for nucleic acid sequences implementing the Needleman and Wunsch algorithm (J. Mol. Biol. (1979) 48, p. 443-453) is “NEEDLE” (The European Molecular Biology Open Software Suite (EMBOSS)) with the programs default parameters (gapopen=10.0, gapextend=0.5 and matrix=EDNAFULL).

The term “encoded protein” or “encoded amino acid” refers a protein that consists of a chain of amino acids, which results from a sequence that is encoded by a nucleic acid molecule comprising three-nucleotide codons.

As used herein, the term “cellulose” refers to a polysaccharide consisting of a linear chain of β(1→4)-linked D-glucose units. Cellulose is a structural component of the primary cell wall of plants and is also found in algae, oomycetes of bacteria. In one embodiment, the cellulose used in the method of the invention is derived from raw plant material. The term “raw plant material” refers to a plant material that is minimally processed or unprocessed, a grass, stalk, fruit, seed, leaf, wood, petal, fiber or any other plant part, often a feedstock or raw biomass, a plant-derived biomaterial or a plant which has undergone the transformation required to prepare it for further processing or for transport, e.g. milling, pressing, shaping, flaking.

Cellulose may include any type of cellulose. There are four different polymorphs of cellulose: cellulose I, II, III, and IV. Naturally occurring cellulose is known as cellulose I, which exists in parallel strands without intersheet hydrogen bonding. Cellulose II is thermodynamically more stable and exists in antiparallel strains with intersheet hydrogen bonding. Cellulose III is amorphous and obtained by treatment of cellulose I or II with amines. Cellulose IV is obtained after treatment of cellulose III with glycerol at very high temperatures. Types of cellulose include, for example, materials comprising cellulose and additional components. Cellulose also includes derivatives of cellulose such as cellulose esters and cellulose ethers. In one embodiment, cellulose is any type of cellulose. Hence, in one embodiment, the itaconic acid is derived from fermentation of a raw material comprising any type of cellulose, hemicellulose and/or starch by the recombinant cell or organism.

The term “hemicellulose” or “polyose” refers to any heterpolymer of cellulose and other polysaccharides. Hemicelluloses include xylan, glucuronoxylan, arabinoxylan, glucomannan and xyloglucan. Whereas cellulose is derived exclusively from glucose, hemicelluloses are composed of diverse sugars, and can include the five-carbon sugars xylose and arabinose, the six-carbon sugars glucose, mannose and galactose, and the six-carbon deoxy sugar rhamnose.

As used herein the term “starch” refers to any material composed of amylose and amylopectin. Amylose is a polysaccharide made of glucose units, bonded to each other through α(1→4) glycosidic bonds. Amylopectin is a water-soluble polysaccharide and highly branched polymer of glucose units. In amylopectin, glucose units are linked in a linear way with α(1→4) glycosidic bonds and branching takes place with α(1→6) bonds occurring every 24 to 30 glucose units. In particular, the term “starch” refers to the amylose and/or amylopectin from any plant-based material including but not limited to grains, grasses, tubers and roots and more specifically wheat, barley, corn, rye, oats, sorghum, milo, rice, sorghum, brans, cassava, millet, potato, sweet potato and tapioca. In one embodiment, the starch used in the method of the invention is derived from raw plant material.

The term “fermenting” or “fermentation” refers to a process which converts sugars, such as glucose, into cellular energy under anaerobic conditions, producing ATP, fermentation product and CO₂. A “fermentation product” is one of the products of the fermentation process including organic acids or alcohols.

### Methods of the Invention

The method of the invention relates to a method for producing tulipalin A from itaconic acid, the method comprising contacting a reaction mixture comprising itaconic acid with a first enzyme of the invention. In another embodiment, the method comprises contacting a reaction mixture comprising itaconic acid with a first and a second enzyme of the invention. In another embodiment, the method comprises contacting a reaction mixture comprising itaconic acid with a first, second and third enzyme of the invention. In another embodiment, the method comprises contacting a reaction mixture comprising itaconic acid with a first, second, third and fourth enzyme of the invention. In another embodiment, the method comprises contacting a reaction mixture comprising itaconic acid with a first and third enzyme of the invention. In another embodiment, the method comprises contacting a reaction mixture comprising itaconic acid with a first, third and fourth enzyme of the invention. The enzymes of the invention are defined in the following chapter.

The term “reaction mixture” describes a product formed by the combination of two or more elements, compounds or substances together causing reactions or interactions of substances that may involve the transformation of original substances. A reaction mixture may comprise additional substances that enable specific chemical reactions to occur.

As used herein, the term “contacting” means bringing two compounds or molecules close enough to each other that they can chemically, electrically or physically interact. This interaction may be due to forces between molecules or may involve bonding and unbonding of molecules. Contacting may take place in solution or between solutions, on a solid support, within or on one or more gaseous phases or within a cell.

The terms “producing” and “synthesizing” as used herein may be used interchangeably and refer to the chemical synthesis of a molecule. Chemical synthesis of a molecule can comprise one or more chemical reactions that can be catalyzed by one or more enzymes. Chemical synthesis of a molecule can take place within a cell or organism or within a cell-free environment.

The term “catalyzing” or “catalyze” as used herein when referring to an enzymatic reaction means to cause or accelerate the initiation or the progression of a chemical reaction. Enzymes may use cellular or thermal energy and/or proton or electron donors and acceptors while catalyzing reactions. Catalyzing means reducing the activation energy needed to start a reaction by weakening the chemical bonds, usually by temporarily bonding with the reacting molecules.

“ATP”, “ADP” and “AMP” refer to adenosine triphosphate, adenosine diphosphate and adenosine monophosphate, respectively. Pᵢ refers to phosphate and PPᵢ to pyrophosphate. ATP is a cellular energy storage molecule found in prokaryotic and eukaryotic cells which releases energy by cleaving phosphate or pyrophosphate. ATP, ADP and AMP can be used as cofactors in enzymatic reactions.

The terms “NADH/H+”, “NADH”, “NAD” or “NAD+” are used interchangeably and refer to nicotinamide adenine dinucleotide. Nicotinamide adenine dinucleotide is involved in redox reactions, carrying electrons from one reaction to another, and is found in prokaryotic and eukaryotic cells. NAD+ refers to the oxidized form and accepts electrons from other molecules and becomes reduced. NADH is the reduced form, which can be used as a reducing agent to donate electrons. The terms “NADPH/H+”, “NADPH”, “NAPD” or “NADP+” are used interchangeably and refer to nicotinamide adenine dinucleotide phosphate and can fulfil the same functions as nicotinamide adenine dinucleotide. Both are used as cofactors in enzymatic reactions.

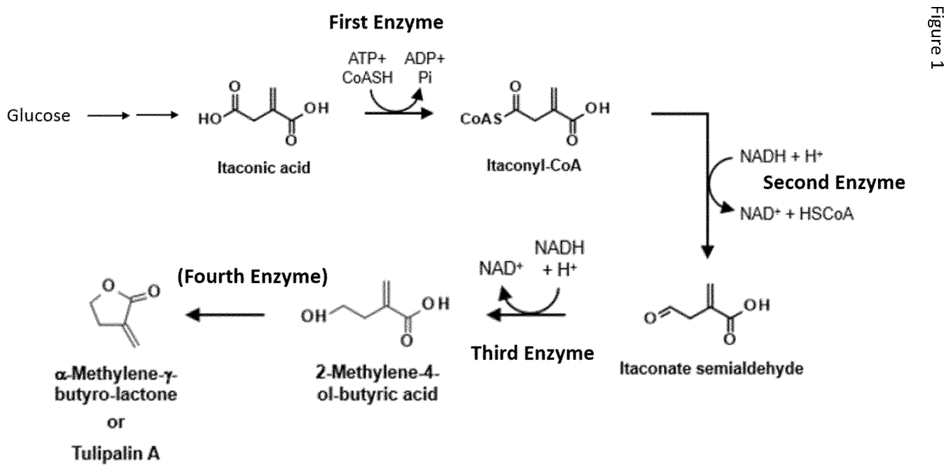

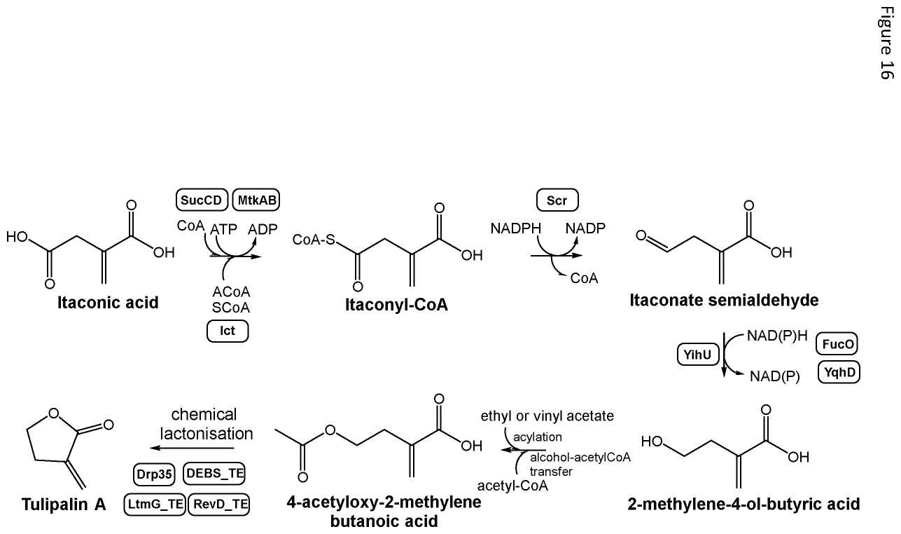

In one embodiment, the first enzyme of the invention catalyzes the formation of Itaconyl-CoA from itaconic acid (FIG. 1). In another embodiment, the second enzyme of the invention catalyzes the formation of itaconate semialdehyde from Itaconyl-CoA (FIG. 1). In a further embodiment, the third enzyme of the invention catalyzes the formation of 2-Methylene-4-ol-butyric acid from itaconate semialdehyde (FIG. 1). In a further optional embodiment, the fourth enzyme of the invention catalyzes lactone formation of 2-Methylene-4-ol-butyric acid to produce tulipalin A (FIG. 1). In an alternative optional embodiment, a fourth enzyme of the invention catalyzes the ester formation of 2-Methylene-4-ol-butyric acid to produce 4-acetyloxy-2-methylene butanoic acid (FIG. 16). Alternatively, in another embodiment, the lactone formation of tulipalin A may occur spontaneously without the catalyzing function of any enzyme.

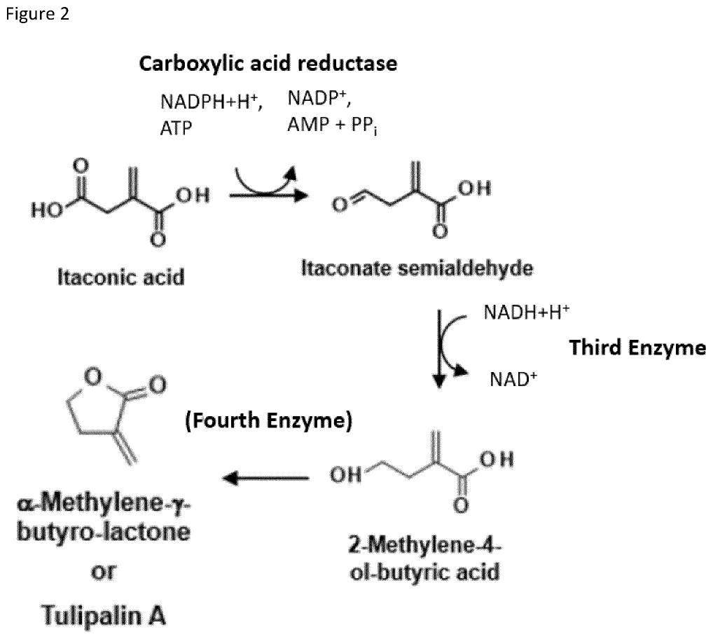

In another embodiment, the first enzyme of the invention is Carboxylic acid reductase which catalyzes the formation of itaconate semialdehyde from itaconic acid (FIG. 2).

In one embodiment, the method of production is performed within a recombinant cell or organism. Hence, the reaction mixture may be comprised within the cytosol of a recombinant cell or organism. In another embodiment, the method of production is performed within a cell-free environment. Hence, the reaction mixture may be comprised in a reaction vessel.

### Recombinant Cell or Organism

Another aspect of the invention is providing a recombinant cell or organism capable of producing tulipalin A. The invention provides recombinant cells or organisms capable of producing tulipalin A from itaconic acid. In one embodiment, the method of the invention is carried out within a recombinant cell or organism. Thus, one aspect of the invention relates to a recombinant cell or organism capable of carrying out the method of the invention.

Recombinant cells or organisms useful in the method of the invention are cells or organisms that produce itaconic acid, either naturally or through genetic engineering. In one embodiment, the recombinant cell or organism produces itaconic acid. Cells or organisms that naturally produce itaconic acid include Aspergillus terreus, Aspergillus niger and Ustilago maydis.

In another embodiment, the recombinant cell or organism is genetically engineered to produce itaconic acid. Such organisms include Escherichia coli strain Ita23, Escherichia coli Ita36A and Pseudozyma tsukubaensis (described in WO 2019/233853).

Other recombinant cells or organisms can be engineered to produce itaconic acid, including bacteria such as Escherichia coli, Gluconobacter oxydans, Streptomyces coelicolor, Streptococcus thermophiles, Pseudomonas putida, Bacillus licheniformis, Bacillus subtilis, Corynebacterium glutamicum, fungi or yeast such as Pseudozyma tsukubaensis, Ustilago maydis, Aspergillus niger, Aspergillus terreus, Trichoderma reesei, Pichia pastoris, Saccharomyces cerevisiae, Saccharomyces pombe, Yarrowia (candida) lipolytica, or mammalian cell lines such as Chinese Hamster Ovary (CHO) cells, HeLa cells or human embryonic kidney (HEK) 293 cells.

The recombinant cell or organism capable of producing tulipalin A is selected from the group consisting of Escherichia coli, Gluconobacter oxydans, Streptomyces coelicolor, Streptococcus thermophiles, Pseudomonas putida, Bacillus licheniformis, Bacillus subtilis, Corynebacterium glutamicum, Pseudozyma tsukubaensis, Ustilago maydis, Aspergillus niger, Aspergillus terreus, Trichoderma reesei, Pichia pastoris, Saccharomyces cerevisiae, Saccharomyces pombe and Yarrowia (candida) lipolytica.

In one embodiment, the Escherichia coli cell is selected from the strains wild type, MG1655, B121, 60E4, Ita23 and Ita36A. In a preferred embodiment, the Escherichia coli cell is Escherichia coli Ita23 or Ita36A.

In a preferred embodiment, the recombinant cell or organism is selected from the group consisting of Escherichia coli, Pseudomonas putida, Corynebacterium glutamicum, Pseudozyma tsukubaensis, Ustilago maydis, Aspergillus niger, Pichia pastoris, Saccharomyces cerevisiae and Saccharomyces pombe.

In a particularly preferred embodiment, the recombinant cell or organism is Escherichia coli wild-type, Escherichia coli strain Ita23, Escherichia coli strain Ita36A or Pseudoyzma tsukubaensis.

Itaconic acid, also called methylenesuccinic acid, is a dicarboxylic acid that can be produced by fermentation. Starting material of fermentation is raw plant material comprising cellulose, hemicellulose and/or starch. Raw materials may be cereal crops, grasses, grains, sugar beets, sugar cane, sugar palm, potato, sweet potato or fruit. Cellulose, hemicellulose and starch are broken down into smaller carbohydrates including sucrose, glucose, lactose and fructose during liquefaction and saccharification of raw plant materials using amylolytic microorganisms or enzymes including α-amylases and glucoamylases. Glucose, or other starting materials such as sucrose, lactose, corn syrup, sugar beets, sugar cane, sugar palm or molasses, are then fermented by fermenting microorgansims that either naturally produce itaconic acid or have been engineered to produce itaconic acid from glucose. Microorganisms or cells that naturally produce itaconic acid include Aspergillus terreus, Aspergillus niger and Ustilago maydis. Methods for producing itaconic acid and organisms producing itaconic acid are known in the art (Regestein et al. Biotechnol. Biofuels 2018, Hossain et al. Fungal Biol. Biotechnol. 2019, Yang et al. JB&B 2019, Nemestothy et al. Waste Biomass Valorization 2020).

Instead of fermenting raw materials to produce glucose or other starting materials for itaconic acid production, the recombinant cell or organism in culture may be fed with glucose or molasses. In one embodiment, the recombinant cell or organism of the invention is cultured in a batch culture. Preferably, the recombinant cell or organism is cultured in a medium comprising glucose. In another embodiment, the recombinant cell or organism of the invention is cultured in a fed-batch culture. Preferably, the fed-batch culture is fed with a medium comprising glucose.

In yet another embodiment, the recombinant cell or organism is cultured in a batch or fed-batch culture, preferably wherein the cultivation medium and the feeding medium comprise itaconic acid.

In order to ensure tulipalin A formation and to stabilize intermediates of the tulipalin A synthesis pathway, it is useful to reduce expression of aldose/aldehyde reductase (EC 1.1.1.21) in the recombinant cell or organism. In one embodiment, the recombinant cell or organism expresses reduced levels of endogenous aldehyde reductases compared to wild-type endogenous levels. Methods of engineering a cell or organism with reduced or abolished endogenous aldehyde reductase expression are known in the art (Kunjapur et al. J Am Chem Soc. 2014). In one embodiment, the recombinant cell or organism with reduced aldehyde reductase expression is Escherichia coli strain K12 MG1655.

In particular, the recombinant cell or organism of the invention comprises heterologous polypeptides for the expression of enzymes. These heterologous polypeptides comprise nucleic acid molecules encoding enzymes. In one embodiment, the recombinant cell or organism comprises the first enzyme of the invention. In another embodiment, the recombinant cell or organism comprises the first and second enzyme of the invention. In another embodiment, the recombinant cell or organism comprises the first, second and third enzyme of the invention. In another embodiment, the recombinant cell or organism comprises the first, second, third and fourth enzyme of the invention. In another embodiment, the recombinant cell or organism comprises the first and third enzyme of the invention. In another embodiment, the recombinant cell or organism comprises the first, third and fourth enzyme of the invention.

The enzymes of the invention are defined in the following chapter.

### Enzymes Used in the Methods of the Invention

The “UniProt numbers” or “UniProt” or “UniProt Accession numbers” provided herein refer to the unique identifiers given to individual genes and proteins by the UniProt Consortium, which are available from their database at www.uniprot.org and commonly used as references in the field. UniProtKB (UniProt Knowledgebase) is a freely accessible database of protein sequence and functional information. The UniProt database includes manually annotated and reviewed entries (provided by the Swiss-Prot database) and automatically annotated and not manually reviewed entries (provided by TrEMBL database), many of which are derived from genome sequencing projects. TrEMBL includes translated coding sequences from the EMBL-Bank/GenBank/DDBJ nucleotide sequence database, and others.

The “EC numbers” as provided herein refer to the Enzyme Commission number, a numerical classification scheme for enzymes based on the chemical reactions they catalyze, including a system of enzyme nomenclature. If different enzymes catalyze the same reaction, they receive the same EC number, for example homologous enzymes from different organisms or non-homologous isofunctional enzymes. A database of EC numbers can be accessed for example at https://iubmb.qmul.mc.uk/enzyme/ provided by the Nomenclature Committee of the International Union of Biochemistry and Molecular Biology.

The terms “first enzyme”, “second enzyme”, “third enzyme” and “fourth enzyme” as used herein refer to the order in which reaction steps of the production of tulipalin A from itaconic acid are described, as a matter of convenience. When adding enzymes to a solution or expressing enzymes in a cell, the terms “first enzyme”, “second enzyme”, “third enzyme” and “fourth enzyme” do not refer to a specific order or sequence in which the enzymes are added or expressed. The enzymes may be provided, mixed, synthesized or expressed in any order. The enzymes may also be provided, mixed, synthesized or expressed in the order of first, second, third and fourth enzyme.

Itaconyl-CoA Synthesis from Itaconic Acid

The first enzyme of the invention catalyzes the formation of Itaconyl-CoA from itaconic acid. This reaction can by catalyzed by any enzyme capable of forming a carbon-sulfur bond or thioester bond between a dicarboxylic acid and Coenzyme A.

The term “dicarboxylic acid” or “dicarboxylate” refers to an organic compound containing two carboxyl functional groups (—COOH).

The terms “Coenzyme A”, “CoA”, “SHCoA” or “CoASH” as used herein are used interchangeably and refer to the thiol Coenzyme A, a coenzyme used as a substrate by cellular enzymes, for example for oxidation of acids, such as during fatty acid synthesis or in the citric acid cycle. It occurs in both prokaryotic and eukaryotic genomes. CoA can react with carboxylic acids to form thioesters, thus functioning as an acyl group carrier. A molecule of Coenzyme A carrying an acyl group is referred to as “acyl-CoA”, for example Succinyl-CoA, Itaconyl-CoA, or Malonyl-CoA.

Thus, in a first aspect, the first enzyme of the invention is an Acyl-CoA synthetase or a CoA-transferase.

The terms “ligase” and “synthetase” are used interchangeably and refer to an enzyme that can catalyze the joining (“ligation”) of two molecules by forming a new chemical bond, typically via hydrolysis.

The terms “Dicarboxylate-CoA ligase”, “Carboxyl-CoA synthetase”, “Acyl-CoA synthetase” or “Dicarboxyl-CoA synthetase” as used herein are used interchangeably and refer to a ligase or synthetase enzyme capable of forming carbon-sulfur bonds. This includes enzymes belonging to Enzyme Commission number EC 6.2.1 “Acid-Thiol Ligases”. Acyl-CoA synthetases are enzymes that can catalyze the formation of a bond between, i.e. ligate, a dicarboxylic acid and CoA.

In one embodiment, the first enzyme of the invention is an Acyl-CoA synthetase. Acyl-CoA synthetases of the invention are used to catalyze the formation of Itaconyl-CoA from Itaconic acid.

In one embodiment, the Acyl-CoA synthetase of the invention is selected from the group consisting of Acetate-CoA ligase (EC 6.2.1.1 or EC 6.2.1.13), Succinyl-CoA synthetase (EC 6.2.1.4 or EC 6.2.1.5), Glutarate-CoA ligase (EC 6.2.1.6), Malate-CoA ligase (EC 6.2.1.9), Acid-CoA ligase (EC 6.2.1.10), 6-carboxyhexanoate-CoA ligase (EC 6.2.1.14), Arachidonate-CoA ligase (EC.6.2.1.15), Acetoactetate-CoA ligase (EC 6.2.1.16), Propionate-CoA ligase (EC 6.2.1.17), Citrate-CoA ligase (EC 6.2.1.18), Dicarboxylate-CoA ligase (EC 6.2.1.23), Phytanate-CoA ligase (EC 6.2.1.24), and 4-Hydroxybutyrate-CoA ligase (EC 6.2.1.40 or EC 6.2.1.56).

In a preferred embodiment, the Acyl-CoA synthetase of the invention is selected from the group consisting of Succinyl-CoA synthetase (ADP-forming, EC 6.2.1.5) and Malate-CoA ligase (EC 6.2.1.9).

In one embodiment, the Succinyl-CoA synthetase is formed of two subunits beta and alpha. In one embodiment, the Succinyl-CoA synthetase is of bacterial origin, preferably Succinyl-CoA synthetase is isolated from a bacterium of the genus Escherichia, Advenella, Alcanivorax or Thermobifida. Succinyl-CoA synthetases are known to accept itaconic acid as a substrate (Schurmann et al. J Bacteriol. 2011).

More preferably, the Succinyl-CoA synthetase is from Escherichia coli (SucCD, subunit beta: SucC UniProt POA836 (SEQ ID NO: 2) and subunit alpha: SucD POAGE9 (SEQ ID NO: 4), Nolte et al. Appl Environ Microbiol. 2014), Advenella mimigardefordensis (SucCD, subunit beta: SucC Uniprot WOPFR9 (SEQ ID NO: 6) and subunit alpha: SucD Uniprot WOPAN5 (SEQ ID NO: 8)), Alcanivorax borkumensis (SucCD, subunit beta: SucC Uniprot Q0VPF7 (SEQ ID NO: 10) and subunit alpha: SucD UniProt Q0VPF8 (SEQ ID NO: 12), Schwander et al. Science 2016) or Thermobifida fusca (subunit beta: Tfu_2577 Uniprot Q47LR2 (SEQ ID NO: 18) and subunit alpha: Tfu_2576 UniProt Q47LR3 (SEQ ID NO: 20), Yang et al. Biotechnol Lett. 2020).

In one embodiment, the Malate-CoA ligase is formed of two subunits beta and alpha. In one embodiment, the Malate-CoA ligase is of bacterial origin, preferably from the genus Methylorubrum. More preferably, the Malate-CoA ligase is from Methylorubrum extorquens (MtkAB, subunit alpha: MtkA, UniProt P53594 (SEQ ID NO: 14), subunit beta: MtkB, UniProt P53595 (SEQ ID NO: 16), Schürmann et al. J Bacteriol., 2011).

The term “CoA-transferase” describes a class of enzymes that catalyze the transfer of specific functional groups, in this case the functional group being CoA, from a donor molecule to an acceptor molecule. CoA-transferases are a type of transferase belonging to the class of sulfur transferases EC 2.8, more specifically class EC 2.8.3., “CoA-transferases”.

In one embodiment, the first enzyme of the invention is a CoA-transferase. CoA-transferases of the invention are used to catalyze the formation of Itaconyl-CoA from itaconic acid.

In one embodiment, the CoA-transferase of the invention is selected from the group consisting of Itaconate-CoA transferase (Ict), Propionate CoA-transferase (EC 2.8.3.1), Malonate CoA-transferase (EC 2.8.3.3), 3-oxoacid CoA-transferase (EC 2.8.3.5), 3-oxoadipate CoA-transferase (EC 2.8.3.6), Acetate CoA-transferase (EC 2.8.3.8), Butyrate-acetoacetate CoA-transferase (EC 2.8.3.9), Citrate CoA-transferase (EC 2.8.3.10), Citramalate CoA-transferase (EC 2.8.3.11), Glutaconate CoA-transferase (EC 2.8.3.12), Succinate-hydroxymethylglutarate CoA-transferase (EC 2.8.3.13), Succinyl-CoA:(R)-benzylsuccinate CoA-transferase (EC 2.8.3.15), Formyl-CoA transferase (EC 2.8.3.16), Succinyl-CoA:acetate CoA-transferase (EC 2.8.3.18), CoA:oxalate CoA-transferase (EC 2.8.3.19), Succinyl-CoA-D-citramalate CoA-transferase (EC 2.8.3.20) and Succinyl-CoA-L-malate CoA-transferase (EC 2.8.3.22), (R)-2-hydroxy-4-methylpentanoate CoA-transferase (EC 2.8.3.24), Succinyl-CoA:mesaconate CoA transferase (EC 2.8.3.26) and 4-hydroxybutyrate CoA transferase (EC 2.8.3.-).

In a preferred embodiment, the CoA-transferase of the invention is selected from the group consisting of Itaconate-CoA transferase (Ict), 4-hydroxybutyrate CoA-transferase (RpiA), Succinyl-CoA-D-citramalate CoA-transferase (Sct, EC 2.8.3.20) and Succinyl-CoA-L-malate CoA-transferase (SmtAB, EC 2.8.3.22).

In one embodiment, the CoA-transferase is Succinyl-CoA-D-citramalate CoA-transferase from the bacterium Clostridium tetanomorphum (UniProt Q1KLK0) and can use itaconic acid as an acceptor. In another embodiment, the CoA transferase is Succinyl-CoA-L-malate CoA-transferase from Chloroflexus aurantiacus (UniProt A9WGE3) and can use itaconic acid as an acceptor.

In another embodiment, the CoA-transferase is Itaconate-CoA transferase (Ict). The Itaconate-CoA transferase of the invention is selected from Itaconate-CoA transferase/4-hydroxybutyrate CoA-transferase from Yersinia pestis (Yplct/RpiA, Uniprot YPO1926 (SEQ ID NO: 24) Sasikaran et al. Nat Chem Biol. 2014) and Itaconate-CoA transferase from Pseudomonas aeruginosa (Palct, UniProt Q91563 (SEQ ID NO: 22), Sasikaran et al. Nat Chem Biol. 2014).

Itaconate Semialdehyde Synthesis from Itaconyl-CoA

The second enzyme of the invention catalyzes the formation of Itaconate semialdehyde from Itaconyl-CoA. This reaction can by catalyzed by any enzyme capable of forming an aldehyde from Acyl-CoA using NAD(P)H/H+ as a cofactor.

The term “semialdehyde” refers to the monoaldehyde of a dicarboxylic acid, i.e. wherein one of the two carboxylic acid functional groups forms an aldehyde functional group.

Thus, in a further aspect, the second enzyme of the invention is an Oxidoreductase. Oxidoreductase is an enzyme that catalyzes the transfer of electrons from an electron donor to an electron acceptor. Oxidoreductases useful for the invention are those of class EC 1.1.1. or EC 1.2.1, which use NADP+ or NAD+ as cofactors and act on the CH—OH group of donors or the aldehyde or oxo group of donors, respectively.

In one embodiment, the Oxidoreductase of the invention is selected from the group consisting of Hydroxymethylglutaryl-CoA reductase (HMG-CoA reductase, EC 1.1.1.34 or EC 1.1.1.88), Cinnamoyl-CoA reductase (EC 1.2.1.44), Malonyl-CoA reductase (EC 1.2.1.75), Succinyl-CoA reductase (EC 1.2.1.76), and 3-oxo-5,6-dehydrosuberyl-CoA semialdehyde dehydrogenase (EC 1.2.1.91).

In one embodiment, the HMG-CoA reductase is derived from a bacterium selected from the group consisting of Polaribacter filamentous (UniProt: A0A2S7KW32), Lactobacillus kefiranofaciens (UniProt: A0A269ZLZ0), Pseudobacteriovorax antillogorgiicola (UniProt: A0A1Y6BIN7), Lactobacillus paracasei NRIC 0644 (UniProt: A0A0C9PS41), Methanocella sp. (UniProt: A0A1V4Z2S1), Capnocytophaga sp (UniProt: L1P7W3), and Longimonas halophile (UniProt: A0A2H3NXD1). In one embodiment, the HMG-CoA reductase is derived from Methanothermococcus thermolithotrophicus (UniProt: A0A4V8GZY0).

In a preferred embodiment, the Oxidoreductase is Succinyl-CoA reductase from Clostridium kluyveri (Scr, UniProt P38947 (SEQ ID NO: 26), Schürmann et al. J Bacteriol. 2011), Succinate semialdehyde dehydrogenase from Methylobacterium radiodurans (Scr, UniProt A0A2U8VWW1, SEQ ID NO: 100), Malonyl-CoA reductase from Chloroflexus aurantiacus (Mcr, UniProt Q6QQP7, SEQ ID NO: 28), Malonyl-CoA reductase from Sulfolobus tokodali or mutants thereof (Mcr, UnitProt Q96YK1, SEQ ID NOs: 30, 32, 34 and 36) or Malonyl-CoA reductase from Porphyrobacter dokdonensis (Mcr, UniProt. A0A1A7BFR5, SEQ ID NO: 38). More preferably, the Oxidoreductase is Succinyl-CoA reductase from Clostridium kluyveri (Scr, UniProt P38947, SEQ ID NO: 26).

Itaconate Semialdehyde Synthesis from Itaconic Acid

Alternatively to the first and second enzymes of the invention a Carboxylic acid reductase (Car) can be used to catalyze the formation of itaconate semialdehyde from Itaconic acid directly, without the intermediate step of producing Itaconyl-CoA.

Hence, in another aspect of the invention, the first enzyme is selected from at least one Acyl-CoA synthetase, at least one CoA-transferase and at least one Carboxylic acid reductase. If Carboxylic acid reductase is used as first enzyme, no second enzyme is used, i.e. no Oxidoreductase is used.

The term “Carboxylic acid reductase” or “Carboxylate reductase” refers to a group of enzymes that catalyze the ATP- and NADPH-dependent reduction of a wide range of acids to the corresponding aldehydes. These enzymes belong for example to the class EC 1.2.1.30. Carboxylic acid reductase contains an adenylation domain, a phosphopantetheinyl binding domain, and a reductase domain, and requires activation by attachment of a phosphopantetheinyl group, therefore, Carboxylic acid reductase is often co-expressed with a phosphopantetheinyl transferase (EC 2.7.8.7).

Domains with Carboxylic acid reductase activity can also be found within other polypeptides. Hence, in one embodiment, the Carboxylic acid reductase can be Carboxylic acid reductase derived from a bacterium selected from the group consisting of Mycobacterium phlei, Mycobacterium smegmatix, Nocardia iowensis, Nocardia otitidiscaviarum, and Tsukamurella paurometabola, or Carboxylic acid reductase can be comprised within Amino acid adenylation domain-containing protein of Enterovibrio norvegicus (UniProt; A0A1I5LHH4, SEQ ID NO: 82), Nostoc carneum NIES-2107 (UniProt: A0A1Z4I0C0, SEQ ID NO: 84), Pseudomonas sp. NFACC24-1 (UniProt: A0A1I5M4E6, SEQ ID NO: 88) or Clostridium sp. CAG:508, (UniProt: R6Q743, SEQ ID NO: 90) or the Carboxylic acid reductase can be comprised within Thioester reductase-domain containing protein of Xenorhabdus japonica (UniProt: A0A1I5ADW4, SEQ ID NO: 86), or the Carboxylic acid reductase can be comprised within Non-ribosomal peptide synthase of Minicystis rosea (UniProt: A0A1L6L9N8, SEQ ID NO: 92) or the Carboxylic acid reductase can comprised within an Oxidoreductase of Mycobacteroides chelonae (UniProt: A0A1S1KMX6; SEQ ID NO: 94) or carboxylic acid reductase of Mycobacterium abscessus (Genbank: ALM18851.1, SEQ ID NO: 96).

In one embodiment, the Carboxylic acid reductase is derived from a bacterium selected from the group consisting of Mycobacterium phlei, Mycobacterium smegmatix, Nocardia iowensis, Nocardia otitidiscaviarum, and Tsukamurella paurometabola. In a preferred embodiment, the Carboxylic acid reductase of the invention is from Nocardia iowensis (NiCar, UniProt Q6RKB1, SEQ ID NO: 52, He et al. AEM 2004). NiCar is proposed to act on itaconic acid (Winkler et al. Curr Opin Chem Biol. 2018).

Another alternative enzyme useful to catalyze the formation of itaconate semialdehyde from itaconic acid is Aspartate-semialdehyde dehydrogenase. Aspartate-semialdehyde dehydrogenases describe a group of oxidoreductases of EC class 1.2.1.11 that can catalyze the formation of semialdehydes from carboxylic acids.

In one embodiment, the first enzyme is selected from at least one Acyl-CoA synthetase, at least one CoA-transferase, at least one Carboxylic acid reductase and at least one Aspartate-semialdehyde dehydrogenase. If Aspartate-semialdehyde dehydrogenase is used as first enzyme, no second enzyme is used, i.e. no Oxidoreductase is used.

In one embodiment, the Aspartate-semialdehyde dehydrogenase is derived from a bacterium selected form the group consisting of Chlamydia pneumonia (UniProt: A0A0F7X0T3, SEQ ID NO: 54) Enhygromyxa salina (UniProt: A0A0C1ZL33, SEQ ID NO: 56) Planctomycetes bacterium (UniProt: A0A2A5D7X3, SEQ ID NO: 58) Cuniculiplasma divulgatum (UniProt: A0A1N5SZX4, SEQ ID NO: 60), Geobacillus sp. WSUCF1 (UniProt: S7SRU6, SEQ ID NO: 62), Roseovarius azorensis (UniProt: A0A1H7XPA2, SEQ ID NO: 64), and Chlamydiae (UniProt: A0A1F8J1K7, SEQ ID NO: 66).

2-Methylene-4-ol-Butyric Acid Synthesis from Itaconate Semialdehyde

The third enzyme of the invention catalyzes the formation of 2-Methylene-4-ol-butyric acid from Itaconate semialdehyde. Enzymes useful for this purpose are Oxidoreductases of the class EC 1.1.1. which act on the CH—OH group of donors with NAD+ or NADP+ as acceptor.

Hence, in a further aspect of the invention, the third enzyme of the invention is an Oxidoreductase selected from the group consisting of Alcohol dehydrogenase, Lactaldehyde reductase, 3-sulfolactaldehyde reductase, succinate semialdehyde reductase and Aldose/Aldehyde reductase. The term “Alcohol dehydrogenase” refers to a group of enzymes of class EC 1.1.1.1 that catalyze the interconversion between alcohols and aldehydes or ketones with the reduction of NAD+ to NADH. Alcohol dehydrogenases from yeasts, plants and bacteria catalyze the opposite reaction as part of fermentation, catalyzing the reaction of an aldehyde and NADH to a primary alcohol and NAD+.

In one embodiment, the third enzyme of the invention is an Alcohol dehydrogenase (EC 1.1.1.1). In a preferred embodiment, the Alcohol dehydrogenase is from Escherichia coli (YqhD, UniProt Q46856).

The term “Lactaldehyde reductase” refers to a group of enzymes of class EC 1.1.1.77 that is able to catalyze the reduction of an aldehyde to a primary alcohol. In one embodiment, the third enzyme of the invention is a Lactaldehyde reductase (EC 1.1.1.77). In a preferred embodiment, the Lactaldehyde reductase is from Escherichia coli (FucO, UniProt P0A9S1, Kim et al. J Ind Microbiol Biotechnol. 2015).

The term “3-sulfolactaldehyde reductase” refers to a group of enzymes of class EC 1.1.1.373 that is able to catalyze the reduction of an aldehyde to a primary alcohol. In one embodiment, the third enzyme of the invention is a 3-sulfolactaldehyde reductase (EC 1.1.1.373). In a preferred embodiment, the 3-sulfolactaldehyde reductase is from Escherichia coli (YihU, UniProt POA9V8).

The term “succinate semialdehyde reductase” refers to a group of enzymes of class EC 1.1.1.11 that is able to catalyze the NADPH-dependent reduction of succinic semialdehyde to gamma-hydroxybutyrate. In a preferred embodiment, the succinate semialdehyde reductase is from Homo sapiens (AKR7A2, UniProt 043488, SEQ ID NO: 118 or AKR7A3, UniProt 095154, SEQ ID NO: 124).

The term “Aldehyde reductase” or “Aldose reductase” refers to a group of enzymes of class EC 1.1.1.21 that catalyze the NADPH-dependent reduction of aldehydes to produce a primary alcohols. In one embodiment, the third enzyme of the invention is an Aldehyde reductase (EC 1.1.1.21).

### Formation of Tulipalin A (α-Methylene-γ-Butyro-Lactone)

The final step of tulipalin A synthesis is cyclic esterification of 2-Methylene-4-ol-butyric acid to form tulipalin A (α-Methylene-γ-butyro-lactone). Lactones are formed by intramolecular esterification of hydroxycarboxylic acids, which takes place spontaneously if the ring that is formed is five- or six-membered.

Formation of Tulipalin A from 2-Methylene-4-ol-Butyric Acid

In one embodiment, the formation of tulipalin A from 2-Methylene-4-ol-butyric acid occurs spontaneously through intramolecular esterification.

This step can also be catalyzed enzymatically. In one embodiment, the intramolecular esterification of 2-Methylene-4-ol-butyric acid is catalyzed by an enzyme selected from Mevalonolactone lactonase of Staphylococcus aureus (Drp35, UniProt Q99QV3, SEQ ID NO: 44, Reichert et al. Front Microbiol. 2018), 6-deoxyerythronolide synthase thioesterase from Saccharopolyspora erythraea (DEBS-TE, UniProt Q03133, SEQ ID NO: 46), Lactimidomycin thioesterase from Streptomyces amphibiosporus (LtmG-TE, UniProt D8UYP5, SEQ ID NO: 48) and Reveromycin thioesterase from Streptomyces sp. SN-593 (RevD-TE, UniProt G1 UDV4, SEQ ID NO: 50). In one embodiment, the intramolecular esterification of 2-Methylene-4-ol-butyric acid involves the formation of a 2-methylene-4-ol-butyryl-CoA intermediate.

### Formation of Tulipalin A Via 4-Acetyloxy-2-Methylene Butanoic Acid

In an alternative embodiment, the fourth enzyme used in the invention catalyzes the formation of 4-acetyloxy-2-methylene butanoic acid from 2-methylene-4-ol-butyric acid. Enzymes useful for this purpose are acyl transferases (family VIII carboxyesterases) of the class EC 3.1.1. which catalyze the acyl transfer from acyl donors like ethyl- or vinyl-acetate to the primary OH of 2-methylene-4-ol-butyric acid or the alcohol acetyl-CoA transferases of the class EC 2.3.1.84 which transfer an acyl group from acetyl-CoA to the primary OH of 2-methylene-4-ol-butyric acid. Hence, in an alternative aspect of the invention the fourth enzyme used in the invention is an acyltransferase selected from the group consisting of acyltransferase, carboxyesterase, carnitine acetyltransferase, galactoside O-acetyltransferase and alcohol acetyltransferase.

The term “acyl transferase” refers to a group of enzymes of class EC 3.1.1. that catalyze the acyl transfer between alcohols and acyl donors like ethyl acetate or vinyl acetate. Some carboxyesterases catalyze the reverse reaction of acyl transfer over hydrolysis.

In one embodiment, the acyl transfer to 2-Methylene-4-ol-butyric acid is catalyzed by an enzyme selected from acyltransferase MsAcT from Mycolicibacterium smegmatis (UniProt: AOR5U7, SEQ ID NO: 112), alcohol acetyl transferase ATF1 from Saccharomyces cerevisiae (UniProt: P40353, SEQ ID NO: 106), alcohol acetyl transferase ATF2 from Saccharomyces cerevisiae (UniProt: P53296, SEQ ID NO: 108), alcohol acetyl transferase Eat1 from Saccharomyces cerevisiae (UniProt: P53208, SEQ ID NO: 110), carnitine acetyltransferase YAT2 from Saccharomyces cerevisiae (UniProt: P40017, SEQ ID NO: 120) and galactoside O-acetyltransferase LacA from Escherichia coli (UniProt: P07464, SEQ ID NO: 122).

### Enzyme Combinations

In one embodiment, the method comprises contacting a reaction mixture comprising itaconic acid with a first enzyme of the invention and a second enzyme of the invention, or the recombinant cell or organism of the invention comprises a first enzyme of the invention and a second enzyme of the invention, wherein the first enzyme is Succinyl-CoA synthetase (SucCD) and the second enzyme is Succinyl-CoA reductase Scr. In one embodiment, subunit SucC of the Succinyl-CoA synthetase (SucCD) is at least 70% identical with an amino acid sequence according to SEQ ID NO: 2 and subunit SucD of the Succinyl-CoA synthetase (SucCD) is at least 70% identical with an amino acid sequence according to SEQ ID NO: 4 and Succinyl-CoA reductase Scr is at least 70% identical with an amino acid sequence according to SEQ ID NO: 26.

In another embodiment, subunit SucC of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 2 and subunit SucD of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 4 and Succinyl-CoA reductase Scr is at least 75%, at least 80%, at least 85%, at least 90%, at least 95%, at least 98%, at least 99% or 100% identical with an amino acid sequence according to SEQ ID NO: 26.

In one embodiment, the method comprises contacting a reaction mixture comprising itaconic acid with a first enzyme of the invention and a second enzyme of the invention, or the recombinant cell or organism of the invention comprises a first enzyme of the invention and a second enzyme of the invention, wherein the first enzyme is Succinyl-CoA synthetase (SucCD) and the second enzyme is Malonyl-CoA reductase Mcr. In one embodiment, subunit SucC of the Succinyl-CoA synthetase (SucCD) is at least 70% identical with an amino acid sequence according to SEQ ID NO: 2 and subunit SucD of the Succinyl-CoA synthetase (SucCD) is at least 70% identical with an amino acid sequence according to SEQ ID NO: 4 and Malonyl-CoA reductase Mcr is at least 70% identical with an amino acid sequence according to SEQ ID NO: 28.

In another embodiment, subunit SucC of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 2 and subunit SucD of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 4 and Malonyl-CoA reductase Mcr is at least 75%, at least 80%, at least 85%, at least 90%, at least 95%, at least 98%, at least 99% or 100% identical with an amino acid sequence according to SEQ ID NO: 28.

In one embodiment, the method comprises contacting a reaction mixture comprising itaconic acid with a first enzyme of the invention and a second enzyme of the invention, or the recombinant cell or organism of the invention comprises a first enzyme of the invention and a second enzyme of the invention, wherein the first enzyme is Succinyl-CoA synthetase (SucCD) and the second enzyme is HMG-CoA reductase HMGR. In one embodiment, subunit SucC of the Succinyl-CoA synthetase (SucCD) is at least 70% identical with an amino acid sequence according to SEQ ID NO: 2 and subunit SucD of the Succinyl-CoA synthetase (SucCD) is at least 70% identical with an amino acid sequence according to SEQ ID NO: 4 and HMG-CoA reductase HMGR is at least 70% identical with an amino acid sequence according to SEQ ID NO: 98.

In another embodiment, subunit SucC of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 2 and subunit SucD of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 4 and HMG-CoA reductase HMGR is at least 75%, at least 80%, at least 85%, at least 90%, at least 95%, at least 98%, at least 99% or 100% identical with an amino acid sequence according to SEQ ID NO: 98.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) and the second enzyme is Succinyl-CoA reductase Scr and the third enzyme is Alcohol dehydrogenase. In one embodiment, subunit SucC of the Succinyl-CoA synthetase (SucCD) is at least 70% identical with an amino acid sequence according to SEQ ID NO: 2 and subunit SucD of the Succinyl-CoA synthetase (SucCD) is at least 70% identical with an amino acid sequence according to SEQ ID NO: 4; Succinyl-CoA reductase Scr is at least 70% identical with an amino acid sequence according to SEQ ID NO: 26 and Alcohol dehydrogenase is at least 70% identical with an amino acid sequence according to SEQ ID NO: 42.

In another embodiment, subunit SucC of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 2 and subunit SucD of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 4; Succinyl-CoA reductase Scr is at least 75%, at least 80%, at least 85%, at least 90%, at least 95%, at least 98%, at least 99% or 100% identical with an amino acid sequence according to SEQ ID NO: 26 and Alcohol dehydrogenase is at least 75%, at least 80%, at least 85%, at least 90%, at least 95%, at least 98%, at least 99% or 100% identical with an amino acid sequence according to SEQ ID NO: 42.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) and the second enzyme is Succinyl-CoA reductase Scr and the third enzyme is 3-sulfolactaldehyde reductase. In one embodiment, subunit SucC of the Succinyl-CoA synthetase (SucCD) is at least 70% identical with an amino acid sequence according to SEQ ID NO: 2 and subunit SucD of the Succinyl-CoA synthetase (SucCD) is at least 70% identical with an amino acid sequence according to SEQ ID NO: 4; Succinyl-CoA reductase Scr is at least 70% identical with an amino acid sequence according to SEQ ID NO: 26 and 3-sulfolactaldehyde reductase is at least 70% identical with an amino acid sequence according to SEQ ID NO: 102.

In another embodiment, subunit SucC of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 2 and subunit SucD of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 4; Succinyl-CoA reductase Scr is at least 75%, at least 80%, at least 85%, at least 90%, at least 95%, at least 98%, at least 99% or 100% identical with an amino acid sequence according to SEQ ID NO: 26 and 3-sulfolactaldehyde reductase is at least 75%, at least 80%, at least 85%, at least 90%, at least 95%, at least 98%, at least 99% or 100% identical with an amino acid sequence according to SEQ ID NO: 102.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) and the second enzyme is Succinyl-CoA reductase Scr and the third enzyme is succinate semialdehyde reductase. In one embodiment, subunit SucC of the Succinyl-CoA synthetase (SucCD) is at least 70% identical with an amino acid sequence according to SEQ ID NO: 2 and subunit SucD of the Succinyl-CoA synthetase (SucCD) is at least 70% identical with an amino acid sequence according to SEQ ID NO: 4; Succinyl-CoA reductase Scr is at least 70% identical with an amino acid sequence according to SEQ ID NO: 26 and succinate semialdehyde reductase is at least 70% identical with an amino acid sequence according to SEQ ID NO: 118.

In another embodiment, subunit SucC of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 2 and subunit SucD of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 4; Succinyl-CoA reductase Scr is at least 75%, at least 80%, at least 85%, at least 90%, at least 95%, at least 98%, at least 99% or 100% identical with an amino acid sequence according to SEQ ID NO: 26 and succinate semialdehyde reductase is at least 75%, at least 80%, at least 85%, at least 90%, at least 95%, at least 98%, at least 99% or 100% identical with an amino acid sequence according to SEQ ID NO: 118.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) and the second enzyme is Malonyl-CoA reductase Mcr and the third enzyme is Alcohol dehydrogenase. In one embodiment, subunit SucC of the Succinyl-CoA synthetase (SucCD) is at least 70% identical with an amino acid sequence according to SEQ ID NO: 2 and subunit SucD of the Succinyl-CoA synthetase (SucCD) is at least 70% identical with an amino acid sequence according to SEQ ID NO: 4 and Malonyl-CoA reductase Mcr is at least 70% identical with an amino acid sequence according to SEQ ID NO: 28 and Alcohol dehydrogenase is at least 70% identical with an amino acid sequence according to SEQ ID NO: 42.

In another embodiment, subunit SucC of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 2 and subunit SucD of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 4 and Malonyl-CoA reductase Mcr is at least 75%, at least 80%, at least 85%, at least 90%, at least 95%, at least 98%, at least 99% or 100% identical with an amino acid sequence according to SEQ ID NO: 28 and Alcohol dehydrogenase is at least 75%, at least 80%, at least 85%, at least 90%, at least 95%, at least 98%, at least 99% or 100% identical with an amino acid sequence according to SEQ ID NO: 42.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) and the second enzyme is Malonyl-CoA reductase Mcr and the third enzyme is 3-sulfolactaldehyde reductase. In one embodiment, subunit SucC of the Succinyl-CoA synthetase (SucCD) is at least 70% identical with an amino acid sequence according to SEQ ID NO: 2 and subunit SucD of the Succinyl-CoA synthetase (SucCD) is at least 70% identical with an amino acid sequence according to SEQ ID NO: 4 and Malonyl-CoA reductase Mcr is at least 70% identical with an amino acid sequence according to SEQ ID NO: 28 and 3-sulfolactaldehyde reductase is at least 70% identical with an amino acid sequence according to SEQ ID NO: 102.

In another embodiment, subunit SucC of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 2 and subunit SucD of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 4 and Malonyl-CoA reductase Mcr is at least 75%, at least 80%, at least 85%, at least 90%, at least 95%, at least 98%, at least 99% or 100% identical with an amino acid sequence according to SEQ ID NO: 28 and 3-sulfolactaldehyde reductase is at least 75%, at least 80%, at least 85%, at least 90%, at least 95%, at least 98%, at least 99% or 100% identical with an amino acid sequence according to SEQ ID NO: 102.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) and the second enzyme is Malonyl-CoA reductase Mcr and the third enzyme is succinate semialdehyde reductase. In one embodiment, subunit SucC of the Succinyl-CoA synthetase (SucCD) is at least 70% identical with an amino acid sequence according to SEQ ID NO: 2 and subunit SucD of the Succinyl-CoA synthetase (SucCD) is at least 70% identical with an amino acid sequence according to SEQ ID NO: 4 and Malonyl-CoA reductase Mcr is at least 70% identical with an amino acid sequence according to SEQ ID NO: 28 and succinate semialdehyde reductase is at least 70% identical with an amino acid sequence according to SEQ ID NO: 118.

In another embodiment, subunit SucC of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 2 and subunit SucD of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 4 and Malonyl-CoA reductase Mcr is at least 75%, at least 80%, at least 85%, at least 90%, at least 95%, at least 98%, at least 99% or 100% identical with an amino acid sequence according to SEQ ID NO: 28 and succinate semialdehyde reductase is at least 75%, at least 80%, at least 85%, at least 90%, at least 95%, at least 98%, at least 99% or 100% identical with an amino acid sequence according to SEQ ID NO: 118.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) and the second enzyme is Malonyl-CoA reductase Mcr and the third enzyme is succinate semialdehyde reductase. In one embodiment, subunit SucC of the Succinyl-CoA synthetase (SucCD) is at least 70% identical with an amino acid sequence according to SEQ ID NO: 2 and subunit SucD of the Succinyl-CoA synthetase (SucCD) is at least 70% identical with an amino acid sequence according to SEQ ID NO: 4 and Malonyl-CoA reductase Mcr is at least 70% identical with an amino acid sequence according to SEQ ID NO: 28 and succinate semialdehyde reductase is at least 70% identical with an amino acid sequence according to SEQ ID NO: 124.

In another embodiment, subunit SucC of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 2 and subunit SucD of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 4 and Malonyl-CoA reductase Mcr is at least 75%, at least 80%, at least 85%, at least 90%, at least 95%, at least 98%, at least 99% or 100% identical with an amino acid sequence according to SEQ ID NO: 28 and succinate semialdehyde reductase is at least 75%, at least 80%, at least 85%, at least 90%, at least 95%, at least 98%, at least 99% or 100% identical with an amino acid sequence according to SEQ ID NO: 124.

In one embodiment, the method comprises contacting a reaction mixture comprising itaconic acid with a first enzyme of the invention and a second enzyme of the invention, or the recombinant cell or organism of the invention comprises a first enzyme of the invention and second enzyme of the invention, wherein the first enzyme is Succinyl-CoA synthetase (SucCD) and the second enzyme is Succinyl-CoA reductase Scr. In one embodiment, subunit SucC of the Succinyl-CoA synthetase (SucCD) is at least 70% identical with an amino acid sequence according to SEQ ID NO: 6 and subunit SucD of the Succinyl-CoA synthetase (SucCD) is at least 70% identical with an amino acid sequence according to SEQ ID NO: 8 and Succinyl-CoA reductase Scr is at least 70% identical with an amino acid sequence according to SEQ ID NO: 26.

In another embodiment, subunit SucC of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 6 and subunit SucD of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 8 and Succinyl-CoA reductase Scr is at least 75%, at least 80%, at least 85%, at least 90%, at least 95%, at least 98%, at least 99% or 100% identical with an amino acid sequence according to SEQ ID NO: 26.

In one embodiment, the method comprises contacting a reaction mixture comprising itaconic acid with a first enzyme of the invention and a second enzyme of the invention, or the recombinant cell or organism of the invention comprises a first enzyme of the invention and a second enzyme of the invention, wherein the first enzyme is Succinyl-CoA synthetase (SucCD) and the second enzyme is Malonyl-CoA reductase Mcr. In one embodiment, subunit SucC of the Succinyl-CoA synthetase (SucCD) is at least 70% identical with an amino acid sequence according to SEQ ID NO: 6 and subunit SucD of the Succinyl-CoA synthetase (SucCD) is at least 70% identical with an amino acid sequence according to SEQ ID NO: 8 and Malonyl-CoA reductase Mcr is at least 70% identical with an amino acid sequence according to SEQ ID NO: 28.

In another embodiment, subunit SucC of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 6 and subunit SucD of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 8 and Malonyl-CoA reductase Mcr is at least 75%, at least 80%, at least 85%, at least 90%, at least 95%, at least 98%, at least 99% or 100% identical with an amino acid sequence according to SEQ ID NO: 28.

In one embodiment, the method comprises contacting a reaction mixture comprising itaconic acid with a first enzyme of the invention and a second enzyme of the invention, or the recombinant cell or organism of the invention comprises a first enzyme of the invention and a second enzyme of the invention, wherein the first enzyme is Succinyl-CoA synthetase (SucCD) and the second enzyme is HMG-CoA reductase HMGR. In one embodiment, subunit SucC of the Succinyl-CoA synthetase (SucCD) is at least 70% identical with an amino acid sequence according to SEQ ID NO: 6 and subunit SucD of the Succinyl-CoA synthetase (SucCD) is at least 70% identical with an amino acid sequence according to SEQ ID NO: 8 and HMG-CoA reductase HMGR is at least 70% identical with an amino acid sequence according to SEQ ID NO: 98.

In another embodiment, subunit SucC of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 6 and subunit SucD of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 8 and HMG-CoA reductase HMGR is at least 75%, at least 80%, at least 85%, at least 90%, at least 95%, at least 98%, at least 99% or 100% identical with an amino acid sequence according to SEQ ID NO: 98.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) and the second enzyme is Succinyl-CoA reductase Scr and the third enzyme is Alcohol dehydrogenase. In one embodiment, subunit SucC of the Succinyl-CoA synthetase (SucCD) is at least 70% identical with an amino acid sequence according to SEQ ID NO: 6 and subunit SucD of the Succinyl-CoA synthetase (SucCD) is at least 70% identical with an amino acid sequence according to SEQ ID NO: 8; Succinyl-CoA reductase Scr is at least 70% identical with an amino acid sequence according to SEQ ID NO: 26 and Alcohol dehydrogenase is at least 70% identical with an amino acid sequence according to SEQ ID NO: 42.

In another embodiment, subunit SucC of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 6 and subunit SucD of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 8; Succinyl-CoA reductase Scr is at least 75%, at least 80%, at least 85%, at least 90%, at least 95%, at least 98%, at least 99% or 100% identical with an amino acid sequence according to SEQ ID NO: 26 and Alcohol dehydrogenase is at least 75%, at least 80%, at least 85%, at least 90%, at least 95%, at least 98%, at least 99% or 100% identical with an amino acid sequence according to SEQ ID NO: 42.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) and the second enzyme is Succinyl-CoA reductase Scr and the third enzyme is 3-sulfolactaldehyde reductase. In one embodiment, subunit SucC of the Succinyl-CoA synthetase (SucCD) is at least 70% identical with an amino acid sequence according to SEQ ID NO: 6 and subunit SucD of the Succinyl-CoA synthetase (SucCD) is at least 70% identical with an amino acid sequence according to SEQ ID NO: 8; Succinyl-CoA reductase Scr is at least 70% identical with an amino acid sequence according to SEQ ID NO: 26 and 3-sulfolactaldehyde reductase is at least 70% identical with an amino acid sequence according to SEQ ID NO: 102.

In another embodiment, subunit SucC of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 6 and subunit SucD of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 8; Succinyl-CoA reductase Scr is at least 75%, at least 80%, at least 85%, at least 90%, at least 95%, at least 98%, at least 99% or 100% identical with an amino acid sequence according to SEQ ID NO: 26 and 3-sulfolactaldehyde reductase is at least 75%, at least 80%, at least 85%, at least 90%, at least 95%, at least 98%, at least 99% or 100% identical with an amino acid sequence according to SEQ ID NO: 102.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) and the second enzyme is Succinyl-CoA reductase Scr and the third enzyme is succinate semialdehyde reductase. In one embodiment, subunit SucC of the Succinyl-CoA synthetase (SucCD) is at least 70% identical with an amino acid sequence according to SEQ ID NO: 6 and subunit SucD of the Succinyl-CoA synthetase (SucCD) is at least 70% identical with an amino acid sequence according to SEQ ID NO: 8; Succinyl-CoA reductase Scr is at least 70% identical with an amino acid sequence according to SEQ ID NO: 26 and succinate semialdehyde reductase is at least 70% identical with an amino acid sequence according to SEQ ID NO: 118.

In another embodiment, subunit SucC of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 6 and subunit SucD of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 8; Succinyl-CoA reductase Scr is at least 75%, at least 80%, at least 85%, at least 90%, at least 95%, at least 98%, at least 99% or 100% identical with an amino acid sequence according to SEQ ID NO: 26 and succinate semialdehyde reductase is at least 75%, at least 80%, at least 85%, at least 90%, at least 95%, at least 98%, at least 99% or 100% identical with an amino acid sequence according to SEQ ID NO: 118.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) and the second enzyme is Succinyl-CoA reductase Scr and the third enzyme is succinate semialdehyde reductase. In one embodiment, subunit SucC of the Succinyl-CoA synthetase (SucCD) is at least 70% identical with an amino acid sequence according to SEQ ID NO: 6 and subunit SucD of the Succinyl-CoA synthetase (SucCD) is at least 70% identical with an amino acid sequence according to SEQ ID NO: 8; Succinyl-CoA reductase Scr is at least 70% identical with an amino acid sequence according to SEQ ID NO: 26 and succinate semialdehyde reductase is at least 70% identical with an amino acid sequence according to SEQ ID NO: 124.

In another embodiment, subunit SucC of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 6 and subunit SucD of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 8; Succinyl-CoA reductase Scr is at least 75%, at least 80%, at least 85%, at least 90%, at least 95%, at least 98%, at least 99% or 100% identical with an amino acid sequence according to SEQ ID NO: 26 and succinate semialdehyde reductase is at least 75%, at least 80%, at least 85%, at least 90%, at least 95%, at least 98%, at least 99% or 100% identical with an amino acid sequence according to SEQ ID NO: 124.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) and the second enzyme is Malonyl-CoA reductase Mcr and the third enzyme is Alcohol dehydrogenase. In one embodiment, subunit SucC of the Succinyl-CoA synthetase (SucCD) is at least 70% identical with an amino acid sequence according to SEQ ID NO: 6 and subunit SucD of the Succinyl-CoA synthetase (SucCD) is at least 70% identical with an amino acid sequence according to SEQ ID NO: 8 and Malonyl-CoA reductase Mcr is at least 70% identical with an amino acid sequence according to SEQ ID NO: 28 and Alcohol dehydrogenase is at least 70% identical with an amino acid sequence according to SEQ ID NO: 42.

In another embodiment, subunit SucC of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 6 and subunit SucD of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 8 and Malonyl-CoA reductase Mcr is at least 75%, at least 80%, at least 85%, at least 90%, at least 95%, at least 98%, at least 99% or 100% identical with an amino acid sequence according to SEQ ID NO: 28 and Alcohol dehydrogenase is at least 75%, at least 80%, at least 85%, at least 90%, at least 95%, at least 98%, at least 99% or 100% identical with an amino acid sequence according to SEQ ID NO: 42.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) and the second enzyme is Malonyl-CoA reductase Mcr and the third enzyme is 3-sulfolactaldehyde reductase. In one embodiment, subunit SucC of the Succinyl-CoA synthetase (SucCD) is at least 70% identical with an amino acid sequence according to SEQ ID NO: 6 and subunit SucD of the Succinyl-CoA synthetase (SucCD) is at least 70% identical with an amino acid sequence according to SEQ ID NO: 8 and Malonyl-CoA reductase Mcr is at least 70% identical with an amino acid sequence according to SEQ ID NO: 28 and 3-sulfolactaldehyde reductase is at least 70% identical with an amino acid sequence according to SEQ ID NO: 102.

In another embodiment, subunit SucC of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 6 and subunit SucD of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 8 and Malonyl-CoA reductase Mcr is at least 75%, at least 80%, at least 85%, at least 90%, at least 95%, at least 98%, at least 99% or 100% identical with an amino acid sequence according to SEQ ID NO: 28 and 3-sulfolactaldehyde reductase is at least 75%, at least 80%, at least 85%, at least 90%, at least 95%, at least 98%, at least 99% or 100% identical with an amino acid sequence according to SEQ ID NO: 102.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) and the second enzyme is Malonyl-CoA reductase Mcr and the third enzyme is succinate semialdehyde reductase. In one embodiment, subunit SucC of the Succinyl-CoA synthetase (SucCD) is at least 70% identical with an amino acid sequence according to SEQ ID NO: 6 and subunit SucD of the Succinyl-CoA synthetase (SucCD) is at least 70% identical with an amino acid sequence according to SEQ ID NO: 8 and Malonyl-CoA reductase Mcr is at least 70% identical with an amino acid sequence according to SEQ ID NO: 28 and succinate semialdehyde reductase is at least 70% identical with an amino acid sequence according to SEQ ID NO: 118.

In another embodiment, subunit SucC of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 6 and subunit SucD of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 8 and Malonyl-CoA reductase Mcr is at least 75%, at least 80%, at least 85%, at least 90%, at least 95%, at least 98%, at least 99% or 100% identical with an amino acid sequence according to SEQ ID NO: 28 and succinate semialdehyde reductase is at least 75%, at least 80%, at least 85%, at least 90%, at least 95%, at least 98%, at least 99% or 100% identical with an amino acid sequence according to SEQ ID NO: 118.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) and the second enzyme is Malonyl-CoA reductase Mcr and the third enzyme is succinate semialdehyde reductase. In one embodiment, subunit SucC of the Succinyl-CoA synthetase (SucCD) is at least 70% identical with an amino acid sequence according to SEQ ID NO: 6 and subunit SucD of the Succinyl-CoA synthetase (SucCD) is at least 70% identical with an amino acid sequence according to SEQ ID NO: 8 and Malonyl-CoA reductase Mcr is at least 70% identical with an amino acid sequence according to SEQ ID NO: 28 and succinate semialdehyde reductase is at least 70% identical with an amino acid sequence according to SEQ ID NO: 124.

In another embodiment, subunit SucC of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 6 and subunit SucD of the Succinyl-CoA synthetase (SucCD) is at least 75%, at least 80%, at least 85%, at least 90%, at least 95%, at least 98%, at least 99% or 100% identical with an amino acid sequence according to SEQ ID NO: 8 and Malonyl-CoA reductase Mcr is at least 75%, at least 80%, at least 85%, at least 90%, at least 95%, at least 98%, at least 99% or 100% identical with an amino acid sequence according to SEQ ID NO: 28 and succinate semialdehyde reductase is at least 75%, at least 80%, at least 85%, at least 90%, at least 95%, at least 98%, at least 99% or 100% identical with an amino acid sequence according to SEQ ID NO: 124.

In one embodiment, the method comprises contacting a reaction mixture comprising itaconic acid with a first enzyme of the invention and a second enzyme of the invention, or the recombinant cell or organism of the invention comprises a first enzyme of the invention and a second enzyme of the invention, wherein the first enzyme is Malate-CoA ligase (MtkAB) from Methylorubrum extorquens and the second enzyme is Succinyl-CoA reductase Scr. In one embodiment, subunit alpha of the Malate-CoA ligase (MtkA) from Methylorubrum extorquens is at least 70% identical with an amino acid sequence according to SEQ ID NO: 14 and subunit beta of the Malate-CoA ligase (MtkB) is at least 70% identical with an amino acid sequence according to SEQ ID NO: 16 and Succinyl-CoA reductase Scr is at least 70% identical with an amino acid sequence according to SEQ ID NO: 26.

In another embodiment, subunit alpha of the Malate-CoA ligase (MtkA) from Methylorubrum extorquens is at least 75%, at least 80%, at least 85%, at least 90%, at least 95%, at least 98%, at least 99% or 100% identical with an amino acid sequence according to SEQ ID NO: 14 and subunit beta of the Malate-CoA ligase (MtkB) is at least 75%, at least 80%, at least 85%, at least 90%, at least 95%, at least 98%, at least 99% or 100% identical with an amino acid sequence according to SEQ ID NO: 16 and Succinyl-CoA reductase Scr is at least 75%, at least 80%, at least 85%, at least 90%, at least 95%, at least 98%, at least 99% or 100% identical with an amino acid sequence according to SEQ ID NO: 26.

In one embodiment, the method comprises contacting a reaction mixture comprising itaconic acid with a first enzyme of the invention and a second enzyme of the invention, or the recombinant cell or organism of the invention comprises a first enzyme of the invention and a second enzyme of the invention, wherein the first enzyme is Malate-CoA ligase (MtkAB) from Methylorubrum extorquens and the second enzyme is Malonyl-CoA reductase Mcr. In one embodiment, subunit alpha of the Malate-CoA ligase (MtkA) from Methylorubrum extorquens is at least 70% identical with an amino acid sequence according to SEQ ID NO: 14 and subunit beta of the Malate-CoA ligase (MtkB) is at least 70% identical with an amino acid sequence according to SEQ ID NO: 16 and Malonyl-CoA reductase Mcr is at least 70% identical with an amino acid sequence according to SEQ ID NO: 28.

In another embodiment, subunit alpha of the Malate-CoA ligase (MtkA) from Methylorubrum extorquens is at least 75%, at least 80%, at least 85%, at least 90%, at least 95%, at least 98%, at least 99% or 100% identical with an amino acid sequence according to SEQ ID NO: 14 and subunit beta of the Malate-CoA ligase (MtkB) is at least 75%, at least 80%, at least 85%, at least 90%, at least 95%, at least 98%, at least 99% or 100% identical with an amino acid sequence according to SEQ ID NO: 16 and Malonyl-CoA reductase Mcr is at least 75%, at least 80%, at least 85%, at least 90%, at least 95%, at least 98%, at least 99% or 100% identical with an amino acid sequence according to SEQ ID NO: 28.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Malate-CoA ligase (MtkAB) from Methylorubrum extorquens and the second enzyme is Succinyl-CoA reductase Scr and the third enzyme is Alcohol dehydrogenase. In one embodiment, subunit alpha of the Malate-CoA ligase (MtkA) from Methylorubrum extorquens is at least 70% identical with an amino acid sequence according to SEQ ID NO: 14 and subunit beta of the Malate-CoA ligase (MtkB) is at least 70% identical with an amino acid sequence according to SEQ ID NO: 16; Succinyl-CoA reductase Scr is at least 70% identical with an amino acid sequence according to SEQ ID NO: 26 and Alcohol dehydrogenase is at least 70% identical with an amino acid sequence according to SEQ ID NO: 42.

In another embodiment, subunit alpha of the Malate-CoA ligase (MtkA) from Methylorubrum extorquens is at least 75%, at least 80%, at least 85%, at least 90%, at least 95%, at least 98%, at least 99% or 100% identical with an amino acid sequence according to SEQ ID NO: 14 and subunit beta of the Malate-CoA ligase (MtkB) is at least 75%, at least 80%, at least 85%, at least 90%, at least 95%, at least 98%, at least 99% or 100% identical with an amino acid sequence according to SEQ ID NO: 16; Succinyl-CoA reductase Scr is at least 75%, at least 80%, at least 85%, at least 90%, at least 95%, at least 98%, at least 99% or 100% identical with an amino acid sequence according to SEQ ID NO: 26 and Alcohol dehydrogenase is at least 75%, at least 80%, at least 85%, at least 90%, at least 95%, at least 98%, at least 99% or 100% identical with an amino acid sequence according to SEQ ID NO: 42.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Malate-CoA ligase (MtkAB) from Methylorubrum extorquens and the second enzyme is Succinyl-CoA reductase Scr and the third enzyme is 3-sulfolactaldehyde reductase. In one embodiment, subunit alpha of the Malate-CoA ligase (MtkA) from Methylorubrum extorquens is at least 70% identical with an amino acid sequence according to SEQ ID NO: 14 and subunit beta of the Malate-CoA ligase (MtkB) is at least 70% identical with an amino acid sequence according to SEQ ID NO: 16; Succinyl-CoA reductase Scr is at least 70% identical with an amino acid sequence according to SEQ ID NO: 26 and 3-sulfolactaldehyde reductase is at least 70% identical with an amino acid sequence according to SEQ ID NO: 102.

In another embodiment, subunit alpha of the Malate-CoA ligase (MtkA) from Methylorubrum extorquens is at least 75%, at least 80%, at least 85%, at least 90%, at least 95%, at least 98%, at least 99% or 100% identical with an amino acid sequence according to SEQ ID NO: 14 and subunit beta of the Malate-CoA ligase (MtkB) is at least 75%, at least 80%, at least 85%, at least 90%, at least 95%, at least 98%, at least 99% or 100% identical with an amino acid sequence according to SEQ ID NO: 16; Succinyl-CoA reductase Scr is at least 75%, at least 80%, at least 85%, at least 90%, at least 95%, at least 98%, at least 99% or 100% identical with an amino acid sequence according to SEQ ID NO: 26 and 3-sulfolactaldehyde reductase is at least 75%, at least 80%, at least 85%, at least 90%, at least 95%, at least 98%, at least 99% or 100% identical with an amino acid sequence according to SEQ ID NO: 102.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Malate-CoA ligase (MtkAB) from Methylorubrum extorquens and the second enzyme is Succinyl-CoA reductase Scr and the third enzyme is succinate semialdehyde reductase. In one embodiment, subunit alpha of the Malate-CoA ligase (MtkA) from Methylorubrum extorquens is at least 70% identical with an amino acid sequence according to SEQ ID NO: 14 and subunit beta of the Malate-CoA ligase (MtkB) is at least 70% identical with an amino acid sequence according to SEQ ID NO: 16; Succinyl-CoA reductase Scr is at least 70% identical with an amino acid sequence according to SEQ ID NO: 26 and succinate semialdehyde reductase is at least 70% identical with an amino acid sequence according to SEQ ID NO: 118.

In another embodiment, subunit alpha of the Malate-CoA ligase (MtkA) from Methylorubrum extorquens is at least 75%, at least 80%, at least 85%, at least 90%, at least 95%, at least 98%, at least 99% or 100% identical with an amino acid sequence according to SEQ ID NO: 14 and subunit beta of the Malate-CoA ligase (MtkB) is at least 75%, at least 80%, at least 85%, at least 90%, at least 95%, at least 98%, at least 99% or 100% identical with an amino acid sequence according to SEQ ID NO: 16; Succinyl-CoA reductase Scr is at least 75%, at least 80%, at least 85%, at least 90%, at least 95%, at least 98%, at least 99% or 100% identical with an amino acid sequence according to SEQ ID NO: 26 and succinate semialdehyde reductase is at least 75%, at least 80%, at least 85%, at least 90%, at least 95%, at least 98%, at least 99% or 100% identical with an amino acid sequence according to SEQ ID NO: 118.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Malate-CoA ligase (MtkAB) from Methylorubrum extorquens and the second enzyme is Succinyl-CoA reductase Scr and the third enzyme is succinate semialdehyde reductase. In one embodiment, subunit alpha of the Malate-CoA ligase (MtkA) from Methylorubrum extorquens is at least 70% identical with an amino acid sequence according to SEQ ID NO: 14 and subunit beta of the Malate-CoA ligase (MtkB) is at least 70% identical with an amino acid sequence according to SEQ ID NO: 16; Succinyl-CoA reductase Scr is at least 70% identical with an amino acid sequence according to SEQ ID NO: 26 and succinate semialdehyde reductase is at least 70% identical with an amino acid sequence according to SEQ ID NO: 124.

In another embodiment, subunit alpha of the Malate-CoA ligase (MtkA) from Methylorubrum extorquens is at least 75%, at least 80%, at least 85%, at least 90%, at least 95%, at least 98%, at least 99% or 100% identical with an amino acid sequence according to SEQ ID NO: 14 and subunit beta of the Malate-CoA ligase (MtkB) is at least 75%, at least 80%, at least 85%, at least 90%, at least 95%, at least 98%, at least 99% or 100% identical with an amino acid sequence according to SEQ ID NO: 16; Succinyl-CoA reductase Scr is at least 75%, at least 80%, at least 85%, at least 90%, at least 95%, at least 98%, at least 99% or 100% identical with an amino acid sequence according to SEQ ID NO: 26 and succinate semialdehyde reductase is at least 75%, at least 80%, at least 85%, at least 90%, at least 95%, at least 98%, at least 99% or 100% identical with an amino acid sequence according to SEQ ID NO: 124.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Malate-CoA ligase (MtkAB) and the second enzyme is Malonyl-CoA reductase Mcr and the third enzyme is Alcohol dehydrogenase. In one embodiment, subunit alpha of the Malate-CoA ligase (MtkA) is at least 70% identical with an amino acid sequence according to SEQ ID NO: 14 and subunit beta of the Malate-CoA ligase (MtkB) is at least 70% identical with an amino acid sequence according to SEQ ID NO: 16 and Malonyl-CoA reductase Mcr is at least 70% identical with an amino acid sequence according to SEQ ID NO: 28 and Alcohol dehydrogenase is at least 70% identical with an amino acid sequence according to SEQ ID NO: 42.

In another embodiment, subunit alpha of the Malate-CoA ligase (MtkA) is at least 75%, at least 80%, at least 85%, at least 90%, at least 95%, at least 98%, at least 99% or 100% identical with an amino acid sequence according to SEQ ID NO: 14 and subunit beta of the Malate-CoA ligase (MtkB) is at least 75%, at least 80%, at least 85%, at least 90%, at least 95%, at least 98%, at least 99% or 100% identical with an amino acid sequence according to SEQ ID NO: 16 and Malonyl-CoA reductase Mcr is at least 75%, at least 80%, at least 85%, at least 90%, at least 95%, at least 98%, at least 99% or 100% identical with an amino acid sequence according to SEQ ID NO: 28 and Alcohol dehydrogenase is at least 75%, at least 80%, at least 85%, at least 90%, at least 95%, at least 98%, at least 99% or 100% identical with an amino acid sequence according to SEQ ID NO: 42.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Malate-CoA ligase (MtkAB) and the second enzyme is Malonyl-CoA reductase Mcr and the third enzyme is 3-sulfolactaldehyde reductase. In one embodiment, subunit alpha of the Malate-CoA ligase (MtkA) is at least 70% identical with an amino acid sequence according to SEQ ID NO: 14 and subunit beta of the Malate-CoA ligase (MtkB) is at least 70% identical with an amino acid sequence according to SEQ ID NO: 16 and Malonyl-CoA reductase Mcr is at least 70% identical with an amino acid sequence according to SEQ ID NO: 28 and 3-sulfolactaldehyde reductase is at least 70% identical with an amino acid sequence according to SEQ ID NO: 102.

In another embodiment, subunit alpha of the Malate-CoA ligase (MtkA) is at least 75%, at least 80%, at least 85%, at least 90%, at least 95%, at least 98%, at least 99% or 100% identical with an amino acid sequence according to SEQ ID NO: 14 and subunit beta of the Malate-CoA ligase (MtkB) is at least 75%, at least 80%, at least 85%, at least 90%, at least 95%, at least 98%, at least 99% or 100% identical with an amino acid sequence according to SEQ ID NO: 16 and Malonyl-CoA reductase Mcr is at least 75%, at least 80%, at least 85%, at least 90%, at least 95%, at least 98%, at least 99% or 100% identical with an amino acid sequence according to SEQ ID NO: 28 and 3-sulfolactaldehyde reductase is at least 75%, at least 80%, at least 85%, at least 90%, at least 95%, at least 98%, at least 99% or 100% identical with an amino acid sequence according to SEQ ID NO: 102.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Malate-CoA ligase (MtkAB) and the second enzyme is Malonyl-CoA reductase Mcr and the third enzyme is succinate semialdehyde reductase. In one embodiment, subunit alpha of the Malate-CoA ligase (MtkA) is at least 70% identical with an amino acid sequence according to SEQ ID NO: 14 and subunit beta of the Malate-CoA ligase (MtkB) is at least 70% identical with an amino acid sequence according to SEQ ID NO: 16 and Malonyl-CoA reductase Mcr is at least 70% identical with an amino acid sequence according to SEQ ID NO: 28 and succinate semialdehyde reductase is at least 70% identical with an amino acid sequence according to SEQ ID NO: 118.

In another embodiment, subunit alpha of the Malate-CoA ligase (MtkA) is at least 75%, at least 80%, at least 85%, at least 90%, at least 95%, at least 98%, at least 99% or 100% identical with an amino acid sequence according to SEQ ID NO: 14 and subunit beta of the Malate-CoA ligase (MtkB) is at least 75%, at least 80%, at least 85%, at least 90%, at least 95%, at least 98%, at least 99% or 100% identical with an amino acid sequence according to SEQ ID NO: 16 and Malonyl-CoA reductase Mcr is at least 75%, at least 80%, at least 85%, at least 90%, at least 95%, at least 98%, at least 99% or 100% identical with an amino acid sequence according to SEQ ID NO: 28 and succinate semialdehyde reductase is at least 75%, at least 80%, at least 85%, at least 90%, at least 95%, at least 98%, at least 99% or 100% identical with an amino acid sequence according to SEQ ID NO: 118.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Malate-CoA ligase (MtkAB) and the second enzyme is Malonyl-CoA reductase Mcr and the third enzyme is succinate semialdehyde reductase. In one embodiment, subunit alpha of the Malate-CoA ligase (MtkA) is at least 70% identical with an amino acid sequence according to SEQ ID NO: 14 and subunit beta of the Malate-CoA ligase (MtkB) is at least 70% identical with an amino acid sequence according to SEQ ID NO: 16 and Malonyl-CoA reductase Mcr is at least 70% identical with an amino acid sequence according to SEQ ID NO: 28 and succinate semialdehyde reductase is at least 70% identical with an amino acid sequence according to SEQ ID NO: 124.

In another embodiment, subunit alpha of the Malate-CoA ligase (MtkA) is at least 75%, at least 80%, at least 85%, at least 90%, at least 95%, at least 98%, at least 99% or 100% identical with an amino acid sequence according to SEQ ID NO: 14 and subunit beta of the Malate-CoA ligase (MtkB) is at least 75%, at least 80%, at least 85%, at least 90%, at least 95%, at least 98%, at least 99% or 100% identical with an amino acid sequence according to SEQ ID NO: 16 and Malonyl-CoA reductase Mcr is at least 75%, at least 80%, at least 85%, at least 90%, at least 95%, at least 98%, at least 99% or 100% identical with an amino acid sequence according to SEQ ID NO: 28 and succinate semialdehyde reductase is at least 75%, at least 80%, at least 85%, at least 90%, at least 95%, at least 98%, at least 99% or 100% identical with an amino acid sequence according to SEQ ID NO: 124.

In one embodiment, the method comprises contacting a reaction mixture comprising itaconic acid with a first enzyme of the invention and a second enzyme of the invention, or the recombinant cell or organism of the invention comprises a first enzyme of the invention and a second enzyme of the invention, wherein the first enzyme is Itaconate-CoA transferase (Ict) and the second enzyme is Succinyl-CoA reductase Scr. In one embodiment, Itaconate-CoA transferase (Ict) is at least 70% identical with an amino acid sequence according to SEQ ID NO: 18 and Succinyl-CoA reductase Scr is at least 70% identical with an amino acid sequence according to SEQ ID NO: 26.

In another embodiment, Itaconate-CoA transferase (Ict) is at least 75%, at least 80%, at least 85%, at least 90%, at least 95%, at least 98%, at least 99% or 100% identical with an amino acid sequence according to SEQ ID NO: 18 and Succinyl-CoA reductase Scr is at least 75%, at least 80%, at least 85%, at least 90%, at least 95%, at least 98%, at least 99% or 100% identical with an amino acid sequence according to SEQ ID NO: 26.

In one embodiment, the method comprises contacting a reaction mixture comprising itaconic acid with a first enzyme of the invention and a second enzyme of the invention, or the recombinant cell or organism of the invention comprises a first enzyme of the invention and a second enzyme of the invention, wherein the first enzyme is Itaconate-CoA transferase (Ict) and the second enzyme is Malonyl-CoA reductase Mcr. In one embodiment, Itaconate-CoA transferase (Ict) is at least 70% identical with an amino acid sequence according to SEQ ID NO: 22 and Malonyl-CoA reductase Mcr is at least 70% identical with an amino acid sequence according to SEQ ID NO: 28.

In another embodiment, Itaconate-CoA transferase (Ict) is at least 75%, at least 80%, at least 85%, at least 90%, at least 95%, at least 98%, at least 99% or 100% identical with an amino acid sequence according to SEQ ID NO: 22 and Malonyl-CoA reductase Mcr is at least 75%, at least 80%, at least 85%, at least 90%, at least 95%, at least 98%, at least 99% or 100% identical with an amino acid sequence according to SEQ ID NO: 28.

In one embodiment, the method comprises contacting a reaction mixture comprising itaconic acid with a first enzyme of the invention and a second enzyme of the invention, or the recombinant cell or organism of the invention comprises a first enzyme of the invention and a second enzyme of the invention, wherein the first enzyme is Itaconate-CoA transferase (Ict) and the second enzyme is HMG-CoA reductase HMGR. In one embodiment, Itaconate-CoA transferase (Ict) is at least 70% identical with an amino acid sequence according to SEQ ID NO: 22 and HMG-CoA reductase HMGR Mcr is at least 70% identical with an amino acid sequence according to SEQ ID NO: 98.

In another embodiment, Itaconate-CoA transferase (Ict) is at least 75%, at least 80%, at least 85%, at least 90%, at least 95%, at least 98%, at least 99% or 100% identical with an amino acid sequence according to SEQ ID NO: 22 and HMG-CoA reductase HMGR is at least 75%, at least 80%, at least 85%, at least 90%, at least 95%, at least 98%, at least 99% or 100% identical with an amino acid sequence according to SEQ ID NO: 98.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Itaconate-CoA transferase (Ict) and the second enzyme is Succinyl-CoA reductase Scr and the third enzyme is Alcohol dehydrogenase. In one embodiment, Itaconate-CoA transferase (Ict) is at least 70% identical with an amino acid sequence according to SEQ ID NO: 22; Succinyl-CoA reductase Scr is at least 70% identical with an amino acid sequence according to SEQ ID NO: 26 and Alcohol dehydrogenase is at least 70% identical with an amino acid sequence according to SEQ ID NO: 42.

In another embodiment, Itaconate-CoA transferase (Ict) is at least 75%, at least 80%, at least 85%, at least 90%, at least 95%, at least 98%, at least 99% or 100% identical with an amino acid sequence according to SEQ ID NO: 22; Succinyl-CoA reductase Scr is at least 75%, at least 80%, at least 85%, at least 90%, at least 95%, at least 98%, at least 99% or 100% identical with an amino acid sequence according to SEQ ID NO: 26 and Alcohol dehydrogenase is at least 75%, at least 80%, at least 85%, at least 90%, at least 95%, at least 98%, at least 99% or 100% identical with an amino acid sequence according to SEQ ID NO: 42.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Itaconate-CoA transferase (Ict) and the second enzyme is Succinyl-CoA reductase Scr and the third enzyme is 3-sulfolactaldehyde reductase. In one embodiment, Itaconate-CoA transferase (Ict) is at least 70% identical with an amino acid sequence according to SEQ ID NO: 22; Succinyl-CoA reductase Scr is at least 70% identical with an amino acid sequence according to SEQ ID NO: 26 and 3-sulfolactaldehyde reductase is at least 70% identical with an amino acid sequence according to SEQ ID NO: 102.

In another embodiment, Itaconate-CoA transferase (Ict) is at least 75%, at least 80%, at least 85%, at least 90%, at least 95%, at least 98%, at least 99% or 100% identical with an amino acid sequence according to SEQ ID NO: 22; Succinyl-CoA reductase Scr is at least 75%, at least 80%, at least 85%, at least 90%, at least 95%, at least 98%, at least 99% or 100% identical with an amino acid sequence according to SEQ ID NO: 26 and 3-sulfolactaldehyde reductase is at least 75%, at least 80%, at least 85%, at least 90%, at least 95%, at least 98%, at least 99% or 100% identical with an amino acid sequence according to SEQ ID NO: 102.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Itaconate-CoA transferase (Ict) and the second enzyme is Succinyl-CoA reductase Scr and the third enzyme is succinate semialdehyde reductase. In one embodiment, Itaconate-CoA transferase (Ict) is at least 70% identical with an amino acid sequence according to SEQ ID NO: 22; Succinyl-CoA reductase Scr is at least 70% identical with an amino acid sequence according to SEQ ID NO: 26 and succinate semialdehyde reductase is at least 70% identical with an amino acid sequence according to SEQ ID NO: 118.

In another embodiment, Itaconate-CoA transferase (Ict) is at least 75%, at least 80%, at least 85%, at least 90%, at least 95%, at least 98%, at least 99% or 100% identical with an amino acid sequence according to SEQ ID NO: 22; Succinyl-CoA reductase Scr is at least 75%, at least 80%, at least 85%, at least 90%, at least 95%, at least 98%, at least 99% or 100% identical with an amino acid sequence according to SEQ ID NO: 26 and succinate semialdehyde reductase is at least 75%, at least 80%, at least 85%, at least 90%, at least 95%, at least 98%, at least 99% or 100% identical with an amino acid sequence according to SEQ ID NO: 118.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Itaconate-CoA transferase (Ict) and the second enzyme is Succinyl-CoA reductase Scr and the third enzyme is succinate semialdehyde reductase. In one embodiment, Itaconate-CoA transferase (Ict) is at least 70% identical with an amino acid sequence according to SEQ ID NO: 22; Succinyl-CoA reductase Scr is at least 70% identical with an amino acid sequence according to SEQ ID NO: 26 and succinate semialdehyde reductase is at least 70% identical with an amino acid sequence according to SEQ ID NO: 124.

In another embodiment, Itaconate-CoA transferase (Ict) is at least 75%, at least 80%, at least 85%, at least 90%, at least 95%, at least 98%, at least 99% or 100% identical with an amino acid sequence according to SEQ ID NO: 22; Succinyl-CoA reductase Scr is at least 75%, at least 80%, at least 85%, at least 90%, at least 95%, at least 98%, at least 99% or 100% identical with an amino acid sequence according to SEQ ID NO: 26 and succinate semialdehyde reductase is at least 75%, at least 80%, at least 85%, at least 90%, at least 95%, at least 98%, at least 99% or 100% identical with an amino acid sequence according to SEQ ID NO: 124.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Itaconate-CoA transferase (Ict) and the second enzyme is Malonyl-CoA reductase Mcr and the third enzyme is Alcohol dehydrogenase. In one embodiment, Itaconate-CoA transferase (Ict) is at least 70% identical with an amino acid sequence according to SEQ ID NO: 22 and Malonyl-CoA reductase Mcr is at least 70% identical with an amino acid sequence according to SEQ ID NO: 28 and Alcohol dehydrogenase is at least 70% identical with an amino acid sequence according to SEQ ID NO: 42.

In another embodiment, Itaconate-CoA transferase (Ict) is at least 75%, at least 80%, at least 85%, at least 90%, at least 95%, at least 98%, at least 99% or 100% identical with an amino acid sequence according to SEQ ID NO: 22 and Malonyl-CoA reductase Mcr is at least 75%, at least 80%, at least 85%, at least 90%, at least 95%, at least 98%, at least 99% or 100% identical with an amino acid sequence according to SEQ ID NO: 28 and Alcohol dehydrogenase is at least 75%, at least 80%, at least 85%, at least 90%, at least 95%, at least 98%, at least 99% or 100% identical with an amino acid sequence according to SEQ ID NO: 42.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Itaconate-CoA transferase (Ict) and the second enzyme is Malonyl-CoA reductase Mcr and the third enzyme is 3-sulfolactaldehyde reductase. In one embodiment, Itaconate-CoA transferase (Ict) is at least 70% identical with an amino acid sequence according to SEQ ID NO: 22 and Malonyl-CoA reductase Mcr is at least 70% identical with an amino acid sequence according to SEQ ID NO: 28 and 3-sulfolactaldehyde reductase is at least 70% identical with an amino acid sequence according to SEQ ID NO: 102.

In another embodiment, Itaconate-CoA transferase (Ict) is at least 75%, at least 80%, at least 85%, at least 90%, at least 95%, at least 98%, at least 99% or 100% identical with an amino acid sequence according to SEQ ID NO: 22 and Malonyl-CoA reductase Mcr is at least 75%, at least 80%, at least 85%, at least 90%, at least 95%, at least 98%, at least 99% or 100% identical with an amino acid sequence according to SEQ ID NO: 28 and 3-sulfolactaldehyde reductase is at least 75%, at least 80%, at least 85%, at least 90%, at least 95%, at least 98%, at least 99% or 100% identical with an amino acid sequence according to SEQ ID NO: 102.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Itaconate-CoA transferase (Ict) and the second enzyme is Malonyl-CoA reductase Mcr and the third enzyme is succinate semialdehyde reductase. In one embodiment, Itaconate-CoA transferase (Ict) is at least 70% identical with an amino acid sequence according to SEQ ID NO: 22 and Malonyl-CoA reductase Mcr is at least 70% identical with an amino acid sequence according to SEQ ID NO: 28 and succinate semialdehyde reductase is at least 70% identical with an amino acid sequence according to SEQ ID NO: 118.

In another embodiment, Itaconate-CoA transferase (Ict) is at least 75%, at least 80%, at least 85%, at least 90%, at least 95%, at least 98%, at least 99% or 100% identical with an amino acid sequence according to SEQ ID NO: 22 and Malonyl-CoA reductase Mcr is at least 75%, at least 80%, at least 85%, at least 90%, at least 95%, at least 98%, at least 99% or 100% identical with an amino acid sequence according to SEQ ID NO: 28 and succinate semialdehyde reductase is at least 75%, at least 80%, at least 85%, at least 90%, at least 95%, at least 98%, at least 99% or 100% identical with an amino acid sequence according to SEQ ID NO: 118.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Itaconate-CoA transferase (Ict) and the second enzyme is Malonyl-CoA reductase Mcr and the third enzyme is succinate semialdehyde reductase. In one embodiment, Itaconate-CoA transferase (Ict) is at least 70% identical with an amino acid sequence according to SEQ ID NO: 22 and Malonyl-CoA reductase Mcr is at least 70% identical with an amino acid sequence according to SEQ ID NO: 28 and succinate semialdehyde reductase is at least 70% identical with an amino acid sequence according to SEQ ID NO: 124.

In another embodiment, Itaconate-CoA transferase (Ict) is at least 75%, at least 80%, at least 85%, at least 90%, at least 95%, at least 98%, at least 99% or 100% identical with an amino acid sequence according to SEQ ID NO: 22 and Malonyl-CoA reductase Mcr is at least 75%, at least 80%, at least 85%, at least 90%, at least 95%, at least 98%, at least 99% or 100% identical with an amino acid sequence according to SEQ ID NO: 28 and succinate semialdehyde reductase is at least 75%, at least 80%, at least 85%, at least 90%, at least 95%, at least 98%, at least 99% or 100% identical with an amino acid sequence according to SEQ ID NO: 124.

In one embodiment, the method comprises contacting a reaction mixture comprising itaconic acid with a first enzyme of the invention and a second enzyme of the invention, or the recombinant cell or organism of the invention comprises a first enzyme of the invention and a second enzyme of the invention, wherein the first enzyme is Succinyl-CoA synthetase (SucCD) from Escherichia coli and the second enzyme is Succinyl-CoA reductase Scr from Clostridium kluyveri.

In one embodiment, the method comprises contacting a reaction mixture comprising itaconic acid with a first enzyme of the invention and a second enzyme of the invention, or the recombinant cell or organism of the invention comprises a first enzyme of the invention and a second enzyme of the invention, wherein the first enzyme is Succinyl-CoA synthetase (SucCD) from Escherichia coli and the second enzyme is Malonyl-CoA reductase Mcr from Chloroflexus aurantiacus.

In one embodiment, the method comprises contacting a reaction mixture comprising itaconic acid with a first enzyme of the invention and a second enzyme of the invention, or the recombinant cell or organism of the invention comprises a first enzyme of the invention and a second enzyme of the invention, wherein the first enzyme is Succinyl-CoA synthetase (SucCD) from Escherichia coli and the second enzyme is HMG-CoA reductase HMGR from Methanothermococcus thermolithotrophicus.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) from Escherichia coli and the second enzyme is Succinyl-CoA reductase Scr from Clostridium kluyveri and the third enzyme is Alcohol dehydrogenase YqhD from Escherichia coli.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) from Escherichia coli and the second enzyme is Succinyl-CoA reductase Scr from Clostridium kluyveri and the third enzyme is 3-sulfolactaldehyde reductase YihU from Escherichia coli strain K12.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) from Escherichia coli and the second enzyme is Succinyl-CoA reductase Scr from Clostridium kluyveri and the third enzyme is succinate semialdehyde reductase ARK7A2 from Homo sapiens.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) from Escherichia coli and the second enzyme is Succinyl-CoA reductase Scr from Clostridium kluyveri and the third enzyme is succinate semialdehyde reductase ARK7A3 from Homo sapiens.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) from Escherichia coli and the second enzyme is Malonyl-CoA reductase Mcr from Chloroflexus aurantiacus and the third enzyme is Alcohol dehydrogenase YqhD from Escherichia coli.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) from Escherichia coli and the second enzyme is Malonyl-CoA reductase Mcr from Chloroflexus aurantiacus and the third enzyme is 3-sulfolactaldehyde reductase YihU from Escherichia coli strain K12.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) from Escherichia coli and the second enzyme is Malonyl-CoA reductase Mcr from Chloroflexus aurantiacus and the third enzyme is succinate semialdehyde reductase ARK7A2 from Homo sapiens.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) from Escherichia coli and the second enzyme is Malonyl-CoA reductase Mcr from Chloroflexus aurantiacus and the third enzyme is succinate semialdehyde reductase ARK7A3 from Homo sapiens.

In one embodiment, the method comprises contacting a reaction mixture comprising itaconic acid with a first enzyme of the invention and a second enzyme of the invention, or the recombinant cell or organism of the invention comprises a first enzyme of the invention and second enzyme of the invention, wherein the first enzyme is Succinyl-CoA synthetase (SucCD) from Advenella mimigardefordensis and the second enzyme is Succinyl-CoA reductase Scr from Clostridium kluyveri.

In one embodiment, the method comprises contacting a reaction mixture comprising itaconic acid with a first enzyme of the invention and a second enzyme of the invention, or the recombinant cell or organism of the invention comprises a first enzyme of the invention and a second enzyme of the invention, wherein the first enzyme is Succinyl-CoA synthetase (SucCD) from Advenella mimigardefordensis and the second enzyme is Malonyl-CoA reductase Mcr from Chloroflexus aurantiacus.

In one embodiment, the method comprises contacting a reaction mixture comprising itaconic acid with a first enzyme of the invention and a second enzyme of the invention, or the recombinant cell or organism of the invention comprises a first enzyme of the invention and a second enzyme of the invention, wherein the first enzyme is Succinyl-CoA synthetase (SucCD) from Advenella mimigardefordensis and the second enzyme is HMG-CoA reductase HMGR from Methanothermococcus thermolithotrophicus.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) from Advenella mimigardefordensis and the second enzyme is Succinyl-CoA reductase Scr from Clostridium kluyveri and the third enzyme is Alcohol dehydrogenase YqhD from Escherichia coli.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) from Advenella mimigardefordensis and the second enzyme is Succinyl-CoA reductase Scr from Clostridium kluyveri and the third enzyme is 3-sulfolactaldehyde reductase YihU from Escherichia coli strain K12.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) from Advenella mimigardefordensis and the second enzyme is Succinyl-CoA reductase Scr from Clostridium kluyveri and the third enzyme is succinate semialdehyde reductase ARK7A2 from Homo sapiens.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) from Advenella mimigardefordensis and the second enzyme is Succinyl-CoA reductase Scr from Clostridium kluyveri and the third enzyme is succinate semialdehyde reductase ARK7A3 from Homo sapiens.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) from Advenella mimigardefordensis and the second enzyme is Malonyl-CoA reductase Mcr from Chloroflexus aurantiacus and the third enzyme is Alcohol dehydrogenase YqhD from Escherichia coli.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) from Advenella mimigardefordensis and the second enzyme is Malonyl-CoA reductase Mcr from Chloroflexus aurantiacus and the third enzyme is 3-sulfolactaldehyde reductase YihU from Escherichia coli strain K12.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) from Advenella mimigardefordensis and the second enzyme is Malonyl-CoA reductase Mcr from Chloroflexus aurantiacus and the third enzyme is succinate semialdehyde reductase ARK7A2 from Homo sapiens.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Succinyl-CoA synthetase (SucCD) from Advenella mimigardefordensis and the second enzyme is Malonyl-CoA reductase Mcr from Chloroflexus aurantiacus and the third enzyme is succinate semialdehyde reductase ARK7A3 from Homo sapiens.

In one embodiment, the method comprises contacting a reaction mixture comprising itaconic acid with a first enzyme of the invention and a second enzyme of the invention, or the recombinant cell or organism of the invention comprises a first enzyme of the invention and a second enzyme of the invention, wherein the first enzyme is Malate-CoA ligase (MtkAB) from Methylorubrum extorquens and the second enzyme is Succinyl-CoA reductase Scr from Clostridium kluyveri.

In one embodiment, the method comprises contacting a reaction mixture comprising itaconic acid with a first enzyme of the invention and a second enzyme of the invention, or the recombinant cell or organism of the invention comprises a first enzyme of the invention and a second enzyme of the invention, wherein the first enzyme is Malate-CoA ligase (MtkAB) from Methylorubrum extorquens and the second enzyme is Malonyl-CoA reductase Mcr from Chloroflexus aurantiacus.

In one embodiment, the method comprises contacting a reaction mixture comprising itaconic acid with a first enzyme of the invention and a second enzyme of the invention, or the recombinant cell or organism of the invention comprises a first enzyme of the invention and a second enzyme of the invention, wherein the first enzyme is Malate-CoA ligase (MtkAB) from Methylorubrum extorquens and the second enzyme is HMG-CoA reductase HMGR from Methanothermococcus thermolithotrophicus.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Malate-CoA ligase (MtkAB) from Methylorubrum extorquens and the second enzyme is Succinyl-CoA reductase Scr from Clostridium kluyveri and the third enzyme is Alcohol dehydrogenase YqhD from Escherichia coli.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Malate-CoA ligase (MtkAB) from Methylorubrum extorquens and the second enzyme is Succinyl-CoA reductase Scr from Clostridium kluyveri and the third enzyme is 3-sulfolactaldehyde reductase YihU from Escherichia coli strain K12.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Malate-CoA ligase (MtkAB) from Methylorubrum extorquens and the second enzyme is Succinyl-CoA reductase Scr from Clostridium kluyveri and the third enzyme is succinate semialdehyde reductase ARK7A2 from Homo sapiens.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Malate-CoA ligase (MtkAB) from Methylorubrum extorquens and the second enzyme is Succinyl-CoA reductase Scr from Clostridium kluyveri and the third enzyme is succinate semialdehyde reductase ARK7A3 from Homo sapiens.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Malate-CoA ligase (MtkAB) from Methylorubrum extorquens and the second enzyme is Malonyl-CoA reductase Mcr from Chloroflexus aurantiacus and the third enzyme is Alcohol dehydrogenase YqhD from Escherichia coli.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Malate-CoA ligase (MtkAB) from Methylorubrum extorquens and the second enzyme is Malonyl-CoA reductase Mcr from Chloroflexus aurantiacus and the third enzyme is 3-sulfolactaldehyde reductase YihU from Escherichia coli strain K12.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Malate-CoA ligase (MtkAB) from Methylorubrum extorquens and the second enzyme is Malonyl-CoA reductase Mcr from Chloroflexus aurantiacus and the third enzyme is succinate semialdehyde reductase ARK7A2 from Homo sapiens.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Malate-CoA ligase (MtkAB) from Methylorubrum extorquens and the second enzyme is Malonyl-CoA reductase Mcr from Chloroflexus aurantiacus and the third enzyme is succinate semialdehyde reductase ARK7A3 from Homo sapiens.

In one embodiment, the method comprises contacting a reaction mixture comprising itaconic acid with a first enzyme of the invention and a second enzyme of the invention, or the recombinant cell or organism of the invention comprises a first enzyme of the invention and a second enzyme of the invention, wherein the first enzyme is Itaconate-CoA transferase (Ict) from Pseudomonas aeruginosa and the second enzyme is Succinyl-CoA reductase Scr from Clostridium kluyveri.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Itaconate-CoA transferase (Ict) from Pseudomonas aeruginosa and the second enzyme is Succinyl-CoA reductase Scr from Clostridium kluyveri and the third enzyme is Alcohol dehydrogenase YqhD from Escherichia coli.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Itaconate-CoA transferase (Ict) from Pseudomonas aeruginosa and the second enzyme is Succinyl-CoA reductase Scr from Clostridium kluyveri and the third enzyme is 3-sulfolactaldehyde reductase YihU from Escherichia coli strain K12.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Itaconate-CoA transferase (Ict) from Pseudomonas aeruginosa and the second enzyme is Succinyl-CoA reductase Scr from Clostridium kluyveri and the third enzyme is succinate semialdehyde reductase ARK7A2 from Homo sapiens.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Itaconate-CoA transferase (Ict) from Pseudomonas aeruginosa and the second enzyme is Succinyl-CoA reductase Scr from Clostridium kluyveri and the third enzyme is succinate semialdehyde reductase ARK7A3 from Homo sapiens.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Itaconate-CoA transferase (Ict) from Pseudomonas aeruginosa and the second enzyme is Malonyl-CoA reductase Mcr from Chloroflexus aurantiacus and the third enzyme is Alcohol dehydrogenase YqhD from Escherichia coli.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Itaconate-CoA transferase (Ict) from Pseudomonas aeruginosa and the second enzyme is Malonyl-CoA reductase Mcr from Chloroflexus aurantiacus and the third enzyme is 3-sulfolactaldehyde reductase YihU from Escherichia coli strain K12.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Itaconate-CoA transferase (Ict) from Pseudomonas aeruginosa and the second enzyme is Malonyl-CoA reductase Mcr from Chloroflexus aurantiacus and the third enzyme is succinate semialdehyde reductase ARK7A2 from Homo sapiens.

In one embodiment, the method comprises contacting a reaction mixture comprising itaconic acid with a first enzyme of the invention, a second enzyme of the invention and a third enzyme of the invention, or the recombinant cell or organism of the invention comprises a first enzyme of the invention, a second enzyme of the invention and a third enzyme of the invention, wherein the first enzyme is Itaconate-CoA transferase (Ict) from Pseudomonas aeruginosa and the second enzyme is Malonyl-CoA reductase Mcr from Chloroflexus aurantiacus and the third enzyme is succinate semialdehyde reductase ARK7A3 from Homo sapiens.

Isolation of tulipalin A After completion of the production process, the tulipalin A produced by the method of the present invention may be isolated by known methods, in particular by using organic solvents such as heptane, ethyl acetate, cylohexanol and 2-tertiary butylphenol. Preferably, the tulipaline A is isolated using heptane and 2-tertiary butylphenol.

### EXAMPLES

The following examples are provided for illustrative purposes. It is thus understood that the examples are not to be construed as limiting. The skilled person will clearly be able to envisage further modifications of the principles laid out herein.

### Materials and Methods

Itaconic acid and Tulipalin A were purchased from Sigma Aldrich (Munich, Germany). Chemicals and materials used for the protein expression were purchased from New England Biolabs GmbH (Frankfurt am Main, Germany), Macharey-Nagel GmbH (Düren, Germany) and GE Healthcare. Identity of all the recombinant proteins was confirmed using SDS-PAGE.

### Enzymes

Enzymes were isolated from the source organism and cloned into plasmid vectors as indicated in Table 1. Table 1 provides the enzyme name, full name, source organism, UniProt accession number, vector and SEQ ID NO of enzymes used in the examples and enzymes useful for the invention.

TABLE 1
Exemplary enzymes used in the invention

| | | | UniProt/ | | |
|---|---|---|---|---|---|
| Enzyme | Name | Source | Genbank | Vector | SEQ ID NO: |
| SucC | ADP-forming | Escherichia coli (strain | P0A836 | pCA24N | 1: gene |
| | succinate-CoA | K12) | | | 2: protein |
| | ligase | | | | 3: gene |
| | subunit beta | | | | 4: protein |
| SucD | ADP-forming | | P0AGE9 | | |
| | succinate-CoA | | | | |
| | ligase | | | | |
| | subunit alpha | | | | |
| SucC | ADP-forming | Advenella | W0PFR9 | | 5: gene |
| | succinate-CoA | mimigardefordensis | | | 6: protein |
| | ligase | | | | 7: gene |
| | subunit beta | | | | 8: protein |
| SucD | ADP-forming | | W0PAN5 | | |
| | succinate-CoA | | | | |
| | ligase | | | | |
| | subunit alpha | | | | |
| SucC | ADP-forming | Alcanivorax | Q0VPF7 | pETDuet-1 | 9: gene A |
| | succinate-CoA | borkumensis | | | 10: protein A |
| | ligase | | | | 11: gene B |
| | subunit beta | | | | 12: protein B |
| SucD | ADP-forming | | Q0VPF8 | | |
| | succinate-CoA | | | | |
| | ligase | | | | |
| | subunit alpha | | | | |
| MtkAB | Malate-CoA ligase | Methylorubrum | P53594 | pET28b | 13: gene A |
| | Subunits beta and | extorquens | P53595 | | 14: protein A |
| | alpha | | | | 15: gene B |
| | | | | | 16: protein B |
| Tfu_2577* | ADP-forming | Thermobifida fusca | Q47LR2 | pET28a | 17: gene |
| | succinate-CoA | | | | 18: protein |
| | ligase | | | | |
| | subunit beta | | | | |
| Tfu_2576 | ADP-forming | Thermobifida fusca | Q47LR3 | pET28a | 19: gene |
| | succinate-CoA | | | | 20: protein |
| | ligase | | | | |
| | subunit alpha | | | | |
| Ict | Itaconate-CoA | Pseudomonas | Q9I563 | pET28a | 21: gene |
| | transferase | aeruginosa | | | 22: protein |
| RpiA/YpIct | 4-hydroxybutyrate | Yersinia pestis | YPO1926 | | 23: gene |
| | CoA transferase/ | | | | 24: protein |
| | Itaconate CoA- | | | | |
| | transferase | | | | |
| Scr | Succinyl-CoA | Clostridium kluyveri | P38947 | pCDF- | 25: gene |
| | reductase | | | Duet-1 | 26: protein |
| Mcr | Malonyl-CoA | Chloroflexus | Q6QQP7 | | 27: gene |
| | reductase | aurantiacus | | | 28: protein |
| Mcr | Malonyl-CoA | Sulfolobus tokodaii | Q96YK1 | pDHE | 29: gene |
| | reductase | | | | 30: protein |
| | L152V mutant | | | | 31: gene |
| | L152A mutant | | | | 32: protein |
| | L152T mutant | | | | 33: gene |
| | Wild type | | | | 34: protein |
| | | | | | 35: gene |
| | | | | | 36: protein |
| Mcr | Malonyl-CoA | Porphyrobacter | A0A1A7BFR5 | | 37: gene |
| | reductase | dokdonensis | | | 38: protein |
| FucO | Lactaldehyde | Escherichia coli (strain | P0A9S1 | pCA24N | 39: gene |
| | reductase | K12) | | | 40: protein |
| YqhD | Alcohol | Escherichia coli (strain | Q46856 | pCA24N | 41: gene |
| | dehydrogenase | K12) | | | 42: protein |
| Drp35 | Mevalonolactone | Staphylococcus | Q99QV3 | pET21a | 43: gene |
| | lactonase | aureus | | | 44: protein |
| DEBS_TE | 6- | Saccharopolyspora | Q03133 | pET21a | 45: gene |
| | deoxyerythronolide | erythraea | (2896 . . . 3169) | | 46: protein |
| | synthase | | | | |
| | thioesterase | | | | |
| LtmG_TE | Lactimidomycin | Streptomyces | D8UYP5 | pET21a | 47: gene |
| | thioesterase | amphibiosporus | (1797 . . . 2073) | | 48: protein |
| RevD_TE | Reveromycin | Streptomyces sp. SN- | G1UDV4 | pET21a | 49: gene |
| | thioesterase | 593 | (6807 . . . 7023) | | 50: protein |
| NiCar | Carboxylic acid | Nocardia iowensis | Q6RKB1 | pETDuet1 | 51: gene |
| | reductase | | | | 52: protein |
| Asd | Aspartate- | Chlamydia | A0A0F7X0T3 | pDHE | 53: gene |
| | semialdehyde | pneumoniae | | | 54: protein |
| | dehydrogenase | | | | |
| Asd | Aspartate- | Enhygromyxa salina | A0A0C1ZL33 | pDHE | 55: gene |
| | semialdehyde | | | | 56: protein |
| | dehydrogenase | | | | |
| Asd | Aspartate- | Planctomycetes | A0A2A5D7X3 | pDHE | 57: gene |
| | semialdehyde | bacterium | | | 58: protein |
| | dehydrogenase | | | | |
| Asd | Aspartate- | Cuniculiplasma | A0A1N5SZX4 | pDHE | 59: gene |
| | semialdehyde | divulgatum | | | 60: protein |
| | dehydrogenase | | | | |
| Asd | Aspartate- | Geobacillus sp. | S7SRU6 | pDHE | 61: gene |
| | semialdehyde | WSUCF1 | | | 62: protein |
| | dehydrogenase | | | | |
| Asd | Aspartate- | Roseovarius azorensis | A0A1H7XPA2 | pDHE | 63: gene |
| | semialdehyde | | | | 64: protein |
| | dehydrogenase | | | | |
| Asd | Aspartate- | Chlamydiae | A0A1F8J1K7 | pDHE | 65: gene |
| | semialdehyde | | | | 66: protein |
| | dehydrogenase | | | | |
| HMGR | HMG-CoA | Polaribacter | A0A2S7KW32 | pDHE | 67: gene |
| | reductase | filamentous | | | 68: protein |
| HMGR | HMG-CoA | Lactobacillus | A0A269ZLZ0 | pDHE | 69: gene |
| | reductase | kefiranofaciens | | | 70: protein |
| HMGR | HMG-CoA | Pseudobacteriovorax | A0A1Y6BIN7 | pDHE | 71: gene |
| | reductase | antillogorgiicola | | | 72: protein |
| HMGR | HMG-CoA | Lactobacillus | A0A0C9PS41 | pDHE | 73: gene |
| | reductase | paracasei NRIC 0644 | | | 74: protein |
| HMGR | HMG-CoA | Methanocella sp., | A0A1V4Z2S1 | pDHE | 75: gene |
| | reductase | | | | 76: protein |
| HMGR | HMG-CoA | Capnocytophaga sp | L1P7W3 | pDHE | 77: gene |
| | reductase | | | | 78: protein |
| HMGR | HMG-CoA | Longimonas halophila | A0A2H3NXD1 | pDHE | 79: gene |
| | reductase | | | | 80: protein |
| CAR | Amino acid | Enterovibrio | A0A1I5LHH4 | pETDUET- | 81: gene |
| | adenylation | norvegicus | | 1 | 82: protein |
| | domain-containing | | | | |
| | protein | | | | |
| CAR | Amino acid | Nostoc carneum | A0A1Z4I0C0 | pETDUET- | 83: gene |
| | adenylation | NIES-2107 | | 1 | 84: protein |
| | domain-containing | | | | |
| | protein | | | | |
| CAR | Thioester | Xenorhabdus japonica | A0A1I5ADW4 | pETDUET- | 85: gene |
| | reductase-domain | | | 1 | 86: protein |
| | containing protein | | | | |
| CAR | Amino acid | Pseudomonas sp. | A0A1I5M4E6 | pETDUET- | 87: gene |
| | adenylation | NFACC24-1 | | 1 | 88: protein |
| | domain-containing | | | | |
| | protein | | | | |
| CAR | Amino acid | Clostridium sp. | R6Q743 | pETDUET- | 89: gene |
| | adenylation | CAG: 508 | | 1 | 90: protein |
| | domain-containing | | | | |
| | protein | | | | |
| CAR | Non-ribosomal | Minicystis rosea | A0A1L6L9N8 | pETDUET- | 91: gene |
| | peptide synthase | | | 1 | 92: protein |
| CAR | Oxidoreductase | Mycobacteroides | A0A1S1KMX6 | pETDUET- | 93: gene |
| | | chelonae | | 1 | 94: protein |
| CAR | Carboxylic acid | Mycobacterium | ALM18851.1 | pDHE | 95: gene |
| | reductase | abscessus | | | 96: protein |
| HMGR | HMG-CoA | Methanothermococcus | A0A4V8GZY0 | pET28b | 97: gene |
| | reductase | thermolithotrophicus | | | 98: protein |
| Scr | Succinate | Methylobacterium | A0A2U8VWW1 | pCDF- | 99: gene |
| | semialdehyde | radiodurans | | Duet-1 | 100: protein |
| | dehydrogenase | | | | |
| YihU | 3- | Escherichia coli (strain | P0A9V8 | pCA24N | 101: gene |
| | sulfolactaldehyde | K12) | | | 102: protein |
| | reductase | | | | |
| ATF1 | Alcohol O- | Saccharomyces | P40353 | | 105: gene |
| | acetyltransferase | cerevisiae | | | 106: protein |
| ATF2 | Alcohol O- | Saccharomyces | P53296 | | 107: gene |
| | acetyltransferase | cerevisiae | | | 108: protein |
| Eat1 | Ethanol | Saccharomyces | P53208 | | 109: gene |
| | acetyltransferase | cerevisiae | | | 110: protein |
| MsAcT | Acyl transferase | Mycolicibacterium | A0R5U7 | | 111: gene |
| | | smegmatis | | | 112: protein |
| EstA | Esterase | Arthrobacter | K4DIE4 | | 113: gene |
| | | nitroguajacolicus | | | 114: protein |
| EstCE | Esterase | Uncultured bacterium | Q1I192 | | 115: gene |
| | | | | | 116: protein |
| AKR7A2 | Succinate | Homo sapiens | O43488 | | 117: gene |
| | semialdehyde | | | | 118: protein |
| | dehydrogenase | | | | |
| YAT2 | Carnitine | Saccharomyces | P40017 | | 119: gene |
| | acetyltransferase | cerevisiae | | | 120: protein |
| LacA | Galactoside O acetyltransferase | Escherichia coli | P07464 | | 121: gene |
| | | | | | 122: protein |
| AKR7A3 | Succinate | Homo sapiens | O95154 | | 123: gene |
| | semialdehyde | | | | 124: protein |
| | dehydrogenase | | | | |

### Protein Production and Purification

The plasmids harboring the genes listed in Table 1 were expressed in E. coli BL21 (DE3) unless stated otherwise. Transformants bearing these genes were cultivated in LB medium at 37° C. After A600 nm reached ˜0.4-0.5, the cells were induced with 0.1 mM IPTG at 18° C. for 16-20 h. The cell pellet was dissolved (10 ml buffer/g pellet) in 150 mM Tris buffer pH 7.5 containing 0.2 M NaCl. After disrupting the cells by sonication, the cells were centrifuged at 20,000 g at 4° C. for 30 min. The lysed supernatant was then loaded onto a Ni-NTA column (Macherey Nagel) connected to a FPLC machine. After washing the column with the same buffer containing 50 mM imidazole, the proteins were eluted using the same buffer with 0.25 or 0.5 M imidazole. The fraction containing the target protein from Ni-NTA column was loaded on a HiLoad 16/600 Superdex 200 pg size exclusion column (GE Healthcare). The target proteins were eluted using 1.5 column volumes of 50 mM Hepes (pH 7.5) containing 150 mM NaCl and concentrated using Amicon ultra 15 ml centrifugal filters (Merck Millipore). All the purified proteins were stored at −80° C. until further analysis. The subunits of ligases (SucCD and Tfu_2576/77) were purified separately and mixed in equimolar amounts before storage.

### Chemical Synthesis of Itaconyl-CoA

Itaconyl-CoA was synthesized using the symmetric anhydride method (Peter et al. Molecules 2016). 100 mg CoA (0.125 mmol) in 2.5 ml 0.5 M NaHCO₃ is mixed with 25 mg itaconic anhydride (0.2 mmol=1.6 eq.) dissolved in 500 μl DMSO or THF. The mixture is stirred on an ice bath for 45 min to 1 h. The presence of free sulfhydryl group was monitored using DTNB by measuring the absorbance at 412 nm. The synthesized itaconyl-CoA is purified using a HPLC (1260 Infinity, Agilent Technologies GmbH) with a Gemini 10 μm NX-C18 (110 Å, 100×21.2 mm, AXOA packed column (Phenomenex). The concentration of the itaconyl-CoA was quantified by determining the absorption at 260 nm (Δε=16.4 mM-1 cm-1).

### Activity Assay of SucCD, MtkAB and Tfu_2576/2577

The activity of the Acyl-CoA synthethases (SucCD, MtkAB and Tfu_2576/2577) was measured in 200 mM Hepes buffer (pH 8.0) containing 1 mM itaconic acid, 0.4 mM CoA, 0.5 mM ATP, 2 mM PEP, 0.4 mM NADH, 1 U PK/LDH, 10 mM MgCl2. Either 23 μg SucCD, 9 μg MtkAB or 10 μg Tfu_2576/77. The reaction was started upon addition of itaconic acid and the consumption of NADH was monitored at 30° C. and 340 nm (Δε=6.22 M-1 cm-1) with a Cary 60 UV-Vis spectrophotometer.

### Activity Assay of Ict

The activity of Ict was measured in 200 mM Hepes buffer (pH 8.0) containing 1 mM itaconic acid, 0.5 mM acetyl-CoA or succinyl-CoA, 10 mM MgCl2 and 5 μg Ict. The reaction was started upon addition of itaconic acid and the production of itaconyl-CoA NADH was monitored at 30° C. with LC-MS.

### Activity Assay of Scr

The activity of Scr was measured in 200 mM Hepes buffer (pH 8.0) containing 1 mM itaconyl-CoA, 0.4 mM NADPH, 10 mM MgCl2 and 9 μg Scr. The reaction was started upon addition of itaconyl-CoA and the consumption of NADPH was monitored at 30° C. and 340 nm (Δε=6.22 M-1 cm-1) with a Cary 60 UV-Vis spectrophotometer.

### In Vitro Reconstitution of the Tulipalin a Production

The continuous assay to produce tulipalin A was performed in 300 μl of 200 mM Hepes buffer pH 8.0 containing 1 mM itaconic acid, 5 mM ATP, 2 mM PEP, 1 U PK/LDH, 5 mM CoA, 5 mM NADPH, 20 mM formate, 10 mM MgCl2, 125 μg SucCD (Escherichia coli), 75 μg MtkAB or 68 μg Ict (Pseudomonas aeruginosa), 150 μg Scr, 20 μg formate dehydrogenase and 70 μg YqhD or 70 μg YihU. As an alternative to YqhD, 44 μg FucO was also tested supplemented with 5 mM NADH. To the assay mixture, either 60 μg Drp35, 43 μg DEBS_TE, 20 μg LtmG_TE or 12 μg RevD_TE was added. For samples containing Drp35, additionally 10 mM CaCl2 was added. The cofactors NADH, NADPH and ATP are constantly recycled during the assay. The assay was performed at 30° C. with shaking at 400 rpm. At specified intervals 2 h, 4 h, 24 h and 48 h, 50 μl sample was withdrawn and treated with 10% formic acid to stop the reaction. The mix was spun down at 20,000 g at 4° C. for 10 min to precipitate proteins. The supernatant was directly analysed by high resolution mass spectrometry for CoAs, itaconic acid and tulipalin A. All the reactions were set up in duplicates.

### UPLC-High Resolution MS of Itaconyl-CoA

Itaconyl-CoA was analysed using an Agilent 6550 iFunnel Q-TOF LC-MS system equipped with an electrospray ionization source set to positive ionisation mode. RP-18 column (50 mm×2.1 mm, particle size 1.7 μm, Kinetex XB-C18, Phenomenex) was used using a mobile phase system comprised of 50 mM ammonium formate pH 8.1 and methanol. Chromatographic separation was carried out using the following gradient condition at a flow rate of 250 μl/min: 0 min 0% methanol; 1 min 0% methanol, 3 min 2.5% methanol; 9 min 23% methanol; 14 min 80% methanol; 16 min 80% methanol. Capillary voltage was set at 3.5 kV and nitrogen gas was used for nebulizing (20 psig), drying (13 l min-1, 225° C.) and sheath gas (12 l min-1, 400v° C.). The TOF was calibrated using an ESI-L Low Concentration Tuning Mix (Agilent) before measurement (residuals less than 2 ppm for five reference ions). MS data were acquired with a scan range of 200-1200 m/z and analyzed using MassHunter Qualitative Analysis software (Agilent) and eMZed.

### LC-MS Analysis of Itaconic Acid and Tulipalin A

Quantitative determination of itaconic acid and tulipalin was performed using a LC-MS/MS. The chromatographic separation was performed on an Agilent Infinity II 1290 HPLC system using a Kinetex EVO C18 column (150×1.7 mm, 1.7 μm particle size, 100 Å pore size, Phenomenex) connected to a guard column of similar specificity (20×2.1 mm, sub 2 μm particle size, Phenomenex) at a constant flow rate of 0.15 ml/min with mobile phase A being 0.1% formic acid in water and phase B being 0.1% formic acid in methanol (Honeywell, Morristown, New Jersey, USA) at 40° C.

The injection volume was 1 μl. The mobile phase profile consisted of the following steps and linear gradients: 0-7 min 5 to 100% B; 7-9 min constant at 100% B; 9-9.1 min from 100 to 5% B; 9.1-15 min constant at 5% B. An Agilent 6495 ion funnel mass spectrometer was used in positive and negative mode with an electrospray ionization source and the following conditions: ESI spray voltage 2000 V, nozzle voltage 500 V, sheath gas 400° C. at 11l/min, nebulizer pressure 50 psig and drying gas 80° C. at 16l/min. Compounds were identified based on their mass transition and retention time compared to standards. Chromatograms were integrated using MassHunter software (Agilent, Santa Clara, CA, USA). Absolute concentrations were calculated based on an external calibration curve prepared in sample matrix. Mass transitions, collision energies, Cell accelerator voltages and Dwell times have been optimized using chemically pure standards.

TABLE 2
Parameter settings for LC-MS

| | | | | | Collision | Cell | |
|---|---|---|---|---|---|---|---|
| | Precursor | Product | | Fragmentor | energy | Accelerator | |
| Compound | Ion | Ion | Dwell | (V) | (V) | (V) | Polarity |
| Itaconic | 129 | 129 | 70 | 380 | 0 | 5 | Negative |
| acid | | | | | | | |
| Itaconic | 129 | 185 | 70 | 380 | 6 | 5 | Negative |
| acid | | | | | | | |
| Tulipalin A | 99 | 81 | 70 | 380 | 8 | 5 | Positive |
| Tulipalin A | 99 | 53 | 70 | 380 | 14 | 5 | Positive |

### Table 2: MRM Transitions for Itaconic Acid and Tulipalin A

### Example 1: Production of Itaconyl-CoA by SucCD, MtkAB and Ict

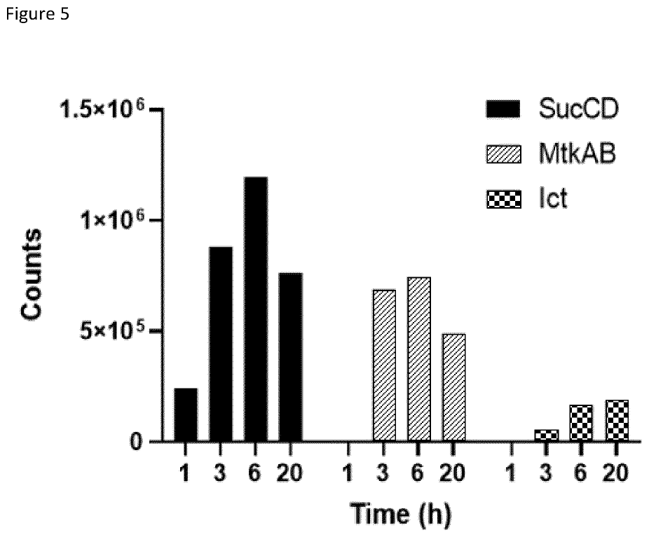

The activity assay of SucCD, MtkAB and Ict was performed as described above, using itaconic acid as a starting material. The production of Itaconyl-CoA was analysed using Agilent 6550 iFunnel Q-TOF LC-MS system as described above. FIG. 5 shows the itaconyl-CoA production over time, sampled at 1 h, 3h, 6 h and 20h. Both C1- and C4-itaconyl-CoA was detected. These results show that the three tested enzymes, SucCD, MtkAB and Ict were able to catalyze the production of Itaconyl-CoA from itaconic acid.

### Example 2: One-Pot Tulipalin a Production

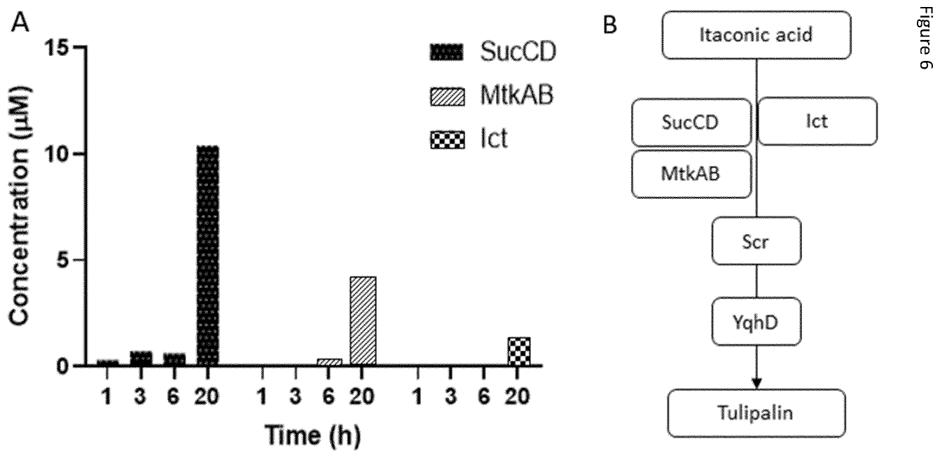

The continuous assay to produce tulipalin A was performed as described above. Either SucCD, MtkAB or Ict was used as the first enzyme to catalyze the formation of itaconyl-CoA, followed by Scr as the second enzyme and YqhD as the third enzyme (FIG. 6B). Around 2 to 10 μM tulipalin A is formed in 20 h with the combination of SucCD/MtkAB/lct+Scr+YqhD (FIG. 6A), thus proving that the lactonisation of -methylene-4-ol butyric acid to tulipalin A is spontaneous. Assays without Scr did not result in any tulipalin proving that the formation of tulipalin is possible only via this route.

### Example 3: One-Pot In Vitro Tulipalin Production Upto 48 h in the Presence of Thioesterases and Lactonase and with SucCD as the First Enzyme

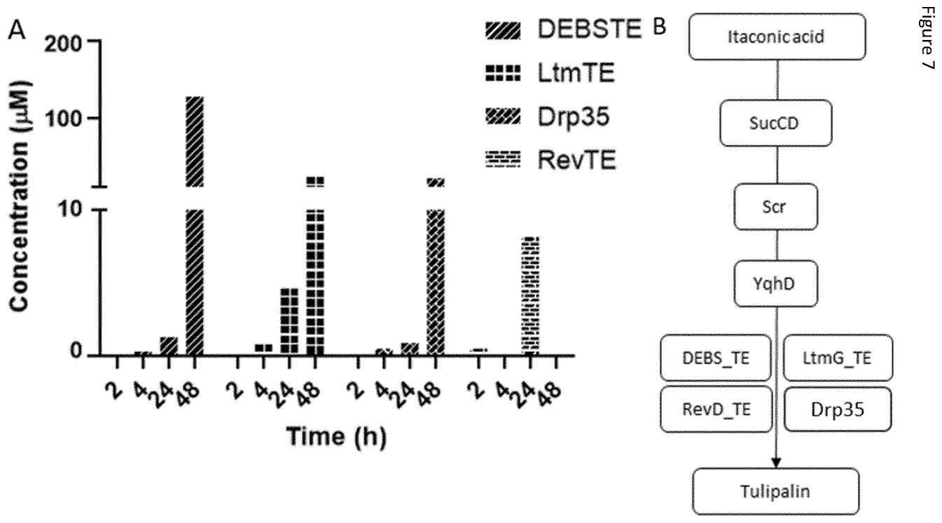

The continuous assay to produce tulipalin A was performed as described above. With SucCD as the first enzyme, we added thioesterases and a lactonase to facilitate lactonisation (FIG. 7B). The production of tulipalin increased to 128 μM in 48 h with DEBS_TE (FIG. 7A). These results show that the addition of thioesterases and a lactonase facilitate lactonisation of tulipalin A with SucCD as the first enzyme.

### Example 4: One-Pot In Vitro Tulipalin Production Upto 48 h in the Presence of Thioesterases and Lactonase and with Ict as the First Enzyme

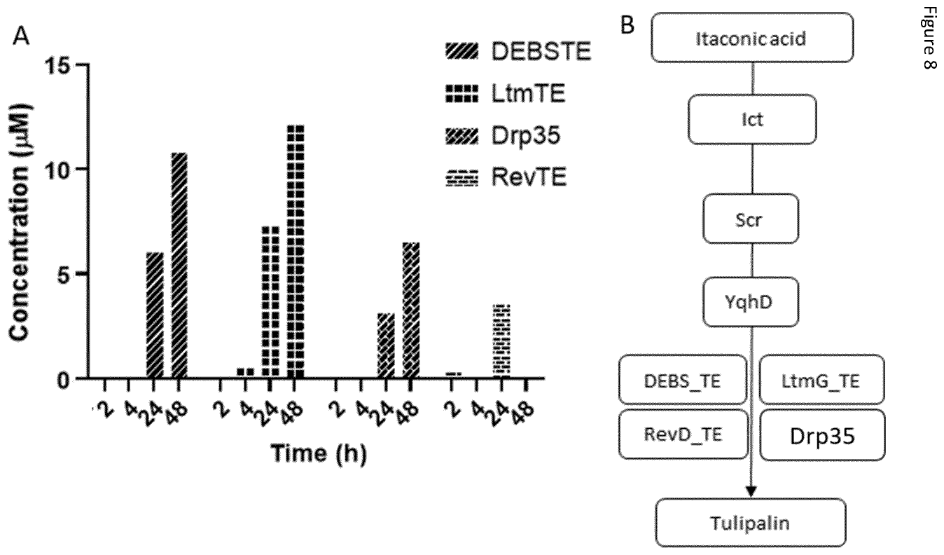

The continuous assay to produce tulipalin A was performed as described above. With Ict as the first enzyme, we added thioesterases and a lactonase to facilitate lactonisation (FIG. 8B). The production of tulipalin increased to 10 or 12 μM in 48 h with DEBS_TE or LtmTE (FIG. 8A). These results show that the addition of thioesterases and a lactonase facilitate lactonisation of tulipalin A with Ict as the first enzyme.

### Example 5: Expression of Succinyl-CoA Synthetase in E. Coli

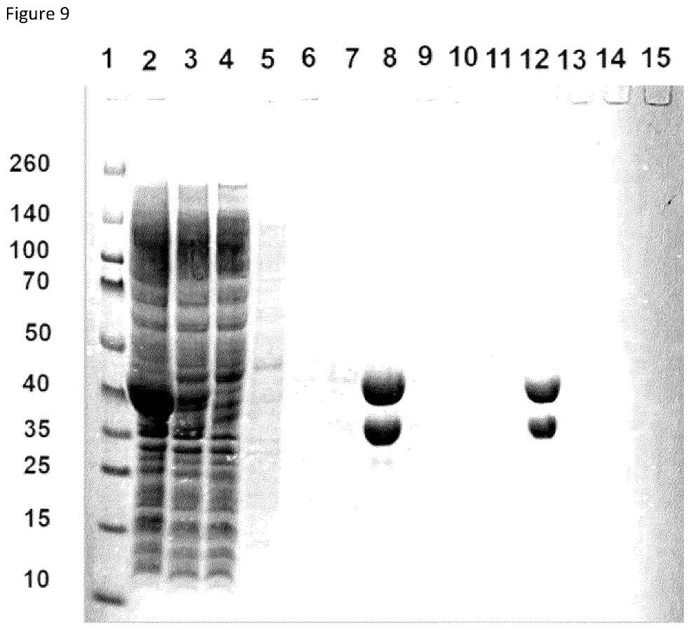

Succinyl-CoA synthetase subunits SucC and SucD were expressed simultaneously from pETDuet1 vector using NcoI and HindIII of the first multiple cloning site for SucD and NdeI and XhoI of the second multiple cloning site for SucC. Proteins from three different organisms, E. coli, Alcanivorax borkumensis SK2 and Advenella mimigardefordensis DPN7T were expressed in E. coli BL21 cells. Additionally, a version of each gene with C-terminal His Tag linked via a GS linker using a BamHI restriction site for cloning was cloned. After purification, SDS-PAGE showed two bands corresponding to the SucC and SucD subunits of E. coli SucCD at 41 and 30 kDa, demonstrating that all Succinyl-CoA synthetase subunits were successfully expressed in this system (FIG. 9).

### Example 6: Expression and Activity of Itaconate CoA-Transferase from Yersinia pestis (Yplct) and Pseudomonas aeruginosa (Palct) in E. coli

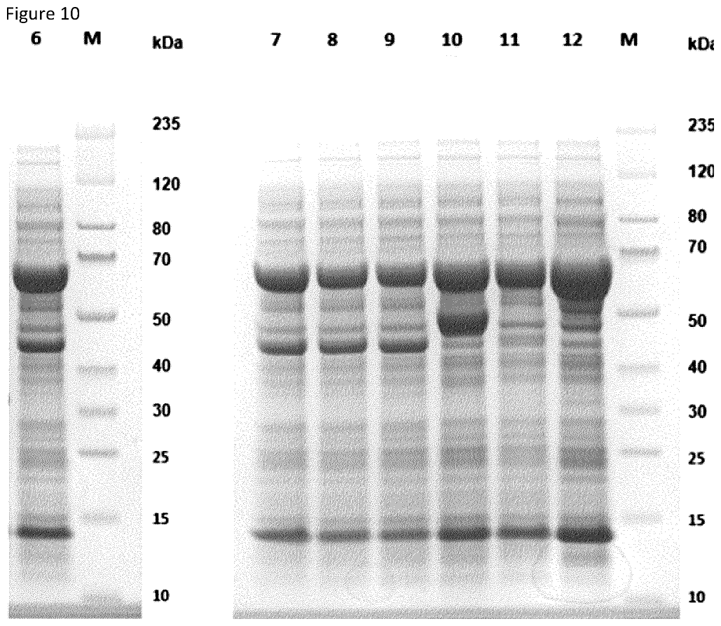

Itaconate CoA-transferase from Yersinia pestis (Yplct) and Pseudomonas aeruginosa (Palct) were synthesized in a pDHE vector and expressed in E. coli. Expression of Yplct was confirmed by SDS-PAGE (FIG. 10).

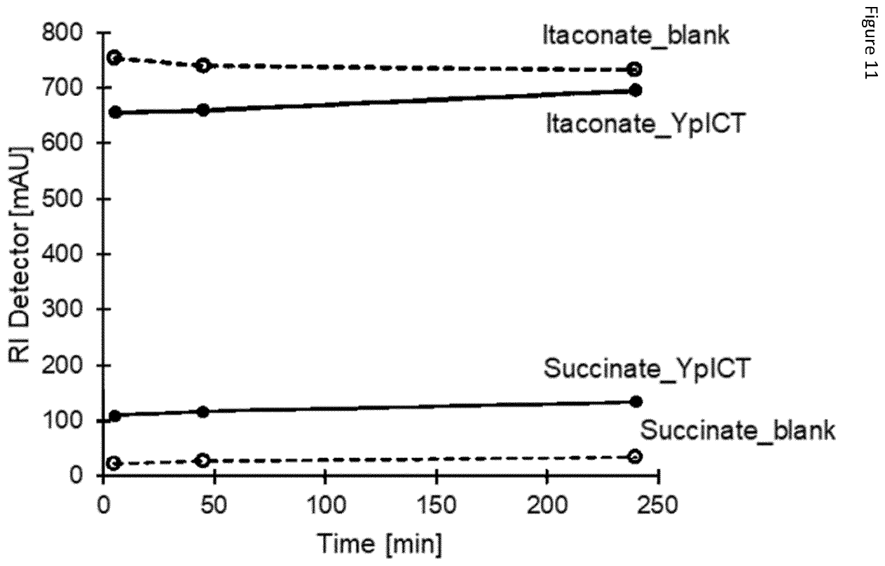

To test the activity of Yplct, an activity assay was performed. In a total volume of 200 μl, a reaction was set up containing 100 mmol/l MOPS-KOH pH 7.0, 109 μl demineralized water, 5 mmol/l MgCl₂, 10 mmol/l itaconic acid, 1-4 mmol/l Succinyl-CoA sodium salt, 5 mmol/l dithiothreitol and 10 μl of enzyme lysate diluted 1:10 in water. Blanks contained water instead of lysate. The reaction mixture was incubated at 25° C. at 1000 rpm for 4 h and samples were taken at 5 min, 45 min and 240 min. The reaction was stopped on ice and diluted 1:1 with demineralized water before measurements were taken. Succinyl-CoA, Itaconyl-CoA, CoA, Succinate and Itaconate were measured by HPLC. Succinyl-CoA concentration increased over time, and with addition of Yplct, succinate concentrations are increased while itaconate concentrations are decreased (FIG. 11). These results show that Yplct can be expressed in the selected system and is active.

### Example 7: Expression of Malonyl-CoA Reductase in E. Coli

Wildtype and three mutants of Malonyl-CoA reductase (Mcr), L152V, L152A and L152T mutants, were cloned into pDHE and expressed in E. coli. Expression was confirmed by SDS-PAGE (FIG. 10). This shows that Mcr can be expressed in the selected system.

### Example 8: Expression and Activity of Carboxylic Acid Reductase in E. Coli

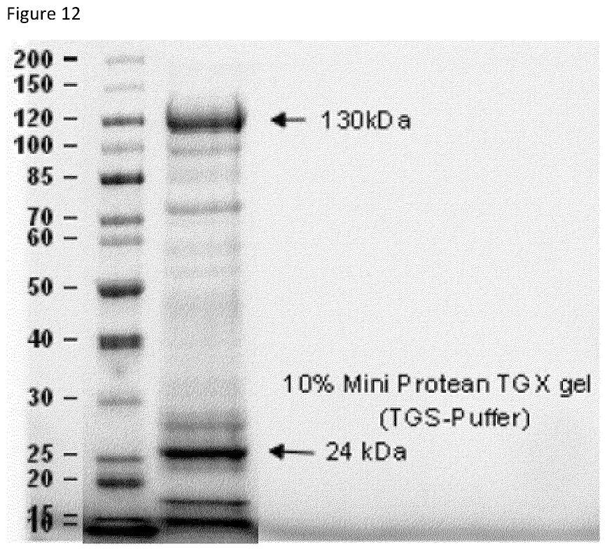

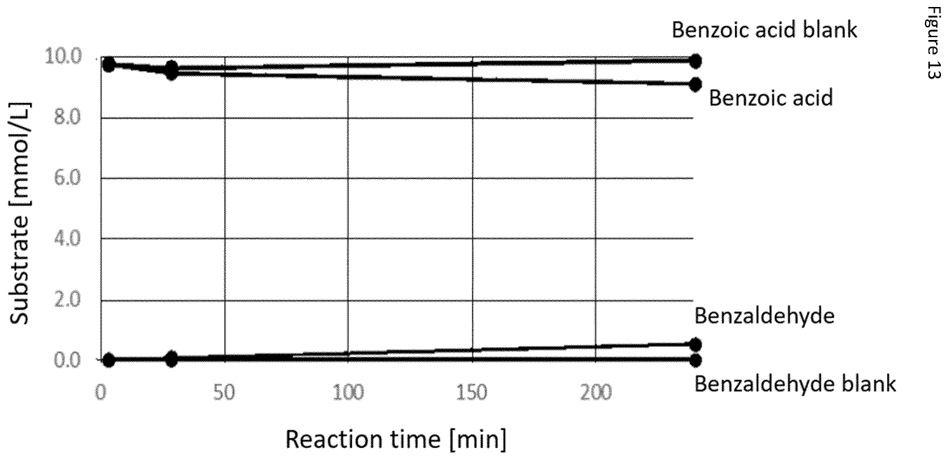

The Carboxylic acid reductase NiCar from Nocardia iowensis was co-expressed with phosphopantetheinyl transferase from E. coli (spf gene) from a pET-Duet vector and purified using Ni-NTA purification using a PD10 column with 50 mM Tris HCl pH 7.5, 1 mM EDTA, 1 mM DTT and 10% Glycerol (FIG. 12). The activity assay was performed with benzoic acid to confirm NiCar activity. In a total volume of 200 μl, a reaction was set up containing 100 mmol/l MOPS-KOH pH 7.0, 64.72 μl demineralized water, 5 mmol/l MgCl₂, 10 mmol/l benzoic acid, 10 mmol/l NADPH, 10 mmol/l ATP, 5 mmol/l dithiothreitol and 14.3 μl of purified enzyme diluted 1:10 in water. Blanks contained water instead of enzyme. The reaction mixture was incubated at 25° C. at 1000 rpm for 4 h and samples were taken at 5 min, 45 min and 240 min. The reaction was stopped on ice and diluted 1:1 with acetonitrile to precipitate protein, followed by centrifugation and dilution in 50 μl demineralized water before measurements were taken. Benzoic acid and benzaldehyde were measured by HPLC. Benzaldehyde formation over time was observed in the sample comprising NiCar, but not in the blank (FIG. 13). These results show that NiCar can be expressed in the selected system and is active.

### Example 9: One-Pot In Vitro Tulipalin Production Upto 48 h in the Presence of YihU as the Third Enzyme and in the Absence of any Fourth Enzyme

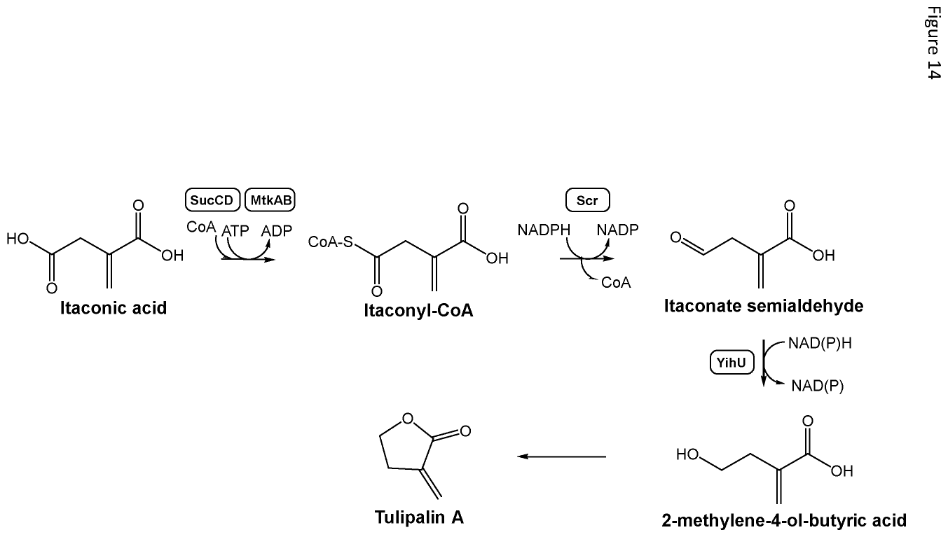

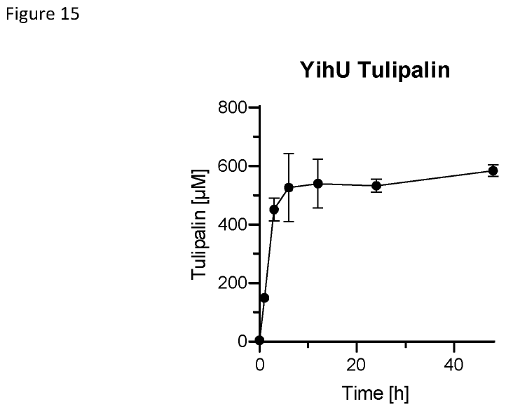

The continuous assay to produce tulipalin A was performed as described above. With SucCD as the first enzyme and Scr as the second enzyme, we added YihU as the third enzyme (FIG. 14) and monitored tulipalin A formation in the absence of any lactonases or thioesterases The production of tulipalin increased to 520 μM in just 8 h (FIG. 15). These results show that even in the absence of any enzyme to promote lactonization, YihU forms the 2-methylene-4-ol-butyric acid faster which can then spontaneously lactonise to tulipalin A.

1. A method for producing tulipalin A (α-methylene-γ-butyrolactone) from itaconic acid, the method comprising contacting a reaction mixture comprising itaconic acid with a first enzyme selected from the group consisting of at least one Acyl-CoA synthetase, at least one CoA-transferase and at least one Carboxylic acid reductase.

2. The method according to claim 1, wherein the first enzyme is at least one Acyl-CoA synthetase or at least one CoA-transferase, wherein the method further comprises contacting the reaction mixture with a second enzyme, wherein the second enzyme is at least one Oxidoreductase.

3. The method according to claim 1, wherein the method further comprises contacting the reaction mixture with a third enzyme, wherein the third enzyme is at least one Oxidoreductase selected from the group consisting of Alcohol dehydrogenase, Lactaldehyde reductase, 3-sulfolactaldehyde reductase, succinate semialdehyde reductase and Aldose/Aldehyde reductase.

4. The method according to claim 1, wherein the method further comprises contacting the reaction mixture with a fourth enzyme selected from the group consisting of at least one thioesterase and at least one lactonase.

5. The method according to claim 1, wherein the method further comprises contacting the reaction mixture with a fourth enzyme selected from the group consisting of at least one acyltransferase, at least one carboxyesterase, at least one carnitine acetyltransferase, at least one galactoside O-acetyltransferase and at least one alcohol acetyl transferase.

6. A recombinant cell or organism capable of producing tulipalin A (α-methylene-γ-butyrolactone), comprising one or more nucleic acid molecules encoding a first enzyme, wherein the first enzyme is selected from the group consisting of at least one Acyl-CoA synthetase, at least one CoA-transferase and at least one Carboxylic acid reductase.

7. The recombinant cell or organism according to claim 6, wherein the recombinant cell or organism further comprises one or more nucleic acid molecules encoding a second enzyme, wherein the second enzyme is at least one Oxidoreductase.

8. The recombinant cell or organism according to claim 6, wherein the recombinant cell or organism further comprises one or more nucleic acid molecules encoding a third enzyme, wherein the third enzyme is at least one Oxidoreductase selected from the group consisting of Alcohol dehydrogenase, Lactaldehyde reductase, 3-sulfolactaldehyde reductase, succinate semialdehyde reductase and Aldose/Aldehyde reductase.

9. The recombinant cell or organism according claim 6, wherein the recombinant cell or organism further comprises one or more nucleic acid molecules encoding a fourth enzyme, wherein the fourth enzyme is selected from the group consisting of at least one acyltransferase, at least one carboxyesterase, at least one carnitine acetyltransferase, at least one galactoside O-acetyltransferase and at least one alcohol acetyl transferase.

10. The recombinant cell or organism according to claim 6, wherein the recombinant cell or organism further comprises one or more nucleic acid molecules encoding a fourth enzyme, wherein the fourth enzyme is selected from the group consisting of at least one thioesterase and at least one lactonase.

11. The recombinant cell or organism according to claim 6, wherein the recombinant cell or organism uses itaconic acid as a substrate for tulipalin A synthesis.

12. The recombinant cell or organism according to claim 6, wherein the recombinant cell organism is selected from the group consisting of Escherichia coli, Gluconobacter oxydans, Streptomyces coelicolor, Streptococcus thermophiles, Pseudomonas putida, Bacillus lichenformis, Bacillus subtilis, Corynebacterium glutamicum, Pseudozyma tsukubaensis, Ustilago maydis, Aspergillus niger, Aspergillus terreus, Trichoderma reesei, Pichia pastoris, Saccharomyces cerevisiae, Saccharomyces pombe and Yarrowia (candida) lipolytica.

13. The method according to claim 1 wherein the first enzyme is
(i) Acyl-CoA synthetase, wherein the Acyl-CoA synthetase is selected from the group consisting of Succinyl-CoA synthetase (SucCD), and Malate-CoA ligase (MtkAB);
(ii) CoA-transferase, wherein the CoA-transferase is Itaconate-CoA transferase (Ict); or
(iii) Carboxylic acid reductase.

14. The method according to claim 1, wherein the first enzyme is Succinyl-CoA synthetase SucCD, wherein SucCD consists of two subunits SucC and SucD, wherein the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4.

15. The method according to claim 2, wherein the second enzyme is Acyl-CoA reductase selected from Succinyl-CoA reductase (Scr) and Malonyl-CoA reductase (Mcr).

16. The method according to claim 3, wherein the third enzyme is an Alcohol dehydrogenase, wherein the Alcohol dehydrogenase comprises an amino acid sequence with at least 70% identity to an amino acid sequence according to SEQ ID NO: 42.

17. The method according to claim 3, wherein the third enzyme is a 3-sulfolactaldehyde reductase, wherein the 3-sulfolactaldehyde reductase comprises an amino acid sequence with at least 70% identity to an amino acid sequence according to SEQ ID NO: 102.

18. A recombinant cell or organism capable of producing tulipalin A (α-methylene-γ-butyrolactone), wherein the cell or organism comprises
(i) a first enzyme catalyzing the production of itaconyl-CoA from itaconic acid, wherein the first enzyme is at least one Acyl-CoA synthetase;
(ii) a second enzyme catalyzing the production of itaconate semialdehyde from itaconyl-CoA, wherein the second enzyme is at least one Acyl-CoA reductase; and
(iii) a third enzyme catalyzing the production of 2-Methylene-4-ol-butyric acid from itaconate semialdehyde, wherein the third enzyme is selected from the group consisting of Alcohol dehydrogenase, Lactaldehyde reductase, 3-sulfolactaldehyde reductase and Aldose/Aldehyde reductase;
wherein the recombinant cell or organism is selected from the group consisting of Escherichia coli wild type, Escherichia coli strain Ita23, Escherichia coli strain Ita36A and Pseudozyma tsukubaensis.

19. The recombinant cell or organism according to claim 18, further comprising a fourth enzyme catalyzing the production of 4-acetyloxy-2-methylene butanoic acid from 2-Methylene-4-ol-butyric acid, wherein the fourth enzyme is selected from the group consisting of acyltransferases, carboxyesterases, carnitine acetyltransferases, galactoside O-acetyltransferases and alcohol acetyl transferases.

20. The recombinant cell or organism according to claim 18, further comprising a fourth enzyme catalyzing the intramolecular esterification of 2-Methylene-4-ol-butyric acid, wherein the fourth enzyme is selected from the group consisting of at least one thioesterase and at least one lactonase.
